(19) United States
(12) Patent Application Publication
(10) Pub. No.: US 2023/0083623 A1
(43) Pub. Date: Mar. 16, 2023

(54) NUCLEIC ACID PROBES

(71) Applicant: Agency for Science, Technology and Research, Singapore (SG)

(72) Inventors: Kok Hao Chen, Singapre (SG); Jie Lin Jolene GOH, Singapore (SG); Shijie Nigel Chou, Singapore (SG); Wan Yi Seow, Singapore (SG); Norbert Ha, Singapore (SG); Ziqing Zhao, Singapore (SG); Christabelle Goh, Singapore (SG)

(21) Appl. No.: 17/904,348

(22) PCT Filed: Jun. 24, 2020
(86) PCT No.: PCT/SG2020/050353

§ 371 Date: Aug. 16, 2022

(30) Foreign Application Priority Data
Feb. 18, 2020 (SG) 10202001453Y

(51) Int. Cl. C12Q 1/6876; C12Q 1/6841

(52) U.S. Cl. CPC C12Q 1/6876; C12Q 1/6841

(57) ABSTRACT

The present invention relates to a pair of non-naturally occurring nucleic acid probes for detecting a polynucleotide analyte for fluorescence in situ hybridization (FISH) wherein the probes comprise a first nucleic acid probe comprising a first probe binding arm that is complementary to a first probe target region of a bridge probe and a first polynucleotide analyte binding arm that is complementary to a first analyte target region of a polynucleotide analyte and a second nucleic acid probe comprising a second probe binding arm that is complementary to a second probe target region of the bridge probe. The binding of the pair of probes to target polynucleotides permits the binding of the bridge probe to allow detection of the polynucleotide analyte. It also provides a probe system comprising said pair of nucleic acid probes and methods of detecting polynucleotide analytes in a sample.

### FIELD

The present invention relates to fluorescence in situ hybridization (FISH). In particular, the invention relates to a pair of non-naturally occurring nucleic acid probes for detecting a polynucleotide analyte for fluorescence in situ hybridization.

### BACKGROUND

As an attractive approach to spatial transcriptomics, multiplexed fluorescent in situ hybridization (FISH) allows combinatorial imaging of the transcriptome, and promises to reveal the state-to-function relationships of single cells in native tissues. A key challenge to making multiplexed FISH more broadly applicable to all tissue types is the difficulty in accurately detecting individual RNA molecules in complex tissue environments, which often suffer from low signals and tissue-dependent background. To address this limitation, much effort has been focused on signal amplification to generate brighter RNA spots. However, such approaches can only improve the signal relative to the tissue auto-fluorescence. In addition, since all probes are equally amplified, these amplification methods do not help to distinguish between real RNA spots (true positives) from non-specifically bound probes (false positives).

Off-target binding of FISH probes generates background fluorescence and spurious signals. These problems are exacerbated in multiplexed FISH because of the use of highly diverse (usually consisting of thousands of sequences) and concentrated probe solutions. One approach to solve these problems is to use customized tissue clearing approaches to remove cellular proteins and lipids, thereby reducing non-specific probe binding. However, clearing does not remove the non-specific binding of probes to non-target RNAs inside the cells and tissues. In addition, tissue clearing creates another source of technical variability from sample to sample, and it entails lengthy protocols that may require customization for each tissue type.

Accordingly, it is generally desirable to overcome or ameliorate one or more of the above mentioned difficulties.

### SUMMARY

In one aspect, there is provided a pair of non-naturally occurring nucleic acid probes for detecting a polynucleotide analyte, comprising:
i. a first nucleic acid probe comprising:
   a) a first probe binding arm that is complementary to a first probe target region of a bridge probe; and
   b) a first polynucleotide analyte binding arm that is complementary to a first analyte target region of the polynucleotide analyte, and
ii. a second nucleic acid probe comprising:
   a) a second probe binding arm that is complementary to a second probe target region of the bridge probe; wherein the first probe target region is located downstream of the second probe target region on the bridge probe, and
   b) a second polynucleotide analyte binding arm that is complementary to a second analyte target region of the polynucleotide analyte, wherein the second analyte target region is located downstream of the first analyte target region on the polynucleotide analyte,
   wherein binding of the first polynucleotide analyte binding arm to the first analyte target region and binding of the second polynucleotide analyte binding arm to the second analyte target region permit binding of the first probe binding arm to the first probe target region and binding of the second probe binding arm to the second probe target region, thereby detecting the polynucleotide analyte.

In one aspect, there is provided a probe system as defined herein.

In one embodiment, there is provided a probe system comprising:
i. a first nucleic acid probe that comprises:
   a) a first probe binding arm that is complementary to a first probe target region of a bridge probe, and
   b) a first polynucleotide analyte binding arm that is complementary to a first analyte target region of a polynucleotide analyte; and
ii. a second nucleic acid probe that comprises:
   a) a second probe binding arm that is complementary to a second probe target region of the bridge probe, wherein the first probe target region is located downstream of the second probe target region on the bridge probe, and
   b) a second polynucleotide analyte binding arm that is complementary to a second analyte target region of the polynucleotide analyte, wherein the second analyte target region is located downstream of the first analyte target region on the polynucleotide analyte;
   wherein binding of the first polynucleotide analyte binding arm to the first analyte target region and binding of the second polynucleotide analyte binding arm to the second analyte target region permit binding of the first probe binding arm to the first probe target region and binding of the second probe binding arm to the second probe target region, thereby detecting the polynucleotide analyte.

In one embodiment, the probe binding arm in the first and/or second nucleic acid probe comprises an identification portion for binding to a unique bridge probe. The identification portion may allow a pair (or multiple pairs) of nucleic acid probes to be recognized by a unique bridge probe. Multiple pairs of nucleic acid probes may comprise the same identification portion for binding to the same unique bridge probe, this may allow each pair of nucleic acid probes (or a set of nucleic acid probe pairs) to be distinguishable from one another in a library comprising a plurality of nucleic acid probe pairs.

In one aspect, there is provided a method of detecting a polynucleotide analyte in a sample, the method comprising:
(a) contacting the sample with a pair of non-naturally occurring nucleic acid probes or a probe system as defined herein; and
(b) detecting the polynucleotide analyte based on hybridization to a unique bridge probe in the presence of the polynucleotide analyte.

In one aspect, there is provided a library for detecting two or more polynucleotide analytes in a sample; the library comprising two or more pairs of non-naturally occurring nucleic acid probes or a plurality of probe systems as defined herein,
wherein each pair of nucleic acid probes is specific to each polynucleotide analyte; and
wherein each pair of nucleic acid probes is configured to hybridize to a unique bridge probe in the presence of the polynucleotide analyte.

In one aspect, there is provided a method of detecting two or more polynucleotide analytes in a sample, the method comprising:
a) contacting a sample with a library as defined herein, and
b) detecting each polynucleotide analyte based on hybridization to a unique bridge probe in the presence of the polynucleotide analyte.

The method may comprise providing a unique bridge probe that is configured to bind to a specific pair (or multiple pairs) of nucleic acid probes prior to step b). A plurality of unique bridge probes may be provided either concurrently, sequentially or combinatorically to enable detection of a plurality of polynucleotide analytes.

In one aspect, there is provided a method of detecting or visualising the expression of one or more polynucleotide analytes in a sample, the method comprising
a) contacting a sample with a library as defined herein, and
b) detecting or visualising each polynucleotide analyte based on hybridisation to a unique bridge probe.

In one aspect, there is provided a kit comprising a pair of non-naturally occurring nucleic acid probes as defined herein or a plurality of probe systems or a library as defined herein.

In one embodiment, the kit further comprises one or more bridge probes.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1: Optimization of the bridge sequence length (a) Split probes were designed to target a polymorphic repeat region (SEQ ID NO: 591) of the MUC5AC transcripts in A549 cell lines.

RNA FISH images of split bridge sequence length (x) 7-12 nucleotides (nt) in (b) unpaired and (c) paired split probes (orange and light blue sequences). Shorter (7-9 nucleotides) bridge lengths were able to suppress the binding of unpaired probes. However, using bridge lengths that were too short (7+7 nucleotides) resulted in poor binding even in paired probes. 9+9 nucleotides appeared to be the most optimal length.

FIG. 2: Optimization of split-FISH workflow. Split-FISH image (a) with, and (b) without amplification primers removed from the probes via restriction digestion. (c) Same as b, but at 10× contrast. (d) Normalized RNA brightness after hybridization of bridge probe for split-FISH (blue) versus conventional readout probe (red) for 1, 5, 10, 30, and 60 minutes. Additional round of dye labelled readout probe hybridization (10 minutes) is needed for split-FISH.

FIG. 3: Optimization of the split probe construct. (a-f) Six different constructs—circular, cruciform, double ‘C’, and double ‘Z’, conventional, and unpaired were tested (SEQ ID NOs; 344-353). The targeted RNA (SEQ ID NO: 591) and probe sequences are shown. (g-k) Example RNA FISH images of the tested constructs with DAPI nucleus (blue) staining. It was found that the circular construct (g) resulted in the best RNA signals, which achieved similar brightness to the conventional scheme (k). (1) In contrast, unpaired probe showed no signal (negative control). (m) Box plots of the brightness of single RNA molecules (n=1,000 randomly selected RNAs from 5 FOVs) for each of the probe constructs. Center line, median; box limits, upper and lower quartiles; whiskers, 1.5× interquartile range.

FIG. 4: Two channels co-localization control for the split probe construct. (a) 75 unique probes (Cy3) against non-repeat regions on MUC5AC transcripts were simultaneously hybridized with split probe constructs (Cy5)—circular, cruciform, double ‘C’, double ‘Z’, and conventional. (b-e) Sample RNA FISH images from Cy3 and Cy5 channels for the circular and double ‘Z’ constructs, with DAPI staining (blue) for cell nucleus. Double ‘Z’ Cy5 is displayed at 4× enhanced contrast compared to ‘circular’. This experiment was repeated twice with similar results. (f) Box plots of the fraction of Cy5 spots that co-localized with Cy3 spots (n=10 FOVs) for each of the probe constructs. Center line, median; box limits, upper and lower quartiles; whiskers, 1.5× interquartile range.

FIG. 5: Split-probes eliminate false positive signals associated with known off targets (a) Using conventional read-out, the false positive signals were also observed in the nucleus (blue). (b) Removal of the ‘rogue’ probe (red) eliminated the false positive signals in the nucleus. (c) Using split readouts, no false positive signals were observed in the nucleus despite the presence of the ‘rogue’ sequence. (d) Readout probes are unable to bind to the unpaired RECTIFICATION SHEET RULE 91 ‘rogue’ sequence.

FIG. 6. Split probe-based multiplexed FISH (split-FISH) in mammalian cell line and tissues. (a) Scheme of multiplexed split-FISH protocol. Encoding probes are hybridized first.

At each round of imaging, bridge probes are introduced and allowed to hybridize, followed by dye-labelled readout probes. After imaging, both bridge and readout probes are washed out in preparation for the next round. (b) Decoded transcript locations for the region in FIG. 8d from split-FISH in AML12 cells. Maximum intensity projections across all rounds of hybridization are shown with decoded transcript locations overlaid. Each dot denotes a single transcript.

Colors represent different genes. Length of the scale bar is 10 μm. Scatter plot of total counts per gene vs bulk RNA-sequencing FPKM values for AML12, with Log Pearson correlation in red. Scatter plot of counts per cell between split-FISH and conventional, for the 10 genes common to both schemes. The y=x line is shown in red. (c) Scatter plot of total counts per gene vs bulk RNA-sequencing FPKM values for brain, kidney, ovary, and liver tissues. Log Pearson correlation values in red. (d) Comparison of ‘blank’ counts per cell between conventional multiplexed FISH and split-FISH for mouse brain and liver tissues. Eight and seven ‘blank’ barcodes were tested for split-FISH (317 genes) and conventional (133 genes) schemes respectively. Centre line, median; box limits, upper and lower quartiles; whiskers, 1.5× interquartile range; all data points shown in blue.

FIG. 7: Optimized split-FISH allows repeated cycles of hybridization and wash (a) Alternating hybridization and wash of the FLNA transcripts in the same A549 cells for 20 cycles. (b) Box plots of number of spots detected per cell (n=38 cells) over the 20 hybridization and wash cycles. Center line, median; box limits, upper and lower quartiles; whiskers, 1.5× interquartile range. (c) Box plots of RNA brightness (n=1,000 randomly selected RNAs from 4 FOVs) over the 20 hybridizations. Center line, median; box limits, upper and lower quartiles; whiskers, 1.5× interquartile range.

FIG. 8. Comparison of conventional and split probe approaches to multiplexed FISH. (a) Schematic comparison of the two approaches. Cellular RNA in black, encoding probes in red, dye-labelled readout probes in orange. Bridge probes (split scheme only) are in green, which bind only when two matching encoding probes are coincident within close proximity. (b to e) Unprocessed images from a single imaging round of multiplexed FISH, from AML12 cells and mouse brain slices using conventional (b and c) and split probe (d and e) schemes. Images in b, c, d and e are scaled to the same camera intensity range (30 k, orange dashed box on histogram). Inset shows full field of view, of which the main image shows a zoomed-in region (red box). The length of the scale bars are 10 μm. Histograms show distribution of raw pixel intensity from the entire field of view. X-axis of histograms are scaled to the maximum camera sensor output of 65535. Red lines show median.

FIG. 9: Tissue auto-fluorescence was negligible compared to real RNA signals (a) Representative image from split-FISH with DAPI stain (blue). (b) Post-wash images, showing no detectable RNA spots. (c) Same image as b, but at 10× contrast, to highlight tissue auto-fluorescence and un-washed single fluorescent dye molecules.

FIG. 10. Distinct transcriptomic localization patterns in four types of un-cleared mouse tissue revealed by split-FISH. Decoded transcript locations of selected genes overlaid on stitched image from one round of imaging. The length of the scale bars are 100 μm. (a) Brain tissue showing differential localization of transcripts in neuronal processes (Map4) and regions containing cell bodies (e.g. Itpr1). (b) Zonation patterns of 5 genes (Pp1, Sptbn2, Irs1, Notch3, and Osbpl8) in a kidney section. (c) Compartmentalized localization of Plxnc1, Dsp, and Slc12a7 transcripts within ovarian follicles, localization of Myh11 transcripts surrounding follicles and Rnf213 transcripts near the outer surface of the ovary (d) Localization of genes around portal veins of the liver section.

FIG. 11: Correlations between total counts and bulk RNA-sequencing FPKM values for conventional multiplexed FISH. (a) AML-12 (b) Liver (c) Brain.

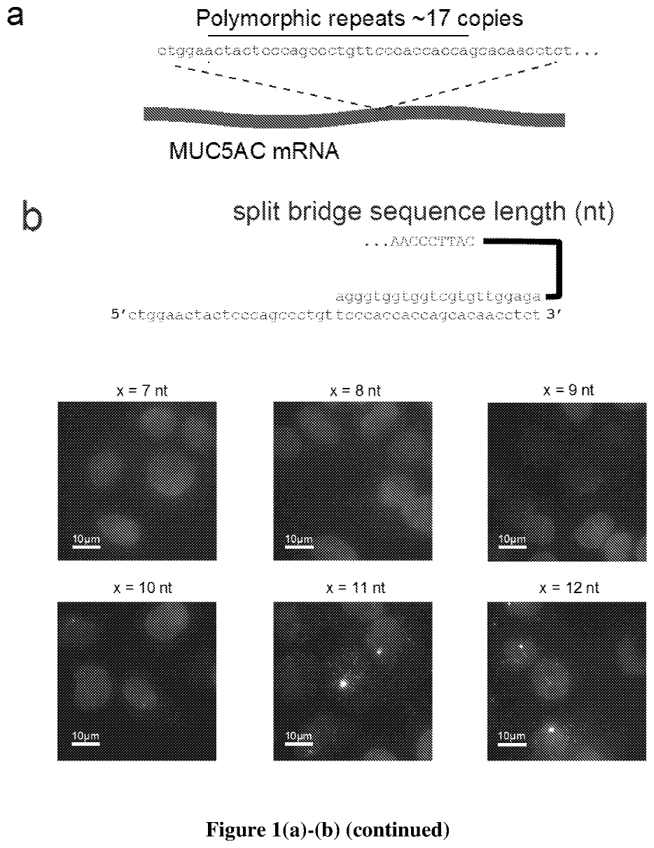

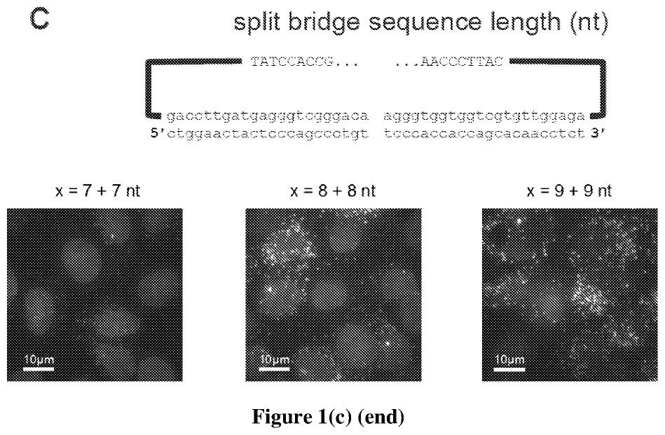

FIG. 12: Additional images from 5 bits of the AML-12 dataset shown in FIG. 1. In the bottom right images, detected genes in the same region are annotated by gene name, with different colors for each gene. (a) Conventional (b) Split-FISH.

FIG. 13: Additional images from 5 bits of the mouse brain dataset shown in FIG. 1. In the bottom right images, detected genes in the same region are annotated by gene name, with different colors for each gene. (a) Conventional (b) Split-FISH.

FIG. 14: Additional images from 5 bits of the mouse liver dataset. In the bottom right images, detected genes in the same region are annotated by gene name, with different colors for each gene. (a) Conventional (b) Split-FISH.

### DETAILED DESCRIPTION

The specification discloses a pair of non-naturally occurring nucleic acid probes for detecting a polynucleotide analyte.

Provided herein is a pair of non-naturally occurring nucleic acid probes for detecting a polynucleotide analyte, comprising
i. a first nucleic acid probe comprising:
   a) a first probe binding arm that is complementary to a first probe target region of a bridge probe; and
   b) a first polynucleotide analyte binding arm that is complementary to a first analyte target region of a polynucleotide analyte, and
ii. a second nucleic acid probe comprising:
   a) a second probe binding arm that is complementary to a second probe target region of the bridge probe, wherein the first probe target region is located downstream of the second probe target region on the bridge probe, and
   b) a second polynucleotide analyte binding arm that is complementary to a second analyte target region of the polynucleotide analyte wherein the second analyte target region is located downstream of the first analyte target region on the polynucleotide analyte,

wherein binding of the first polynucleotide analyte binding arm to the first analyte target region and binding of the second polynucleotide analyte binding arm to the second analyte target region permit binding of the first probe binding arm to the first probe target region and binding of the second probe binding arm to the second probe target region, thereby detecting the polynucleotide analyte.

In one aspect, there is provided a probe system comprising:
i. a first nucleic acid probe that comprises:
   a) a first probe binding arm that is complementary to a first probe target region of a bridge probe, and
   b) a first polynucleotide analyte binding arm that is complementary to a first analyte target region of a polynucleotide analyte; and
ii. a second nucleic acid probe that comprises:
   a) a second probe binding arm that is complementary to a second probe target region of the bridge probe, wherein the first probe target region is located downstream of the second probe target region on the bridge probe, and
   b) a second polynucleotide analyte binding arm that is complementary to a second analyte target region of the polynucleotide analyte, wherein the second analyte target region is located downstream of the first analyte target region on the polynucleotide analyte;
   wherein binding of the first polynucleotide analyte binding arm to the first analyte target region and binding of the second polynucleotide analyte binding arm to the second analyte target region permit binding of the first probe binding arm to the first probe target region and binding of the second probe binding arm to the second probe target region, thereby detecting the polynucleotide analyte.

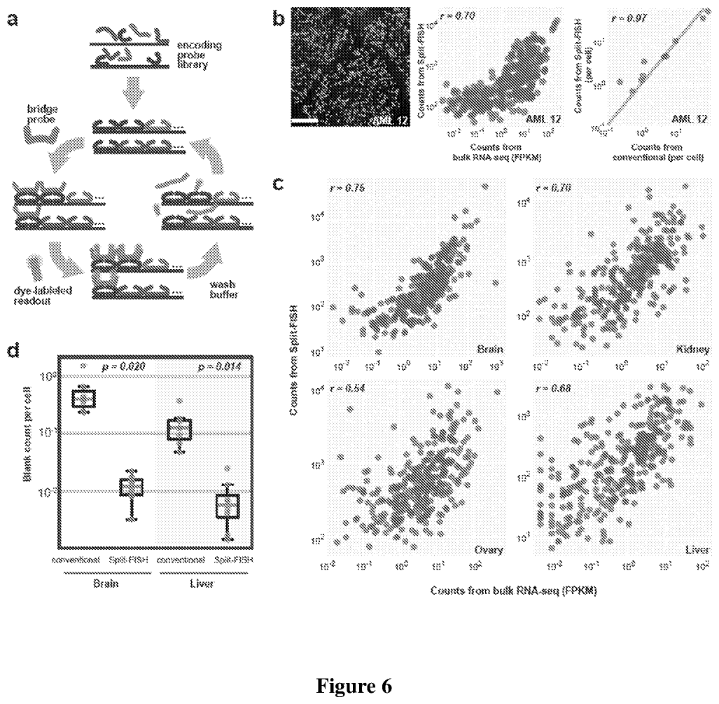

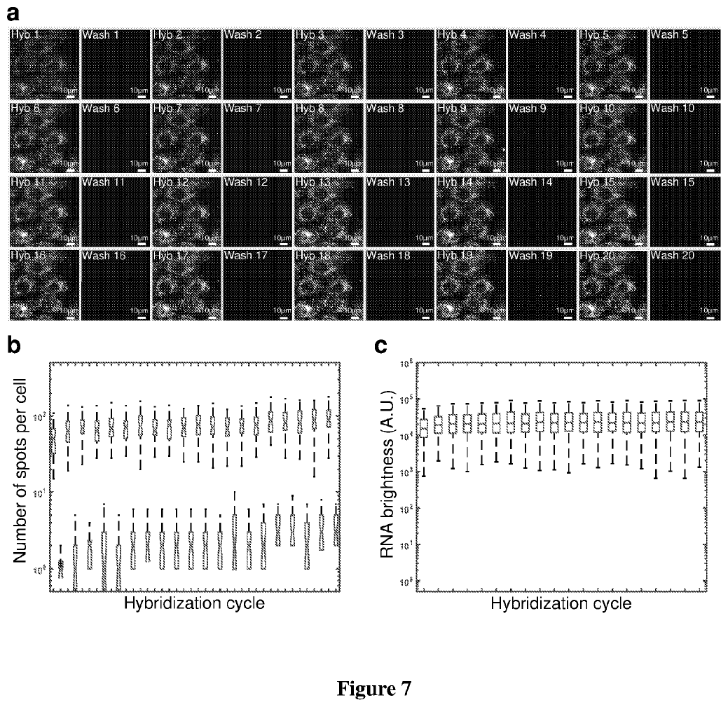

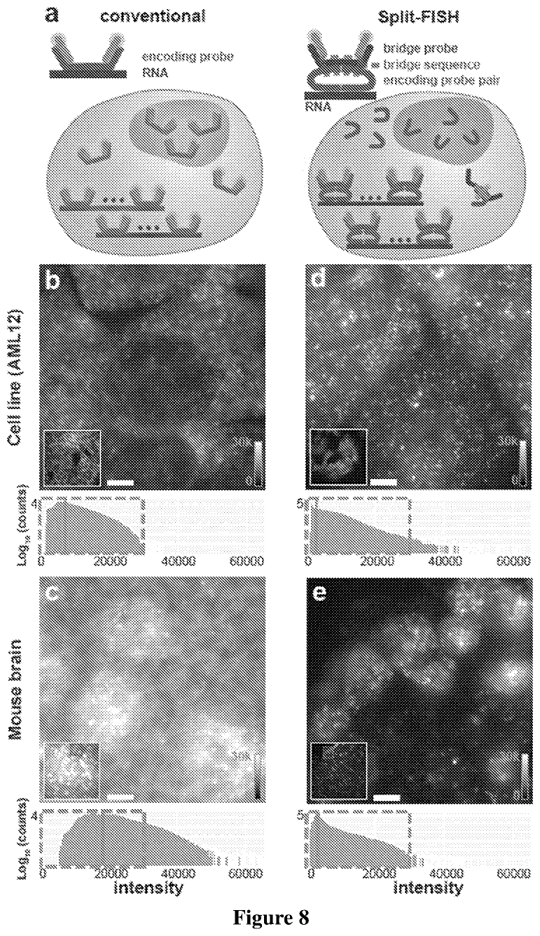

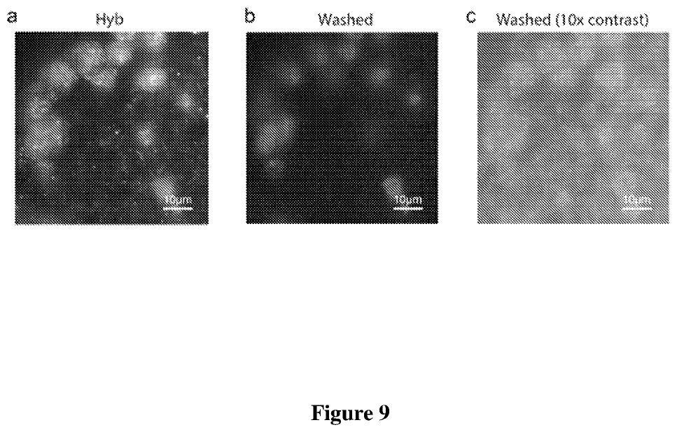

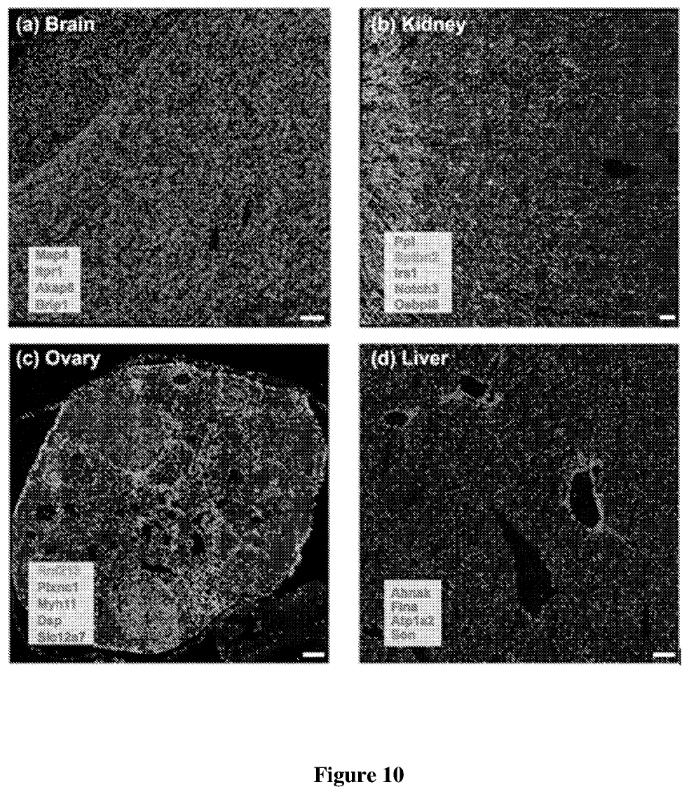

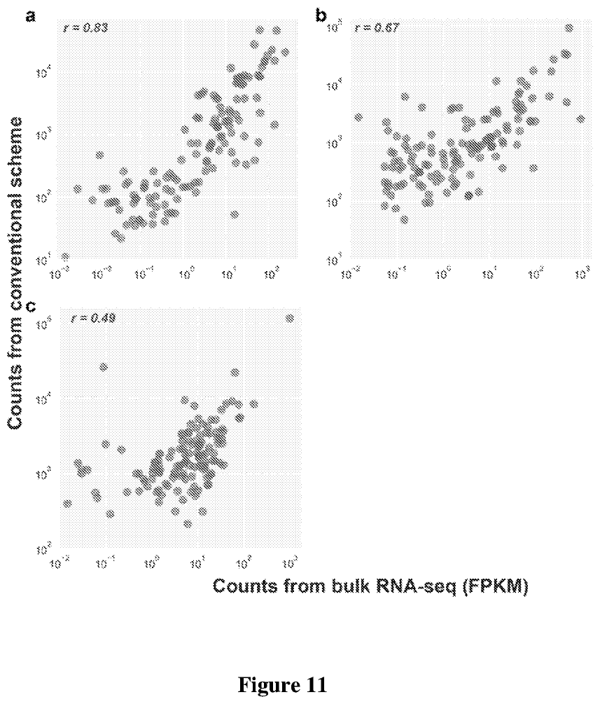

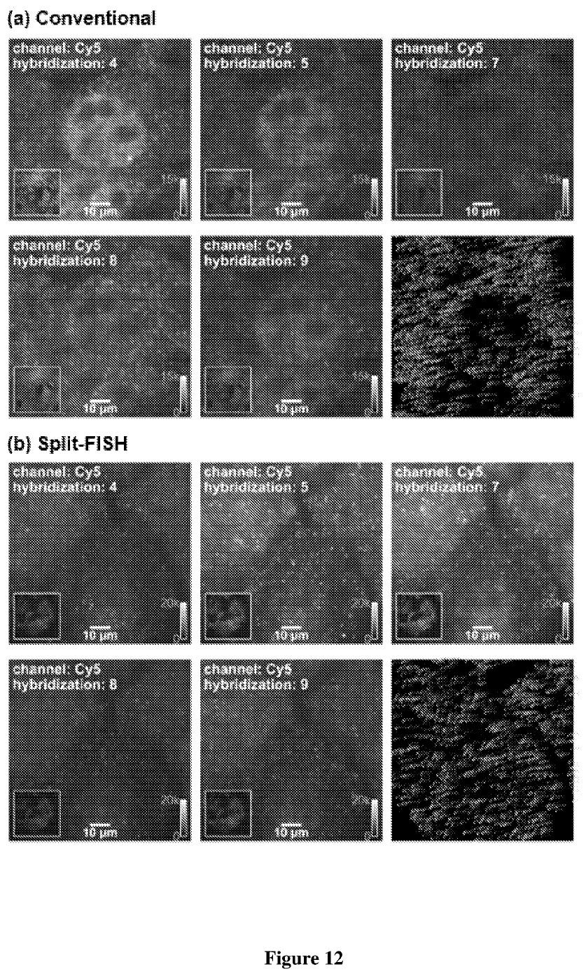

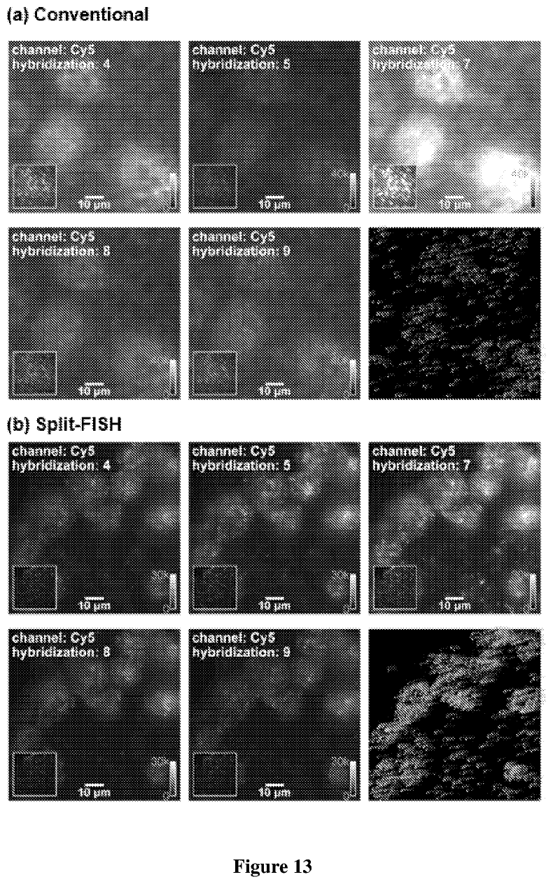

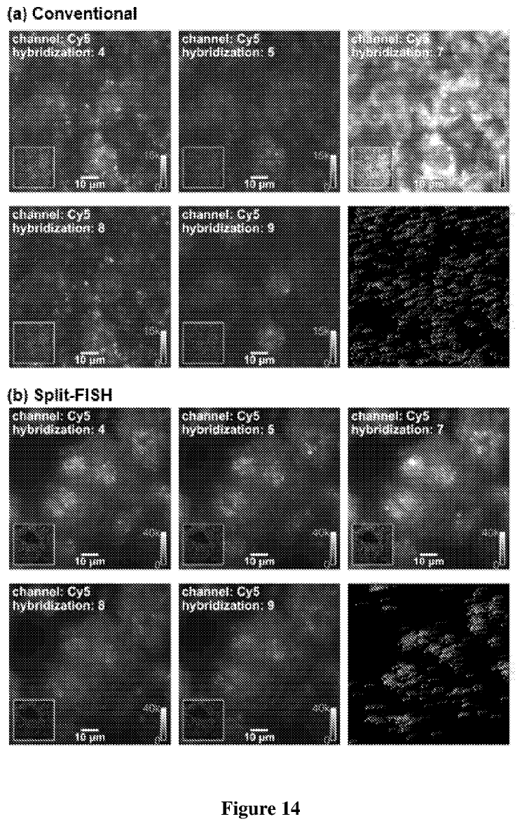

Without being bound by theory, the inventors have found a way to decrease non-specific background when detecting polynucleotide analytes in a cell or tissue (such as using Fluorescence in-situ hybridization). This can be done by using a set of split probes whereby a fluorescence signal is generated only when two independent hybridization events are co-localized (termed as split-FISH). In the split-FISH scheme (FIGS. 6a and 8a), a bridge sequence is shared between a pair of adjoining encoding probes. The bridge probe can be designed to be unable to hybridize with sufficient affinity to any single encoding probe. Only when a pair of encoding probes is hybridized at adjacent locations on the polynucleotide analyte (such as a target RNA) will there be sufficient complementary base pairing in close proximity to enable the bridge probe to bind efficiently. A fluorescently labeled readout probe may then hybridize to the bridge probes to generate on-target signals. By improving the probe design at the single-molecule level and designing custom-barcoded bridge sequences, split-FISH can be used for accurate transcriptomic profiling even in uncleared tissues.

The probe system may further comprise the bridge probe.

The pair of non-naturally occurring nucleic acid probes for detecting a polynucleotide analyte may also be referred to a pair of non-naturally occurring nucleic acid split probes.

The pair of non-naturally occurring nucleic acid probes may also be referred to as “encoding probes”.

The pair of nucleic acid probes may be a pair of single-stranded nucleic acid probes.

The “bridge probe” may hybridize to the nucleic acid probes when the first and second nucleic acid probes hybridizes with the polynucleotide analyte. The “bridge probe” may therefore detect the binding of the first and second nucleic acid probes to the polynucleotide analyte.

Each pair of nucleic acid probes may be configured to hybridize to a unique bridge probe. In one embodiment, the probe binding arm in the first and/or second nucleic acid probes comprises an identification portion for binding to a unique bridge probe. The identification portion may allow a pair (or multiple pairs) of nucleic acid probes to be recognized by a unique bridge probe. This may allow each pair of nucleic acid probes (or a set of nucleic acid probe pairs) to be distinguishable from one another in a library comprising a plurality of nucleic acid probe pairs.

Also provided herein is the use of a pair of non-naturally occurring nucleic acid probes for detecting a polynucleotide analyte. Also provided herein is a pair of non-naturally occurring nucleic acid probes when used to detect a polynucleotide analyte

In one embodiment, the probe binding arm in the first and/or second nucleic acid probes consists of 9 or 10 nucleotides. In one embodiment, the probe binding arm in the first and/or second nucleic acid probes consists of 9 nucleotides. It was found that the length of the split bridge may affect non-specific background signal and a length of about 9 nucleotides was surprisingly able to produce a level of non-specific background signal that is virtually undetectable. For example, the first nucleic acid probe may comprise a first probe binding arm at the 3′ terminus that is complementary to and selectively hybridizes to a first probe target region of a bridge probe, wherein the first probe binding arm is ATTTAACCG (SEQ ID NO: 592) (see Table 9). The second nucleic acid probe may comprise a second probe binding arm at the 5′ terminus that is complementary to and selectively hybridizes with a second probe target region of the bridge probe, wherein the second probe binding arm is CCCATTACC (SEQ ID NO: 593). The bridge probe may have a sequence of GGTAATGGGCGGTTAAAT (SEQ ID NO: 594). The bridge probe may further comprise one or two readout sequences (e.g. ATTGTAAAGCGTGAGAAA (SEQ ID NO: 595)) that allows the bridge probe to be detected or recognised by a readout probe.

In one embodiment, the polynucleotide analyte binding arm in the first or second nucleic acid probes consists of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. In one embodiment, the polynucleotide analyte binding arm in the first or second nucleic acid probes consists of 25 nucleotides.

In one embodiment, a linker is positioned between the probe binding arm and the polynucleotide analyte binding arm. The linker may be a short linker that is about 1 to 10 nucleotides. The linker may be a short linker of 1, 2, 3, 4, 5, 6, 7, 8, 9 or 10 nucleobases. In one embodiment, the linker is about 1 to 10, 1 to 9, 1 to 8; 1 to 7; 1 to 6; 1 to 5, 1 to 4, 1 to 3, 1 to 2 nucleobases. In one embodiment, the linker is about 1 to 5 nucleobases. In one embodiment, the linker is 1, 2, 3, 4 or 5 nucleobases. In one embodiment, the linker is 2 or 3 nucleobases. In one example, the linker is TAT (see Table 8a under Paired (circular) split probe sequences).

The term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides.

As used herein, the term “nucleic acid”, and equivalent terms such as “polynucleotide”, refer to a polymeric form of nucleotides of any length, such as ribonucleotides, deoxyribonucleotides or peptide nucleic acids (PNAs), that comprise purine and pyrimidine bases, or other natural, chemically or biochemically modified, non-natural, or derivatized nucleotide bases. The nucleic acid may be double stranded or single stranded. References to single stranded nucleic acids include references to the sense or antisense strands. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. The terms nucleoside, nucleotide, deoxynucleoside and deoxynucleotide generally include complements, fragments and variants of the nucleoside, nucleotide, deoxynucleoside and deoxynucleotide, or analogs thereof.

In one embodiment, the first analyte target region is immediately adjacent to the second analyte target region. In another embodiment, the first analyte target region is spaced from the second analyte target region by no more than 1, 2, 3, 4, 5, 6, 7, 8, 9, 10, 11, 12, 13, 14, 15, 16, 17, 18, 19 or 20 nucleobases.

In one embodiment, the first probe target region is immediately adjacent to the second probe target region. In another embodiment, the first probe target region is spaced from the second probe target region by no more than 20, 19, 18, 17, 16, 15, 14, 13, 12, 11, 10, 9, 8, 7, 6, 5, 4, 3, 2 or 1 nucleobases.

An “oligonucleotide” as used herein is a single stranded molecule which may be used in hybridization or amplification technologies. In general, an oligonucleotide may be any integer from about 15 to about 100 nucleotides in length, but may also be of greater length.

The term “probe” refers to any molecule which is capable of selectively binding to a specifically intended target molecule, for example, a nucleotide transcript. Probes can be either synthesized by one skilled in the art, or derived from appropriate biological preparations.

The nucleic acid probes (or nucleic acid split probes) of the present invention may be useful for detecting the presence or absence of one or more polynucleotide analytes in one or more samples known to contain or suspected of containing the polynucleotide analytes. The nucleic acid probes can also be used to quantify the amount of polynucleotide analytes within the sample. The nucleic acid probes are useful for detecting unamplified polynucleotide target in a sample such as for example RNA, MRNA, rRNA, plasmid DNA, viral DNA, bacterial DNA, and chromosomal DNA. Additionally, the nucleic acid probes may be useful in conjunction with the amplification of a polynucleotide target by well-known methods such as PCR, ligase chain reaction, Q-B replicase, strand-displacement amplification (SDA), rolling-circle amplification (RCA), nucleic acid sequence-based amplification (NASBA), and the like.

In one embodiment, the bridge probe is coupled or conjugated to a label (such as a fluorescent label). Such a bridge probe may be referred to as a readout probe. In one embodiment, the bridge probe is detected via hybridization to a secondary detection probe (or readout probe) that is conjugated to a label (such as a fluorescent label). The bridge probe may comprise a specific (or unique) tag or barcode sequences that enable it to be recognised via hybridisation to a secondary detection probe (or readout probe).

Examples of fluorescent labels include, but are not limited to, rare earth chelates (europium chelates), Texas Red, rhodamine, fluorescein, dansyl, phycocrytherin, phycocyanin, spectrum orange, spectrum green, and/or derivatives of any one or more of the above. Multiple probes used in the assay may be labeled with more than one distinguishable fluorescent or pigment color. These color differences provide a means to identify, for example, the hybridization positions of specific probes. Moreover, probes that are not separated spatially can be identified by a different color light or pigment resulting from mixing two other colors (e.g., light red+green=yellow) pigment (e.g., blue+yellow=green) or by using a filter set that passes only one color at a time. Probes can be labeled directly or indirectly with the fluorophore, utilizing conventional methodology. Additional probes and colors may be added to refine and extend this general procedure to include more genetic abnormalities or serve as internal controls.

In one embodiment, the secondary detection probe (or readout probe) hybridizes to a terminal region of the bridge probe.

In one embodiment, two secondary detection probes hybridize to both terminal regions of the bridge probe.

In one embodiment, the secondary detection probe or probes (or readout probes) hybridize to a central region of the bridge probe.

In one embodiment, the bridge probe has the same sequence as the polynucleotide analyte.

In one embodiment, the readout probe has the same sequence as the polynucleotide analyte.

In one embodiment, there is provided a pair of non-naturally occurring nucleic acid probes for detecting a polynucleotide analyte, the pair of nucleic acid probes comprising two anti-parallel nucleic acid strands, wherein:
i. a first nucleic acid strand comprises:
   a) a readout binding arm at the 3′ terminus that is complementary to and selectively hybridizes to a first region of a readout probe; and
   b) a polynucleotide analyte binding arm at the 5′ terminus that is complementary to and selectively hybridizes with a first region of the polynucleotide analyte, and
ii. a second nucleic acid strand comprises:
   a) a readout binding arm at the 5′ terminus that is complementary to and selectively hybridizes with a second region of a readout probe; and
   b) a polynucleotide analyte binding arm at the 3′ terminus that is complementary to and selectively hybridizes with a second region of the polynucleotide analyte positioned at the 3′ end of the first region;

wherein hybridization of the first and second nucleic acid strands with the polynucleotide analyte enables hybridization to the readout probe and detection of the polynucleotide analyte.

The term “complementary” refers to the base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100% of the nucleotides of the other strand. Alternatively, complementarity exists when an RNA or DNA strand will hybridize under selective hybridization conditions to its complement. Typically, selective hybridization will occur when there is at least about 65% complementarity over a stretch of at least 14 to 25 nucleotides, preferably at least about 75%, and more preferably at least about 90% complementarity.

As used herein, the term “hybridization” or “hybridizes” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid”. The proportion of the population of polynucleotides that forms stable hybrids is referred to herein as the “degree of hybridization.”

Hybridization conditions will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target. Stringent conditions are sequence-dependent and are different under different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point (Tₘ) for the specific sequence at a defined ionic strength and pH. The Tₘ is the temperature (under defined ionic strength, pH and nucleic acid composition) at which 50% of the probes complementary to the target sequence hybridize to the target sequence at equilibrium. Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations.

A “label” refers to a reporter molecule or enzyme that is capable of generating a measurable signal and is covalently or non-covalently joined to a polynucleotide.

The term “labelled”, with regard to, for example, a probe, is intended to encompass direct labelling of the probe by coupling (i.e., physically linking) a detectable substance to the probe, as well as indirect labelling of the probe by reactivity with another reagent that is directly labelled. Examples of indirect labelling include detection of a bridge probe (bound to a nucleic acid pair in the presence of a polynucleotide analyte) using a fluorescently labelled secondary probe (or readout probe).

The term “polynucleotide analyte” may be any polynucleotide that may be detected or analyzed by a pair of nucleic acid probes or probe system as defined herein. The analyte may be naturally-occurring or synthetic. A polynucleotide analyte may be present in a sample obtained using any methods known in the art. In some cases, a sample may be processed before analyzing it for a polynucleotide analyte. The polynucleotide may include DNA, RNA, peptide nucleic acids, and any hybrid thereof, where the polynucleotide contains any combination of deoxyribo- and/or ribo-nucleotides. Polynucleotides may be single stranded or double stranded, or contain portions of both double stranded or single stranded sequence. Polynucleotides may contain any combination of nucleotides or bases, including, for example, uracil, adenine, thymine, cytosine, guanine, inosine, xanthine, hypoxanthine, isocytosine, isoguanine and any nucleotide derivative thereof. As used herein, the term “nucleotide” may include nucleotides and nucleosides, as well as nucleoside and nucleotide analogs, and modified nucleotides, including both synthetic and naturally occurring species. Polynucleotides may be any suitable polynucleotide, including but not limited to cDNA, mitochondrial DNA (mtDNA), messenger RNA (mRNA), ribosomal RNA (rRNA), transfer RNA (tRNA), nuclear RNA (nRNA), small interfering RNA (siRNA), small nuclear RNA (snRNA), small nucleolar RNA (snoRNA), small Cajal body-specific RNA (scaRNA), microRNA (miRNA), double stranded (dsRNA), ribozyme, riboswitch or viral RNA. Polynucleotides may be contained within any suitable vector, such as a plasmid, cosmid, fragment, chromosome, or genome. The polynucleotide analyte can be a nucleic acid endogenous to the cell. As another example, the polynucleotide analyte can be a nucleic acid introduced to or expressed in the cell by infection of the cell with a pathogen, for example, a viral or bacterial genomic RNA or DNA, a plasmid, a viral or bacterial mRNA, or the like.

Genomic DNA may be obtained from naturally occurring or genetically modified organisms or from artificially or synthetically created genomes. Polynucleotide analytes comprising genomic DNA may be obtained from any source and using any methods known in the art. For example, genomic DNA may be isolated with or without amplification. Amplification may include PCR amplification, rolling circle amplification and other amplification methods. Genomic DNA may also be obtained by cloning or recombinant methods, such as those involving plasmids and artificial chromosomes or other conventional methods (see Sambrook and Russell, Molecular Cloning: A Laboratory Manual., cited supra.) Polynucleotide analytes may be isolated using other methods known in the art, for example as disclosed in Genome Analysis: A Laboratory Manual Series (Vols. I-IV) or Molecular Cloning: A Laboratory Manual. If the isolated polynucleotide analyte is an mRNA, it may be reverse transcribed into cDNA using conventional techniques, as described in Sambrook and Russell, Molecular Cloning: A Laboratory Manual., cited supra.

The term “gene” is used broadly to refer to any nucleic acid associated with a biological function. Genes typically include coding sequences and/or the regulatory sequences required for expression of such coding sequences. The term gene can apply to a specific genomic sequence, as well as to a cDNA or an mRNA encoded by that genomic sequence. Genes also include non-expressed nucleic acid segments that, for example, form recognition sequences for other proteins. Non-expressed regulatory sequences include promoters and enhancers, to which regulatory proteins such as transcription factors bind, resulting in transcription of adjacent or nearby sequences.

As used herein, the term “sample” includes tissues, cells, body fluids and isolates thereof etc., isolated from a subject, as well as tissues, cells and fluids etc. present within a subject (i.e. the sample is in vivo). Examples of samples include: whole blood, blood fluids (e.g. serum and plasm), lymph and cystic fluids, sputum, stool, tears, mucus, hair, skin, ascitic fluid, cystic fluid, urine, nipple exudates, nipple aspirates, sections of tissues such as biopsy and autopsy samples, frozen sections taken for histologic purposes, archival samples, explants and primary and/or transformed cell cultures derived from patient tissues etc.

The sample (such as a tissue or cell sample) may be fixed and permeabilized before hybridization with a pair of nucleic acid probe as defined herein, to retain the polynucleotide analytes in the cell and to permit the nucleic acid probes, bridge probes, etc. to enter the sample. The sample is optionally washed to remove materials not captured to one of the polynucleotide analytes. The sample can be washed after any of the various steps, for example, after hybridization of the nucleic acid probes to the polynucleotide analytes to remove unbound nucleic acid probes or after hybridization with the nucleic acid probes and bridge probes, before removing unbound nucleic acid probe and bridge probes.

The terms “restriction enzyme” and “restriction endonuclease” as used herein means an endonuclease enzyme that recognises and cleaves a specific sequence of DNA (recognition sequence).

In one aspect, there is provided a method of detecting a polynucleotide analyte in a sample, the method comprising:
(a) contacting the sample with a pair of non-naturally occurring nucleic acid probes or a probe system as defined herein; and
(b) detecting the polynucleotide analyte based on hybridization to a bridge probe in the presence of the polynucleotide analyte.

In one embodiment, there is provided a method of determining the level of a polynucleotide analyte in a sample, the method comprising:
(a) contacting the sample with a pair of non-naturally occurring nucleic acid probes or a probe system as defined herein; and
(b) detecting the polynucleotide analyte based on hybridization to a bridge probe in the presence of the polynucleotide analyte.

The various hybridization steps can be performed simultaneously or sequentially, in essentially any convenient order. In one embodiment, a hybridization step with the multiple pairs (or library) of nucleic acid probes is accomplished for all of the polynucleotide analytes at the same time. For example, all the nucleic acid probes can be added to the sample at once and permitted to hybridize to their corresponding targets, the sample can then be washed. Corresponding bridge probes can be hybridized to the nucleic acid probes and sample can be washed again prior to detection of the bridge probes. It will be evident that double-stranded polynucleotide analyte(s) are preferably denatured, e.g., by heat, prior to hybridization of the corresponding pair(s) of nucleic acid probes to the polynucleotide analyte.

The method may comprise the step of hybridizing a bridge probe to the pair of non-naturally occurring nucleic acid probes that are bound to the polynucleotide analyte that is present. Any unbound bridge probe may be removed or washed off.

The bridge probe may be coupled or conjugated to a label (such as a fluorescent label) that enables detection of the bridge probe and thus enables detection of the polynucleotide analyte. Such a bridge probe may also be referred to as a “readout probe”.

Alternatively, a secondary detection probe (i.e. a readout probe) may be hybridized to the bridge probe and allows the bridge probe (and the polynucleotide analyte) to be detected.

The bridge probe may comprise a specific tag or barcode sequence (such as a 6 nucleotide sequence). This may enable to bridge probe to be recognised by the secondary detection probe (or readout probe).

The method may allow the detection of the presence or levels of the polynucleotide analyte based on the signal that is detected.

The method may involve detecting one or more polynucleotide analytes. The polynucleotide analytes may be detected concurrently or sequentially.

In the case where the polynucleotide analytes are detected sequentially, this may involve multiple rounds of hybridization for each polynucleotide analyte with a specific pair of nucleic acid probes, and subsequent detection with bridge and/or readout probes. There may also be a step of washing or removal of signal (by, for example, bleaching) in between detection of each polynucleotide analyte.

In one aspect, there is provided a library for detecting two or more polynucleotide analytes in a sample; the library comprising two or more pairs of non-naturally occurring nucleic acid probes or a plurality of probe systems as defined herein, wherein each pair of nucleic acid probes is specific to each polynucleotide analyte; and wherein each pair of nucleic acid probes is configured to hybridize to a unique bridge probe in the presence of the polynucleotide analyte.

The term “unique bridge probe” may refer to the ability of a bridge probe to recognise a specific pair of nucleic acid probes. Each pair of nucleic acid probes in a library may comprise an “identification portion” (or barcode) in the probe binding arm of either the first or second nucleic acid probe (or both) for binding to a unique bridge probe. In one embodiment, the identification portion consists of 6 nucleotides (e.g. actcta). The bridge probe may have a corresponding barcode sequence that recognises the identification portion in the pair of nucleic acid probes.

More than one pair of nucleic acid probes (e.g. a set of nucleic acid probes) may comprise the same identification portion (or barcode) that allows them to bind to a unique bridge probe. A library of nucleic acid probe pairs may be grouped according to nucleic acid probe pairs that share the same identification portion (or barcode). This may allow for the combinatorial detection of polynucleotide analytes based on addition of a corresponding unique bridge probe that recognises nucleic acid probe pairs that share the same identification portion.

A library of identification portions (or barcodes) may be used in certain embodiments, e.g., containing at least 10, at least 10², at least 10³, at least 10⁴, at least 10⁵, at least 10⁶, at least 10⁷, at least 10⁸, etc. unique sequences. The unique sequences may be all individually determined (e.g., randomly), although in some cases, the identification portion may be defined as a plurality of variable portions (or “bits”). e.g., in sequence. For example, an identification portion may include at least 2, at least 3, at least 5, at least 6, at least 7, at least 10, at least 15, at least 20, at least 25, at least 30, at least 40, or at least 50 variable portions. Each of the variable portions may include at least 2, at least 3, at least 4, at least 5, at least 7, at least 10, at least 15, at least 20, at least 25, at least 30, at least 40, at least 50, or more possibilities. In one embodiment, the identification portion consists of 6 variable portions.

Thus, for example, an identification portion defined with 22 variable regions and 2 unique possibilities per variable region would define a library of identification portions with 2=4,194,304 members. As another non-limiting example, an identification portion may be defined with 10 variable regions and 7 unique possibilities per variable region to define a library of identification portions with 710 members. It should be understood that a variable portion may include any suitable number of nucleotides, and different variable portions within an identification portion may independently have the same or different numbers of nucleotides. Different variable regions also may have the same or different numbers of unique possibilities. For example, a variable portion may be defined having a length of at least 2, at least 3, at least 4, at least 5, at least 7, at least 10, at least 15, at least 20, at least 25, at least 30, at least 40, at least 50, or more nucleotides, and/or a maximum length of no more than 50, no more than 40, no more than 30, no more than 25, no mom than 20, no more than 15, no more than 10, no more than 7, no more than 5, no more than 4, no more than 3, or no more than 2 nucleotides. Combinations of these are also possible, e.g., a variable portion may have a length of between 5 and 50 nt, or between 15 and 25 nt, etc. A non-limiting example of a library is illustrated with identification sequences 1-1, 1-0, 2-1, 2-0, etc. through 22-1 and 22-0, which may be concatenated together (e.g., identification sequence 1- identification sequence 2-identification sequence 3- . . . —identification 22) to produce an bridge sequence (in this non-limiting example, each sequence position 1, 2, . . . 22 may have one of two possibilities, identified with −0 and −1. e.g., sequence position 1 can be either identification sequence 1-1 or 1-0, sequence position 2 can be either identification sequence 2-1 or 2-0, etc.). Similarly, according to certain embodiments, information could also be included in the absence of such sequences. For example, the same information included in the presence of one sequence (e.g. sequence 1-0), could also be determined from the absence of another sequence (e.g., sequence 1-1) Each identification sequence position may be thought of as a “bit” (e.g., 1 or 0 in this example), although it should be understood that the number of possibilities for each “bit” is not necessarily limited to only 2, unlike in a computer. In other embodiments, there may be 3 possibilities (i.e., a “trit”), 4 possibilities (i.e., a “quad-bit”), 5 possibilities, etc., instead of only 2 possibilities as in some embodiments.

The method for generating a library may comprise (a) associating barcode sequences with a plurality of oligonucleotide sequences and a plurality of codewords, wherein the codewords comprise a number of positions that is less than the number of targets, and b) grouping the pairs of nucleic acid probes based on a plurality of codewords, wherein each of the bridge probe corresponds to a specific value of a unique position within the codewords. The method may comprise exposing a sample to one of the bridge probes; imaging the sample; and repeating the exposing and imaging steps one or more times, before repeating with a different bridge probe. This process may be repeated for at least 10, 15, 20, 50, 80, 100, 500 repetitions.

In one aspect, there is provided a method of detecting two or more polynucleotide analytes in a sample, the method comprising:
a) contacting a sample with a library or a probe system as defined herein, and
b) detecting each polynucleotide analyte based on hybridization to a unique bridge probe in the presence of the polynucleotide analyte.

In one embodiment, there is provided a method for combinatorial detection of two or more polynucleotide analytes in a sample, the method comprising:
a) contacting a sample with a library or a probe system as defined herein, and
b) detecting the two or more polynucleotide analytes based on hybridization to a unique bridge probe in the presence of the two or more polynucleotide analyte.

In one embodiment, there is provided a method of determining the levels of two or more polynucleotide analytes in a sample, the method comprising:
a) contacting a sample with a library or a probe system as defined herein, and
b) detecting each polynucleotide analyte based on hybridization to a unique bridge probe in the presence of the polynucleotide analyte.

In one embodiment, two or more nucleic acid probe pairs may be configured to bind to the same unique bridge probe to allow the two or more polynucleotide analytes to be detected combinatorically.

The terms “detecting”, “determining”, “measuring”, “evaluating”, “assessing” and “assaying” are used interchangeably herein to refer to any form of measurement, and include determining if an element is present or not. These terms include both quantitative and/or qualitative determinations. Assessing may be relative or absolute. The method as defined herein may comprise measuring or visualising the levels of two or more polynucleotide analytes in a sample.

In one embodiment, the method comprises contacting the sample with a unique (or bar-coded) bridge probe for each polynucleotide analyte.

In one embodiment, the multiple polynucleotide analytes are detected concurrently based on hybridization to a unique bridge probe for each polynucleotide analyte.

In one embodiment, the multiple polynucleotide analytes are detected sequentially based on multiple rounds of hybridization to a unique bridge probe for each polynucleotide analyte.

In one embodiment, the method comprises detecting the unique bridge probe via hybridization to a readout probe that is conjugated to a label.

In one embodiment, the method comprises contacting the sample with a unique readout probe for each polynucleotide analyte.

The method may comprise removing any bound or unbound bridge and/or readout probe (such as by washing) in between detection of each polynucleotide analyte.

The method may comprise removing any signal from any bound or unbound readout probe in between detection of each polynucleotide analyte. This may be done by, for example, bleaching or quenching a signal.

In one aspect, there is provided a kit comprising a pair of non-naturally occurring nucleic acid probes as defined herein or a library as defined herein. The kit may further comprise bridge probes for detecting nucleic acid probes that are bound to polynucleotide analytes. The bridge probes may be labelled to enable detection or measurement of the analyte. Alternatively, the kit may further comprise readout probes that bind to the bridge probes. The kit optionally also includes instructions for detecting one or more polynucleotide analytes in a sample, one or more buffered solutions (e.g., diluent, hybridization buffer, and/or wash buffer), reference cell(s) comprising one or more of the polynucleotide analytes.

In one embodiment, there is provided a method of performing an array-based assay. Provided herein is also an array-based assay. The term “array” encompasses the term “microarray” and refers to an ordered array presented for binding to nucleic acids and the like. An “array,” includes any two-dimensional or substantially two-dimensional (as well as a three-dimensional) arrangement of spatially addressable regions bearing nucleic acids, particularly oligonucleotides or synthetic mimetics thereof, and the like.

Provided herein is a method of performing a multiplex fluorescence in situ hybridisation (FISH) assay.

Provided herein is a composition, the composition comprising a pair of non-naturally occurring nucleic acid probes as defined herein.

Essentially any type of cell that can be differentiated based on its nucleic acid content (presence, absence, expression level or copy number of one or more nucleic acids) can be detected and identified using the nucleic acid probes as defined herein to detect a suitable selection of polynucleotide analytes. The cell can, for example, be a circulating tumor cell, a virally infected cell, a fetal cell in maternal blood, a bacterial cell or other microorganism in a biological sample (e.g., blood or other body fluid), an endothelial cell, precursor endothelial cell, or myocardial cell in blood, a stem cell, or a T-cell. Rare cell types can be enriched prior to performing the methods, if necessary, by methods known in the art (e.g., lysis of red blood cells, isolation of peripheral blood mononuclear cells, further enrichment of rare target cells through magnetic-activated cell separation (MACS), etc.). The methods are optionally combined with other techniques, such as DAPI staining for nuclear DNA. It will be evident that a variety of different types of nucleic acid markers are optionally detected simultaneously by the methods and used to identify the cell. For example, a cell can be identified based on the presence or relative expression level of one nucleic acid target in the cell and the absence of another nucleic acid target from the cell; e.g., a circulating tumor cell can be identified by the presence or level of one or more markers found in the tumor cell and not found (or found at different levels) in blood cells, and its identity can be confirmed by the absence of one or more markers present in blood cells and not circulating tumor cells. The principle may be extended to using any other type of markers such as protein based markers in single cells.

Provided herein are methods of diagnosis of a disease. The disease may be cancer, or viral or bacterial infection or a genetic disorder due to the presence of a defective gene. The method may comprise detecting the presence or absence of one or more polynucleotide analytes in a sample obtained from a subject. Provided herein are also methods of treating the disease following detection of the disease.

By “subject” or “patient” is meant any single subject for which therapy is desired, including humans, cattle, horses, pigs, goats, sheep, dogs, cats, guinea pigs, rabbits, chickens, insects and so on. Also intended to be included as a subject are any subjects involved in clinical research trials not showing any clinical sign of disease, or subjects involved in epidemiological studies, or subjects used as controls.

One or more polynucleotide analytes associated with cancer can be detected using the nucleic acid probes as defined herein, e.g., those that encode over expressed or mutated polypeptide growth factors (e.g., sis), overexpressed or mutated growth factor receptors (e.g., erb-B1), over expressed or mutated signal transduction proteins such as G-proteins (e.g., Ras), or non-receptor tyrosine kinases (e.g., abl), or over expressed or mutated regulatory proteins (e.g., myc, myb, jun, fos, etc.) and/or the like. In general, cancer can often be linked to signal transduction molecules and corresponding oncogene products, e.g., nucleic acids encoding Mos, Ras, Raf, and Met; and transcriptional activators and suppressors, e.g., p53, Tat, Fos, Myc, Jun, Myb, Rel, and/or nuclear receptors, p53. For detection of circulating tumor cells (CTC), a variety of suitable polynucleotide analytes are known. For example, a multiplex panel of markers for CTC detection could include one or more of the following markers: epithelial cell-specific (e.g. CK19, Mucl, EpCAM), blood cell-specific as negative selection (e.g. CD45), tumor origin-specific (e.g. PSA, PSMA, HPN for prostate cancer and mam, mamB, her-2 for breast cancer), proliferating potential-specific (e.g. Ki-67, CEA, CA15-3), apoptosis markers (e.g. BCL-2, BCL-XL), and other markers for metastatic, genetic and epigenetic changes.

Similarly, one or more polynucleotide analytes from pathogenic or infectious organisms can be detected by the nucleic acid probes as defined herein, e.g., for infectious fungi, e.g., Aspergillus, or Candida species; bacteria, particularly E. coli, which serves a model for pathogenic bacteria (and, of course certain strains of which are pathogenic), as well as medically important bacteria such as Staphylococci (e.g., aureus), or Streptococci (e.g., pneunoniae); protozoa such as sporozoa (e.g., Plasmodia), rhizopods (e.g., Entamoeba) and flagellates (Trypanosona, Leislunania, Trichonmonas, Giardia, etc.); viruses such as (+) RNA viruses (examples include Poxviruses e.g., vaccinia; Picornaviruses. e.g., polio; Togaviruses, e.g., rubella; Flaviviruses, e.g., HCV; and Coronaviruses), (−) RNA viruses (e.g., Rhabdoviruses. e.g., VSV; Paramyxovimses. e.g., RSV; Orthomyxovimses, e.g., influenza; Bunyaviruses; and Arenaviruses), dsDNA viruses (e.g. Reoviruses), RNA to DNA viruses, i.e., Retroviruses, e.g., HIV and HTLV, and certain DNA to RNA viruses such as Hepatitis B.

Gene amplification or deletion events can be detected at a chromosomal level using the nucleic acid probes as described herein, as can altered or abnormal expression levels. Some polynucleotide analytes include oncogenes or tumor suppressor genes subject to such amplification or deletion. Exemplary nucleic acid targets include, integrin (e.g., deletion), receptor tyrosine kinases (RTKs; e.g., amplification, point mutation, translkcation, or increased expression), NF1 (e.g., deletion or point mutation), Akt (e.g., amplification, point mutation, or increased expression). PTEN (e.g., deletion or point mutation), MDM2 (e.g., amplification), SOX (e.g., amplification), RAR (e.g., amplification), CDK2 (e.g., amplification or increased expression). Cyclin D (e.g., amplification or translocation), Cyclin E (e.g., amplification), Aurora A (e.g., amplification or increased expression), P53 (e.g., deletion or point mutation), NBS1 (e.g., deletion or point mutation). Gli (e.g., amplification or translocation). Myc (e.g., amplification or point mutation). HPV-E7 (e.g., viral infection), and HPV-E6 (e.g., viral infection).

If a polynucleotide analyte is used as a reference, suitable reference nucleic acids have similarly been described in the art or can be determined. For example, a variety of genes whose copy number is stably maintained in various tumor cells is known in the art. Housekeeping genes whose transcripts can serve as references in gene expression analyses include, for example, 18S rRNA, 28S rRNA, GAPD, ACTB, and PPIB.

Provided herein is a method of detecting or visualising the expression of one or more polynucleotide analytes in a sample, the method comprising a) contacting a sample with a library as defined herein, and b) detecting or visualising the expression of each polynucleotide analyte based on hybridisation to a unique bridge probe in the presence of the one or more polynucleotide analytes.

The method may comprise detecting the presence or level of mRNA in a sample.

The sample may be a cell or tissue sample.

Throughout this specification, unless the context requires otherwise, the word “comprise”, or variations such as “comprises” or “comprising”, will be understood to imply the inclusion of a stated element or integer or method step or group of elements or integers or method steps but not the exclusion of any other element or integer or method steps or group of elements or integers or method steps.

As used in the subject specification, the singular forms “a”, “an” and “the” include plural aspects unless the context clearly dictates otherwise. Thus, for example, reference to “a method” includes a single method, as well as two or more methods; reference to “an agent” includes a single agent, as well as two or more agents; reference to “the disclosure” includes a single and multiple aspects taught by the disclosure; and so forth. Aspects taught and enabled herein are encompassed by the term “invention”. Any variants and derivatives contemplated herein are encompassed by “forms” of the invention.

### EXAMPLES

### Materials and Methods

SPLIT-FISH library design. Targeting regions (pairs of 25-nt sequences with 2-nt spacing in between the pair) were identified using a previously published algorithm. First, reference transcript sequences were downloaded from the GENCODE website (human v24 and mouse m4 respectively). A specificity table was calculated using 15-nt seed and 0.2 specificity cut-off was used. Quartet repeats (‘AAAA’, ‘TITT’, ‘GGGG’, and ‘CCCC’), KpnI restriction sites (‘GGTACC’ (SEQ ID NO: 1) and ‘CCATGG’ (SEQ ID NO: 2)), and EcoRI restriction sites (‘GAATTC’(SEQ ID NO: 3) and ‘CTTAAG’ (SEQ ID NO: 4)) were excluded from the possible target regions. Then, the right targeting region pairs were concatenated with the right bridge sequence (e.g. ‘CactctaCCTAT’ (SEQ ID NO: 5), lowercase indicates variable bases that form the 6-nt barcode, TAT is a linker between the bridge sequence and the targeting region). The left targeting region pairs were concatenated with the left bridge sequence ‘TAT ATTTAACCG’ (SEQ ID NO: 6). Finally, KpnI and EcoRI restriction sites, as well as the forward and reverse PCR primers were introduced at both ends of each side of the probes.

Removal of the PCR primers via restriction digestion is required for efficient subsequent hybridization of the bridge sequence. The list of encoding probes can be found in Table 1. The bridge sequences were flanked by readout sequences at both ends. The list of bridge sequence can be found in Table 3. The readout sequences used were ‘/5Cy5/TTACTCACGCACCCATCA’ (SEQ ID NO: 7) and ‘/5Alex750NfTTCTCACGCTTTACAAT’ (SEQ ID NO: 8). To construct the 317-genes combinatorial library, a ‘26 choose 2’ coding scheme was used. Eight of the 325 possible code-words were blanks, which are not assigned to any gene (no encoding probes), to act as negative controls that estimate the levels of the false-positive background. For each gene, 72 pairs of target regions were split into two pools. Each pool was assigned a 6-nt barcode according to the gene's ‘on’ bits. The gene codebook assignment for the 317-genes library can be found in Table 2. The conventional multiplexed FISH probe and library were designed as previously described. The conventional encoding probe library and readout sequences can be found in Table 4 and 5 respectively. The conventional codebook can be found in Table 6.

Probe amplification and preparation. Probe library (Twist Bioscience) was made using a slightly modified version of a previously published protocol. Briefly, the oligopool was first amplified by limited cycle PCR using Phusion Hot Start Flex 2× master mix (NEB, Cat: M0536L) with an annealing temperature of 66° C., followed by an overnight in vitro transcription using a high yield in vitro transcription kit (NEB, Cat: E2050S). T7 promoter sequence was introduced on the reverse primer during the PCR. Next, reverse transcription from the RNA template (ThermoFisher Cat: EP0753) was performed. The RNA was then cleaved off using alkaline hydrolysis, leaving behind ssDNA which was then purified via spin column purification (Zymo Cat: C1016-50), and eluted in nuclease free water (Ambion, Cat: AM9930). Cut primers, complementary to the EcoRI and KpnI restriction sites were then annealed to the ssDNA probes before performing a double restriction digest for 16 hours at 37° C. using high fidelity enzymes (NEB Cat: R3101M, R3142M) to cleave off the forward and reverse primers. Finally, the ssDNA probes were purified using a spin column (Zymol, Cat: C1016-50) or magnetic beads (Beckman Coulter, Cat: A63882) and eluted in nuclease-free water. Probes were dried and stored at −20° C. The primers used for PCR are f‘AACGAACGGAGGGTCATTGG’ (SEQ ID NO: 9) and ‘TAATACGACTCACTATAGGGAGGCTCTACTCGCATTAGGG’ (SEQ ID NO: 10); the primers used for restriction digestion are ‘TACTCGCATTAGGGGAATTCNN’ (SEQ ID NO: 11) and ‘NNGTACCCCAATGACCCTCCGT’ (SEQ ID NO: 12).

Cell culture sample preparation. Human foreskin fibroblasts (ATCC® CRL-2097™), human A549 (ATCC® CCL-185™), and AML12 (ATCC® CRL-2254™) cells were cultured in Dulbecco's High Glucose Modified Eagles Medium (Hyclone™ Cat: SH30022.01), supplemented with 10% fetal bovine serum (Thermofisher, Cat: 26140079). A549 cells were cultured in DMEM/F12 1:1, supplemented with 10% fetal bovine serum. Cells were grown in 6-well plates on 22 mm×22 mm No. 1 coverslips (Marienfeld-Superior Cat: 0101050) for the XLOC_010514 and MUC5AC experiments, or 40 mm diameter No. 1 coverslips (Warner Instruments Cat: 64-1500) for the FLNA experiments. Cells were grown to ˜80% confluency before fixation in 4% vol/vol paraformaldehyde (Electron Microscopy Sciences Cat: 15714) in 1× PBS for 15 minutes at room temperature. Following fixation, the samples were quenched in 0.1 M Glycine (1st BASE) for 1 minute at room temperature. The cells were then permeabilized in 70% ethanol overnight at 4° C.

Tissue sample preparation and coverslip functionalization. All animal care and experiments were carried out in accordance with Agency for Science, Technology and Research (A*STAR) Institutional Animal Care and Use Committee (IACUC) guidelines. Coverslip functionalization and tissue processing were based on a slightly modified version of a previously published protocol³. Briefly, coverslips (Warner Instruments Cat: 64-1500) were cleaned with 1 M KOH in an ultrasonic water bath for 20 minutes, rinsed thrice with MilliQ water followed by 100% methanol. Then, the coverslips were immersed in an amino-silane solution (3% vol/vol (3-Aminopropyl)triethoxysilane [MERCK Cat: 440140] 5% vol/vol acetic acid [Sigma Cat: 537020] in methanol) for 2 minutes at room temperature before rinsing thrice with MilliQ water and air dried. Functionalized coverslips can then be used immediately or stored in a dry, desiccated environment at room temperature for several weeks. Histology work was performed by the Advanced Molecular Pathology Laboratory, IMCB, A*STAR, Singapore. Briefly, C57BL/6NTac mice aged 8 weeks (InVivos) were euthanized with ketamine, the kidney, liver, brain, and ovary were quickly harvested, cut to smaller pieces, and frozen immediately in Optimal Cutting Temperature compound (Tissue-Tek O.C.T.; VWR, 25608-930), and stored at −80° C. 7 μm sections of fresh frozen tissues were cut using a cryotome onto functionalized coverslips. Sections were air-dried for 5 minutes at room temperature prior to fixation in 4% vol/vol paraformaldehyde in 1× PBS for 15 minutes. Following fixation, samples were rinsed once with 1× PBS and either permeabilized in 70% ethanol overnight at 4° C. or stored at −80° C.

XLOC_010514, MUC5AC, and FLNA experiments. After permeabilization, the cultured cells were equilibrated to room temperature before rehydration in 2× saline-sodium citrate (SSC, Axil Scientific Cat: BUF-3050-20X1L) for 5 minutes. Samples were incubated in a 10% formamide wash buffer, containing 10% deionized formamide (Ambion™ Cat: AM9342, AM9344) and 2×SSC, for 30 minutes at room temperature. The split probes were diluted in a 10% hybridization buffer to a final concentration of 20 nM per probe. The 10% hybridization buffer composed of 10% deionized formamide (vol/vol) and 10% dextran sulfate (Sigma Cat: D8906) (wt/vol) in 2×SSC. The encoding probes were stained overnight at 37° C. in a humidified chamber. Following hybridization of the encoding probes, the samples were washed in a 10% formamide wash buffer twice, incubating for 15 minutes at 37° C. per wash. The samples were then removed from the 10% formamide wash buffer and stained with either the bridge probe or the conventional readout probe. The probes were diluted to a concentration of 10 nM in 10% hybridization buffer and stained for 20 minutes at room temperature. The cells were then washed once with 10% formamide wash buffer and then twice with 2×SSC at room temperature. DAPI (Sigma Cat: D9564) was stained at a concentration of 1 μg/mL in 2×SSC for 10 minutes at room temperature. The samples were then washed twice with 2×SSC and either imaged immediately or stored for no longer than 12 hours at 4° C. in 2×SSC before imaging. The list of XLOC_010514, MUC5AC, and FLNA sequences can be found in Table 7, 8, and 9 respectively.

Multiplexed FISH experiments in tissue. Tissue samples were stained as described above, using 20% formamide concentration in the hybridization and wash buffers instead of 10%. For tissue samples, pre-hybridization was also extended to 3 hours at 37° C. in 20% formamide wash buffer. The samples were stained overnight or longer at a final probe concentration of 500 μM (2 to 3 fold higher concentration than used in the conventional experiment) in 20% hybridization buffer. After two 20% formamide washes, the samples were washed twice with 2×SSC and either imaged immediately or stored in 2×SSC for no longer than one week at 4° C. prior to imaging.

Split-FISH imaging cycle. Samples were then mounted into a flow chamber (Bioptechs Cat: FCS2), which was secured to the microscope stage. Hybridization of the bridge and readout probes in the flow chamber was done sequentially by buffer exchange controlled by a custom-built, computer-controlled fluidics system. The system consisted of three daisy-chained eight-way valves for buffer selection and a peristaltic pump providing the driving force for fluid flow, as previously described. The bridge probe solution contained 5 nM of each bridge sequence in a 10% hybridization buffer. The sample was incubated in the solution for 10 minutes at room temperature. Next, 5 nM of fluorescently labeled readout probe in 10% hybridization buffer was flowed into the chamber and incubated for another 10 minutes at room temperature. Following hybridization, the sample was washed with 10% formamide wash buffer to remove unbound probes. Imaging buffer was then flowed into the chamber before images were acquired. The imaging buffer consisted of 2×SSC, 50 mM Tris-HCl pH 8, 10% glucose, 2 mM Trolox (Sigma, Cat: 238813), 0.5 mg/ml glucose oxidase (Sigma, Cat: G2133) and 40 μg/ml catalase (Sigma, Cat: C30). To remove the fluorescent signals, the samples were washed with 40% formamide wash buffer. This hybridization and wash cycle was repeated until all the bits were imaged. With two-color imaging, 26 bits were completed in 13 cycles. 133-genes (Modified Hamming Distance 4) multiplexed FISH imaging using the conventional probes was performed as previously described. The conventional probe library correlated well with bulk RNA-seq (FIG. 11).

Imaging Setup 1. The XLOC_010514 and MUC5AC experiments were performed using a custom-built microscope that was constructed around a Nikon Ti-E body, MS-200 ASI X-Y stage, CFI Plan Apo Lambda 100×1.45 N.A, oil-immersion objective, and Andor iXon Ultra 888 EMCCD camera. DAPI was excited by 405 nm (LuxX, 405-20), and Cy5 was excited by 638 nm (LuxX, 638-100) solid-state lasers (Omicron). Z-stacks, of 400 nm apart, were obtained for each laser excitation for five different Z positions. The exposure time was 1 second.

Imaging Setup 2. The FLNA and multiplexed FISH experiments were performed using a second custom-built microscope that was constructed around a Nikon Ti2-E body, Marzhauser SCANplus IM 130×85 motorized X-Y stage, a Nikon CFI Plan Apo Lambda 60×1.4 N.A, oil-immersion objective, and an Andor Sona 4.2B-11 sCMOS camera. Focus was maintained using the Nikon Perfect Focus system and only one Z position was imaged per field of view per cycle. The DAPI channel was excited by a Coherent Obis 405 100 mW laser. The following two fiber lasers from MPB Communications: 2RU-VFL-P-1000-647-B1R (1000 mW), 2RU-VFL-P-500-750-BIR (500 mW) were used as illumination for Cy5 (647 nm) and Alexa750 (750 nm) respectively. All laser channels were combined and launched into a Newport F-SM8-C-2FCA fiber. The resulting beam was collimated and flattened using an AdlOptica 6_6 series Pi-shaper, then expanded before being sent into a 300 mm lens near the back-port of the Ti-2 to illuminate an approximately 230 um×230 um field of view. Custom multi-wavelength filters ZET488/532/592/647/750m (Chroma) and ZT488/532/592/647/750rpc-UF2 (Chroma) were used. A Finger Lakes Instrumentation HS-632 High Speed Filter Wheel, containing FF01-433/24-32, FF02-684/24-32 and FFO1-776/LP-32 emission filters (Semrock), was attached to the output port between the microscope and the camera, allowing different emission filters to be used when imaging respective channels. The exposure time was 500 ms.

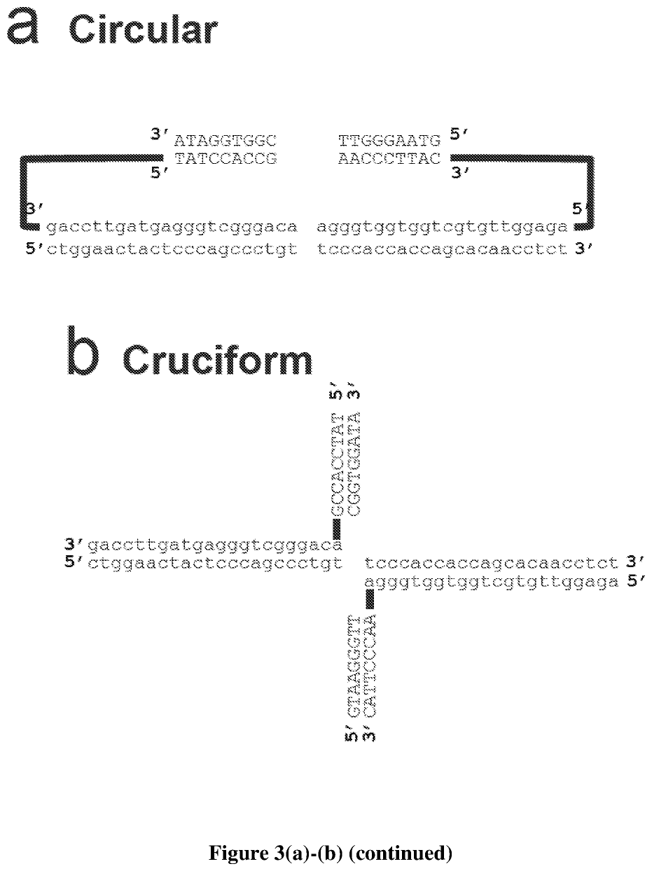

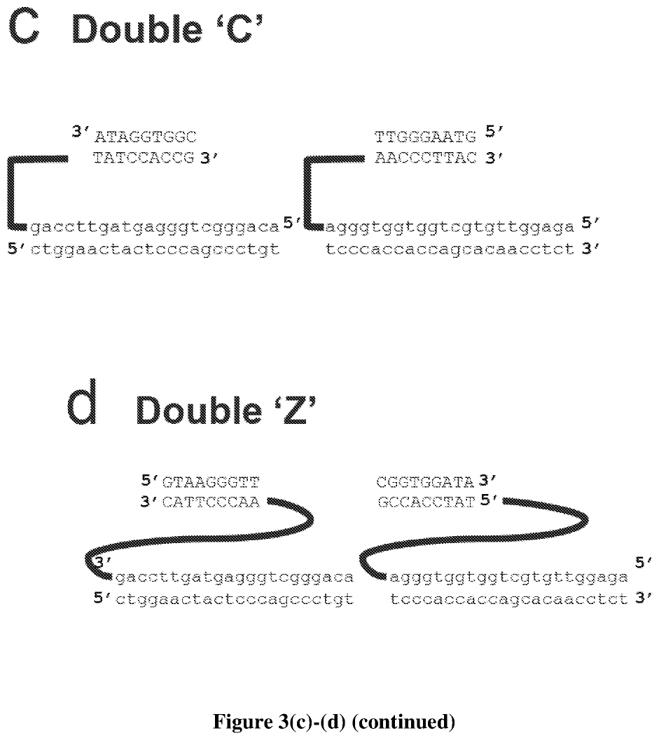

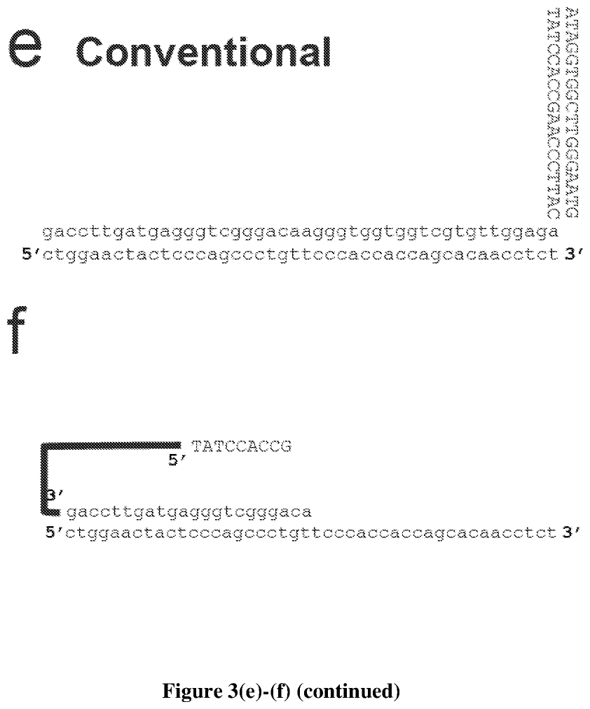

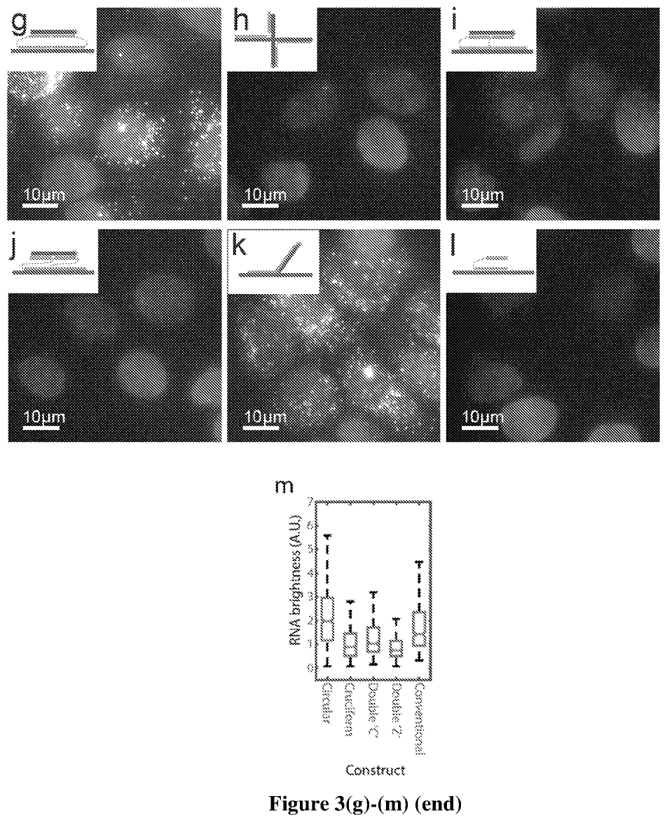

Image analysis. The multiplexed FISH images were processed by a custom Python pipeline, following a previously published approach but with modified pre-processing, gene callout filtering, and mosaic-stitching procedures. Briefly, the images from each hybridization cycle were first corrected for field and chromatic distortion. Images were then registered for translation relative to a selected frame in the Cy5 channel by phase correlation using a subpixel registration algorithm provided in the Scikit-image package. For each dataset, a global bit-wise normalization was performed by pooling all pixels above the 99.9^{th} percentile of intensity in each field of view, then taking the 50^{th} percentile of the pooled pixel intensities as a normalization value for the bit. Images were filtered in the frequency domain using a second order 2D band-pass Butterworth filter to remove cell background (low frequency cutoff) and camera noise (high frequency cutoff). The n-dimensional vector (where n is the number of bits) for each aligned pixel is then normalized to the unit length by dividing by its magnitude (L2 norm). The same normalization was done for each code-word in the set of genes. The Euclidean distance from the pixel vector to each gene's code-word was then calculated. All pixels were filtered for maximum Euclidean distance (distance threshold) to a gene's code-word, using a threshold of 0.52 for conventional and 0.33 for split-FISH. The L2 norm of each pixel vector was used as a second filter (magnitude threshold) to remove called pixels with too low intensities. The called and filtered pixels were then grouped into connected regions (4-connected neighbourhood) for each gene. Regions with only 1 pixel were subject to a second more stringent intensity threshold. Sets of parameters which yielded both good correlation to bulk FPKM counts and high gene counts were chosen. The number of regions for each gene across all fields of view was then summed, and total counts for each gene compared to the respective FPKM values by calculating the Pearson correlation. The FPKM values from bulk RNA sequencing of mouse tissues were downloaded from the ENCODE portal (https://www.encodeproect.org/) with the following identifiers: ENCSR000BZC (ovary), ENCFF478QMU (kidney replicate 1), ENCFF638NYA (kidney replicate 2), ENCFF844MJF (liver replicate 1), ENCFF271DWG (liverreplicate 2), ENCFF653BKJ (frontal cortex replicate 1), and ENCFF703SOK (frontal cortex replicate 2). The FPKM values of AML12 cell line was obtained by performing bulk RNA sequencing in-house. Briefly, RNA was extracted using Isolate II RNA Mini Kit (Bioline), sequencing was performed at the GIS next generation sequencing platform, A*STAR, Singapore, and the sequences were analyzed using Salmon. The list of FPKM values (or their mean if the tissue has sequencing replicates) used for the Pearson correlation analysis is listed in Table 10. Cells were manually counted using the DAPI and RNA images. For the split-FISH library, 789, 4043, 7484, 13405, and 26001 cells were imaged for the AML-12, brain, liver, ovary and kidney experiments respectively. For the conventional library, 1382, 2581 and 2729 cells were imaged for the AML-12, brain and liver experiments respectively. Brightness and spot counting analysis for the MUC5AC and FLNA experiments (for FIGS. 3 and 7) were done using a multi-Gaussian-fitting algorithm, as previously described. For mosaic stitching in tissue samples, adjacent field of view (FOV) alignments were estimated using the phase correlation algorithm from Scikit-Image modified to output a value for the phase correlation peak magnitude, which is an indication of registration accuracy. A graph with FOVs as vertices and edges weighted by the negative of the phase correlation peak value was generated. The full mosaic was then stitched by calculating the minimum spanning tree (SciPy) and shifting each field of view accordingly. Overlapping regions were blended using maximum intensity projection.

### Example 1

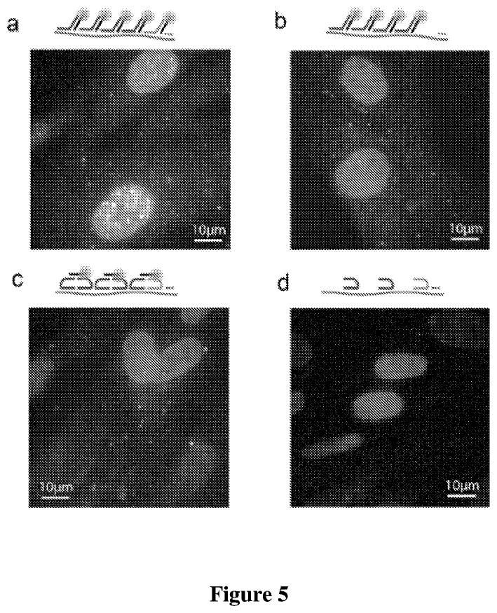

First, the split probe sequence was optimized using single-molecule FISH on MUC5AC transcripts in A549 cells (FIG. 1). It was reasoned that the length of the complementary sequences between the bridge probe and either of the encoding probes has to be shorter in length than in conventional multiplexed FISH to prevent any single and unpaired off-target encoding probe from binding to the readout probe. Thus, the length of the split bridge sequence was titrated and it was discovered that nine or fewer nucleotides is required to produce a level of non-specific background signal that is virtually undetectable (FIG. 1). Several pairing schemes were further screened, including circular, cruciform, double ‘C’, and double ‘Z’ (FIG. 3), and it was found that the circular construct produces the brightest on-target signal. It had a mean brightness that was ˜4.7 fold higher than the double ‘Z’ construct. Importantly, the circular construct scheme produced a signal intensity that is comparable to the conventional readout scheme, indicating that RNA brightness was not compromised as a result of eliminating non-specific probe binding. To further test the optimized split probe construct, single-molecule FISH was performed on the long non-coding RNA XLOC_010514, for which one of the probes is known to non-specifically bind to off-targets within the cell nuclei, which was shown in a previous study (FIG. 5). The split probe approach successfully quells the signals arising from the non-specific binding, suggesting that there is no need to remove or even know the non-specific ‘rogue’ sequence a priori.

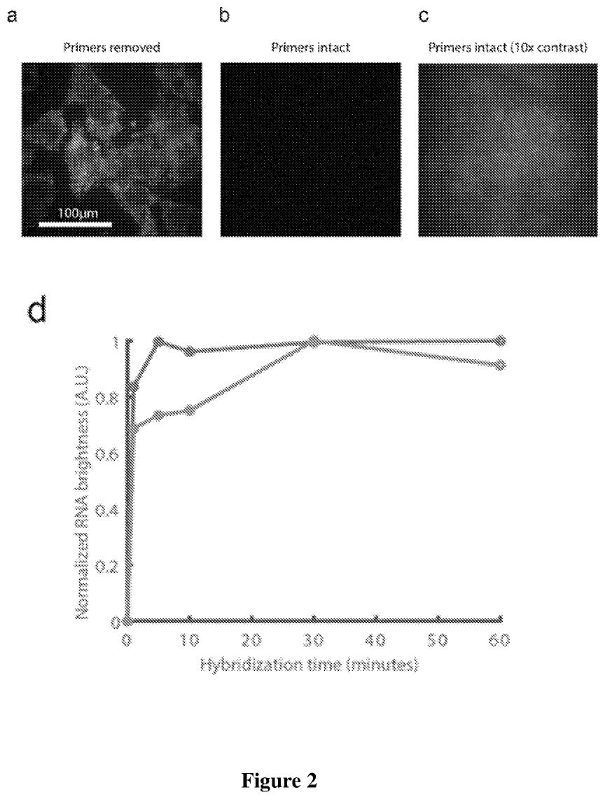

Next, the inventors focused on optimizing the split-FISH workflow (FIG. 6a). It was found that the primers used for oligo library amplification impeded the circularization of the adjoining probe pairs, so restriction sites adjacent to primer sequences were incorporated, allowing the primers to be cleaved off by restriction digestion (FIG. 2). It was also observed that different bridge probe sequences yielded varying RNA spot brightness. Thus, several sequences were screened, and those that yielded the highest brightness within 10 minutes of hybridization time were selected. With the optimized design, the inventors were able to perform multiple iterations of hybridization and washing (at least 20 rounds) without any observable loss of FISH signal or RNA counts (FIG. 7).

The performance of split-FISH was then compared against conventional multiplexed FISH in mouse cell cultures and mouse tissue slices. To demonstrate the combinatorial labelling of RNAs, 317 genes were randomly selected as targets, and 26 barcoded bridge sequences were designed. An ‘N Choose 2’ barcoding scheme (Table 2) was designed by assigning each of the two required barcodes to half of the available encoding probes (Table 1). Compared with samples stained with the conventional probe library, samples stained with the split probe library showed decreases in non-specific background (estimated as the median value of all the raw images) that was about 16% in cultured mouse hepatocytes (AML12, FIG. 8b, c) and about 44% in brain tissue slices (FIG. 8d, e). The number of detected RNAs in AML12 correlated well with bulk RNA-seq (log Pearson correlation of 0.7) and conventional multiplexed FISH (10 common genes, log Pearson correlation of 0.97) (FIG. 5a). The average false positive rate (estimated using number of blank code-words detected per cell) in AML12 (0.13±0.015 per cell, S.E.M, n=8 replicates) was comparable to that previously reported while using conventional multiplexed FISH in a cleared U-2OS cell-line sample (0.08 ±0.03 per cell).

To demonstrate that split-FISH works robustly without any tissue-specific clearing, the same probe set for the 317 genes was used and split-FISH imaging of three additional mouse tissues-kidney, liver, and ovary was performed. The transcript counts from all the tissues also correlated strongly with bulk RNA-seq results, with log Pearson correlation values between 0.54 and 0.75 (FIG. 5b). Images taken after washing also confirmed that off-target binding is the main contributor to background signal, and tissue auto-fluorescence in our detection channels was insignificant in comparison (FIG. 9). The average false positive rates of split-FISH in brain, kidney, liver, and ovary were 0.012±0.002, 0.0042±0.0004, 0.008±0.003, and 0.03±0.009 per cell respectively (S.E.M., n=8 replicates). In fact, the false positive rates were lower by ˜44 fold (in brain tissue), and ˜19 fold (in liver tissue) compared to the conventional multiplexed FISH (Welch's t-test, p-values of 0.020 and 0.014 respectively, FIG. 5a), despite employing a barcoding scheme with lower Hamming distance. This confirmed that non-specific probe binding was contributing to false positive signals.

For each tissue type that was imaged, diverse localization patterns of the single-cell transcriptome was observed. For example, Map4 transcripts were found to be highly enriched in the neuronal processes in the frontal cortex (FIG. 10a), and Ahnak was found predominantly lining the portal veins in the liver (FIG. 10d). Distinct zonation patterns of certain transcripts (e.g., Osbp18, Pp1, and Notch 3) in the kidney tissue (FIG. 10b) suggest a spatial division of labor previously observed in liver via single molecule FISH. Some transcripts, such as Slc12a7, Plxnc1, and Dsp, were highly compartmentalized in the mouse ovary, possibly corresponding to different maturation stages of the follicles (FIG. 10c). In the mouse liver tissue, the transcripts of Son and Abcc2 were found to be highly localized to the nucleus in the cells, highlighting the power of multiplexed FISH to distinguish subcellular features in tissue samples.

In conclusion, the inventors showed accurate multiplexed FISH of 317 genes in diverse mouse tissues without requiring tissue clearing, demonstrating the prowess of split-FISH not only in simplifying tissue preparation protocols for multiplexed FISH, but also in broadening the range of accessible tissue types.

TABLE 1
317-genes split library template sequences. Template sequences include the forward
and reverse primer sequences necessary for library amplification. The template
sequences for 1 target gene is shown below.

| Target Gene | Transcript ID | Template Sequence |
|---|---|---|
| Ccdc177 | ENSMUST00000073251.6 | AACGAACGGAGGGTCATTGG GGTAC CACTCTACC TAT |
| | | GGGTTACTGCCGGCTGCAGCAGCCA GAATTC CCCTAATGCGAGTAGAGCCT |
| | | (SEQ ID NO: 13) |
| Ccdc177 | ENSMUST00000073251.6 | AACGAACGGAGGGTCATTGG GGTAC CACTCTACC TAT |
| | | CGGAGGCGGAGGCCGAGGAGGCAGC GAATTC CCCTAATGCGAGTAGAGCCT |
| | | (SEQ ID NO: 14) |
| Ccdc177 | ENSMUST00000073251.6 | AACGAACGGAGGGTCATTGG GGTAC CACTCTACC TAT |
| | | AGCAGCTCCACGGGCTTGACCGCGC GAATTC CCCTAATGCGAGTAGAGCCT |
| | | (SEQ ID NO: 15) |
| Ccdc177 | ENSMUST00000073251.6 | AACGAACGGAGGGTCATTGG GGTAC CACTCTACC TAT |
| | | GCGCATGATGCGCTCGCGCTCGGCG GAATTC CCCTAATGCGAGTAGAGCCT |
| | | SEQ ID NO: 16) |
| Ccdc177 | ENSMUST00000073251.6 | AACGAACGGAGGGTCATTGG GGTAC CACTCTACC TAT |
| | | GGCTCTTTCTACCTGTCCGAGAACC GAATTC CCCTAATGCGAGTAGAGCCT |
| | | SEQ ID NO: 17) |
| Ccdc177 | ENSMUST00000073251.6 | AACGAACGGAGGGTCATTGG GGTAC CACTCTACC TAT |
| | | CTGTTCCTGGCGGAAGCTCGAGGTG GAATTC CCCTAATGCGAGTAGAGCCT |
| | | (SEQ ID NO: 18) |
| Ccdc177 | ENSMUST00000073251.6 | AACGAACGGAGGGTCATTGG GGTAC CACTCTACC TAT |
| | | AGTGGCTCAGGTCTCCGAGGCTGAA GAATTC CCCTAATGCGAGTAGAGCCT |
| | | (SEQ ID NO: 19) |
| Ccdc177 | ENSMUST00000073251.6 | AACGAACGGAGGGTCATTGG GGTAC CACTCTACC TAT |
| | | TGGTGCCGGGCCAGCATGAGGGCGG GAATTC CCCTAATGCGAGTAGAGCCT |
| | | (SEQ ID NO: 20) |
| Ccdc177 | ENSMUST00000073251.6 | AACGAACGGAGGGTCATTGG GGTAC CACTCTACC TAT |
| | | CGCTGCTTCTCTCGCTCCTCGCGCT GAATTC CCCTAATGCGAGTAGAGCCT |
| | | (SEQ ID NO: 21) |
| Ccdc177 | ENSMUST00000073251.6 | AACGAACGGAGGGTCATTGG GGTAC CACTCTACC TAT |
| | | CTCCCGCCGCAGCAGTCCCTGACGC GAATTC CCCTAATGCGAGTAGAGCCT |
| | | (SEQ ID NO: 22) |
| Ccdc177 | ENSMUST00000073251.6 | AACGAACGGAGGGTCATTGG GGTAC CACTCTACC TAT |
| | | GCGCCGCCCGCTGGGCGCGCTCCCG GAATTC CCCTAATGCGAGTAGAGCCT |
| | | (SEQ ID NO: 23) |
| Ccdc177 | ENSMUST00000073251.6 | AACGAACGGAGGGTCATTGG GGTAC CACTCTACC TAT |
| | | GCTGCTGCCGGACTCGGCCTCTAAG GAATTC CCCTAATGCGAGTAGAGCCT |
| | | (SEQ ID NO: 24) |
| Ccdc177 | ENSMUST00000073251.6 | AACGAACGGAGGGTCATTGG GGTAC CACTCTACC TAT |
| | | CGCGCTCCCGCAGCTCCCGGGCGCG GAATTC CCCTAATGCGAGTAGAGCCT |
| | | (SEQ ID NO: 25) |
| Ccdc177 | ENSMUST00000073251.6 | AACGAACGGAGGGTCATTGG GGTAC CACTCTACC TAT |
| | | GCCGTCGTTCTCCCGCCCGCGCCAG GAATTC CCCTAATGCGAGTAGAGCCT |
| | | (SEQ ID NO: 26) |
| Ccdc177 | ENSMUST00000073251.6 | AACGAACGGAGGGTCATTGG GGTAC CACTCTACC TAT |
| | | GTCTTCGTCTCTCTCTACCTTCTCC GAATTC CCCTAATGCGAGTAGAGCCT |
| | | (SEQ ID NO: 27) |
| Ccdc177 | ENSMUST00000073251.6 | AACGAACGGAGGGTCATTGG GGTAC CACTCTACC TAT |
| | | CCGGGCCGTGGAGCGAGCGCTCTCC GAATTC CCCTAATGCGAGTAGAGCCT |
| | | (SEQ ID NO: 28) |
| Ccdc177 | ENSMUST00000073251.6 | AACGAACGGAGGGTCATTGG GGTAC CACTCTACC TAT |
| | | AGAGAGGCCACACCTGGCCATATCC GAATTC CCCTAATGCGAGTAGAGCCT |
| | | (SEQ ID NO: 29) |
| Ccdc177 | ENSMUST00000073251.6 | AACGAACGGAGGGTCATTGG GGTAC CACTCTACC TAT |
| | | AATACCATGTCTGCCTTCTCAGTGC GAATTC CCCTAATGCGAGTAGAGCCT |
| | | (SEQ ID NO: 30) |
| Ccdc177 | ENSMUST00000073251.6 | AACGAACGGAGGGTCATTGG GGTAC CACTCTACC TAT |
| | | CTACAGGGACCTTCCTCCTCCCAGC GAATTC CCCTAATGCGAGTAGAGCCT |
| | | (SEQ ID NO: 31) |
| Ccdc177 | ENSMUST00000073251.6 | AACGAACGGAGGGTCATTGG GGTAC CACTCTACC TAT |
| | | AAGATAGAGAGGTAAGTCTGGGAGA GAATTC CCCTAATGCGAGTAGAGCCT |
| | | (SEQ ID NO: 32) |
| Ccdc177 | ENSMUST00000073251.6 | AACGAACGGAGGGTCATTGG GGTAC CACTCTACC TAT |
| | | GGGAACAGGATAATTTGTTCACGTG GAATTC CCCTAATGCGAGTAGAGCCT |
| | | (SEQ ID NO: 33) |
| Ccdc177 | ENSMUST00000073251.6 | AACGAACGGAGGGTCATTGG GGTAC CACTCTACC TAT |
| | | CTGGAGGTTGGAGGGCTTCCATTAA GAATTC CCCTAATGCGAGTAGAGCCT |
| | | (SEQ ID NO: 34) |
| Ccdc177 | ENSMUST00000073251.6 | AACGAACGGAGGGTCATTGG GGTAC CACTCTACC TAT |
| | | GAAGGTAGCTCTACTTCGTAAGGCA GAATTC CCCTAATGCGAGTAGAGCCT |
| | | (SEQ ID NO: 35) |
| Ccdc177 | ENSMUST00000073251.6 | AACGAACGGAGGGTCATTGG GGTAC CACTCTACC TAT |
| | | GCAGCCAAGGATCCTAAATTGTCTT GAATTC CCCTAATGCGAGTAGAGCCT |
| | | (SEQ ID NO: 36) |
| Ccdc177 | ENSMUST00000073251.6 | AACGAACGGAGGGTCATTGG GGTAC CACTCTACC TAT |
| | | CTTCAGGTATAAGTGAGAGGGTACC GAATTC CCCTAATGCGAGTAGAGCCT |
| | | (SEQ ID NO: 37) |
| Ccdc177 | ENSMUST00000073251.6 | AACGAACGGAGGGTCATTGG GGTAC CACTCTACC TAT |
| | | GCCTCCCAACTCCAGGTCAAGAAAG GAATTC CCCTAATGCGAGTAGAGCCT |
| | | (SEQ ID NO: 38) |
| Ccdc177 | ENSMUST00000073251.6 | AACGAACGGAGGGTCATTGG GGTAC CACTCTACC TAT |
| | | CCACACACACCTTGGATGCTAAATG GAATTC CCCTAATGCGAGTAGAGCCT |
| | | (SEQ ID NO: 39) |
| Ccdc177 | ENSMUST00000073251.6 | AACGAACGGAGGGTCATTGG GGTAC CACTCTACC TAT |
| | | GACTTTCCACCTTCCCTGGTGAAGA GAATTC CCCTAATGCGAGTAGAGCCT |
| | | (SEQ ID NO: 40) |
| Ccdc177 | ENSMUST00000073251.6 | AACGAACGGAGGGTCATTGG GGTAC CACTCTACC TAT |
| | | CATTATCTCACACGTGACACCTAAA GAATTC CCCTAATGCGAGTAGAGCCT |
| | | (SEQ ID NO: 41) |
| Ccdc177 | ENSMUST00000073251.6 | AACGAACGGAGGGTCATTGG GGTAC CACTCTACC TAT |
| | | CTGTCTTAGAGACAACACCAGTTAT GAATTC CCCTAATGCGAGTAGAGCCT |
| | | (SEQ ID NO: 42) |
| Ccdc177 | ENSMUST00000073251.6 | AACGAACGGAGGGTCATTGG GGTAC CACTCTACC TAT |
| | | AATGCTCATGCTTTGAAGTAAGTAC GAATTC CCCTAATGCGAGTAGAGCCT |
| | | (SEQ ID NO: 43) |
| Ccdc177 | ENSMUST00000073251.6 | AACGAACGGAGGGTCATTGG GGTAC CACTCTACC TAT |
| | | GTCGGTGAGGACTAAGAAAGGTGAC GAATTC CCCTAATGCGAGTAGAGCCT |
| | | (SEQ ID NO: 44) |
| Ccdc177 | ENSMUST00000073251.6 | AACGAACGGAGGGTCATTGG GGTAC CACTCTACC TAT |
| | | CCGTGTACGGCAACTGCACCTTCAG GAATTC CCCTAATGCGAGTAGAGCCT |
| | | (SEQ ID NO: 45) |
| Ccdc177 | ENSMUST00000073251.6 | AACGAACGGAGGGTCATTGG GGTAC CACTCTACC TAT |
| | | ACGTCACATGTGACTCTGGATCTGA GAATTC CCCTAATGCGAGTAGAGCCT |
| | | (SEQ ID NO: 46) |
| Ccdc177 | ENSMUST00000073251.6 | AACGAACGGAGGGTCATTGG GGTAC CACTCTACC TAT |
| | | ATATGGTCTGGAACTTGAAGGTACC GAATTC CCCTAATGCGAGTAGAGCCT |
| | | (SEQ ID NO: 47) |
| Ccdc177 | ENSMUST00000073251.6 | AACGAACGGAGGGTCATTGG GGTAC CACTCTACC TAT |
| | | CTATCTGGTATGGGCTGACTCAATA GAATTC CCCTAATGCGAGTAGAGCCT |
| | | (SEQ ID NO: 48) |
| Ccdc177 | ENSMUST00000073251.6 | AACGAACGGAGGGTCATTGG GGTACC GTCTCTCCATGGGAGACTACGAACT TAT |
| | | ATTTAACCG AATTC CCCTAATGCGAGTAGAGCCT |
| | | (SEQ ID NO: 49) |
| Ccdc177 | ENSMUST00000073251.6 | AACGAACGGAGGGTCATTGG GGTACC GCTGCTGGGCTCCCTGGGCATCGGG |
| | | TAT ATTTAACCG AATTC CCCTAATGCGAGTAGAGCCT |
| | | (SEQ ID NO: 50) |
| Ccdc177 | ENSMUST00000073251.6 | AACGAACGGAGGGTCATTGG GGTACC CGGGCGCAGGCCTCCAGAGAGCGGG |
| | | TAT ATTTAACCG AATTC CCCTAATGCGAGTAGAGCCT |
| | | (SEQ ID NO: 51) |
| Ccdc177 | ENSMUST00000073251.6 | AACGAACGGAGGGTCATTGG GGTACC GCACTGCTGCAGTTTGGCCAGGCGC |
| | | TAT ATTTAACCG AATTC CCCTAATGCGAGTAGAGCCT |
| | | (SEQ ID NO: 52) |
| Ccdc177 | ENSMUST00000073251.6 | AACGAACGGAGGGTCATTGG GGTAGC CGGGCGGAGGCCGGCTCCTGGCGGG |
| | | TAT ATTTAACCG AATTC CCCTAATGCGAGTAGAGCCT |
| | | (SEQ ID NO: 53) |
| Ccdc177 | ENSMUST00000073251.6 | AACGAACGGAGGGTCATTGG GGTAGC CAGCGAAGCTCCCGAAGGCTCTCCC |
| | | TAT ATTTAACCG AATTC CCCTAATGCGAGTAGAGCCT |
| | | (SEQ ID NO: 54) |
| Ccdc177 | ENSMUST00000073251.6 | AACGAACGGAGGGTCATTGG GGTACC TGCGGGCCGTGAGCGGAACCAGAGC |
| | | TAT ATTTAACCG AATTC CCCTAATGCGAGTAGAGCCT |
| | | (SEQ ID NO: 55) |
| Ccdc177 | ENSMUST00000073251.6 | AACGAACGGAGGGTCATTGG GGTACC ATCTTGCGGTCGCGCTCGGGTACCC |
| | | TAT ATTTAACCG AATTC CCCTAATGCGAGTAGAGCCT |
| | | (SEQ ID NO: 56) |
| Ccdc177 | ENSMUST00000073251.6 | AACGAACGGAGGGTCATTGG GGTACC CGCCGCTGTTCGGCGCGCACGCGCT |
| | | TAT ATTTAACCG AATTC CCCTAATGCGAGTAGAGCCT |
| | | (SEQ ID NO: 57) |
| Ccdc177 | ENSMUST00000073251.6 | AACGAACGGAGGGTCATTGG GGTACC CGCCAGCTCCCGCCGCCGCTCCTCG |
| | | TAT ATTTAACCG AATTC CCCTAATGCGAGTAGAGCCT |
| | | (SEQ ID NO: 58) |
| Ccdc177 | ENSMUST00000073251.6 | AACGAACGGAGGGTCATTGG GGTACC GGACCAGCTCCGCGCGCTCCCGCCC |
| | | TAT ATTTAACCG AATTC CCCTAATGCGAGTAGAGCCT |
| | | (SEQ ID NO: 59) |
| Ccdc177 | ENSMUST00000073251.6 | AACGAACGGAGGGTCATTGG GGTACC GCGCCTCGTGGCGCGCCCGCTCGGC |
| | | TAT ATTTAACCG AATTC CCCTAATGCGAGTAGAGCCT |
| | | (SEQ ID NO: 60) |
| Ccdc177 | ENSMUST00000073251.6 | AACGAACGGAGGGTCATTGG GGTACC GTTCCTGCAGCTGCTCGTAGTTCTC TAT |
| | | ATTTAACCG AATTC CCCTAATGCGAGTAGAGCCT |
| | | (SEQ ID NO: 61) |
| Ccdc177 | ENSMUST00000073251.6 | AACGAACGGAGGGTCATTGG GGTACC CCTCCAAGTGCGCCTGATGCTCCCG |
| | | TAT ATTTAACCG AATTC CCCTAATGCGAGTAGAGCCT |
| | | (SEQ ID NO: 62) |
| Ccdc177 | ENSMUST00000073251.6 | AACGAACGGAGGGTCATTGG GGTACC GTTGGCACGCTGGGCCCTCTCCTTT TAT |
| | | ATTTAACCG AATTC CCCTAATGCGAGTAGAGCCT |
| | | (SEQ ID NO: 63) |
| Ccdc177 | ENSMUST00000073251.6 | AACGAACGGAGGGTCATTGG GGTACC CGCGCTTCGCCGTTCTCGAGAGAGC |
| | | TAT ATTTAACCG AATTC CCCTAATGCGAGTAGAGCCT |
| | | (SEQ ID NO: 64) |
| Ccdc177 | ENSMUST00000073251.6 | AACGAACGGAGGGTCATTGG GGTACC CAGCACGGTCTGTCCAGGGCGGCTG |
| | | TAT ATTTAACCG AATTC CCCTAATGCGAGTAGAGCCT |
| | | (SEQ ID NO: 65) |
| Ccdc177 | ENSMUST00000073251.6 | AACGAACGGAGGGTCATTGG GGTACC CATTAATGGTTCATTAGTCTGGAGA TAT |
| | | ATTTAACCG AATTC CCCTAATGCGAGTAGAGCCT |
| | | (SEQ ID NO: 66) |
| Ccdc177 | ENSMUST00000073251.6 | AACGAACGGAGGGTCATTGG GGTACC CTTTCTATTCCAACCCAAAGACACC TAT |
| | | ATTTAACCG AATTC CCCTAATGCGAGTAGAGCCT |
| | | (SEQ ID NO: 67) |
| Ccdc177 | ENSMUST00000073251.6 | AACGAACGGAGGGTCATTGG GGTACC TTCCCTCCGCTGCTGCTACTTAAAT TAT |
| | | ATTTAACCG AATTC CCCTAATGCGAGTAGAGCCT |
| | | (SEQ ID NO: 68) |
| Ccdc177 | ENSMUST00000073251.6 | AACGAACGGAGGGTCATTGG GGTACC CAATTCACAGTCTAGCGATAGCTTT TAT |
| | | ATTTAACCG AATTC CCCTAATGCGAGTAGAGCCT |
| | | (SEQ ID NO: 69) |
| Ccdc177 | ENSMUST00000073251.6 | AACGAACGGAGGGTCATTGG GGTACC CTAAGCAGCCTCACCGATCCAGCCC |
| | | TAT ATTTAACCG AATTC CCCTAATGCGAGTAGAGCCT |
| | | (SEQ ID NO: 70) |
| Ccdc177 | ENSMUST00000073251.6 | AACGAACGGAGGGTCATTGG GGTACC GGCTTCTGTCTGCAGGAGCTGGCCC |
| | | TAT ATTTAACCG AATTC CCCTAATGCGAGTAGAGCCT |
| | | (SEQ ID NO: 71) |
| Ccdc177 | ENSMUST00000073251.6 | AACGAACGGAGGGTCATTGG GGTACC GAACGCTGAACTAAGTCACAGAGGG |
| | | TAT ATTTAACCG AATTC CCCTAATGCGAGTAGAGCCT |
| | | (SEQ ID NO: 72) |
| Ccdc177 | ENSMUST00000073251.6 | AACGAACGGAGGGTCATTGG GGTACC GAATTTAAAGAGGGAAGACTCCTTT TAT |
| | | ATTTAACCG AATTC CCCTAATGCGAGTAGAGCCT |
| | | (SEQ ID NO: 73) |
| Ccdc177 | ENSMUST00000073251.6 | AACGAACGGAGGGTCATTGG GGTACC ACAAAGGAGACCAACAAATAACACT TAT |
| | | ATTTAACCG AATTC CCCTAATGCGAGTAGAGCCT |
| | | (SEQ ID NO: 74) |
| Ccdc177 | ENSMUST00000073251.6 | AACGAACGGAGGGTCATTGG GGTACC CATTTGTCTGTGATGCACTGCCCTG TAT |
| | | ATTTAACCG AATTC CCCTAATGCGAGTAGAGCCT |
| | | (SEQ ID NO: 75) |
| Ccdc177 | ENSMUST00000073251.6 | AACGAACGGAGGGTCATTGG GGTACC AAGCTGACGGGTGGATCTGAGTGGG |
| | | TAT ATTTAACCG AATTC CCCTAATGCGAGTAGAGCCT |
| | | (SEQ ID NO: 76) |
| Ccdc177 | ENSMUST00000073251.6 | AACGAACGGAGGGTCATTGG GGTACC CAGCTTTCTTCTACACAAGACTCCC TAT |
| | | ATTTAACCG AATTC CCCTAATGCGAGTAGAGCCT |
| | | (SEQ ID NO: 77) |
| Ccdc177 | ENSMUST00000073251.6 | AACGAACGGAGGGTCATTGG GGTACC CAGCTCACTGACAAGGAAATTAAGG TAT |
| | | ATTTAACCG AATTC CCCTAATGCGAGTAGAGCCT |
| | | (SEQ ID NO: 78) |
| Ccdc177 | ENSMUST00000073251.6 | AACGAACGGAGGGTCATTGG GGTACC TCAAGGGTAGAACTTTCCATGGAGG TAT |
| | | ATTTAACCG AATTC CCCTAATGCGAGTAGAGCCT |
| | | (SEQ ID NO: 79) |
| Ccdc177 | ENSMUST00000073251.6 | AACGAACGGAGGGTCATTGG GGTACC ACCTGAAGTCGCTCAGTAAGGTGGG |
| | | TAT ATTTAACCG AATTC CCCTAATGCGAGTAGAGCCT |
| | | (SEQ ID NO: 80) |
| Ccdc177 | ENSMUST00000073251.6 | AACGAACGGAGGGTCATTGG GGTACC CCAAGGTGACAGAAACAGGAAATAT TAT |
| | | ATTTAACCG AATTC CCCTAATGCGAGTAGAGCCT |
| | | (SEQ ID NO: 81) |
| Ccdc177 | ENSMUST00000073251.6 | AACGAACGGAGGGTCATTGG GGTACC GAGCTTCTCAAGACTGTTCCTTCTG TAT |
| | | ATTTAACCG AATTC CCCTAATGCGAGTAGAGCCT |
| | | (SEQ ID NO: 82) |
| Ccdc177 | ENSMUST00000073251.6 | AACGAACGGAGGGTCATTGG GGTACC TTCTTACTGTCACTAGGTCTGAATT TAT |
| | | ATTTAACCG AATTC CCCTAATGCGAGTAGAGCCT |
| | | (SEQ ID NO: 83) |
| Ccdc177 | ENSMUST00000073251.6 | AACGAACGGAGGGTCATTGG GGTACC ATTATCCATCTACTTCAGCATCCTC TAT |
| | | ATTTAACCG AATTC CCCTAATGCGAGTAGAGCCT |
| | | (SEQ ID NO: 84) |
| Ccdc177 | ENSMUST00000073251.6 | AACGAACGGAGGGTCATTGG GGTAC CACTTACCC TAT |
| | | TCTTCTGGCACCGGGTCCACCATAG GAATTC CCCTAATGCGAGTAGAGCCT |
| | | (SEQ ID NO: 85) |
| Ccdc177 | ENSMUST00000073251.6 | AACGAACGGAGGGTCATTGG GGTAC CACTTACCC TAT |
| | | AAGAGATCGAGGTGCAGCAACGGAG GAATTC CCCTAATGCGAGTAGAGCCT |
| | | (SEQ ID NO: 86) |
| Ccdc177 | ENSMUST00000073251.6 | AACGAACGGAGGGTCATTGG GGTAC CACTTACCC TAT |
| | | CGCCTCGTAGGCCTCGTACAGGCCG GAATTC CCCTAATGCGAGTAGAGCCT |
| | | (SEQ ID NO: 87) |
| Ccdc177 | ENSMUST00000073251.6 | AACGAACGGAGGGTCATTGG GGTAC CACTTACCC TAT |
| | | GGCGGGCGAGGCCGGGAGGCTGCTG GAATTC CCCTAATGCGAGTAGAGCCT |
| | | (SEQ ID NO: 88) |
| Ccdc177 | ENSMUST00000073251.6 | AACGAACGGAGGGTCATTGG GGTAC CACTTACCC TAT |
| | | CGGACTCGGAGCTCAGGGCACCGTC GAATTC CCCTAATGCGAGTAGAGCCT |
| | | (SEQ ID NO: 89) |
| Ccdc177 | ENSMUST00000073251.6 | AACGAACGGAGGGTCATTGG GGTAC CACTTACCC TAT |
| | | GGGCCGAAGGGCGGCCCAGTGGGTT GAATTC CCCTAATGCGAGTAGAGCCT |
| | | (SEQ ID NO: 90) |
| Ccdc177 | ENSMUST00000073251.6 | AACGAACGGAGGGTCATTGG GGTAC CACTTACCC TAT |
| | | GCTCGGCGCGCACTTGGCGGACGAT GAATTC CCCTAATGCGAGTAGAGCCT |
| | | (SEQ ID NO: 91) |
| Ccdc177 | ENSMUST00000073251.6 | AACGAACGGAGGGTCATTGG GGTAC CACTTACCC TAT |
| | | TGCTCCCACTGGCCGTGGGCCGCGG GAATTC CCCTAATGCGAGTAGAGCCT |
| | | (SEQ ID NO: 92) |
| Ccdc177 | ENSMUST00000073251.6 | AACGAACGGAGGGTCATTGG GGTAC CACTTACCC TAT |
| | | GCGCTCGCACTGCTGCTGCCGCCTC GAATTC CCCTAATGCGAGTAGAGCCT |
| | | (SEQ ID NO: 93) |
| Ccdc177 | ENSMUST00000073251.6 | AACGAACGGAGGGTCATTGG GGTAC CACTTACCC TAT |
| | | CTGCTCCTGCTGCAGCTTGCGCAGC GAATTC CCCTAATGCGAGTAGAGCCT |
| | | (SEQ ID NO: 94) |
| Ccdc177 | ENSMUST00000073251.6 | AACGAACGGAGGGTCATTGG GGTAC CACTTACCC TAT |
| | | GGCTCAGCTCGCGCTTCTCGCGCTG GAATTC CCCTAATGCGAGTAGAGCCT |
| | | (SEQ ID NO: 95) |
| Ccdc177 | ENSMUST00000073251.6 | AACGAACGGAGGGTCATTGG GGTAC CACTTACCC TAT |
| | | GCGCGCGGCCCAAGCTGGCCTCCAG GAATTC CCCTAATGCGAGTAGAGCCT |
| | | (SEQ ID NO: 96) |
| Ccdc177 | ENSMUST00000073251.6 | AACGAACGGAGGGTCATTGG GGTAC CACTTACCC TAT |
| | | CTTTGCGCTCGGCCGCTTCCTTCGC GAATTC CCCTAATGCGAGTAGAGCCT |
| | | (SEQ ID NO: 97) |
| Ccdc177 | ENSMUST00000073251.6 | AACGAACGGAGGGTCATTGG GGTAC CACTTACCC TAT |
| | | CGCGCCGGGCCTTCTGCTGCACAGC GAATTC CCCTAATGCGAGTAGAGCCT |
| | | (SEQ ID NO: 98) |
| Ccdc177 | ENSMUST00000073251.6 | AACGAACGGAGGGTCATTGG GGTAC CACTTACCC TAT |
| | | CTCACTGCGCTCCAGCTTGCGCCCA GAATTC CCCTAATGCGAGTAGAGCCT |
| | | (SEQ ID NO: 99) |
| Ccdc177 | ENSMUST00000073251.6 | AACGAACGGAGGGTCATTGG GGTAC CACTTACCC TAT |
| | | ATTTGCGGTCCAGGCTGGCATGTAG GAATTC CCCTAATGCGAGTAGAGCCT |
| | | (SEQ ID NO: 100) |
| Ccdc177 | ENSMUST00000073251.6 | AACGAACGGAGGGTCATTGG GGTAC CACTTACCC TAT |
| | | TCATAGTGTGGCAACTGGACTGGAG GAATTC CCCTAATGCGAGTAGAGCCT |
| | | (SEQ ID NO: 101) |
| Ccdc177 | ENSMUST00000073251.6 | AACGAACGGAGGGTCATTGG GGTAC CACTTACCC TAT |
| | | TGGATGTGGGTAGATTGTGCCATGT GAATTC CCCTAATGCGAGTAGAGCCT |
| | | (SEQ ID NO: 102) |
| Ccdc177 | ENSMUST00000073251.6 | AACGAACGGAGGGTCATTGG GGTAC CACTTACCC TAT |
| | | TCTTGAACACAATTGTTAGAAAGAT GAATTC CCCTAATGCGAGTAGAGCCT |
| | | (SEQ ID NO: 103) |
| Ccdc177 | ENSMUST00000073251.6 | AACGAACGGAGGGTCATTGG GGTAC CACTTACCC TAT |
| | | AGACGAGCTGTGCTTTCTTGGGAAA GAATTC CCCTAATGCGAGTAGAGCCT |
| | | (SEQ ID NO: 104) |
| Ccdc177 | ENSMUST00000073251.6 | AACGAACGGAGGGTCATTGG GGTAC CACTTACCC TAT |
| | | TTATTATCTGTAGATGATTGTCTAA GAATTC CCCTAATGCGAGTAGAGCCT |
| | | (SEQ ID NO: 105) |
| Ccdc177 | ENSMUST00000073251.6 | AACGAACGGAGGGTCATTGG GGTAC CACTTACCC TAT |
| | | CATCAGACACTGCCTCCACTTACGT GAATTC CCCTAATGCGAGTAGAGCCT |
| | | (SEQ ID NO: 106) |
| Ccdc177 | ENSMUST00000073251.6 | AACGAACGGAGGGTCATTGG GGTAC CACTTACCC TAT |
| | | TCCAGACTGCATTCAAGGCTGGAGC GAATTC CCCTAATGCGAGTAGAGCCT |
| | | (SEQ ID NO: 107) |
| Ccdc177 | ENSMUST00000073251.6 | AACGAACGGAGGGTCATTGG GGTAC CACTTACCC TAT |
| | | AACAATAGCACAGCTCCACACAGGC GAATTC CCCTAATGCGAGTAGAGCCT |
| | | (SEQ ID NO: 108) |
| Ccdc177 | ENSMUST00000073251.6 | AACGAACGGAGGGTCATTGG GGTAC CACTTACCC TAT |
| | | AGTGATGCTTTGGATCCAAGAGACC GAATTC CCCTAATGCGAGTAGAGCCT |
| | | (SEQ ID NO: 109) |
| Ccdc177 | ENSMUST00000073251.6 | AACGAACGGAGGGTCATTGG GGTAC CACTTACCC TAT |
| | | TAATGCCAGCAATCCATGTCACCAG GAATTC CCCTAATGCGAGTAGAGCCT |
| | | (SEQ ID NO: 110) |
| Ccdc177 | ENSMUST00000073251.6 | AACGAACGGAGGGTCATTGG GGTAC CACTTACCC TAT |
| | | AACTGAACCTTCTGACCAGGGAGCG GAATTC CCCTAATGCGAGTAGAGCCT |
| | | (SEQ ID NO: 111) |
| Ccdc177 | ENSMUST00000073251.6 | AACGAACGGAGGGTCATTGG GGTAC CACTTACCC TAT |
| | | AGACACACTAACACGTCTAATGCAG GAATTC CCCTAATGCGAGTAGAGCCT |
| | | (SEQ ID NO: 112) |
| Ccdc177 | ENSMUST00000073251.6 | AACGAACGGAGGGTCATTGG GGTAC CACTTACCC TAT |
| | | GAACCCACCCACTCAAGAAACAAGC GAATTC CCCTAATGCGAGTAGAGCCT |
| | | (SEQ ID NO: 113) |
| Ccdc177 | ENSMUST00000073251.6 | AACGAACGGAGGGTCATTGG GGTAC CACTTACCC TAT |
| | | TCAGAAGCAACCCAGGGACCACAAG GAATTC CCCTAATGCGAGTAGAGCCT |
| | | (SEQ ID NO: 114) |
| Ccdc177 | ENSMUST00000073251.6 | AACGAACGGAGGGTCATTGG GGTAC CACTTACCC TAT |
| | | TCGCTCGCGTGCTTCCCAGTGCTTG GAATTC CCCTAATGCGAGTAGAGCCT |
| | | (SEQ ID NO: 115) |
| Ccdc177 | ENSMUST00000073251.6 | AACGAACGGAGGGTCATTGG GGTAC CACTTACCC TAT |
| | | CCGTCATTCTCGTTTGCACAAACTA GAATTC CCCTAATGCGAGTAGAGCCT |
| | | (SEQ ID NO: 116) |
| Ccdc177 | ENSMUST00000073251.6 | AACGAACGGAGGGTCATTGG GGTAC CACTTACCC TAT |
| | | CCAAATCCTGATGGTACATTACACT GAATTC CCCTAATGCGAGTAGAGCCT |
| | | (SEQ ID NO: 117) |
| Ccdc177 | ENSMUST00000073251.6 | AACGAACGGAGGGTCATTGG GGTAC CACTTACCC TAT |
| | | TTACATAGTGGTTCTCTCAGTGTCT GAATTC CCCTAATGCGAGTAGAGCCT |
| | | (SEQ ID NO: 118) |
| Ccdc177 | ENSMUST00000073251.6 | AACGAACGGAGGGTCATTGG GGTAC CACTTACCC TAT |
| | | GGTTAGGTATAGAGATGTGAGCCAC GAATTC CCCTAATGCGAGTAGAGCCT |
| | | (SEQ ID NO: 119) |
| Ccdc177 | ENSMUST00000073251.6 | AACGAACGGAGGGTCATTGG GGTAC CACTTACCC TAT |
| | | GAAAGAAACATGAACATTTCTTTAG GAATTC CCCTAATGCGAGTAGAGCCT |
| | | (SEQ ID NO: 120) |
| Ccdc177 | ENSMUST00000073251.6 | AACGAACGGAGGGTCATTGG GGTACC GAACCTCGTTCTTTGTTGGAGTCCC TAT |
| | | ATTTAACCG AATTC CCCTAATGCGAGTAGAGCCT |
| | | (SEQ ID NO: 121) |
| Ccdc177 | ENSMUST00000073251.6 | AACGAACGGAGGGTCATTGG GGTACC TGCTCCCGACGTCCACCTTCTGCCC TAT |
| | | ATTTAACCG AATTC CCCTAATGCGAGTAGAGCCT |
| | | (SEQ ID NO: 122) |
| Ccdc177 | ENSMUST00000073251.6 | AACGAACGGAGGGTCATTGG GGTACC GGCCACGCGCATGGAGCGGCCCGGG |
| | | TAT ATTTAACCG AATTC CCCTAATGCGAGTAGAGCCT |
| | | (SEQ ID NO: 123) |
| Ccdc177 | ENSMUST00000073251.6 | AACGAACGGAGGGTCATTGG GGTACC GCTGCTGCAGCTGCTGCTGCTGCCC |
| | | TAT ATTTAACCG AATTC CCCTAATGCGAGTAGAGCCT |
| | | (SEQ ID NO: 124) |
| Ccdc177 | ENSMUST00000073251.6 | AACGAACGGAGGGTCATTGG GGTACC TCCGGCGGGAGAGCGAGTCCAGCGA |
| | | TAT ATTTAACCG AATTC CCCTAATGCGAGTAGAGCCT |
| | | (SEQ ID NO: 125) |
| Ccdc177 | ENSMUST00000073251.6 | AACGAACGGAGGGTCATTGG GGTAGC GTGCAGACGACGCCGAGCCCGCGGG |
| | | TAT ATTTAACCG AATTC CCCTAATGCGAGTAGAGCCT |
| | | (SEQ ID NO: 126) |
| Ccdc177 | ENSMUST00000073251.6 | AACGAACGGAGGGTCATTGG GGTAGC GCTCCACATGCTGGGCTGTCTGCGG |
| | | TAT ATTTAACCG AATTC CCCTAATGCGAGTAGAGCCT |
| | | (SEQ ID NO: 127) |
| Ccdc177 | ENSMUST00000073251.6 | AACGAACGGAGGGTCATTGG GGTACC CGCTGCTCCAGCAACAGGCGCTCTT |
| | | TAT ATTTAACCG AATTC CCCTAATGCGAGTAGAGCCT |
| | | (SEQ ID NO: 128) |
| Ccdc177 | ENSMUST00000073251.6 | AACGAACGGAGGGTCATTGG GGTACC CGCGGCCTCGCGCTCCTCCCGGCCC |
| | | TAT ATTTAACCG AATTC CCCTAATGCGAGTAGAGCCT |
| | | (SEQ ID NO: 129) |
| Ccdc177 | ENSMUST00000073251.6 | AACGAACGGAGGGTCATTGG GGTACC GTCGTCCCGCGCGGCGCGTTCCACC |
| | | TAT ATTTAACCG AATTC CCCTAATGCGAGTAGAGCCT |
| | | (SEQ ID NO: 130) |
| Ccdc177 | ENSMUST00000073251.6 | AACGAACGGAGGGTCATTGG GGTACC GTTGGCCCTCTTGCCGCTCTCGGGC |
| | | TAT ATTTAACCG AATTC CCCTAATGCGAGTAGAGCCT |
| | | (SEQ ID NO: 131) |
| Ccdc177 | ENSMUST00000073251.6 | AACGAACGGAGGGTCATTGG GGTACC AGCTCCGCAGGCCCTCCCGCTCCTC |
| | | TAT ATTTAACCG AATTC CCCTAATGCGAGTAGAGCCT |
| | | (SEQ ID NO: 132) |
| Ccdc177 | ENSMUST00000073251.6 | AACGAACGGAGGGTCATTGG GGTACC GCCGACCCTGGAGCTCCTCTCTCCG |
| | | TAT ATTTAACCG AATTC CCCTAATGCGAGTAGAGCCT |
| | | (SEQ ID NO: 133) |
| Ccdc177 | ENSMUST00000073251.6 | AACGAACGGAGGGTCATTGG GGTACC CTTCGGCCACCTGCGCCGCGTGCTG |
| | | TAT ATTTAACCG AATTC CCCTAATGCGAGTAGAGCCT |
| | | (SEQ ID NO: 134) |
| Ccdc177 | ENSMUST00000073251.6 | AACGAACGGAGGGTCATTGG GGTACC GGCCTGCAGCAGCTCGCGGCGGCGG |
| | | TAT ATTTAACCG AATTC CCCTAATGCGAGTAGAGCCT |
| | | (SEQ ID NO: 135) |
| Ccdc177 | ENSMUST00000073251.6 | AACGAACGGAGGGTCATTGG GGTACC GGGCCTCTCGCACCATGCGGTCAAA |
| | | TAT ATTTAACCG AATTC CCCTAATGCGAGTAGAGCCT |
| | | (SEQ ID NO: 136) |
| Ccdc177 | ENSMUST00000073251.6 | AACGAACGGAGGGTCATTGG GGTACC GCCTGGAGTTTGGTCAAGGTGACTA TAT |
| | | ATTTAACCG AATTC CCCTAATGCGAGTAGAGCCT |
| | | (SEQ ID NO: 137) |
| Ccdc177 | ENSMUST00000073251.6 | AACGAACGGAGGGTCATTGG GGTACC TTTCCAAATGGAGTAAGTCTGTAAA TAT |
| | | ATTTAACCG AATTC CCCTAATGCGAGTAGAGCCT |
| | | (SEQ ID NO: 138) |
| Ccdc177 | ENSMUST00000073251.6 | AACGAACGGAGGGTCATTGG GGTACC CCAAGGCAGCTGTGGCAGAGAGCCC |
| | | TAT ATTTAACCG AATTC CCCTAATGCGAGTAGAGCCT |
| | | (SEQ ID NO: 139) |
| Ccdc177 | ENSMUST00000073251.6 | AACGAACGGAGGGTCATTGG GGTACC TTCAAGTCCTGTGCAACTTGTATTT TAT |
| | | ATTTAACCG AATTC CCCTAATGCGAGTAGAGCCT |
| | | (SEQ ID NO: 140) |
| Ccdc177 | ENSMUST00000073251.6 | AACGAACGGAGGGTCATTGG GGTACC AGAAAGCCAAGGGACTCAACTGAAA TAT |
| | | ATTTAACCG AATTC CCCTAATGCGAGTAGAGCCT |
| | | (SEQ ID NO: 141) |
| Ccdc177 | ENSMUST00000073251.6 | AACGAACGGAGGGTCATTGG GGTACC TCACCTTCCATGGAAGCCTCATGGG TAT |
| | | ATTTAACCG AATTC CCCTAATGCGAGTAGAGCCT |
| | | (SEQ ID NO: 142) |
| Ccdc177 | ENSMUST00000073251.6 | AACGAACGGAGGGTCATTGG GGTACC TAAGCAAGAGTGGCCTGGGCAGGCA |
| | | TAT ATTTAACCG AATTC CCCTAATGCGAGTAGAGCCT |
| | | (SEQ ID NO: 143) |
| Ccdc177 | ENSMUST00000073251.6 | AACGAACGGAGGGTCATTGG GGTACC TTCTAGCCCTTCAGCCACCACAGGCTAT |
| | | ATTTAACCG AATTC CCCTAATGCGAGTAGAGCCT |
| | | (SEQ ID NO: 144) |
| Ccdc177 | ENSMUST00000073251.6 | AACGAACGGAGGGTCATTGG GGTACC GAGGTACCAGAAGAGTTGATGGCCC |
| | | TAT ATTTAACCG AATTC CCCTAATGCGAGTAGAGCCT |
| | | (SEQ ID NO: 145) |
| Ccdc177 | ENSMUST00000073251.6 | AACGAACGGAGGGTCATTGG GGTACC ACAAACTAGGCTAACAGCTGTCCTC TAT |
| | | ATTTAACCG AATTC CCCTAATGCGAGTAGAGCCT |
| | | (SEQ ID NO: 146) |
| Ccdc177 | ENSMUST00000073251.6 | AACGAACGGAGGGTCATTGG GGTACC TTGGTATTGCAAGTACCAAGTACCC TAT |
| | | ATTTAACCG AATTC CCCTAATGCGAGTAGAGCCT |
| | | (SEQ ID NO: 147) |
| Ccdc177 | ENSMUST00000073251.6 | AACGAACGGAGGGTCATTGG GGTACC CGTCTCTACCCTGATTCCTTTCTTT TAT |
| | | ATTTAACCG AATTC CCCTAATGCGAGTAGAGCCT |
| | | (SEQ ID NO: 148) |
| Ccdc177 | ENSMUST00000073251.6 | AACGAACGGAGGGTCATTGG GGTACC CAGTACATAGCTCCTTTCCTGGTAC TAT |
| | | ATTTAACCG AATTC CCCTAATGCGAGTAGAGCCT |
| | | (SEQ ID NO: 149) |
| Ccdc177 | ENSMUST00000073251.6 | AACGAACGGAGGGTCATTGG GGTACC CAAAGGACCATAATCACATTAATTT TAT |
| | | ATTTAACCG AATTC CCCTAATGCGAGTAGAGCCT |
| | | (SEQ ID NO: 150) |
| Ccdc177 | ENSMUST00000073251.6 | AACGAACGGAGGGTCATTGG GGTAOC TCAGAACACCCGATTGCCTCCTCAA TAT |
| | | ATTTAACCG AATTC CCCTAATGCGAGTAGAGCCT |
| | | (SEQ ID NO: 151) |
| Ccdc177 | ENSMUST00000073251.6 | AACGAACGGAGGGTCATTGG GGTAOC GCTCAGGAACCATGATCTCAGTTCT TAT |
| | | ATTTAACCG AATTC CCCTAATGCGAGTAGAGCCT |
| | | (SEQ ID NO: 152) |
| Ccdc177 | ENSMUST00000073251.6 | AACGAACGGAGGGTCATTGG GGTACC ACAGTACGGCGCAGGAGCCATGGAG |
| | | TAT ATTTAACCG AATTC CCCTAATGCGAGTAGAGCCT |
| | | (SEQ ID NO: 153) |
| Ccdc177 | ENSMUST00000073251.6 | AACGAACGGAGGGTCATTGG GGTAOC TTTGTCAACATTCTGGCCTGGGCCT TAT |
| | | ATTTAACCG AATTC CCCTAATGCGAGTAGAGCCT |
| | | (SEQ ID NO: 154) |
| Ccdc177 | ENSMUST00000073251.6 | AACGAACGGAGGGTCATTGG GGTACC TGTCCAACTTCAGTAGAGTATCCTA TAT |
| | | ATTTAACCG AATTC CCCTAATGCGAGTAGAGCCT |
| | | (SEQ ID NO: 155) |
| Ccdc177 | ENSMUST00000073251.6 | AACGAACGGAGGGTCATTGG GGTACC TATACAGGTGATTCTCCTTGCATTT TAT |
| | | ATTTAACCG AATTC CCCTAATGCGAGTAGAGCCT |
| | | (SEQ ID NO: 156) |

TABLE 2
Codebook for each gene in the 317-genes split probe library.
The binary code word assigned to each gene in the 317-genes
split probe library.

| Gene | Code-word |
|---|---|
| Ccdc177 | 0 1 0 0 1 0 0 0 0 0 0 0 0 0 0 0 0 0 0 0 0 0 0 0 0 0 |
| RP23-383N15.1 | 0 0 0 0 0 0 0 0 0 0 0 0 0 0 0 0 0 0 0 1 0 1 0 0 0 0 |
| RP24-338G10.1 | 0 0 0 0 0 0 0 1 0 0 0 0 0 0 0 0 0 0 0 0 1 0 0 0 0 0 |
| Myh11 | 0 0 0 0 0 0 0 0 1 0 0 0 0 1 0 0 0 0 0 0 0 0 0 0 0 0 |
| Xlrp1 | 0 0 0 0 0 0 0 0 0 0 0 0 0 0 0 0 0 0 0 0 0 0 0 0 1 1 |
| Col6a5 | 0 0 0 0 0 0 0 0 0 0 0 0 0 0 0 1 0 0 0 0 0 0 0 1 0 0 |
| Abca9 | 0 0 0 0 0 0 1 0 0 0 0 0 0 0 0 0 0 0 1 0 0 0 0 0 0 0 |
| Pkdrej | 0 0 0 0 1 0 0 0 0 0 0 0 0 0 0 1 0 0 0 0 0 0 0 0 0 0 |
| Col6a6 | 0 0 0 0 0 0 1 0 0 0 1 0 0 0 0 0 0 0 0 0 0 0 0 0 0 0 |
| Igsf10 | 0 1 0 1 0 0 0 0 0 0 0 0 0 0 0 0 0 0 0 0 0 0 0 0 0 0 |
| Tmc3 | 0 0 0 0 0 0 0 0 0 0 1 0 0 1 0 0 0 0 0 0 0 0 0 0 0 0 |
| Wdfy4 | 0 0 0 0 0 0 0 0 0 0 0 0 0 0 0 0 0 0 0 0 1 0 0 1 0 0 |
| Scn10a | 0 0 0 0 0 1 0 0 1 0 0 0 0 0 0 0 0 0 0 0 0 0 0 0 0 0 |
| Fat4 | 0 0 0 0 0 0 0 0 0 0 0 0 0 0 0 0 0 0 0 0 0 1 0 0 0 1 |
| Hmcn2 | 0 0 0 0 0 0 0 0 0 1 0 0 0 0 1 0 0 0 0 0 0 0 0 0 0 0 |
| Zfp83l | 0 0 1 0 0 0 0 0 0 0 0 0 0 0 0 1 0 0 0 0 0 0 0 0 0 0 |
| Map3k9 | 0 0 0 0 1 0 0 0 0 0 0 0 0 0 0 0 0 0 0 0 0 1 0 0 0 0 |
| Scn7a | 0 0 0 0 0 0 0 0 0 0 1 0 0 0 0 0 1 0 0 0 0 0 0 0 0 0 |
| Nat8l | 0 0 0 1 0 1 0 0 0 0 0 0 0 0 0 0 0 0 0 0 0 0 0 0 0 0 |
| Sdk1 | 0 0 0 1 0 0 0 0 0 0 0 0 0 0 0 0 0 0 0 0 0 0 0 0 1 0 |
| Dnah8 | 0 0 0 0 0 0 0 0 0 0 0 0 1 0 1 0 0 0 0 0 0 0 0 0 0 0 |
| Dnah11 | 0 0 1 0 0 0 0 0 0 0 0 1 0 0 0 0 0 0 0 0 0 0 0 0 0 0 |
| Pappa | 0 0 1 0 0 0 0 0 0 0 0 0 0 1 0 0 0 0 0 0 0 0 0 0 0 0 |
| Spag17 | 0 0 0 0 0 0 0 0 1 0 0 0 0 0 0 0 0 0 1 0 0 0 0 0 0 0 |
| Trabd2b | 0 0 0 0 0 0 0 0 0 0 0 0 0 0 1 0 0 0 0 1 0 0 0 0 0 0 |
| Prtg | 0 0 0 1 0 0 0 0 0 0 1 0 0 0 0 0 0 0 0 0 0 0 0 0 0 0 |
| Grik3 | 0 0 0 0 0 0 0 0 0 0 1 0 1 0 0 0 0 0 0 0 0 0 0 0 0 0 |
| Gpr179 | 0 0 0 0 0 0 0 0 0 1 0 0 0 1 0 0 0 0 0 0 0 0 0 0 0 0 |
| Edaradd | 0 0 0 0 0 0 0 0 0 0 1 0 0 0 0 0 0 0 0 0 0 0 1 0 0 0 |
| Akap6 | 0 0 0 0 0 0 0 0 0 0 0 1 0 0 0 0 0 0 0 0 0 0 0 1 0 0 |
| Adamts17 | 0 0 0 0 0 0 0 0 0 0 0 0 1 1 0 0 0 0 0 0 0 0 0 0 0 0 |
| Map1a | 0 0 0 0 0 0 1 0 1 0 0 0 0 0 0 0 0 0 0 0 0 0 0 0 0 0 |
| Bend4 | 0 0 0 1 1 0 0 0 0 0 0 0 0 0 0 0 0 0 0 0 0 0 0 0 0 0 |
| Reps2 | 0 0 0 0 0 0 0 0 0 0 0 0 0 0 1 0 0 0 0 0 0 1 0 0 0 0 |
| Armcx4 | 0 0 1 0 0 0 0 0 0 0 0 0 1 0 0 0 0 0 0 0 0 0 0 0 0 0 |
| Plcb2 | 0 0 0 0 0 0 0 0 0 0 1 1 0 0 0 0 0 0 0 0 0 0 0 0 0 0 |
| Adam19 | 0 0 0 0 0 0 1 0 0 0 0 0 0 0 0 1 0 0 0 0 0 0 0 0 0 0 |
| Galnt15 | 0 0 1 0 1 0 0 0 0 0 0 0 0 0 0 0 0 0 0 0 0 0 0 0 0 0 |
| Pygo1 | 0 0 0 0 0 0 0 0 0 0 0 0 0 0 0 0 0 0 0 1 0 0 0 0 1 0 |
| Tnxb | 1 0 0 1 0 0 0 0 0 0 0 0 0 0 0 0 0 0 0 0 0 0 0 0 0 0 |
| Stab2 | 1 0 0 0 0 0 0 0 0 0 0 0 0 1 0 0 0 0 0 0 0 0 0 0 0 0 |
| Ptprz1 | 0 0 1 0 0 0 0 0 0 0 0 0 0 0 0 0 0 0 0 0 0 0 0 1 0 0 |
| Kif5a | 0 0 0 0 0 0 0 0 0 0 0 0 0 1 0 0 0 0 0 0 1 0 0 0 0 0 |
| Dnah1 | 0 0 0 0 1 1 0 0 0 0 0 0 0 0 0 0 0 0 0 0 0 0 0 0 0 0 |
| Chst2 | 0 0 0 0 0 0 1 0 0 0 0 0 1 0 0 0 0 0 0 0 0 0 0 0 0 0 |
| Cacna1c | 0 0 1 0 0 0 0 0 0 0 0 0 0 0 0 0 0 0 0 0 0 0 0 0 1 0 |
| Col4a4 | 0 0 0 0 0 0 0 0 0 1 0 0 0 0 0 0 0 0 0 0 0 0 0 0 1 0 |
| Scn4a | 0 0 0 0 0 0 0 0 0 0 0 0 0 0 0 0 1 0 0 0 0 0 1 0 0 0 |
| Sdk2 | 0 0 0 0 0 0 0 0 0 0 0 0 0 1 1 0 0 0 0 0 0 0 0 0 0 0 |
| Pcdhb19 | 0 0 0 0 0 0 0 0 0 0 0 0 0 0 0 0 0 0 1 1 0 0 0 0 0 0 |
| Myom2 | 0 1 0 0 0 0 0 0 0 1 0 0 0 0 0 0 0 0 0 0 0 0 0 0 0 0 |
| Fgd5 | 0 0 0 0 0 0 0 0 0 0 1 0 0 0 0 0 0 0 0 0 0 1 0 0 0 0 |
| Kif1a | 0 1 0 0 0 1 0 0 0 0 0 0 0 0 0 0 0 0 0 0 0 0 0 0 0 0 |
| Caskin1 | 0 0 0 0 0 1 0 0 0 0 0 0 0 0 0 1 0 0 0 0 0 0 0 0 0 0 |
| Csmd1 | 0 0 0 0 0 0 0 0 0 0 0 0 0 0 0 1 0 0 0 1 0 0 0 0 0 0 |
| Acacb | 0 1 0 0 0 0 0 0 0 0 0 0 0 0 0 1 0 0 0 0 0 0 0 0 0 0 |
| Ksr2 | 0 0 0 0 1 0 0 0 0 0 0 0 1 0 0 0 0 0 0 0 0 0 0 0 0 0 |
| Mgam | 0 0 0 0 0 0 0 0 0 0 0 0 0 0 0 0 0 1 1 0 0 0 0 0 0 0 |
| Plxnc1 | 1 0 0 0 0 0 0 0 0 0 0 0 0 0 1 0 0 0 0 0 0 0 0 0 0 0 |
| Pcdhb22 | 0 0 0 0 0 0 0 0 0 0 0 0 1 0 0 0 0 0 0 1 0 0 0 0 0 0 |
| Impg2 | 0 0 0 0 0 0 0 0 0 0 0 0 0 1 0 0 0 0 0 0 0 0 0 0 0 1 |
| Sptbn2 | 0 1 0 0 0 0 0 0 0 0 0 0 0 0 0 0 0 0 0 0 0 1 0 0 0 0 |
| Rnf150 | 0 0 0 0 0 0 0 1 0 0 0 0 1 0 0 0 0 0 0 0 0 0 0 0 0 0 |
| Myh7b | 0 0 0 1 0 0 0 0 0 0 0 0 0 0 0 1 0 0 0 0 0 0 0 0 0 0 |
| Adamts2 | 1 0 0 0 0 0 0 0 0 0 0 0 0 0 0 0 0 0 0 0 0 0 0 1 0 0 |
| Ppfia2 | 0 0 0 0 0 1 0 0 0 0 0 0 0 0 0 0 0 0 0 1 0 0 0 0 0 0 |
| Camk4 | 1 0 0 0 0 1 0 0 0 0 0 0 0 0 0 0 0 0 0 0 0 0 0 0 0 0 |
| Rims3 | 0 0 0 0 0 0 0 0 0 0 0 0 0 1 0 0 0 0 0 0 0 0 0 0 1 0 |
| Fign | 0 1 0 0 0 0 0 0 0 0 1 0 0 0 0 0 0 0 0 0 0 0 0 0 0 0 |
| Gpr116 | 1 0 0 0 1 0 0 0 0 0 0 0 0 0 0 0 0 0 0 0 0 0 0 0 0 0 |
| Prune2 | 1 0 0 0 0 0 0 0 0 0 0 0 0 0 0 0 0 0 0 0 0 0 0 0 1 0 |
| Erc2 | 0 0 0 0 0 0 0 1 0 0 0 0 0 0 0 0 0 0 0 0 0 0 1 0 0 0 |
| Abca1 | 0 0 0 0 0 0 0 0 0 0 0 0 1 0 0 0 1 0 0 0 0 0 0 0 0 0 |
| Notch3 | 0 0 0 0 0 0 0 0 0 0 0 0 0 0 0 0 0 1 0 0 0 1 0 0 0 0 |
| Dock10 | 0 0 0 0 0 0 0 0 0 0 0 1 0 0 0 0 0 0 0 0 0 0 0 0 0 1 |
| Ntrk2 | 1 0 0 0 0 0 0 0 0 1 0 0 0 0 0 0 0 0 0 0 0 0 0 0 0 0 |
| Synm | 0 0 0 0 0 0 0 0 0 0 0 0 0 0 0 0 0 0 0 0 1 0 0 0 0 1 |
| Pkd1l3 | 0 0 0 0 0 0 1 0 0 0 0 0 0 0 0 0 0 0 0 0 0 1 0 0 0 0 |
| Mrc2 | 0 0 0 0 0 0 0 0 0 0 0 0 0 0 0 0 1 0 1 0 0 0 0 0 0 0 |
| Foxo1 | 0 0 0 0 1 0 0 0 0 0 0 0 0 0 0 0 0 0 0 1 0 0 0 0 0 0 |
| Il17rd | 1 0 0 0 0 0 0 0 0 0 0 0 0 0 0 0 0 0 0 0 0 1 0 0 0 0 |
| Myom3 | 0 0 0 0 0 0 0 1 0 0 0 1 0 0 0 0 0 0 0 0 0 0 0 0 0 0 |
| Rassf4 | 0 0 0 0 0 0 0 1 0 0 0 0 0 0 0 1 0 0 0 0 0 0 0 0 0 0 |
| Ddr2 | 0 1 0 0 0 0 0 0 0 0 0 0 0 0 0 0 0 1 0 0 0 0 0 0 0 0 |
| Trpm6 | 0 0 0 0 0 0 0 1 0 0 0 0 0 0 0 0 0 0 0 0 0 0 0 0 1 0 |
| Spon1 | 0 0 0 0 0 0 1 0 0 0 0 0 0 0 0 0 0 0 0 0 0 0 0 1 0 0 |
| Celsr1 | 0 0 0 0 0 1 0 1 0 0 0 0 0 0 0 0 0 0 0 0 0 0 0 0 0 0 |
| Aldh1l2 | 0 0 0 0 0 0 0 0 0 0 0 0 1 0 0 0 0 0 0 0 1 0 0 0 0 0 |
| Parp14 | 0 0 0 1 0 0 0 0 0 0 0 0 0 0 0 0 0 0 1 0 0 0 0 0 0 0 |
| Daam2 | 0 0 0 0 0 0 0 0 0 0 0 0 0 0 0 0 1 0 0 0 0 0 0 1 0 0 |
| Ago4 | 0 0 0 0 0 0 0 0 1 0 0 0 0 0 0 0 0 0 0 1 0 0 0 0 0 0 |
| Ctif | 0 0 0 0 0 0 0 0 0 0 0 0 0 0 0 0 0 0 0 0 0 1 0 1 0 0 |
| Setd1b | 0 0 0 0 0 0 1 0 0 0 0 0 0 1 0 0 0 0 0 0 0 0 0 0 0 0 |
| Pla2r1 | 0 0 0 0 0 0 0 0 0 0 1 0 0 0 0 0 0 0 0 0 0 0 0 0 0 1 |
| Gpr161 | 0 0 0 0 0 1 0 0 0 0 0 0 0 0 0 0 0 0 0 0 0 0 0 1 0 0 |
| Dock6 | 0 0 1 0 0 1 0 0 0 0 0 0 0 0 0 0 0 0 0 0 0 0 0 0 0 0 |
| Gm20342 | 0 0 0 0 0 0 0 0 0 0 0 0 0 0 0 0 0 1 0 0 0 0 1 0 0 0 |
| Trpm2 | 0 0 0 0 0 0 0 0 0 0 0 0 0 0 0 0 0 1 0 0 1 0 0 0 0 0 |
| Lgr6 | 0 0 0 0 0 0 0 0 0 0 1 0 0 0 0 0 0 0 0 0 0 0 0 0 1 0 |
| Fry | 0 0 0 0 0 0 0 0 0 1 0 0 1 0 0 0 0 0 0 0 0 0 0 0 0 0 |
| Flrt2 | 0 0 0 0 0 0 0 0 0 0 0 0 0 0 1 0 0 0 0 0 0 0 0 0 0 1 |
| Ptprm | 0 1 0 0 0 0 0 0 0 0 0 0 0 0 0 0 0 0 0 0 0 0 0 0 1 0 |
| Srgap3 | 0 0 0 1 0 0 0 0 0 0 0 0 0 0 0 0 0 0 0 0 0 1 0 0 0 0 |
| Dock8 | 0 0 0 0 0 0 0 0 0 0 0 1 0 0 0 1 0 0 0 0 0 0 0 0 0 0 |
| Shroom3 | 0 0 0 1 0 0 0 0 0 0 0 1 0 0 0 0 0 0 0 0 0 0 0 0 0 0 |
| Sned1 | 0 0 0 0 0 0 0 0 0 0 0 0 0 0 0 0 0 0 0 0 0 0 1 1 0 0 |
| Gm29666 | 0 0 0 0 0 0 0 0 0 0 0 1 0 0 0 0 0 0 0 0 0 1 0 0 0 0 |
| Nhsl2 | 0 0 0 0 0 0 0 0 0 0 0 0 0 0 0 0 0 0 0 0 0 0 1 0 1 0 |
| Arhgap31 | 0 0 0 0 0 0 0 1 0 0 0 0 0 0 0 0 0 1 0 0 0 0 0 0 0 0 |
| Zfp334 | 0 0 0 1 0 0 0 0 0 0 0 0 0 0 0 0 0 0 0 1 0 0 0 0 0 0 |
| Cdyl2 | 0 0 0 0 0 0 0 0 0 0 0 1 0 0 0 0 0 1 0 0 0 0 0 0 0 0 |
| Veph1 | 1 0 0 0 0 0 0 0 0 0 0 0 0 0 0 1 0 0 0 0 0 0 0 0 0 0 |
| Slc38a1 | 0 0 0 0 0 0 0 0 0 0 0 0 0 0 0 0 1 0 0 0 1 0 0 0 0 0 |
| Hspg2 | 0 0 0 0 0 0 0 0 1 0 0 0 0 0 0 0 0 0 0 0 1 0 0 0 0 0 |
| Itga4 | 1 0 0 0 0 0 0 0 0 0 0 0 0 0 0 0 0 0 0 1 0 0 0 0 0 0 |
| Amer1 | 0 0 0 0 0 0 0 0 0 0 0 0 0 0 0 0 0 0 0 0 1 0 0 0 1 0 |
| Dzank1 | 0 0 0 0 0 1 0 0 0 0 0 0 0 0 0 0 0 0 0 0 1 0 0 0 0 0 |
| F8 | 0 0 0 0 0 0 0 0 0 0 0 0 1 0 0 0 0 1 0 0 0 0 0 0 0 0 |
| Nid1 | 0 0 0 0 0 1 0 0 0 0 0 0 0 0 0 0 1 0 0 0 0 0 0 0 0 0 |
| Atp1a2 | 0 0 0 0 0 0 0 0 0 0 0 0 0 0 0 1 0 0 0 0 0 0 0 0 0 1 |
| Kcnq1ot1 | 0 1 0 0 0 0 0 0 0 0 0 0 0 0 0 0 0 0 1 0 0 0 0 0 0 0 |
| Notch4 | 0 0 0 0 1 0 0 0 0 0 0 0 0 0 0 0 0 0 0 0 0 0 0 1 0 0 |
| Myh10 | 0 1 0 0 0 0 1 0 0 0 0 0 0 0 0 0 0 0 0 0 0 0 0 0 0 0 |
| Col4a6 | 0 0 0 1 0 0 0 0 0 0 0 0 0 0 1 0 0 0 0 0 0 0 0 0 0 0 |
| Fbxo32 | 0 0 0 0 0 0 0 1 0 0 0 0 0 0 0 0 0 0 1 0 0 0 0 0 0 0 |
| Hivep2 | 0 0 1 0 0 0 0 0 0 0 1 0 0 0 0 0 0 0 0 0 0 0 0 0 0 0 |
| Plxna3 | 0 0 0 0 0 0 0 0 0 0 0 0 0 0 0 0 0 0 0 0 0 0 1 0 0 1 |
| Cacna1e | 0 0 0 0 0 0 0 0 0 0 0 0 0 0 0 0 0 0 0 1 1 0 0 0 0 0 |
| Phlpp2 | 0 0 0 0 1 0 0 0 1 0 0 0 0 0 0 0 0 0 0 0 0 0 0 0 0 0 |
| Nup210l | 0 0 0 0 0 0 0 0 0 1 0 1 0 0 0 0 0 0 0 0 0 0 0 0 0 0 |
| Helz2 | 0 0 0 0 0 0 0 0 0 0 1 0 0 0 0 0 0 1 0 0 0 0 0 0 0 0 |
| Mixip | 0 0 0 0 1 0 0 0 0 1 0 0 0 0 0 0 0 0 0 0 0 0 0 0 0 0 |
| Mkl2 | 0 0 0 0 0 0 0 0 0 0 0 1 0 0 0 0 0 0 1 0 0 0 0 0 0 0 |
| Prkaa2 | 0 0 1 0 0 0 0 0 0 0 0 0 0 0 0 0 0 0 0 0 0 0 1 0 0 0 |
| Nhsl1 | 0 0 0 0 0 0 0 0 0 0 0 0 0 0 1 1 0 0 0 0 0 0 0 0 0 0 |
| Sox6 | 0 0 0 0 0 0 0 0 0 0 0 0 0 0 0 1 0 1 0 0 0 0 0 0 0 0 |
| Szt2 | 0 0 0 0 0 0 0 0 0 0 0 0 0 0 0 0 0 0 0 0 0 1 0 0 1 0 |
| Nfia | 0 0 0 0 0 0 0 1 1 0 0 0 0 0 0 0 0 0 0 0 0 0 0 0 0 0 |
| Qser1 | 0 0 0 0 0 0 0 0 0 0 0 0 0 0 0 1 1 0 0 0 0 0 0 0 0 0 |
| Elmsan1 | 0 0 0 0 0 0 0 0 0 0 0 0 0 1 0 0 1 0 0 0 0 0 0 0 0 0 |
| Abca4 | 0 0 1 0 0 0 0 0 0 0 0 0 0 0 0 0 0 1 0 0 0 0 0 0 0 0 |
| Dennd4a | 0 0 0 0 0 0 0 0 1 0 0 0 0 0 0 0 0 0 0 0 0 0 0 0 1 0 |
| Nr6a1 | 0 0 0 0 0 0 0 0 0 0 0 0 0 0 1 0 0 0 0 0 1 0 0 0 0 0 |
| Crocc | 0 0 1 0 0 0 0 0 1 0 0 0 0 0 0 0 0 0 0 0 0 0 0 0 0 0 |
| Dennd3 | 0 0 0 0 0 0 0 0 0 0 0 0 0 0 0 0 0 0 0 0 0 0 0 1 0 1 |
| Tmed8 | 0 0 0 0 0 0 0 0 0 0 0 0 0 0 0 0 1 0 0 0 0 1 0 0 0 0 |
| Dock5 | 0 0 0 0 0 0 0 0 1 0 0 0 0 0 0 0 0 0 0 0 0 1 0 0 0 0 |
| Irs1 | 0 0 0 0 0 1 1 0 0 0 0 0 0 0 0 0 0 0 0 0 0 0 0 0 0 0 |
| Kdm7a | 0 0 0 0 0 0 0 0 0 0 0 0 0 0 1 0 0 0 1 0 0 0 0 0 0 0 |
| Nav2 | 0 0 0 0 0 0 0 0 0 1 0 0 0 0 0 0 0 0 0 0 1 0 0 0 0 0 |
| Ppm1l | 1 0 0 0 0 0 0 0 0 0 1 0 0 0 0 0 0 0 0 0 0 0 0 0 0 0 |
| Baz2a | 0 0 0 0 0 1 0 0 0 0 0 0 1 0 0 0 0 0 0 0 0 0 0 0 0 0 |
| Itpr2 | 0 0 0 0 0 0 0 0 0 0 0 0 0 0 1 0 0 1 0 0 0 0 0 0 0 0 |
| Arid1a | 0 0 0 1 0 0 0 0 0 1 0 0 0 0 0 0 0 0 0 0 0 0 0 0 0 0 |
| Mylk | 0 0 0 0 0 0 0 0 0 1 0 0 0 0 0 0 0 0 0 0 0 0 0 1 0 0 |
| Jrk | 0 0 0 0 1 0 0 0 0 0 0 1 0 0 0 0 0 0 0 0 0 0 0 0 0 0 |
| Crebbp | 0 0 0 0 1 0 0 0 0 0 0 0 0 0 0 0 0 1 0 0 0 0 0 0 0 0 |
| Dab2ip | 0 0 0 0 0 0 0 0 0 0 0 0 0 0 0 0 0 1 0 0 0 0 0 0 1 0 |
| Aim1 | 0 0 0 0 0 0 0 1 0 0 0 0 0 1 0 0 0 0 0 0 0 0 0 0 0 0 |
| Lrrk2 | 0 0 0 0 0 0 0 0 0 0 0 0 0 1 0 0 0 0 1 0 0 0 0 0 0 0 |
| Zfp516 | 0 0 0 0 0 0 0 0 0 0 0 0 0 0 0 0 1 1 0 0 0 0 0 0 0 0 |
| Thsd7a | 0 0 0 0 0 0 1 0 0 0 0 1 0 0 0 0 0 0 0 0 0 0 0 0 0 0 |
| Plxnd1 | 0 0 0 0 0 0 0 0 0 0 0 0 1 0 0 0 0 0 0 0 0 0 0 1 0 0 |
| Crb2 | 0 0 0 0 1 0 0 0 0 0 0 0 0 0 1 0 0 0 0 0 0 0 0 0 0 0 |
| Dock4 | 1 0 0 0 0 0 0 1 0 0 0 0 0 0 0 0 0 0 0 0 0 0 0 0 0 0 |
| Tbc1d32 | 0 1 0 0 0 0 0 0 0 0 0 0 0 0 0 0 1 0 0 0 0 0 0 0 0 0 |
| Onecut2 | 0 0 0 0 0 0 0 0 0 0 0 0 0 0 0 0 0 0 1 0 1 0 0 0 0 0 |
| Polr2a | 0 0 1 1 0 0 0 0 0 0 0 0 0 0 0 0 0 0 0 0 0 0 0 0 0 0 |
| Zfyve26 | 0 0 0 1 0 0 0 0 0 0 0 0 0 1 0 0 0 0 0 0 0 0 0 0 0 0 |
| Irgq | 0 1 0 0 0 0 0 0 0 0 0 0 0 0 0 0 0 0 0 0 0 0 0 1 0 0 |
| Cacna1a | 0 1 0 0 0 0 0 0 0 0 0 1 0 0 0 0 0 0 0 0 0 0 0 0 0 0 |
| Alms1 | 0 1 0 0 0 0 0 0 0 0 0 0 0 1 0 0 0 0 0 0 0 0 0 0 0 0 |
| Arhgef10 | 0 1 0 0 0 0 0 0 0 0 0 0 0 0 1 0 0 0 0 0 0 0 0 0 0 0 |
| Zbtb1 | 0 0 0 0 0 0 0 0 0 1 0 0 0 0 0 0 0 0 0 0 0 1 0 0 0 0 |
| Dip2c | 0 0 0 0 0 0 1 0 0 0 0 0 0 0 0 0 0 1 0 0 0 0 0 0 0 0 |
| Rttn | 0 0 0 0 0 0 1 0 0 0 0 0 0 0 0 0 0 0 0 1 0 0 0 0 0 0 |
| Prdm15 | 0 0 0 0 0 0 0 0 0 0 0 0 1 0 0 0 0 0 0 0 0 0 1 0 0 0 |
| Kmt2c | 0 0 0 0 0 0 0 0 0 0 1 0 0 0 1 0 0 0 0 0 0 0 0 0 0 0 |
| Lsm11 | 0 0 0 0 0 0 0 0 0 0 0 0 0 0 1 0 0 0 0 0 0 0 0 1 0 0 |
| Kif13a | 0 0 0 0 0 0 0 0 1 0 0 0 0 0 1 0 0 0 0 0 0 0 0 0 0 0 |
| Usp36 | 0 0 0 0 0 0 0 1 0 0 0 0 0 0 0 0 0 0 0 1 0 0 0 0 0 0 |
| Rnf213 | 0 0 0 0 0 0 0 0 0 0 0 0 0 0 0 0 0 0 0 0 0 0 0 1 1 0 |
| Spg11 | 0 0 0 0 0 0 0 0 0 0 0 0 1 0 0 0 0 0 1 0 0 0 0 0 0 0 |
| Dcaf5 | 1 1 0 0 0 0 0 0 0 0 0 0 0 0 0 0 0 0 0 0 0 0 0 0 0 0 |
| Cmah | 0 0 0 0 0 0 0 0 0 0 0 0 0 0 0 0 0 0 0 0 0 1 1 0 0 0 |
| Trim56 | 0 0 0 0 1 0 0 0 0 0 0 0 0 0 0 0 0 0 1 0 0 0 0 0 0 0 |
| Thada | 0 0 0 0 0 0 0 0 0 0 0 1 0 1 0 0 0 0 0 0 0 0 0 0 0 0 |
| Col7a1 | 0 0 0 0 0 0 0 0 0 0 1 0 0 0 0 1 0 0 0 0 0 0 0 0 0 0 |
| Fancm | 0 0 1 0 0 0 0 0 0 0 0 0 0 0 0 0 0 0 0 1 0 0 0 0 0 0 |
| Rprd2 | 0 0 0 0 1 0 0 0 0 0 0 0 0 1 0 0 0 0 0 0 0 0 0 0 0 0 |
| Rnf217 | 0 0 0 0 0 0 0 0 1 1 0 0 0 0 0 0 0 0 0 0 0 0 0 0 0 0 |
| Med12 | 0 0 0 0 0 0 0 0 0 0 0 1 0 0 0 0 0 0 0 0 1 0 0 0 0 0 |
| Sestd1 | 0 0 0 0 0 0 0 0 0 0 0 0 1 0 0 0 0 0 0 0 0 0 0 0 0 1 |
| Flnc | 0 0 0 0 1 0 0 0 0 0 0 0 0 0 0 0 0 0 0 0 1 0 0 0 0 0 |
| Itpr1 | 0 0 0 0 1 0 0 0 0 0 0 0 0 0 0 0 0 0 0 0 0 0 0 0 1 0 |
| AU022252 | 0 0 0 0 0 1 0 0 0 0 1 0 0 0 0 0 0 0 0 0 0 0 0 0 0 0 |
| Sh3pxd2b | 0 0 0 1 0 0 0 0 0 0 0 0 1 0 0 0 0 0 0 0 0 0 0 0 0 0 |
| Rfx7 | 0 0 0 0 0 0 0 0 0 0 0 0 0 1 0 1 0 0 0 0 0 0 0 0 0 0 |
| Wdr7 | 0 0 0 0 0 1 0 0 0 0 0 0 0 0 0 0 0 0 0 0 0 0 1 0 0 0 |
| Brca2 | 0 1 0 0 0 0 0 0 0 0 0 0 0 0 0 0 0 0 0 0 1 0 0 0 0 0 |
| Wdr81 | 0 0 0 0 0 0 0 0 0 0 0 0 0 0 0 0 0 0 0 0 1 0 1 0 0 0 |
| Tnrc18 | 0 0 0 0 0 0 0 0 0 0 0 0 0 0 0 1 0 0 1 0 0 0 0 0 0 0 |
| Ep300 | 1 0 0 0 0 0 0 0 0 0 0 0 0 0 0 0 0 0 0 0 0 0 1 0 0 0 |
| Piezo1 | 0 0 0 0 0 0 0 0 0 0 0 0 1 0 0 1 0 0 0 0 0 0 0 0 0 0 |
| Atxn1l | 0 0 1 0 0 0 0 0 0 0 0 0 0 0 0 0 0 0 0 0 0 1 0 0 0 0 |
| Plexd2 | 0 0 0 0 0 0 0 0 1 0 0 1 0 0 0 0 0 0 0 0 0 0 0 0 0 0 |
| Atg2b | 0 0 0 0 0 0 0 0 0 1 0 0 0 0 0 0 0 0 1 0 0 0 0 0 0 0 |
| Nf1 | 0 0 0 0 0 0 0 0 0 0 0 0 0 0 0 0 1 0 0 1 0 0 0 0 0 0 |
| Wdr90 | 0 0 0 0 0 0 1 0 0 0 0 0 0 0 1 0 0 0 0 0 0 0 0 0 0 0 |
| Rnf169 | 1 0 0 0 0 0 0 0 0 0 0 0 1 0 0 0 0 0 0 0 0 0 0 0 0 0 |
| Myo9a | 0 0 0 0 1 0 0 0 0 0 0 0 0 0 0 0 0 0 0 0 0 0 0 0 0 1 |
| Ly75 | 0 0 0 0 0 1 0 0 0 0 0 1 0 0 0 0 0 0 0 0 0 0 0 0 0 0 |
| Sox11 | 0 0 1 0 0 0 0 1 0 0 0 0 0 0 0 0 0 0 0 0 0 0 0 0 0 0 |
| Kif21a | 0 0 0 0 0 0 0 0 0 0 0 0 0 0 1 0 0 0 0 0 0 0 0 0 1 0 |
| Btrc | 1 0 0 0 0 0 0 0 1 0 0 0 0 0 0 0 0 0 0 0 0 0 0 0 0 0 |
| Dock9 | 1 0 0 0 0 0 0 0 0 0 0 0 0 0 0 0 1 0 0 0 0 0 0 0 0 0 |
| Ubn1 | 0 0 0 0 0 0 0 0 0 0 0 0 0 0 0 1 0 0 0 0 0 1 0 0 0 0 |
| Pask | 0 0 0 0 0 0 0 0 0 1 0 0 0 0 0 0 0 1 0 0 0 0 0 0 0 0 |
| Svil | 0 0 0 0 0 0 0 0 1 0 1 0 0 0 0 0 0 0 0 0 0 0 0 0 0 0 |
| Rapgef1 | 0 0 0 0 0 0 0 0 0 0 0 0 0 0 0 0 0 0 1 0 0 0 0 0 1 0 |
| Nedd4l | 0 0 0 0 0 0 0 0 0 0 0 0 0 0 0 0 1 0 0 0 0 0 0 0 0 1 |
| Gas2l3 | 0 0 0 0 0 0 0 0 0 0 0 0 0 0 0 0 0 0 1 0 0 1 0 0 0 0 |
| 2410089E03Rik | 0 0 0 0 0 0 1 0 0 0 0 0 0 0 0 0 0 0 0 0 1 0 0 0 0 0 |
| Sec16a | 0 0 0 1 0 0 0 0 0 0 0 0 0 0 0 0 0 0 0 0 0 0 1 0 0 0 |
| Dpy19l4 | 0 0 0 0 0 0 0 0 0 1 0 0 0 0 0 0 0 0 0 1 0 0 0 0 0 0 |
| Ep400 | 0 0 0 0 0 0 0 1 0 1 0 0 0 0 0 0 0 0 0 0 0 0 0 0 0 0 |
| Nin | 0 0 0 0 0 0 0 0 0 0 0 0 0 0 0 0 0 1 0 0 0 0 0 1 0 0 |
| Nipbl | 0 0 0 0 0 0 0 0 0 0 0 0 0 0 0 1 0 0 0 0 0 0 1 0 0 0 |
| Utp14b | 0 0 0 0 0 0 0 0 0 0 0 0 0 1 0 0 0 1 0 0 0 0 0 0 0 0 |
| Slc12a7 | 0 0 0 0 0 0 0 0 0 0 0 0 0 0 1 0 0 0 0 0 0 0 1 0 0 0 |
| Akap11 | 0 0 0 0 0 0 0 0 0 0 0 0 0 0 0 1 0 0 0 0 0 0 0 0 1 0 |
| Smg7 | 0 0 0 1 0 0 0 0 1 0 0 0 0 0 0 0 0 0 0 0 0 0 0 0 0 0 |
| Brip1 | 0 0 0 0 0 0 0 0 0 0 0 1 1 0 0 0 0 0 0 0 0 0 0 0 0 0 |
| Ythdc2 | 0 0 0 0 0 0 0 0 0 0 0 0 0 0 0 0 0 0 1 0 0 0 0 0 0 1 |
| Rc3h1 | 0 0 0 1 0 0 0 0 0 0 0 0 0 0 0 0 0 1 0 0 0 0 0 0 0 0 |
| Utrn | 0 0 0 0 0 0 0 0 0 0 1 0 0 0 0 0 0 0 0 1 0 0 0 0 0 0 |
| Rsf1 | 0 0 0 0 0 0 0 0 0 0 0 0 0 0 0 0 0 0 0 1 0 0 1 0 0 0 |
| Zfyve16 | 0 0 0 1 0 0 0 0 0 0 0 0 0 0 0 0 0 0 0 0 0 0 0 1 0 0 |
| 4932438A13Rik | 0 0 0 0 0 0 0 0 0 0 0 0 0 0 0 0 1 0 0 0 0 0 0 0 1 0 |
| Cdc42bpb | 0 0 0 0 0 0 0 0 0 0 1 0 0 0 0 0 0 0 1 0 0 0 0 0 0 0 |
| Trove2 | 0 0 0 0 0 0 0 0 0 0 0 0 0 0 1 0 1 0 0 0 0 0 0 0 0 0 |
| Abcc2 | 0 0 0 0 0 0 1 0 0 0 0 0 0 0 0 0 1 0 0 0 0 0 0 0 0 0 |
| Gtf3c4 | 1 0 0 0 0 0 0 0 0 0 0 0 0 0 0 0 0 0 0 0 0 0 0 0 0 1 |
| 1700020I14Rik | 0 0 0 0 0 0 0 0 0 0 0 0 0 0 0 0 0 0 1 0 0 0 0 1 0 0 |
| Luzp1 | 1 0 0 0 0 0 0 0 0 0 0 1 0 0 0 0 0 0 0 0 0 0 0 0 0 0 |
| Abca3 | 1 0 0 0 0 0 1 0 0 0 0 0 0 0 0 0 0 0 0 0 0 0 0 0 0 0 |
| Dicer1 | 0 0 1 0 0 0 1 0 0 0 0 0 0 0 0 0 0 0 0 0 0 0 0 0 0 0 |
| Larp1 | 0 0 0 0 0 0 0 0 1 0 0 0 1 0 0 0 0 0 0 0 0 0 0 0 0 0 |
| Map4 | 0 0 0 0 0 0 1 0 0 1 0 0 0 0 0 0 0 0 0 0 0 0 0 0 0 0 |
| Ehbp1l1 | 0 0 0 0 0 0 0 0 0 0 0 0 0 0 0 0 0 1 0 1 0 0 0 0 0 0 |
| Uhrf1bp1l | 0 0 0 0 0 0 0 0 0 0 0 1 0 0 0 0 0 0 0 0 0 0 1 0 0 0 |
| Aspm | 0 0 0 0 0 0 0 0 0 1 1 0 0 0 0 0 0 0 0 0 0 0 0 0 0 0 |
| Osbpl8 | 0 0 0 0 0 0 0 0 0 0 0 1 0 0 0 0 0 0 0 1 0 0 0 0 0 0 |
| Dsp | 0 1 0 0 0 0 0 1 0 0 0 0 0 0 0 0 0 0 0 0 0 0 0 0 0 0 |
| Mysm1 | 0 0 0 0 0 1 0 0 0 1 0 0 0 0 0 0 0 0 0 0 0 0 0 0 0 0 |
| Arhgap35 | 0 0 0 0 0 0 0 0 1 0 0 0 0 0 0 0 0 0 0 0 0 0 1 0 0 0 |
| Plxna1 | 0 0 0 0 0 0 0 0 1 0 0 0 0 0 0 0 0 0 0 0 0 0 0 1 0 0 |
| Ppl | 0 0 0 0 0 0 0 0 0 0 0 0 0 0 0 1 0 0 0 0 1 0 0 0 0 0 |
| Eif5b | 0 0 0 0 0 0 0 1 0 0 0 0 0 0 0 0 1 0 0 0 0 0 0 0 0 0 |
| Atr | 0 0 1 0 0 0 0 0 0 1 0 0 0 0 0 0 0 0 0 0 0 0 0 0 0 0 |
| Psd3 | 0 0 0 0 1 0 0 0 0 0 1 0 0 0 0 0 0 0 0 0 0 0 0 0 0 0 |
| Tmem2 | 0 0 0 0 0 0 0 0 1 0 0 0 0 0 0 0 0 1 0 0 0 0 0 0 0 0 |
| Cenpe | 0 0 0 0 0 0 1 0 0 0 0 0 0 0 0 0 0 0 0 0 0 0 0 0 0 1 |
| Als2cl | 0 0 0 0 0 1 0 0 0 0 0 0 0 0 0 0 0 1 0 0 0 0 0 0 0 0 |
| Kntc1 | 0 0 0 0 0 0 0 0 0 0 1 0 0 0 0 0 0 0 0 0 1 0 0 0 0 0 |
| Zfp26 | 0 0 0 0 0 0 0 0 0 1 0 0 0 0 0 0 0 0 0 0 0 0 1 0 0 0 |
| Tspan7 | 1 0 1 0 0 0 0 0 0 0 0 0 0 0 0 0 0 0 0 0 0 0 0 0 0 0 |
| Nvl | 0 0 0 1 0 0 0 0 0 0 0 0 0 0 0 0 0 0 0 0 1 0 0 0 0 0 |
| Cacna2d1 | 0 0 0 0 0 0 0 1 0 0 0 0 0 0 0 0 0 0 0 0 0 0 0 1 0 0 |
| Arhgef12 | 0 0 0 0 0 0 0 0 0 0 0 0 0 0 0 0 0 0 0 1 0 0 0 1 0 0 |
| Slc4a7 | 0 0 0 0 0 0 0 0 0 0 0 0 0 0 0 0 0 1 0 0 0 0 0 0 0 1 |
| Sart1 | 0 0 0 0 0 0 1 1 0 0 0 0 0 0 0 0 0 0 0 0 0 0 0 0 0 0 |
| Brwd1 | 0 0 0 0 0 0 0 0 1 0 0 0 0 0 0 0 0 0 0 0 0 0 0 0 0 1 |
| Trim33 | 0 0 1 0 0 0 0 0 0 0 0 0 0 0 0 0 0 0 0 0 1 0 0 0 0 0 |
| Letm1 | 0 0 0 0 0 1 0 0 0 0 0 0 0 0 0 0 0 0 1 0 0 0 0 0 0 0 |
| Arfgef1 | 1 0 0 0 0 0 0 0 0 0 0 0 0 0 0 0 0 0 0 0 1 0 0 0 0 0 |
| Nbeal2 | 0 0 0 0 1 0 1 0 0 0 0 0 0 0 0 0 0 0 0 0 0 0 0 0 0 0 |
| Golga4 | 1 0 0 0 0 0 0 0 0 0 0 0 0 0 0 0 0 0 1 0 0 0 0 0 0 0 |
| Zbtb41 | 0 0 0 1 0 0 0 1 0 0 0 0 0 0 0 0 0 0 0 0 0 0 0 0 0 0 |
| Cgnl1 | 0 0 0 0 1 0 0 0 0 0 0 0 0 0 0 0 1 0 0 0 0 0 0 0 0 0 |
| Sbf1 | 0 0 0 0 0 0 0 0 0 1 0 0 0 0 0 1 0 0 0 0 0 0 0 0 0 0 |
| Prrc2c | 0 0 1 0 0 0 0 0 0 0 0 0 0 0 1 0 0 0 0 0 0 0 0 0 0 0 |
| Myo6 | 0 0 0 0 1 0 0 0 0 0 0 0 0 0 0 0 0 0 0 0 0 0 1 0 0 0 |
| Spag9 | 0 1 0 0 0 0 0 0 0 0 0 0 0 0 0 0 0 0 0 0 0 0 1 0 0 0 |
| Edem1 | 0 0 0 0 0 0 0 0 0 0 0 0 0 0 0 0 0 0 0 0 1 1 0 0 0 0 |
| Heatr1 | 0 0 0 0 0 0 0 0 1 0 0 0 0 0 0 1 0 0 0 0 0 0 0 0 0 0 |
| Arhgef5 | 0 0 0 0 0 0 0 0 0 1 0 0 0 0 0 0 1 0 0 0 0 0 0 0 0 0 |
| Dynd1h1 | 0 1 0 0 0 0 0 0 0 0 0 0 0 0 0 0 0 0 0 1 0 0 0 0 0 0 |
| Ogt | 0 0 0 0 0 0 0 0 0 0 0 0 1 0 0 0 0 0 0 0 0 0 0 0 1 0 |
| Macf1 | 0 0 0 0 0 0 0 1 0 0 1 0 0 0 0 0 0 0 0 0 0 0 0 0 0 0 |
| Utp20 | 0 0 0 0 0 0 0 0 0 0 0 0 0 1 0 0 0 0 0 0 0 0 1 0 0 0 |
| Smchd1 | 0 0 0 0 0 0 0 0 0 0 1 0 0 0 0 0 0 0 0 0 0 0 0 1 0 0 |
| Pdzd8 | 0 0 0 0 0 0 0 1 0 0 0 0 0 0 1 0 0 0 0 0 0 0 0 0 0 0 |
| Polr1a | 0 0 0 0 0 1 0 0 0 0 0 0 0 0 0 0 0 0 0 0 0 0 0 0 1 0 |
| Arhgef28 | 0 0 0 0 0 0 0 0 1 0 0 0 0 0 0 0 1 0 0 0 0 0 0 0 0 0 |
| Mki67 | 0 0 0 0 0 0 0 0 0 0 0 0 0 0 0 0 0 0 0 1 0 0 0 0 0 1 |
| Myo5c | 0 0 1 0 0 0 0 0 0 0 0 0 0 0 0 0 0 0 1 0 0 0 0 0 0 0 |
| Tjp1 | 0 0 1 0 0 0 0 0 0 0 0 0 0 0 0 0 0 0 0 0 0 0 0 0 0 1 |
| Son | 0 0 0 1 0 0 0 0 0 0 0 0 0 0 0 0 0 0 0 0 0 0 0 0 0 1 |
| Cnot1 | 0 1 0 0 0 0 0 0 0 0 0 0 1 0 0 0 0 0 0 0 0 0 0 0 0 0 |
| Mlec | 0 0 0 0 0 1 0 0 0 0 0 0 0 1 0 0 0 0 0 0 0 0 0 0 0 0 |
| Mllt4 | 0 0 0 0 0 0 1 0 0 0 0 0 0 0 0 0 0 0 0 0 0 0 1 0 0 0 |
| Prpf8 | 0 0 0 0 0 0 0 1 0 0 0 0 0 0 0 0 0 0 0 0 0 0 0 0 0 1 |
| Abhd | 0 0 0 0 0 0 0 0 0 0 0 1 0 0 0 0 0 0 0 0 0 0 0 0 1 0 |
| Myo18a | 0 0 0 0 0 0 0 0 0 0 0 0 1 0 0 0 0 0 0 0 0 1 0 0 0 0 |
| Sptlc2 | 0 0 0 0 0 0 0 0 0 0 0 1 0 0 1 0 0 0 0 0 0 0 0 0 0 0 |
| Ppp2ca | 0 0 0 0 0 0 0 0 0 0 0 1 0 0 0 0 1 0 0 0 0 0 0 0 0 0 |
| Aars | 0 1 0 0 0 0 0 0 1 0 0 0 0 0 0 0 0 0 0 0 0 0 0 0 0 0 |
| Pten | 0 0 0 0 0 1 0 0 0 0 0 0 0 0 0 0 0 0 0 0 0 1 0 0 0 0 |
| Pik3c2a | 0 0 0 1 0 0 0 0 0 0 0 0 0 0 0 0 1 0 0 0 0 0 0 0 0 0 |
| Slk | 0 1 1 0 0 0 0 0 0 0 0 0 0 0 0 0 0 0 0 0 0 0 0 0 0 0 |
| Cand1 | 0 0 1 0 0 0 0 0 0 0 0 0 0 0 0 0 1 0 0 0 0 0 0 0 0 0 |
| Tnpo1 | 1 0 0 0 0 0 0 0 0 0 0 0 0 0 0 0 0 1 0 0 0 0 0 0 0 0 |
| Myof | 0 0 0 0 1 0 0 1 0 0 0 0 0 0 0 0 0 0 0 0 0 0 0 0 0 0 |
| Flna | 0 0 0 0 0 1 0 0 0 0 0 0 0 0 0 0 0 0 0 0 0 0 0 0 0 1 |
| Kpnb1 | 0 0 0 0 0 1 0 0 0 0 0 0 0 0 1 0 0 0 0 0 0 0 0 0 0 0 |
| Ahnak | 0 0 0 0 0 0 0 0 0 0 0 0 0 1 0 0 0 0 0 0 0 0 0 1 0 0 |
| Biank1 | 0 0 0 0 0 0 0 0 0 0 0 0 0 0 0 0 0 0 1 0 0 0 1 0 0 0 |
| Blank2 | 0 0 0 0 0 0 0 0 0 0 0 0 0 1 0 0 0 0 0 0 0 1 0 0 0 0 |
| Blank3 | 0 1 0 0 0 0 0 0 0 0 0 0 0 0 0 0 0 0 0 0 0 0 0 0 0 1 |
| Blank4 | 0 0 0 0 0 0 1 0 0 0 0 0 0 0 0 0 0 0 0 0 0 0 0 0 1 0 |
| Blank5 | 0 0 0 0 0 0 0 0 0 0 0 0 0 1 0 0 0 0 0 1 0 0 0 0 0 0 |
| Blank6 | 0 0 0 0 0 0 0 1 0 0 0 0 0 0 0 0 0 0 0 0 0 1 0 0 0 0 |
| Blank7 | 0 0 0 0 0 0 0 0 0 1 0 0 0 0 0 0 0 0 0 0 0 0 0 0 0 1 |
| Blank8 | 0 0 0 1 0 0 1 0 0 0 0 0 0 0 0 0 0 0 0 0 0 0 0 0 0 0 |

TABLE 3
Bridge sequence for each bit in the 317-genes split probe library.
Each bridge sequence consists of three blocks (separated by spaces): a split
probe binding block in the centre, flanked by two readout binding blocks.
In the split probe binding block, the barcode sequences are in lowercase.
Bridge sequences used in AML-12, kidney, frontal cortex, and liver experiments
are shown. B1 to B13 were read out by Alexa750, and B14 to B26 were read out
by Cy5. For ovary experiments, B1, B3, B8 to B13, B15, and B17 to B20 were
read out by Cy5 and B2, B4 to B7, B14, B16, and B21 to B26 were
read out by Alexa750.

| Bit | Bridge sequence |
|---|---|
| B1 | ATTGTAAAGCGTGAGAAA GGgatagtgGCGGTTAAAT ATTGTAAAGCGTGAGAAA (SEQ ID NO: 157) |
| B2 | ATTGTAAAGCGTGAGAAA GGtagagtgGCGGTTAAAT ATTGTAAAGCGTGAGAAA (SEQ ID NO: 158) |
| B3 | ATTGTAAAGCGTGAGAAA GGattgaggGCGGTTAAAT ATTGTAAAGCGTGAGAAA (SEQ ID NO: 159) |
| B4 | ATTGTAAAGCGTGAGAAA GGgtgtgggGCGGTTAAAT ATTGTAAAGCGTGAGAAA (SEQ ID NO: 160) |
| B5 | ATTGTAAAGCGTGAGAAA GGgtaagtgGCGGTTAAAT ATTGTAAAGCGTGAGAAA (SEQ ID NO: 161) |
| B6 | ATTGTAAAGCGTGAGAAA GGtttggtgGCGGTTAAAT ATTGTAAAGCGTGAGAAA (SEQ ID NO: 162) |
| B7 | ATTGTAAAGCGTGAGAAA GGaaggttgGCGGTTAAAT ATTGTAAAGCGTGAGAAA (SEQ ID NO: 163) |
| B8 | ATTGTAAAGCGTGAGAAA GGtaatgggGCGGTTAAAT ATTGTAAAGCGTGAGAAA (SEQ ID NO: 164) |
| B9 | ATTGTAAAGCGTGAGAAA GGaagaaggGCGGTTAAAT ATTGTAAAGCGTGAGAAA (SEQ ID NO: 165) |
| B10 | ATTGTAAAGCGTGAGAAA GGagagttgGCGGTTAAAT ATTGTAAAGCGTGAGAAA (SEQ ID NO: 166) |
| B11 | ATTGTAAAGCGTGAGAAA GGtggtttgGCGGTTAAAT ATTGTAAAGCGTGAGAAA (SEQ ID NO: 167) |
| B12 | ATTGTAAAGCGTGAGAAA GGgagattgGCGGTTAAAT ATTGTAAAGCGTGAGAAA (SEQ ID NO: 168) |
| B13 | ATTGTAAAGCGTGAGAAA GGattagggGCGGTTAAAT ATTGTAAAGCGTGAGAAA (SEQ ID NO: 169) |
| B14 | TGATGGGTGCGTGAGTAA GGgtatgagGCGGTTAAAT TGATGGGTGCGTGAGTAA (SEQ ID NO: 170) |
| B15 | TGATGGGTGCGTGAGTAA GGgaagttgGCGGTTAAAT TGATGGGTGCGTGAGTAA (SEQ ID NO: 171) |
| B16 | TGATGGGTGCGTGAGTAA GGagatgtgGCGGTTAAAT TGATGGGTGCGTGAGTAA (SEQ ID NO: 172) |
| B17 | TGATGGGTGCGTGAGTAA GGaggaaagGCGGTTAAAT TGATGGGTGCGTGAGTAA (SEQ ID NO: 173) |
| B18 | TGATGGGTGCGTGAGTAA GGgagagggGCGGTTAAAT TGATGGGTGCGTGAGTAA (SEQ ID NO: 174) |
| B19 | TGATGGGTGCGTGAGTAA GGtaaggtgGCGGTTAAAT TGATGGGTGCGTGAGTAA (SEQ ID NO: 175) |
| B20 | TGATGGGTGCGTGAGTAA GGtgtttggGCGGTTAAAT TGATGGGTGCGTGAGTAA (SEQ ID NO: 176) |
| B21 | TGATGGGTGCGTGAGTAA GGttgttggGCGGTTAAAT TGATGGGTGCGTGAGTAA (SEQ ID NO: 177) |
| B22 | TGATGGGTGCGTGAGTAA GGagtgtagGCGGTTAAAT TGATGGGTGCGTGAGTAA (SEQ ID NO: 178) |
| B23 | TGATGGGTGCGTGAGTAA GGtaggtagGCGGTTAAAT TGATGGGTGCGTGAGTAA (SEQ ID NO: 179) |
| B24 | TGATGGGTGCGTGAGTAA GGttgtgtgGCGGTTAAAT TGATGGGTGCGTGAGTAA (SEQ ID NO: 180) |
| B25 | TGATGGGTGCGTGAGTAA GGttggaagGCGGTTAAAT TGATGGGTGCGTGAGTAA (SEQ ID NO: 181) |
| B26 | TGATGGGTGCGTGAGTAA GGttgagagGCGGTTAAAT TGATGGGTGCGTGAGTAA (SEQ ID NO: 182) |

TABLE 4
133-genes conventional library template sequences. Template sequences include
the forward and reverse primer sequences necessary for library amplification.
The primers used for PCR are ‘TGGTTCAATCGTATGCCCGT’ (SEQ ID NO: 183) and
‘TAATACGACTCACTATAGGGGTCACTTAGCCAACGCCGAT’ (SEQ ID NO: 184).

| Target Gene | Transcript ID | Template Sequence |
|---|---|---|
| Igf1 | ENSMUST00000095360.6 | TGGTTCAATCGTATGCCCGT CCCATGATCGTCCGATCTGGTCGGATTTGT |
| | | GTCTGATTCAAATAAGTCAAACCACACCTT |
| | | TTAGTCGTCGCAACGCCAGGCAACTCATGC ATCGGCGTTGGCTAAGTGAC |
| | | (SEQ ID NO: 185) |
| Igf1 | ENSMUST00000095360.6 | TGGTTCAATCGTATGCCCGT CCCATGATCGTCCGATCTGGTCGGATTTGT |
| | | ACCTGTGATTGTAATAGATACTATTTCAAA |
| | | TTAGTCGTCGCAACGCCAGGCAACTCATGC ATCGGCGTTGGCTAAGTGAC |
| | | (SEQ ID NO: 186) |
| Igf1 | ENSMUST00000095360.6 | TGGTTCAATCGTATGCCCGT CCCATGATCGTCCGATCTGGTCGGATTTGT |
| | | GGAGCTTTCCTGTAAACTTAATATACACTG |
| | | TTAGTCGTCGCAACGCCAGGCAACTCATGC ATCGGCGTTGGCTAAGTGAC |
| | | (SEQ ID NO: 187) |
| Igf1 | ENSMUST00000095360.6 | TGGTTCAATCGTATGCCCGT CCCATGATCGTCCGATCTGGTCGGATTTGT |
| | | AAGGTTCAGACAGTCTTATCTCTTTCCTTT |
| | | TTAGTCGTCGCAACGCCAGGCAACTCATGC ATCGGCGTTGGCTAAGTGAC |
| | | (SEQ ID NO: 188) |
| Igf1 | ENSMUST00000095360.6 | TGGTTCAATCGTATGCCCGT CCCATGATCGTCCGATCTGGTCGGATTTGT |
| | | GGACTAGGAGATATAGTTATATATATATTT |
| | | TTAGTCGTCGCAACGCCAGGCAACTCATGC ATCGGCGTTGGCTAAGTGAC |
| | | (SEQ ID NO: 189) |
| Igf1 | ENSMUST00000095360.6 | TGGTTCAATCGTATGCCCGT CCCATGATCGTCCGATCTGGTCGGATTTGT |
| | | AGCTGGATCCTTCCTTCTAAGCAGACACTA |
| | | TTAGTCGTCGCAACGCCAGGCAACTCATGC ATCGGCGTTGGCTAAGTGAC |
| | | (SEQ ID NO: 190) |
| Igf1 | ENSMUSTO0000095360.6 | TGGTTCAATCGTATGCCCGT CCCATGATCGTCCGATCTGGTCGGATTTGT |
| | | GAGCGTATGCACGCATGTGCAAATGCACAT |
| | | TTAGTCGTCGCAACGCCAGGCAACTCATGC ATCGGCGTTGGCTAAGTGAC |
| | | (SEQ ID NO: 191) |
| Igf1 | ENSMUSTO0000095360.6 | TGGTTCAATCGTATGCCCGT CCCATGATCGTCCGATCTGGTCGGATTTGT |
| | | CAACTGGATCTCTACAACATCCATGCATTT |
| | | TTAGTCGTCGCAACGCCAGGCAACTCATGC ATCGGCGTTGGCTAAGTGAC |
| | | (SEQ ID NO: 192) |
| Igf1 | ENSMUSTO0000095360.6 | TGGTTCAATCGTATGCCCGT CCCATGATCGTCCGATCTGGTCGGATTTGT |
| | | AATGAAATGATTGAGCCATCTAACTTTAAA |
| | | TTAGTCGTCGCAACGCCAGGCAACTCATGC ATCGGCGTTGGCTAAGTGAC |
| | | (SEQ ID NO: 193) |
| Igf1 | ENSMUSTO0000095360.6 | TGGTTCAATCGTATGCCCGT CCCATGATCGTCCGATCTGGTCGGATTTGT |
| | | ACATTCGCCTTCTCTCTCTCTCCCTCTTCT |
| | | TTAGTCGTCGCAACGCCAGGCAACTCATGC ATCGGCGTTGGCTAAGTGAC |
| | | (SEQ ID NO: 194) |
| Igf1 | ENSMUSTO0000095360.6 | TGGTTCAATCGTATGCCCGT CCCATGATCGTCCGATCTGGTCGGATTTGT |
| | | AAACCCAAACCCAACAACAACAACAACAAA |
| | | GTAAGCGCAACGGTGGACCGGAGACGACGG ATCGGCGTTGGCTAAGTGAC |
| | | (SEQ ID NO: 195) |
| Igf1 | ENSMUSTO0000095360.6 | TGGTTCAATCGTATGCCCGT CCCATGATCGTCCGATCTGGTCGGATTTGT |
| | | TGTGATCTCTTTATCATGTGCCTTACGAAT |
| | | GTAAGCGCAACGGTGGACCGGAGACGACGG ATCGGCGTTGGCTAAGTGAC |
| | | (SEQ ID NO: 196) |
| Igf1 | ENSMUSTO0000095360.6 | TGGTTCAATCGTATGCCCGT CCCATGATCGTCCGATCTGGTCGGATTTGT |
| | | AGACAGTCTTTAGTCTGGCCAGCCTCTAAA |
| | | GTAAGCGCAACGGTGGACCGGAGACGACGG ATCGGCGTTGGCTAAGTGAC |
| | | (SEQ ID NO: 197) |
| Igf1 | ENSMUSTO0000095360.6 | TGGTTCAATCGTATGCCCGT CCCATGATCGTCCGATCTGGTCGGATTTGT |
| | | AATACAAGTGTTAGGAAAGGGTGTGTCTAA |
| | | GTAAGCGCAACGGTGGACCGGAGACGACGG ATCGGCGTTGGCTAAGTGAC |
| | | (SEQ ID NO: 198) |
| Igf1 | ENSMUSTO0000095360.6 | TGGTTCAATCGTATGCCCGT CCCATGATCGTCCGATCTGGTCGGATTTGT |
| | | TATTGTTATTTGGTAGGTGTTTCGATGTTT |
| | | GTAAGCGCAACGGTGGACCGGAGACGACGG ATCGGCGTTGGCTAAGTGAC |
| | | (SEQ ID NO: 199) |
| Igf1 | ENSMUSTO0000095360.6 | TGGTTCAATCGTATGCCCGT CCCATGATCGTCCGATCTGGTCGGATTTGT |
| | | ATGAATGACCAAATATTACTTTCAGGTTTC |
| | | GTAAGCGCAACGGTGGACCGGAGACGACGG ATCGGCGTTGGCTAAGTGAC |
| | | (SEQ ID NO: 200) |
| Igf1 | ENSMUSTO0000095360.6 | TGGTTCAATCGTATGCCCGT CCCATGATCGTCCGATCTGGTCGGATTTGT |
| | | TGCGCATCCTCCCAAGTGCACAAACCATAA |
| | | GTAAGCGCAACGGTGGACCGGAGACGACGG ATCGGCGTTGGCTAAGTGAC |
| | | (SEQ ID NO: 201) |
| Igf1 | ENSMUSTO0000095360.6 | TGGTTCAATCGTATGCCCGT CCCATGATCGTCCGATCTGGTCGGATTTGT |
| | | TGTGTTTGGCAAACTGAGTAGAGTCTCATT |
| | | GTAAGCGCAACGGTGGACCGGAGACGACGG ATCGGCGTTGGCTAAGTGAC |
| | | (SEQ ID NO: 202) |
| Igf1 | ENSMUSTO0000095360.6 | TGGTTCAATCGTATGCCCGT CCCATGATCGTCCGATCTGGTCGGATTTGT |
| | | ACAATCTTGCTATATACATAGAGAACATTT |
| | | GTAAGCGCAACGGTGGACCGGAGACGACGG ATCGGCGTTGGCTAAGTGAC |
| | | (SEQ ID NO: 203) |
| Igf1 | ENSMUSTO0000095360.6 | TGGTTCAATCGTATGCCCGT CCCATGATCGTCCGATCTGGTCGGATTTGT |
| | | TTCCTTGCATCCTAGCAATTCAAGGGTTCT |
| | | GTAAGCGCAACGGTGGACCGGAGACGACGG ATCGGCGTTGGCTAAGTGAC |
| | | (SEQ ID NO: 204) |
| Igf1 | ENSMUSTO0000095360.6 | TGGTTCAATCGTATGCCCGT CCCATGATCGTCCGATCTGGTCGGATTTGT |
| | | CATCACCCTTGGCCTTTAAAGTGTGATAAA |
| | | CGGCCGAATTACATCCGTCGTAATCGAGGC ATCGGCGTTGGCTAAGTGAC |
| | | (SEQ ID NO: 205) |
| Igf1 | ENSMUSTO0000095360.6 | TGGTTCAATCGTATGCCCGT CCCATGATCGTCCGATCTGGTCGGATTTGT |
| | | TAAATGTAAGATAATGATTCAGTTAGTTAC |
| | | CGGCCGAATTACATCCGTCGTAATCGAGGC ATCGGCGTTGGCTAAGTGAC |
| | | (SEQ ID NO: 206) |
| Igf1 | ENSMUSTO0000095360.6 | TGGTTCAATCGTATGCCCGT CCCATGATCGTCCGATCTGGTCGGATTTGT |
| | | CTTCCCTTTCTTTGTCAGTAGATAAGCCCA |
| | | CGGCCGAATTACATCCGTCGTAATCGAGGC ATCGGCGTTGGCTAAGTGAC |
| | | (SEQ ID NO: 207) |
| Igf1 | ENSMUSTO0000095360.6 | TGGTTCAATCGTATGCCCGT CCCATGATCGTCCGATCTGGTCGGATTTGT |
| | | TAAGGGAACCATTCATAAACCACCTCTACA |
| | | CGGCCGAATTACATCCGTCGTAATCGAGGC ATCGGCGTTGGCTAAGTGAC |
| | | (SEQ ID NO: 208) |
| Igf1 | ENSMUSTO0000095360.6 | TGGTTCAATCGTATGCCCGT CCCATGATCGTCCGATCTGGTCGGATTTGT |
| | | AGTTGGAGAGAATTAGGTATTGGATAAAGG |
| | | CGGCCGAATTACATCCGTCGTAATCGAGGC ATCGGCGTTGGCTAAGTGAC |
| | | (SEQ ID NO: 209) |
| Igf1 | ENSMUSTO0000095360.6 | TGGTTCAATCGTATGCCCGT CCCATGATCGTCCGATCTGGTCGGATTTGT |
| | | CAAACAAGGGCAAGACTACATCTGCTACAG |
| | | CGGCCGAATTACATCCGTCGTAATCGAGGC ATCGGCGTTGGCTAAGTGAC |
| | | (SEQ ID NO: 210) |
| Igf1 | ENSMUSTO0000095360.6 | TGGTTCAATCGTATGCCCGT CCCATGATCGTCCGATCTGGTCGGATTTGT |
| | | ATTTCAATTGGGCAGTGACATTTAGAATTT |
| | | CGGCCGAATTACATCCGTCGTAATCGAGGC ATCGGCGTTGGCTAAGTGAC |
| | | (SEQ ID NO: 211) |
| Igf1 | ENSMUSTO0000095360.6 | TGGTTCAATCGTATGCCCGT CCCATGATCGTCCGATCTGGTCGGATTTGT |
| | | ATTATTATGATTAATTAGTACAATTAATTT |
| | | CGGCCGAATTACATCCGTCGTAATCGAGGC ATCGGCGTTGGCTAAGTGAC |
| | | (SEQ ID NO: 212) |
| Igf1 | ENSMUSTO0000095360.6 | TGGTTCAATCGTATGCCCGT CCCATGATCGTCCGATCTGGTCGGATTTGT |
| | | AAATCAACAGGAAACCAAACAATCAACAAA |
| | | CGGCCGAATTACATCCGTCGTAATCGAGGC ATCGGCGTTGGCTAAGTGAC |
| | | (SEQ ID NO: 213) |
| Igf1 | ENSMUSTO0000095360.6 | TGGTTCAATCGTATGCCCGT CCCATGATCGTCCGATCTGGTCGGATTTGT |
| | | ACAATTTACCTCCATAGCAAAGTCTACAAA |
| | | CGGCCGAATTACATCCGTCGTAATCGAGGC ATCGGCGTTGGCTAAGTGAC |
| | | (SEQ ID NO: 214) |
| Igf1 | ENSMUSTO0000095360.6 | TGGTTCAATCGTATGCCCGT TTAGTCGTCGCAACGCCAGGCAACTCATGC |
| | | GACTGAGTCCTTAGCTTGTCACTAATTAGT |
| | | GTAAGCGCAACGGTGGACCGGAGACGACGG ATCGGCGTTGGCTAAGTGAC |
| | | (SEQ ID NO: 215) |
| Igf1 | ENSMUSTO0000095360.6 | TGGTTCAATCGTATGCCCGT TTAGTCGTCGCAACGCCAGGCAACTCATGC |
| | | AAACGAAATGAGACTAGGTGATAGATCAAC |
| | | GTAAGCGCAACGGTGGACCGGAGACGACGG ATCGGCGTTGGCTAAGTGAC |
| | | (SEQ ID NO: 216) |
| Igf1 | ENSMUSTO0000095360.6 | TGGTTCAATCGTATGCCCGT TTAGTCGTCGCAACGCCAGGCAACTCATGC |
| | | ATTCCTTCCTACCTATCTTTATAGTTCTTT |
| | | GTAAGCGCAACGGTGGACCGGAGACGACGG ATCGGCGTTGGCTAAGTGAC |
| | | (SEQ ID NO: 217) |
| Igf1 | ENSMUSTO0000095360.6 | TGGTTCAATCGTATGCCCGT TTAGTCGTCGCAACGCCAGGCAACTCATGC |
| | | ACTTCAGATTGGGTGAAGTATTGCCAATTT |
| | | GTAAGCGCAACGGTGGACCGGAGACGACGG ATCGGCGTTGGCTAAGTGAC |
| | | (SEQ ID NO: 218) |
| Igf1 | ENSMUSTO0000095360.6 | TGGTTCAATCGTATGCCCGT TTAGTCGTCGCAACGCCAGGCAACTCATGC |
| | | AATGTAATTACTAAAGAAAGATATACCATT |
| | | GTAAGCGCAACGGTGGACCGGAGACGACGG ATCGGCGTTGGCTAAGTGAC |
| | | (SEQ ID NO: 219) |
| Igf1 | ENSMUSTO0000095360.6 | TGGTTCAATCGTATGCCCGT TTAGTCGTCGCAACGCCAGGCAACTCATGC |
| | | CCCAGTTGAATGCATCCAACACATTTCCAG |
| | | GTAAGCGCAACGGTGGACCGGAGACGACGG ATCGGCGTTGGCTAAGTGAC |
| | | (SEQ ID NO: 220) |
| Igf1 | ENSMUSTO0000095360.6 | TGGTTCAATCGTATGCCCGT TTAGTCGTCGCAACGCCAGGCAACTCATGC |
| | | CAGCTAGACAAGATGATTAGACTCTGTAAA |
| | | GTAAGCGCAACGGTGGACCGGAGACGACGG ATCGGCGTTGGCTAAGTGAC |
| | | (SEQ ID NO: 221) |
| Igf1 | ENSMUSTO0000095360.6 | TGGTTCAATCGTATGCCCGT TTAGTCGTCGCAACGCCAGGCAACTCATGC |
| | | ATACTATAGGATTAAGCACTAGCATTGAAA |
| | | GTAAGCGCAACGGTGGACCGGAGACGACGG ATCGGCGTTGGCTAAGTGAC |
| | | (SEQ ID NO: 222) |
| Igf1 | ENSMUSTO0000095360.6 | TGGTTCAATCGTATGCCCGT TTAGTCGTCGCAACGCCAGGCAACTCATGC |
| | | TCAAGTACTTTCTTAAAGAAACAATAGCAC |
| | | GTAAGCGCAACGGTGGACCGGAGACGACGG ATCGGCGTTGGCTAAGTGAC |
| | | (SEQ ID NO: 223) |
| Igf1 | ENSMUSTO0000095360.6 | TGGTTCAATCGTATGCCCGT TTAGTCGTCGCAACGCCAGGCAACTCATGC |
| | | TCTTAGGCTCCAGGCTTTCGTTTGTTGTTT |
| | | GTAAGCGCAACGGTGGACCGGAGACGACGG ATCGGCGTTGGCTAAGTGAC |
| | | (SEQ ID NO: 224) |
| Igf1 | ENSMUSTO0000095360.6 | TGGTTCAATCGTATGCCCGT TTAGTCGTCGCAACGCCAGGCAACTCATGC |
| | | AGCAAAGGATCCTGCGGTGATGTGGCATTT |
| | | CGGCCGAATTACATCCGTCGTAATCGAGGC ATCGGCGTTGGCTAAGTGAC |
| | | (SEQ ID NO: 225) |
| Igf1 | ENSMUSTO0000095360.6 | TGGTTCAATCGTATGCCCGT TTAGTCGTCGCAACGCCAGGCAACTCATGC |
| | | AGTGTTTAGCAGTAGGTACAATGTAAATAT |
| | | CGGCCGAATTACATCCGTCGTAATCGAGGC ATCGGCGTTGGCTAAGTGAC |
| | | (SEQ ID NO: 226) |
| Igf1 | ENSMUSTO0000095360.6 | TGGTTCAATCGTATGCCCGT TTAGTCGTCGCAACGCCAGGCAACTCATGC |
| | | TAAATAATTGAGTTGGAAGGCTGCTGATTT |
| | | CGGCCGAATTACATCCGTCGTAATCGAGGC ATCGGCGTTGGCTAAGTGAC |
| | | (SEQ ID NO: 227) |
| Igf1 | ENSMUSTO0000095360.6 | TGGTTCAATCGTATGCCCGT TTAGTCGTCGCAACGCCAGGCAACTCATGC |
| | | CTGGCGGAAGGAGAGCTAAAGGTGTCCTTT |
| | | CGGCCGAATTACATCCGTCGTAATCGAGGC ATCGGCGTTGGCTAAGTGAC |
| | | (SEQ ID NO: 228) |
| Igf1 | ENSMUSTO0000095360.6 | TGGTTCAATCGTATGCCCGT TTAGTCGTCGCAACGCCAGGCAACTCATGC |
| | | TTTACACAAGTTCACTTTGGGCAAGAGAAA |
| | | CGGCCGAATTACATCCGTCGTAATCGAGGC ATCGGCGTTGGCTAAGTGAC |
| | | (SEQ ID NO: 229) |
| Igf1 | ENSMUSTO0000095360.6 | TGGTTCAATCGTATGCCCGT TTAGTCGTCGCAACGCCAGGCAACTCATGC |
| | | GAGAACCTCAGGCTTGAGAAGGAAGAATTT |
| | | CGGCCGAATTACATCCGTCGTAATCGAGGC ATCGGCGTTGGCTAAGTGAC |
| | | (SEQ ID NO: 230) |
| Igf1 | ENSMUSTO0000095360.6 | TGGTTCAATCGTATGCCCGT TTAGTCGTCGCAACGCCAGGCAACTCATGC |
| | | CATGTACACAGACATGCCACATTTCACATT |
| | | CGGCCGAATTACATCCGTCGTAATCGAGGC ATCGGCGTTGGCTAAGTGAC |
| | | (SEQ ID NO: 231) |
| Igf1 | ENSMUSTO0000095360.6 | TGGTTCAATCGTATGCCCGT TTAGTCGTCGCAACGCCAGGCAACTCATGC |
| | | ACTTCCTTCTGAGTCTTGGGCATGTCAGTG |
| | | CGGCCGAATTACATCCGTCGTAATCGAGGC ATCGGCGTTGGCTAAGTGAC |
| | | (SEQ ID NO: 232) |
| Igf1 | ENSMUSTO0000095360.6 | TGGTTCAATCGTATGCCCGT TTAGTCGTCGCAACGCCAGGCAACTCATGC |
| | | CAAATGTCTCCTGCCGTGTGACATGCTGGG |
| | | CGGCCGAATTACATCCGTCGTAATCGAGGC ATCGGCGTTGGCTAAGTGAC |
| | | (SEQ ID NO: 233) |
| Igf1 | ENSMUSTO0000095360.6 | TGGTTCAATCGTATGCCCGT TTAGTCGTCGCAACGCCAGGCAACTCATGC |
| | | ACTTGTGAGTGACACATAGTTGAAAGGTGG |
| | | CGGCCGAATTACATCCGTCGTAATCGAGGC ATCGGCGTTGGCTAAGTGAC |
| | | (SEQ ID NO: 234) |
| Igf1 | ENSMUSTO0000095360.6 | TGGTTCAATCGTATGCCCGTGTAAGCGCAACGGTGGACCGGAGACGACGG |
| | | TAAATGTCCCTTTCTATCAATCTTGAGTCA |
| | | CGGCCGAATTACATCCGTCGTAATCGAGGC ATCGGCGTTGGCTAAGTGAC |
| | | (SEQ ID NO: 235) |
| Igf1 | ENSMUSTO0000095360.6 | TGGTTCAATCGTATGCCCGTGTAAGCGCAACGGTGGACCGGAGACGACGG |
| | | TAATTAATTACCCTTTAATGAGAGTGGATA |
| | | CGGCCGAATTACATCCGTCGTAATCGAGGC ATCGGCGTTGGCTAAGTGAC |
| | | (SEQ ID NO: 236) |
| Igf1 | ENSMUSTO0000095360.6 | TGGTTCAATCGTATGCCCGTGTAAGCGCAACGGTGGACCGGAGACGACGG |
| | | AGATGTTAGAGCAATGTCACATTTCAATTT |
| | | CGGCCGAATTACATCCGTCGTAATCGAGGC ATCGGCGTTGGCTAAGTGAC |
| | | (SEQ ID NO: 237) |
| Igf1 | ENSMUSTO0000095360.6 | TGGTTCAATCGTATGCCCGTGTAAGCGCAACGGTGGACCGGAGACGACGG |
| | | ACAGATCTGGAGAAGGCCACCTATGTGTTT |
| | | CGGCCGAATTACATCCGTCGTAATCGAGGC ATCGGCGTTGGCTAAGTGAC |
| | | (SEQ ID NO: 238) |
| Igf1 | ENSMUSTO0000095360.6 | TGGTTCAATCGTATGCCCGTGTAAGCGCAACGGTGGACCGGAGACGACGG |
| | | GGAGCTACATTGTTAGTGGGTTATTTACAG |
| | | CGGCCGAATTACATCCGTCGTAATCGAGGC ATCGGCGTTGGCTAAGTGAC |
| | | (SEQ ID NO: 239) |
| Igf1 | ENSMUSTO0000095360.6 | TGGTTCAATCGTATGCCCGTGTAAGCGCAACGGTGGACCGGAGACGACGG |
| | | CACAGCTTCACCAATAAGAACTTGGATATT |
| | | CGGCCGAATTACATCCGTCGTAATCGAGGC ATCGGCGTTGGCTAAGTGAC |
| | | (SEQ ID NO: 240) |
| Igf1 | ENSMUSTO0000095360.6 | TGGTTCAATCGTATGCCCGTGTAAGCGCAACGGTGGACCGGAGACGACGG |
| | | CTTTATCTTCAAGAAGTCACAGAGGCAGAT |
| | | CGGCCGAATTACATCCGTCGTAATCGAGGC ATCGGCGTTGGCTAAGTGAC |
| | | (SEQ ID NO: 241) |
| Igf1 | ENSMUSTO0000095360.6 | TGGTTCAATCGTATGCCCGTGTAAGCGCAACGGTGGACCGGAGACGACGG |
| | | TAGATTATAATGGAGAGATGTATTACATTT |
| | | CGGCCGAATTACATCCGTCGTAATCGAGGC ATCGGCGTTGGCTAAGTGAC |
| | | (SEQ ID NO: 242) |
| Igf1 | ENSMUSTO0000095360.6 | TGGTTCAATCGTATGCCCGTGTAAGCGCAACGGTGGACCGGAGACGACGG |
| | | AAGGATTGAACCAGATATGGCTTGTTATTT |
| | | CGGCCGAATTACATCCGTCGTAATCGAGGC ATCGGCGTTGGCTAAGTGAC |
| | | (SEQ ID NO: 243) |
| Igf1 | ENSMUSTO0000095360.6 | TGGTTCAATCGTATGCCCGTGTAAGCGCAACGGTGGACCGGAGACGACGG |
| | | AGCCATAGCCTGTGGGCTTGTTGAAGTAAA |
| | | CGGCCGAATTACATCCGTCGTAATCGAGGC ATCGGCGTTGGCTAAGTGAC |
| | | (SEQ ID NO: 244) |

* Only the template sequence for the first gene is shown in this PDF as the table is too large. The full sequence table can be downloaded as excel file.

TABLE 5
Readout probe sequences for each of the 16 bits used in the
133-genes conventional library.

| Bit | Readout Sequence |
|---|---|
| B1 | CGCAACGCTTGGGACGGTTCCAATCGGATC/3Cy5Sp/ (SEQ ID NO: 245) |
| B2 | CGAATGCTCTGGCCTCGAACGAACGATAGC/3Cy5Sp/ (SEQ ID NO: 246) |
| B3 | ACAAATCCGACCAGATCGGACGATCATGGG/3Cy5Sp/ (SEQ ID NO: 247) |
| B4 | CAAGTATGCAGCGCGATTGACCGTCTCGTT/3Cy5Sp/ (SEQ ID NO: 248) |
| B5 | GCGGGAAGCACGTGGATTAGGGCATCGACC/3Cy5Sp/ (SEQ ID NO: 249) |
| B6 | AAGTCGTACGCCGATGCGCAGCAATTCACT/3Cy5Sp/ (SEQ ID NO: 250) |
| B7 | CGAAACATCGGCCACGGTCCCGTTGAACTT/3Cy5Sp/(SEQ ID NO: 251) |
| B8 | ACGAATCCACCGTCCAGCGCGTCAAACAGA/3Cy5Sp/ (SEQ ID NO: 252) |
| B9 | /5IRD800CWN/CGCGAAATCCCCGTAACGAGCGTCCCTTGC (SEQ ID NO: 253) |
| B10 | /5IRD800CWN/GCATGAGTTGCCTGGCGTTGCGACGACTAA (SEQ ID NO: 254) |
| B11 | /5IRD800CWN/CCGTCGTCTCCGGTCCACCGTTGCGCTTAC (SEQ ID NO: 255) |
| B12 | /5IRD800CWN/GGCCAATGGCCCAGGTCCGTCACGCAATTT (SEQ ID NO: 256) |
| B13 | /5IRD800CWN/TTGATCGAATCGGAGCGTAGCGGAATCTGC (SEQ ID NO: 257) |
| B14 | /5IRD800CWN/CGCGCGGATCCGCTTGTCGGGAACGGATAC (SEQ ID NO: 258) |
| B15 | /5IRD800CWN/GCCTCGATTACGACGGATGTAATTCGGCCG (SEQ ID NO: 259) |
| B16 | /5IRD800CWN/GCCCGTATTCCCGCTTGCGAGTAGGGCAAT (SEQ ID NO: 260) |

TABLE 6
Codebook for each gene in the 133-genes
conventional library. The binary code word
assigned to each gene in the 133-genes
conventional library.

| Gene | Code-word |
|---|---|
| Igf1 | 0 0 1 0 0 0 0 0 0 1 1 0 0 0 1 0 |
| Cps1 | 1 0 0 0 0 0 0 1 1 0 0 0 1 0 0 0 |
| Glul | 0 0 0 0 1 1 0 1 0 0 0 1 0 0 0 0 |
| Glud1 | 0 0 0 0 0 0 0 1 0 0 0 0 1 1 1 0 |
| Pigr | 0 1 0 0 0 0 1 0 0 0 0 1 0 0 0 1 |
| Pck1 | 1 0 0 1 0 0 0 0 0 0 1 1 0 0 0 0 |
| Uox | 0 1 0 0 0 0 0 0 1 1 0 0 0 1 0 0 |
| Hnf4a | 0 0 0 0 1 1 0 0 1 0 0 0 0 0 0 1 |
| Abcc3 | 0 1 0 1 0 0 0 0 0 1 0 0 0 0 0 1 |
| G6pc | 0 0 1 0 1 0 1 1 0 0 0 0 0 0 0 0 |
| Paqr9 | 0 0 0 0 0 1 1 0 0 1 0 0 0 0 0 1 |
| Cpt1a | 0 1 0 0 1 0 0 1 0 1 0 0 0 0 0 0 |
| Acly | 1 0 0 0 0 0 1 0 0 1 1 0 0 0 0 0 |
| Ganab | 0 0 0 1 0 0 0 0 1 1 1 0 0 0 0 0 |
| Gpam | 0 0 1 0 1 0 0 0 0 1 0 0 0 1 0 0 |
| Plxnb2 | 0 0 1 0 0 1 0 1 0 0 0 0 0 0 1 0 |
| Dpyd | 0 1 1 1 0 0 0 0 0 0 0 1 0 0 0 0 |
| Clptm1 | 0 0 0 1 0 0 1 0 0 1 0 1 0 0 0 0 |
| Ddx17 | 0 1 0 1 0 1 1 0 0 0 0 0 0 0 0 0 |
| Scap | 0 0 1 0 0 0 0 0 0 0 0 0 0 1 1 1 |
| Uba1 | 0 0 0 1 1 0 1 0 0 0 1 0 0 0 0 0 |
| Gns | 0 1 0 1 1 0 0 0 0 0 0 0 0 0 1 0 |
| Lrrc3 | 0 1 0 0 0 0 1 1 1 0 0 0 0 0 0 0 |
| Fads6 | 0 1 0 0 0 0 0 0 1 0 1 0 0 0 0 1 |
| Hs3st3b1 | 1 0 1 0 0 0 1 0 0 0 0 0 0 0 1 0 |
| Zcchc24 | 0 1 0 0 0 0 1 0 0 0 1 0 0 0 1 0 |
| Cdc42bpb | 1 0 0 0 1 0 0 0 0 0 0 0 1 1 0 0 |
| Zdhhc5 | 1 0 1 0 1 1 0 0 0 0 0 0 0 0 0 0 |
| Pdxk | 0 0 1 0 0 1 0 0 1 0 1 0 0 0 0 0 |
| Vwa8 | 1 0 0 0 0 0 1 0 0 0 0 0 0 1 0 1 |
| Flii | 0 0 1 0 0 0 0 0 0 1 0 1 0 0 0 1 |
| Ube2z | 0 0 0 0 0 0 1 1 0 0 0 1 0 0 1 0 |
| Slc38a2 | 0 0 0 0 0 0 1 1 0 1 0 0 0 1 0 0 |
| Stat2 | 0 1 0 0 0 0 0 1 0 0 1 1 0 0 0 0 |
| Bcl9l | 0 0 0 0 1 0 0 0 0 1 1 1 0 0 0 0 |
| Tomm20 | 1 0 1 1 0 0 0 0 0 0 0 0 0 1 0 0 |
| Il4ra | 0 0 0 0 1 1 1 0 0 0 0 0 0 0 1 0 |
| Nol6 | 0 1 0 0 0 0 0 0 1 0 0 1 0 0 1 0 |
| Ide | 1 1 0 0 0 0 1 0 0 0 0 0 1 0 0 0 |
| Ddx3x | 0 0 0 0 0 0 0 1 1 0 1 0 0 0 1 0 |
| Rnf144b | 1 1 0 0 0 1 0 0 1 0 0 0 0 0 0 0 |
| Mybbp1a | 1 1 0 0 0 0 0 0 0 0 0 1 0 1 0 0 |
| Hnf1b | 1 0 0 0 1 0 0 0 1 0 1 0 0 0 0 0 |
| Flnb | 0 1 0 1 0 0 0 0 0 0 1 0 0 1 0 0 |
| Golga2 | 0 0 0 0 0 0 0 0 0 1 1 0 0 1 0 1 |
| Cdh5 | 0 0 1 1 1 0 0 0 0 0 0 0 1 0 0 0 |
| Tbx3 | 0 0 0 0 1 0 0 0 0 0 1 0 0 1 1 0 |
| Paxip1 | 0 0 1 0 0 0 1 0 0 0 1 0 1 0 0 0 |
| Dennd5a | 1 0 0 0 0 0 0 0 1 1 0 1 0 0 0 0 |
| Rhbdd2 | 0 0 0 0 0 1 0 1 0 0 0 0 0 1 0 1 |
| Mob3b | 0 0 1 0 0 1 0 0 0 0 0 0 1 1 0 0 |
| Plekhm2 | 1 0 0 0 0 0 0 0 0 0 1 0 1 0 1 0 |
| Ube3a | 0 0 0 1 1 1 0 0 0 0 0 0 0 1 0 0 |
| Dgkq | 1 0 0 0 0 1 1 1 0 0 0 0 0 0 0 0 |
| Tecpr1 | 0 0 0 1 0 0 1 0 0 0 0 0 0 1 1 0 |
| Iffo2 | 1 0 0 1 0 0 1 0 1 0 0 0 0 0 0 0 |
| Zhx2 | 0 0 0 0 1 0 1 0 1 1 0 0 0 0 0 0 |
| Gpd2 | 0 1 1 0 0 1 0 0 0 0 0 0 0 0 0 1 |
| Lrat | 0 0 0 1 0 0 0 1 0 1 0 0 0 0 1 0 |
| Lrtm1 | 1 0 0 1 0 1 0 0 0 0 0 0 1 0 0 0 |
| Tbl1xr1 | 0 1 0 0 0 0 0 0 0 0 0 0 1 1 0 1 |
| Pecam1 | 0 0 0 0 1 0 0 0 1 0 0 0 1 0 1 0 |
| Cog3 | 0 0 0 0 0 0 1 0 1 0 1 1 0 0 0 0 |
| Atp11b | 0 0 1 0 0 1 1 0 0 0 0 1 0 0 0 0 |
| Add3 | 0 1 1 0 1 0 0 0 1 0 0 0 0 0 0 0 |
| Mcam | 0 1 0 1 0 0 0 0 1 0 0 0 1 0 0 0 |
| 1700017B05Rik | 0 1 0 0 1 0 1 0 0 0 0 0 0 1 0 0 |
| Megf8 | 1 0 1 0 0 0 0 1 0 0 0 1 0 0 0 0 |
| Tppp | 1 0 0 0 0 0 0 0 1 0 0 0 0 1 1 0 |
| Vps13c | 1 1 0 0 1 0 0 0 0 0 0 0 0 0 0 1 |
| Exoc2 | 0 0 1 0 0 0 0 0 1 0 0 1 1 0 0 0 |
| Taok1 | 1 0 0 0 0 1 0 0 0 1 0 0 0 1 0 0 |
| Enpp4 | 0 0 0 0 0 1 1 0 0 0 1 0 0 1 0 0 |
| Des | 0 0 0 0 0 0 1 0 0 1 0 0 1 0 1 0 |
| Ranbp2 | 0 0 0 1 1 0 0 1 0 0 0 0 0 0 0 1 |
| Golga4 | 1 0 0 0 1 0 0 1 0 0 0 0 0 0 1 0 |
| Sptlc2 | 0 0 0 0 0 0 0 1 1 0 0 1 0 0 0 1 |
| Sox9 | 0 0 1 0 0 0 1 0 1 0 0 0 0 1 0 0 |
| Capn5 | 0 0 0 1 1 0 0 0 1 0 0 1 0 0 0 0 |
| Rasa1 | 0 0 0 0 0 0 1 0 1 0 0 0 0 0 1 1 |
| Vcpip1 | 1 0 1 0 0 0 0 0 0 1 0 0 1 0 0 0 |
| Tln2 | 1 0 0 1 1 0 0 0 0 1 0 0 0 0 0 0 |
| Gpc1 | 0 0 0 0 0 0 1 1 0 0 1 0 0 0 0 1 |
| Adcy7 | 0 1 0 0 0 1 0 1 0 0 0 0 1 0 0 0 |
| Zxdb | 0 0 0 0 0 1 0 0 1 1 0 0 0 0 1 0 |
| Cdk19 | 0 0 0 1 0 0 0 0 1 0 0 0 0 1 0 1 |
| Chic1 | 0 1 0 0 0 1 0 0 0 1 0 1 0 0 0 0 |
| C3ar1 | 0 0 1 0 0 0 0 0 0 0 1 1 0 1 0 0 |
| Ccdc88b | 0 0 1 1 0 1 0 0 0 1 0 0 0 0 0 0 |
| Aatk | 0 0 0 1 0 1 0 0 0 0 0 1 0 0 0 1 |
| Tmed8 | 1 0 0 0 0 0 0 1 0 1 0 0 0 0 0 1 |
| Zfp174 | 1 0 1 0 0 0 0 0 1 0 0 0 0 0 0 1 |
| Fastkd2 | 0 0 0 0 0 1 0 1 0 1 1 0 0 0 0 0 |
| Zfp870 | 0 0 0 1 0 0 0 0 0 0 1 0 1 0 0 1 |
| Dsg1c | 0 0 0 0 1 0 0 0 0 1 0 0 0 0 1 1 |
| Wdr19 | 0 0 0 0 1 1 0 0 0 1 0 0 1 0 0 0 |
| Mphosph9 | 0 0 1 0 1 0 0 0 0 0 0 1 0 0 1 0 |
| Abca5 | 0 0 0 0 0 0 0 0 1 0 1 0 1 1 0 0 |
| Atp8b5 | 0 0 0 1 0 0 0 1 0 0 0 1 0 1 0 0 |
| Ankrd26 | 0 0 1 0 0 0 0 1 0 0 0 0 1 0 0 1 |
| Tbc1d9 | 0 1 1 0 0 0 0 0 0 0 0 0 1 0 1 0 |
| Zfp560 | 0 0 1 0 1 0 0 0 0 0 1 0 0 0 0 1 |
| Ggt1 | 0 1 0 0 0 1 0 0 0 0 0 0 0 1 1 0 |
| AW554918 | 0 1 0 0 0 0 0 0 0 1 1 0 1 0 0 0 |
| Itga4 | 0 0 1 1 0 0 0 1 0 0 1 0 0 0 0 0 |
| Ammecr1 | 0 0 0 0 0 1 0 0 1 0 0 1 0 1 0 0 |
| Hecw2 | 0 0 0 0 0 0 0 0 0 0 1 1 0 0 1 1 |
| Fam102b | 0 0 0 0 1 0 0 0 0 0 0 1 0 1 0 1 |
| Nova2 | 0 0 0 0 0 0 0 0 0 1 0 1 0 1 1 0 |
| Krt7 | 1 0 0 0 1 0 1 0 0 0 0 1 0 0 0 0 |
| Prrg3 | 0 0 0 0 0 1 0 0 0 0 0 0 1 0 1 1 |
| Kif7 | 0 0 0 1 0 0 0 0 0 0 0 1 1 0 1 0 |
| Setbp1 | 1 0 0 0 0 0 0 1 0 0 1 0 0 1 0 0 |
| Pcdh17 | 0 0 1 1 0 0 0 0 1 0 0 0 0 0 1 0 |
| Brip1 | 0 0 0 1 0 0 1 1 0 0 0 0 1 0 0 0 |
| Armcx4 | 0 0 0 0 0 1 1 0 1 0 0 0 1 0 0 0 |
| Scube1 | 0 0 0 0 1 0 0 1 1 0 0 0 0 1 0 0 |
| Alms1 | 0 0 0 0 0 0 0 0 1 1 0 0 1 0 0 1 |
| Dync2h1 | 1 0 0 1 0 0 0 0 0 0 0 0 0 0 1 1 |
| Hcn3 | 1 1 0 1 0 0 0 1 0 0 0 0 0 0 0 0 |
| Sdk1 | 0 1 1 0 0 0 0 1 0 0 0 0 0 1 0 0 |
| Slit3 | 0 0 0 0 0 0 0 1 0 1 0 1 1 0 0 0 |
| Plag1 | 1 0 0 0 0 1 0 0 0 0 0 1 0 0 1 0 |
| Sema3g | 0 0 0 0 0 0 1 0 0 0 0 1 1 1 0 0 |
| Ncs1 | 0 0 0 1 0 0 0 0 0 1 0 0 1 1 0 0 |
| Egflam | 0 1 0 0 1 1 0 0 0 0 1 0 0 0 0 0 |
| Lama3 | 1 0 0 0 0 0 0 0 0 0 0 1 1 0 0 1 |
| Igdcc4 | 0 0 0 0 0 1 0 0 0 0 1 1 1 0 0 0 |
| Sgpp2 | 0 0 0 0 1 0 0 1 0 0 1 0 1 0 0 0 |
| Lrrc16b | 0 0 0 1 0 1 0 0 0 0 1 0 0 0 1 0 |
| Col4a4 | 1 0 0 0 0 1 0 0 0 0 1 0 0 0 0 1 |
| Cnnm1 | 0 1 0 0 0 0 0 1 0 0 0 0 0 0 1 1 |
| Cftr | 0 1 1 0 0 0 1 0 0 1 0 0 0 0 0 0 |
| Biank1 | 0 0 0 1 0 1 0 1 1 0 0 0 0 0 0 0 |
| Blank2 | 0 0 1 0 0 0 0 1 1 1 0 0 0 0 0 0 |
| Blank3 | 1 1 1 0 0 0 0 0 0 0 1 0 0 0 0 0 |
| Blank4 | 0 1 0 0 1 0 0 0 0 0 0 1 1 0 0 0 |
| Blank5 | 0 0 1 1 0 0 1 0 0 0 0 0 0 0 0 1 |
| Blank6 | 1 1 0 0 0 0 0 0 0 1 0 0 0 0 1 0 |
| Blank7 | 0 0 0 0 1 0 1 0 0 0 0 0 1 0 0 1 |

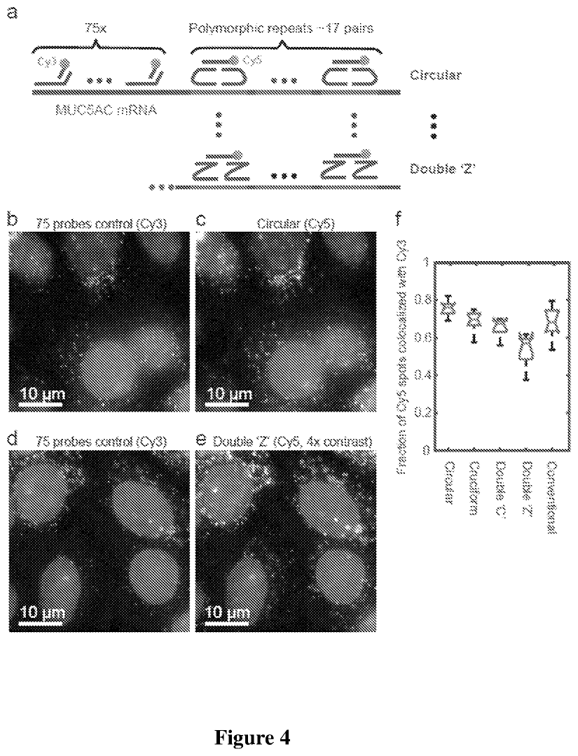

TABLE 7
Probe sequences for the conventional, split probe pairs, and
readout probe used in the XLOC_010514 experiment (FIG. 4).
The known off-target sequence is shown in bold.
XLOC conventional probe sequences

| | |
|---|---|
| XLOC_1 | CATTCCCAGGGACATTCATT TATCCACCGAACCCTTAC (SEQ ID NO: 261) |
| XLOC_2 | GATGACTTCTCTATCCCACTTATCCACCGAACCCTTAC (SEQ ID NO: 262) |
| XLOC_3 | GAAGTGCCTCGTCATTCTGGTATCCACCGAACCCTTAC (SEQ ID NO: 263) |
| XLOC_4 | GCTCCAGTAACTAGCTGACATATCCACCGAACCCTTAC (SEQ ID NO: 264) |
| XLOC_5 | AAGCCCAGGGGTACTCCTTATATCCACCGAACCCTTAC (SEQ ID NO: 265) |
| XLOC_6 | GGAGCCTTGTAAATACTGATTATCCACCGAACCCTTAC (SEQ ID NO: 266) |
| XLOC_7 | GCTGGAACCCTTAGGAAATATATCCACCGAACCCTTAC (SEQ ID NO: 267) |
| XLOC_8 | GAGAATGGCAAGAACTCCATTATCCACCGAACCCTTAC (SEQ ID NO: 268) |
| XLOC_9 | ATGAACAGGCAGAGGCTAGATATCCACCGAACCCTTAC (SEQ ID NO: 269) |
| XLOC_10 | TACTGATCTGAAGCAGCAGGTATCCACCGAACCCTTAC (SEQ ID NO: 270) |
| XLOC_11 | CAGACTAAAAGGAGGCCAGGTATCCACCGAACCCTTAC (SEQ ID NO: 271) |
| XLOC_12 | AGTGAATTGAGAATATGAGGTATCCACCGAACCCTTAC (SEQ ID NO: 272) |
| XLOC_13 | AGGATAGTAACTCAGTAGAATATCCACCGAACCCTTAC (SEQ ID NO: 273) |
| XLOC_14 | TGATAGGAGGCATTGTCATTTATCCACCGAACCCTTAC (SEQ ID NO: 274) |
| XLOC_15 | TTGTATCAAACCAAGTCTCTTATCCACCGAACCCTTAC (SEQ ID NO: 275) |
| XLOC_16 | ACGAGCAGTTGTATGTGAATTATCCACCGAACCCTTAC (SEQ ID NO: 276) |
| XLOC_17 | TTCCCCAGTTATAGAAATCATATCCACCGAACCCTTAC (SEQ ID NO: 277) |
| XLOC_18 | TATCTGGGGTAATGGAGACATATCCACCGAACCCTTAC (SEQ ID NO: 278) |
| XLOC_19 | CGAATTTCCTGCACTGGTAATATCCACCGAACCCTTAC (SEQ ID NO: 279) |
| XLOC_20 | TCAGGTTGTATGATATAGTGTATCCACCGAACCCTTAC (SEQ ID NO: 280) |
| XLOC_21 | ACTGTCTACTTCACCATTGATATCCACCGAACCCTTAC (SEQ ID NO: 281) |
| XLOC_22 | TAAAACATCCATTGCCCTGGTATCCACCGAACCCTTAC (SEQ ID NO: 282) |
| XLOC_23 | ACAGACCGGTTAACCATCCATATCCACCGAACCCTTAC (SEQ ID NO: 283) |

XLOC split probe sequences

| Name | Sequence |
|---|---|
| XLOC_SplitLeft_1 | AACCCTTAC TAT AAATTAACATGCTCGGCCAG (SEQ ID NO: 284) |
| XLOC_SplitLeft_2 | AACCCTTAC TAT AATTCTCCTTGAAGATCATT (SEQ ID NO: 285) |
| XLOC_SplitLeft_3 | AACCCTTAC TAT TCAGACACAGGTGCAGATAG (SEQ ID NO: 286) |
| XLOC_SplitLeft_4 | AACCCTTAC TAT TCCAGTAACTAGCTGACATG (SEQ ID NO: 287) |
| XLOC_SplitLeft_5 | AACCCTTAC TAT CTGATGAAGTGATTAAGGAG (SEQ ID NO: 288) |
| XLOC_SplitLeft_6 | AACCCTTAC TAT AATTACACTGCCTGGCACAT (SEQ ID NO: 289) |
| XLOC_SplitLeft_7 | AACCCTTAC TAT AGTGCCTCAGACACTGCCTG (SEQ ID NO: 290) |
| XLOC_SplitLeft_8 | AACCCTTAC TAT TATCCATGGGCTGCTGGAAC (SEQ ID NO: 291) |
| XLOC_SplitLeft_9 | AACCCTTAC TAT TCTGAGAATGGCAAGAACTC (SEQ ID NO: 292) |
| XLOC_SplitLeft_10 | AACCCTTAC TAT GCAGAGGCTAGAGATGCTCA (SEQ ID NO: 293) |
| XLOC_SplitLeft_11 | AACCCTTAC TAT AGCAGGTCCACGCCCAGGTG (SEQ ID NO: 294) |
| XLOC_SplitLeft_12 | AACCCTTAC TAT CCATCCAACTGAGTTCATTC (SEQ ID NO: 295) |
| XLOC_SplitLeft_13 | AACCCTTAC TAT GTAGAAAGAGTGAATTGAGA (SEQ ID NO: 296) |
| XLOC_SplitLeft_14 | AACCCTTAC TAT TAGATGATAGGAGGCATTGT (SEQ ID NO: 297) |
| XLOC_SplitLeft_15 | AACCCTTAC TAT CGAGCAGTTGTATGTGAATG (SEQ ID NO: 298) |
| XLOC_SplitLeft_16 | AACCCTTAC TAT ACATCCTCCATTGCACCGCA (SEQ ID NO: 299) |
| XLOC_SplitLeft_17 | AACCCTTAC TAT TCGAACGAATTTCCTGCACT (SEQ ID NO: 300) |
| XLOC_SplitLeft_18 | AACCCTTAC TAT TTTCAGGTTGTATGATATAG (SEQ ID NO: 301) |
| XLOC_SplitLeft_19 | AACCCTTAC TAT TCCACCTCATGGCCACAGAC (SEQ ID NO: 302) |
| XLOC_SplitLeft_20 | AACCCTTAC TAT TAACTTGATTGTGATGATGG (SEQ ID NO: 303) |
| XLOC_SplitLeft_21 | AACCCTTAC TAT TTTAGGAGGGAGGAATTACC (SEQ ID NO: 304) |
| XLOC_SplitLeft_22 | AACCCTTAC TAT ACAAACAAATCTATAGTGAT (SEQ ID NO: 305) |
| XLOC_SplitLeft_23 | AACCCTTAC TAT CCGTGGTGGTTTCGGGGCAG (SEQ ID NO: 306) |
| XLOC_SplitRight_1 | GCGCGGTGGCTCACACCTGT TAT TATCCACCG (SEQ ID NO: 307) |
| XLOC_SplitRight_2 | CCCAGGGACATTCATTTGCT TAT TATCCACCG (SEQ ID NO: 308) |
| XLOC_SplitRight_3 | ATGACTTCTCTATCCCACTT TAT TATCCACCG (SEQ ID NO: 309) |
| XLOC_SplitRight_4 | GAAGTGCCTCGTCATTCTGG TAT TATCCACCG (SEQ ID NO: 310) |
| XLOC_SplitRight_5 | CCTTGTAAATACTGATTAAA TAT TATCCACCG (SEQ ID NO: 311) |
| XLOC_SplitRight_6 | AGTAGGTGCTCAATGAATTG TAT TATCCACCG (SEQ ID NO: 312) |
| XLOC_SplitRight_7 | GCACACAGCAGGTGCTCAAT TAT TATCCACCG (SEQ ID NO: 313) |
| XLOC_SplitRight_8 | CCTTAGGAAATAATAGCTAG TAT TATCCACCG (SEQ ID NO: 314) |
| XLOC_SplitRight_9 | CATTAACTCTCTTCTCCAAA TAT TATCCACCG (SEQ ID NO: 315) |
| XLOC_SplitRight_10 | TTCCTAAAGGGATCAAATAA TAT TATCCACCG (SEQ ID NO: 316) |
| XLOC_SplitRight_11 | CAGATTGTCCCAGATGAACA TAT TATCCACCG (SEQ ID NO: 317) |
| XLOC_SplitRight_12 | TGATACCTACTGATCTGAAG TAT TATCCACCG (SEQ ID NO: 318) |
| XLOC_SplitRight_13 | ATATGAGGAATGAGAGTGGG TAT TATCCACCG (SEQ ID NO: 319) |
| XLOC_SplitRight_14 | CATTACTAGGATAGTAACTC TAT TATCCACCG (SEQ ID NO: 320) |
| XLOC_SplitRight_15 | CATTGTATCAAACCAAGTCT TAT TATCCACCG (SEQ ID NO: 321) |
| XLOC_SplitRight_16 | TATTTGCTTTCTGTATTCCC TAT TATCCACCG (SEQ ID NO: 322) |
| XLOC_SplitRight_17 | GGTAACTAGGTGAAGAATTT TAT TATCCACCG (SEQ ID NO: 323) |
| XLOC_SplitRight_18 | TGCCTGACAACTCTCCATTT TAT TATCCACCG (SEQ ID NO: 324) |
| XLOC_SplitRight_19 | CGGTTAACCATCCATATAAA TAT TATCCACCG (SEQ ID NO: 325) |
| XLOC_SplitRight_20 | CTTCACAGGTGTACACAGCT TAT TATCCACCG (SEQ ID NO: 326) |
| XLOC_SplitRight_21 | AAAGGGCATGAGAACACTTT TAT TATCCACCG (SEQ ID NO: 327) |
| XLOC_SplitRight_22 | AGAAAGTTGGTCAGCGGTTT TAT TATCCACCG (SEQ ID NO: 328) |
| XLOC_SplitRight_23 | AAGCCCAGGGGTACTCCTTA TAT TATCCACCG (SEQ ID NO: 329) |

XLOC readout sequence

| Name | Readout Sequence |
|---|---|
| XLOC_Cy5_Readout | /5Cy5/GTAAGGGTT CGGTGGATA (SEQ ID NO: 330) |

TABLE 8
Probe sequences used in the MUC5AC experiment (FIGS. 1, 2 and 3). Sheet 8a:
Sequences of the unpaired, paired (circular), and readout probes used in
FIG. 1. Sheet 8b: Sequences of the MUC5AC split-probe constructs and readout
probe used for FIG. 3. Sheet 8c: Sequences of the MUC5AC split-probe,
conventional probe, bridge probe, and readout probes used for the kinetic
experiment in FIG. 2. Lowercase letters denotes the target gene (MUC5AC)
binding sequence. Uppercase letters denotes the 3 nucleotide linker and
readout binding sequence.
Table 8a
Unpaired split probe sequences

| Name | Sequence |
|---|---|
| 12nt_Split_probe_2 | acagggctgggagtagttccag TAT TATCCACCGAAC (SEQ ID NO: 331) |
| 11nt_Split_probe_2 | acagggctgggagtagttccag TAT TATCCACCGAA (SEQ ID NO: 332) |
| 10nt_Split_probe_2 | acagggctgggagtagttccag TAT TATCCACCGA (SEQ ID NO: 333) |
| 9nt_Split_probe_2 | acagggctgggagtagttccag TAT TATCCACCG (SEQ ID NO: 334) |
| 8nt_Split_probe_2 | acagggctgggagtagttccag TAT ATCCACCG (SEQ ID NO: 335) |
| 7nt_Split_probe_2 | acagggctgggagtagttccag TAT TCCACCG (SEQ ID NO: 336) |

Paired (circular) split probe sequences

| Name | Sequence |
|---|---|
| 9nt_Split_probe_1 | AACCCTTAC TAT agaggttgtgctggtggtggga (SEQ ID NO: 337) |
| 9nt_Split_probe_2 | acagggctgggagtagttccag TAT TATCCACCG (SEQ ID NO: 338) |
| 8nt_Split_probe_1 | AACCCTTA TAT agaggttgtgctggtggtggga (SEQ ID NO: 339) |
| 8nt_Split_probe_2 | acagggctgggagtagttccag TAT ATCCACCG (SEQ ID NO: 340) |
| 7nt_Split_probe_1 | AACCCTT TAT agaggttgtgctggtggtggga (SEQ ID NO: 341) |
| 7nt_Split_probe_2 | acagggctgggagtagttccag TAT TCCACCG (SEQ ID NO: 342) |

Readout sequence

| Name | Readout sequence |
|---|---|
| Readout | /5Cy5/GTAAGGGTT CGGTGGATA (SEQ ID NO: 343) |

Table 8b
MUC5AC split probe construct sequences

| Name | Sequence |
|---|---|
| Circular_probe_1 | AACCCTTAC TAT agaggttgtgctggtggtggga (SEQ ID NO: 344) |
| Circular_probe_2 | acagggctgggagtagttccag TAT TATCCACCG (SEQ ID NO: 345) |
| Cruciform_probe_1 | agaggttgtgctggtggtggga TAT AACCCTTAC (SEQ ID NO: 346) |
| Cruciform_probe_2 | TATCCACCG TAT acagggctgggagtagttccag (SEQ ID NO: 347) |
| Double‘C‘_probe_1 | agaggttgtgctggtggtggga TAT AACCCTTAC (SEQ ID NO: 348) |
| Double‘C‘_probe_2 | acagggctgggagtagttccag TAT TATCCACCG (SEQ ID NO: 349) |
| Double‘Z‘_probe_1 | agaggttgtgctggtggtggga TAT TATCCACCG (SEQ ID NO: 350) |
| Double‘Z‘_probe_2 | acagggctgggagtagttccag TAT AACCCTTAC (SEQ ID NO: 351) |
| Conventional_probe | TATCCACCGAACCCTTAC |
| | agaggttgtgctggtggtgggaacagggctgggagtagttccag (SEQ ID NO: 352) |
| Negative_control | AACCCTTAC TAT agaggttgtgctggtggtggga (SEQ ID NO: 353) |

MUC5AC split probe bridge sequence

| Name | Readout Sequence |
|---|---|
| Cy5_bridge | /5Cy5/GTAAGGGTT CGGTGGATA (SEQ ID NO: 354) |

Table 8c
MUC5AC colocalization Cy3 probe sequences

| Name | Sequence |
|---|---|
| MUC5AC_Cy3_1 | tgtccctcagcagcctctga TAT ATTTAACCGCACTATCCC (SEQ ID NO: 355) |
| MUC5AC_Cy3_2 | actcattgtatggacggcag TAT ATTTAACCGCACTATCCC (SEQ ID NO: 356) |
| MUC5AC_Cy3_3 | tectgggcatggcctatatg TAT ATTTAACCGCACTATCCC (SEQ ID NO: 357) |
| MUC5AC_Cy3_4 | gtagctagattcggaggagc TAT ATTTAACCGCACTATCCC (SEQ ID NO: 358) |
| MUC5AC_Cy3_5 | ataggagagagggcagggtg TAT ATTTAACCGCACTATCCC (SEQ ID NO: 359) |
| MUC5AC_Cy3_6 | agatgggaagacagtcgccc TAT ATTTAACCGCACTATCCC (SEQ ID NO: 360) |
| MUC5AC_Cy3_7 | ttagaggctcgtaccacagg TAT ATTTAACCGCACTATCCC (SEQ ID NO: 361) |
| MUC5AC_Cy3_8 | ggtcttgtagtagaagctac TAT ATTTAACCGCACTATCCC (SEQ ID NO: 362) |
| MUC5AC_Cy3_9 | gaacacgtagttacagaggc TAT ATTTAACCGCACTATCCC (SEQ ID NO: 363) |
| MUC5AC_Cy3_10 | aaatcctcgtaggcggcacc TAT ATTTAACCGCACTATCCC (SEQ ID NO: 364) |
| MUC5AC_Cy3_11 | atgaggaccctgctcagcgt TAT ATTTAACCGCACTATCCC (SEQ ID NO: 365) |
| MUC5AC_Cy3_12 | gatgaccacgccatccacct TAT ATTTAACCGCACTATCCC (SEQ ID NO: 366) |
| MUC5AC_Cy3_13 | ttgaccaggacggagccctt TAT ATTTAACCGCACTATCCC (SEQ ID NO: 367) |
| MUC5AC_Cy3_14 | agactggctgaagggcagca TAT ATTTAACCGCACTATCCC (SEQ ID NO: 368) |
| MUC5AC_Cy3_15 | tactctactgaatgaggacc TAT ATTTAACCGCACTATCCC (SEQ ID NO: 369) |
| MUC5AC_Cy3_16 | gcctccaccttggtgtagct TAT ATTTAACCGCACTATCCC (SEQ ID NO: 370) |
| MUC5AC_Cy3_17 | acatgaggacaaggcccagc TAT ATTTAACCGCACTATCCC (SEQ ID NO: 371) |
| MUC5AC_Cy3_18 | agcaggctatcatcatagtt TAT ATTTAACCGCACTATCCC (SEQ ID NO: 372) |
| MUC5AC_Cy3_19 | gtatttggtatccagctcca TAT ATTTAACCGCACTATCCC (SEQ ID NO: 373) |
| MUC5AC_Cy3_20 | acgggcatcccgttgaagtc TAT ATTTAACCGCACTATCCC (SEQ ID NO: 374) |
| MUC5AC_Cy3_21 | gcttggtattataggagagg TAT ATTTAACCGCACTATCCC (SEQ ID NO: 375) |
| MUC5AC_Cy3_22 | ttcccgaattccatgggtgt TAT ATTTAACCGCACTATCCC (SEQ ID NO: 376) |
| MUC5AC_Cy3_23 | acagggtcctgacactggtc TAT ATTTAACCGCACTATCCC (SEQ ID NO: 377) |
| MUC5AC_Cy3_24 | agccagtggagcagttcctc TAT ATTTAACCGCACTATCCC (SEQ ID NO: 378) |
| MUC5AC_Cy3_25 | aggagctcctcacagatgcc TAT ATTTAACCGCACTATCCC (SEQ ID NO: 379) |
| MUC5AC_Cy3_26 | acgcagccagagaacagctg TAT ATTTAACCGCACTATCCC (SEQ ID NO: 380) |
| MUC5AC_Cy3_27 | tgcaagcctccaggtagctg TAT ATTTAACCGCACTATCCC (SEQ ID NO: 381) |
| MUC5AC_Cy3_28 | tcacagaagcagaggtcttg TAT ATTTAACCGCACTATCCC (SEQ ID NO: 382) |
| MUC5AC_Cy3_29 | tactcagcaagggtatagca TAT ATTTAACCGCACTATCCC (SEQ ID NO: 383) |
| MUC5AC_Cy3_30 | tggtactgcatgttgttggg TAT ATTTAACCGCACTATCCC (SEQ ID NO: 384) |
| MUC5AC_Cy3_31 | ttggagcaggtgtctgcgca TAT ATTTAACCGCACTATCCC (SEQ ID NO: 385) |
| MUC5AC_Cy3_32 | acacagtggtcctcacaggc TAT ATTTAACCGCACTATCCC (SEQ ID NO: 386) |
| MUC5AC_Cy3_33 | atgtcgtcaagcaccgtccc TAT ATTTAACCGCACTATCCC (SEQ ID NO: 387) |
| MUC5AC_Cy3_34 | ttgacacagggacacagccg TAT ATTTAACCGCACTATCCC (SEQ ID NO: 388) |
| MUC5AC_Cy3_35 | ccgttgtagacgcaggcaca TAT ATTTAACCGCACTATCCC (SEQ ID NO: 389) |
| MUC5AC_Cy3_36 | agtctgtggagtaggtggcc TAT ATTTAACCGCACTATCCC (SEQ ID NO: 390) |
| MUC5AC_Cy3_37 | atggaacctcctggcagctc TAT ATTTAACCGCACTATCCC (SEQ ID NO: 391) |
| MUC5AC_Cy3_38 | aagcacagagcaggtacccg TAT ATTTAACCGCACTATCCC (SEQ ID NO: 392) |
| MUC5AC_Cy3_39 | acgttgagaagtaggcacct TAT ATTTAACCGCACTATCCC (SEQ ID NO: 393) |
| MUG5AC_Cy3_40 | accgtgtattgcttcccgtc TAT ATTTAACCGCACTATCCC (SEQ ID NO: 394) |
| MUC5AC_Cy3_41 | cacatagctgcagtcgccgt TAT ATTTAACCGCACTATCCC (SEQ ID NO: 395) |
| MUC5AC_Cy3_42 | tgctgtcacagggcttggtc TAT ATTTAACCGCACTATCCC (SEQ ID NO: 396) |
| MUC5AC_Cy3_43 | tcagccagtacagtgaaggc TAT ATTTAACCGCACTATCCC (SEQ ID NO: 397) |
| MUC5AC_Cy3_44 | ttcaggcaggtctcgctgtc TAT ATTTAACCGCACTATCCC (SEQ ID NO: 398) |
| MUC5AC_Cy3_45 | atccaggctcagtgtcacgc TAT ATTTAACCGCACTATCCC (SEQ ID NO: 399) |
| MUC5AC_Cy3_46 | tgatcaccaccaccgtctac TAT ATTTAACCGCACTATCCC (SEQ ID NO: 400) |
| MUC5AC_Cy3_47 | ctggttcaggaacacttccc TAT ATTTAACCGCACTATCCC (SEQ ID NO: 401) |
| MUC5AC_Cy3_48 | agatgggcagctgggtgtag TAT ATTTAACCGCACTATCCC (SEQ ID NO: 402) |
| MUC5AC_Cy3_49 | aagatggtgacgttagctac TAT ATTTAACCGCACTATCCC (SEQ ID NO: 403) |
| MUC5AC_Cy3_50 | gatgaagaaggttgagggtc TAT ATTTAACCGCACTATCCC (SEQ ID NO: 404) |
| MUC5AC_Cy3_51 | agctacaggttcagctacag TAT ATTTAACCGCACTATCCC (SEQ ID NO: 405) |
| MUC5AC_Cy3_52 | gaacagctgcatggtgggca TAT ATTTAACCGCACTATCCC (SEQ ID NO: 406) |
| MUC5AC_Cy3_53 | ttcccacagagaccgcaggt TAT ATTTAACCGCACTATCCC (SEQ ID NO: 407) |
| MUC5AC_Cy3_54 | atcggcctggatgctgttga TAT ATTTAACCGCACTATCCC (SEQ ID NO: 408) |
| MUC5AC_Cy3_55 | ttgaagaaggccgcagcagt TAT ATTTAACCGCACTATCCC (SEQ ID NO: 409) |
| MUC5AC_Cy3_56 | aagctgttcctgatgttggg TAT ATTTAACCGCACTATCCC (SEQ ID NO: 410) |
| MUC5AC_Cy3_57 | tctccacgctcagagagcag TAT ATTTAACCGCACTATCCC (SEQ ID NO: 411) |
| MUC5AC_Cy3_58 | cagtactgagcatacttctc TAT ATTTAACCGCACTATCCC (SEQ ID NO: 412) |
| MUC5AC_Cy3_59 | agtagattcccagcttcacg TAT ATTTAACCGCACTATCCC (SEQ ID NO: 413) |
| MUC5AC_Cy3_60 | gtatcaaacatgcagttcga TAT ATTTAACCGCACTATCCC (SEQ ID NO: 414) |
| MUC5AC_Cy3_61 | tcctcgctccgctcacagtt TAT ATTTAACCGCACTATCCC (SEQ ID NO: 415) |
| MUC5AC_Cy3_62 | ataggcttcgtgcagacgcc TAT ATTTAACCGCACTATCCC (SEQ ID NO: 416) |
| MUC5AC_Cy3_63 | tagtacgtcattgacttgag TAT ATTTAACCGCACTATCCC (SEQ ID NO: 417) |
| MUC5AC_Cy3_64 | ctggcaggtgctgacatggt TAT ATTTAACCGCACTATCCC (SEQ ID NO: 418) |
| MUC5AC_Cy3_65 | aacactgcaggtgatgtccc TAT ATTTAACCGCACTATCCC (SEQ ID NO: 419) |
| MUC5AC_Cy3_66 | acagatgcagccatccacgg TAT ATTTAACCGCACTATCCC (SEQ ID NO: 420) |
| MUC5AC_Cy3_67 | tcgtccaggaaggtgccctt TAT ATTTAACCGCACTATCCC (SEQ ID NO: 421) |
| MUC5AC_Cy3_68 | tactggcctacacacacttg TAT ATTTAACCGCACTATCCC (SEQ ID NO: 422) |
| MUC5AC_Cy3_69 | cctctgtggtagcagggaca TAT ATTTAACCGCACTATCCC (SEQ ID NO: 423) |
| MUC5AC_Cy3_70 | tgtgcaggtgcagatagccc TAT ATTTAACCGCACTATCCC (SEQ ID NO: 424) |
| MUC5AC_Cy3_71 | cgatgcagctcagcttccca TAT ATTTAACCGCACTATCCC (SEQ ID NO: 425) |
| MUC5AC_Cy3_72 | aacaccatgggcgcagcaca TAT ATTTAACCGCACTATCCC (SEQ ID NO: 426) |
| MUC5AC_Cy3_73 | catagcatttcggcagtcaa TAT ATTTAACCGCACTATCCC (SEQ ID NO: 427) |
| MUC5AC_Cy3_74 | agctcttctgacagccagcc TAT ATTTAACCGCACTATCCC (SEQ ID NO: 428) |
| MUC5AC_Cy3_75 | caggtcatgtccagtgtgtg TAT ATTTAACCGCACTATCCC (SEQ ID NO: 429) |

Colocalization readout sequence

| Name | Readout sequence |
|---|---|
| Readout | /5Cy3/GGGATAGTGCGGTTAAAT (SEQ ID NO: 430) |

Table 8d
Kinetic experiment probe sequences

| Name | Sequence |
|---|---|
| MUG5AC_Left_1 | AACCCTTAC TAT agaggttgtgctggtggtggga (SEQ ID NO: 431) |
| MUC5AC_Right_1 | acagggctgggagtagttccag TAT TATCCACCG (SEQ ID NO: 432) |
| MUC5AC_Conventional | acagggctgggagtagttccag TATCCACCGAACCCTTAC (SEQ ID NO: 433) |

Kinetic experiment bridge sequence

| Name | Sequence |
|---|---|
| Bridge_1 | TGATGGGTGCGTGAGTAAGTAAGGGTTCGGTGGATATGATGGGTGCGTGAGTAA (SEQ ID NO: 434) |

Kinetic experiment readout sequence

| Name | Sequence |
|---|---|
| Bridge Readout | /5Cy5/TTACTCACG CACCCATCA (SEQ ID NO: 435) |
| Conventional Readout | /5Cy5/GTAAGGGTT CGGTGGATA (SEQ ID NO: 436) |

TABLE 9
Probe sequences used in the FLNA experiment (FIG. 2). The template sequence includes
the forward and reverse primer sequences for amplifying the template sequence. The
primers used for PCR amplification are ‘TACCATCTCGTGTTCGTACC’ (SEQ ID NO: 437) and
‘TAATACGACTCACTATAGTTCGTTCCGCTACTCACCAC’ (SEQ ID NO: 438).
FLNA split probe sequences

| Name | Sequence |
|---|---|
| FLNA_1 | TACCATCTCGTGTTCGTACC GGTAC CCCATTACC TAT CAGGGAGCAGAGGTTGCGCTGCTGT GAATTC |
| | GTGGTGAGTAGCGGAACGAA (SEQ ID NO: 439) |
| FLNA_2 | TACCATCTCGTGTTCGTACC GGTAC CCCATTACC TAT GGAGCCGCGCCTGCTGCGCTCTGGC GAATTC |
| | GTGGTGAGTAGCGGAACGAA (SEQ ID NO: 440) |
| FLNA_3 | TACCATCTCGTGTTCGTACC GGTAC CCCATTACC TAT AGGTCCTTCTCGGTGGCCGGCATCT GAATTC |
| | GTGGTGAGTAGCGGAACGAA (SEQ ID NO: 441) |
| FLNA_4 | TACCATCTCGTGTTCGTACC GGTAC CCCATTACC TAT CACCAGCGCGTGAAAGTGTTCTGCT GAATTC |
| | GTGGTGAGTAGCGGAACGAA (SEQ ID NO: 442) |
| FLNA_5 | TACCATCTCGTGTTCGTACC GGTAC CCCATTACC TAT AGGTCCGTCTGCAGGTTGGCGATGC GAATTC |
| | GTGGTGAGTAGCGGAACGAA (SEQ ID NO: 443) |
| FLNA_6 | TACCATCTCGTGTTCGTACC GGTAC CCCATTACC TAT ATCTTCTTCTGGCTGAGCACCTCCA GAATTC |
| | GTGGTGAGTAGCGGAACGAA (SEQ ID NO: 444) |
| FLNA_7 | TACCATCTCGTGTTCGTACC GGTAC CCCATTACC TAT ACGTTCTCAAGCTGCATTTGGCGGA GAATTC |
| | GTGGTGAGTAGCGGAACGAA (SEQ ID NO: 445) |
| FLNA_8 | TACCATCTCGTGTTCGTACC GGTAC CCCATTACC TAT TCGATGGACACCAGTTTGATGCTCT GAATTC |
| | GTGGTGAGTAGCGGAACGAA (SEQ ID NO: 446) |
| FLNA_9 | TACCATCTCGTGTTCGTACC GGTAC CCCATTACC TAT GAGTAGTGCAGGATCAGGGTCCAGA GAATTC |
| | GTGGTGAGTAGCGGAACGAA (SEQ ID NO: 447) |
| FLNA_10 | TACCATCTCGTGTTCGTACC GGTAC CCCATTACC TAT GTCTGCTTCTTGGCCTCCTCATCCT GAATTC |
| | GTGGTGAGTAGCGGAACGAA (SEQ ID NO: 448) |
| FLNA_11 | TACCATCTCGTGTTCGTACC GGTAC CCCATTACC TAT ATGGGCAGCTGCGGCAGCTTGTTCT GAATTC |
| | GTGGTGAGTAGCGGAACGAA (SEQ ID NO: 449) |
| FLNA_12 | TACCATCTCGTGTTCGTACC GGTAC CCCATTACC TAT TCCACCAGGGCGCCCAGGGCCCGGC GAATTC |
| | GTGGTGAGTAGCGGAACGAA (SEQ ID NO: 450) |
| FLNA_13 | TACCATCTCGTGTTCGTACC GGTAC CCCATTACC TAT GGTAACGGGCTTGCTGGCGTCCCAA GAATTC |
| | GTGGTGAGTAGCGGAACGAA (SEQ ID NO: 451) |
| FLNA_14 | TACCATCTCGTGTTCGTACC GGTAC CCCATTACC TAT GGCCCGTAGGCACGGGCTTTCTTCG GAATTC |
| | GTGGTGAGTAGCGGAACGAA (SEQ ID NO: 452) |
| FLNA_15 | TACCATCTCGTGTTCGTACC GGTAC CCCATTACC TAT GGACGTACCAGACGGAGAAGGTGCG GAATTC |
| | GTGGTGAGTAGCGGAACGAA (SEQ ID NO: 453) |
| FLNA_16 | TACCATCTCGTGTTCGTACC GGTAC CCCATTACC TAT GGCTCTTGGCGATGTGCTGGCCAGC GAATTC |
| | GTGGTGAGTAGCGGAACGAA (SEQ ID NO: 454) |
| FLNA_17 | TACCATCTCGTGTTCGTACC GGTAC CCCATTACC TAT GCCGTAAAGATCTCAAAGTAGGTGG GAATTC |
| | GTGGTGAGTAGCGGAACGAA (SEQ ID NO: 455) |
| FLNA_18 | TACCATCTCGTGTTCGTACC GGTAC CCCATTACC TAT AGGGATGGGCACGCCGGCAAACGTG GAATTC |
| | GTGGTGAGTAGCGGAACGAA (SEQ ID NO: 456) |
| FLNA_19 | TACCATCTCGTGTTCGTACC GGTAC CCCATTACC TAT CCGCCCGGCAGGCACTCGGGTTACA GAATTC |
| | GTGGTGAGTAGCGGAACGAA (SEQ ID NO: 457) |
| FLNA_20 | TACCATCTCGTGTTCGTACC GGTAC CCCATTACC TAT CACCTTGAAGTCAGCTGTCTCCTTC GAATTC |
| | GTGGTGAGTAGCGGAACGAA (SEQ ID NO: 458) |
| FLNA_21 | TACCATCTCGTGTTCGTACC GGTAC CCCATTACC TAT CTCGGTGCCCACCTTCACTTCGAAG GAATTC |
| | GTGGTGAGTAGCGGAACGAA (SEQ ID NO: 459) |
| FLNA_22 | TACCATCTCGTGTTCGTACC GGTAC CCCATTACC TAT CACCACAAAGTCTGCTGACTTGCCA GAATTC |
| | GTGGTGAGTAGCGGAACGAA (SEQ ID NO: 460) |
| FLNA_23 | TACCATCTCGTGTTCGTACC GGTAC CCCATTACC TAT TTAGCCTGCGATGGCCCTTCCACCG GAATTC |
| | GTGGTGAGTAGCGGAACGAA (SEQ ID NO: 461) |
| FLNA_24 | TACCATCTCGTGTTCGTACC GGTAC CCCATTACC TAT TGCGGCCAGTAGCGCACATCACAGG GAATTC |
| | GTGGTGAGTAGCGGAACGAA (SEQ ID NO: 462) |
| FLNA_25 | TACCATCTCGTGTTCGTACC GGTAC CCCATTACC TAT AGGCGGATGTCTTCGCTGTTGCACA GAATTC |
| | GTGGTGAGTAGCGGAACGAA (SEQ ID NO: 463) |
| FLNA_26 | TACCATCTCGTGTTCGTACC GGTAC CCCATTACC TAT TTCTCCAATCCAGGCCCACGTGCCT GAATTC |
| | GTGGTGAGTAGCGGAACGAA (SEQ ID NO: 464) |
| FLNA_27 | TACCATCTCGTGTTCGTACC GGTAC CCCATTACC TAT CCGTGCTTGGCATCCACTGTGAACT GAATTC |
| | GTGGTGAGTAGCGGAACGAA (SEQ ID NO: 465) |
| FLNA_28 | TACCATCTCGTGTTCGTACC GGTAC CCCATTACC TAT CAACGCCTCCACAGGGCAGCCTTCA GAATTC |
| | GTGGTGAGTAGCGGAACGAA (SEQ ID NO: 466) |
| FLNA_29 | TACCATCTCGTGTTCGTACC GGTAC CCCATTACC TAT CGGCTTCCTGGGCACGTAGGAGCAG GAATTC |
| | GTGGTGAGTAGCGGAACGAA (SEQ ID NO: 467) |
| FLNA_30 | TACCATCTCGTGTTCGTACC GGTAC CCCATTACC TAT GCCAGCCTCGGCGCAGTCCACAGTG GAATTC |
| | GTGGTGAGTAGCGGAACGAA (SEQ ID NO: 468) |
| FLNA_31 | TACCATCTCGTGTTCGTACC GGTAC CCCATTACC TAT AGGTGTCATTGTCATTGCGGATGAT GAATTC |
| | GTGGTGAGTAGCGGAACGAA (SEQ ID NO: 469) |
| FLNA_32 | TACCATCTCGTGTTCGTACC GGTAC CCCATTACC TAT GTGGGCGTGGCCTGGTCAGCAAAGA GAATTC |
| | GTGGTGAGTAGCGGAACGAA (SEQ ID NO: 470) |
| FLNA_33 | TACCATCTCGTGTTCGTACC GGTAC CCCATTACC TAT CGGCCTTCACCTTACTGGCGTCATG GAATTC |
| | GTGGTGAGTAGCGGAACGAA (SEQ ID NO: 471) |
| FLNA_34 | TACCATCTCGTGTTCGTACC GGTAC CCCATTACC TAT TGAAGTGGGTGGGCTTGCCAAGCTC GAATTC |
| | GTGGTGAGTAGCGGAACGAA (SEQ ID NO: 472) |
| FLNA_35 | TACCATCTCGTGTTCGTACC GGTAC CCCATTACC TAT GTGTAGGTGTTGTCATGGTGGTCGA GAATTC |
| | GTGGTGAGTAGCGGAACGAA (SEQ ID NO: 473) |
| FLNA_36 | TACCATCTCGTGTTCGTACC GGTAC CCCATTACC TAT CCATAAGTGACATTGACGCCTACTG GAATTC |
| | GTGGTGAGTAGCGGAACGAA (SEQ ID NO: 474) |
| FLNA_37 | TACCATCTCGTGTTCGTACC GGTAC CCCATTACC TAT TCCAGGCTTGGAGATACTGCCACTG GAATTC |
| | GTGGTGAGTAGCGGAACGAA (SEQ ID NO: 475) |
| FLNA_38 | TACCATCTCGTGTTCGTACC GGTAC CCCATTACC TAT TCTTTGCCAACGTCCACCTTCTCTC GAATTC |
| | GTGGTGAGTAGCGGAACGAA (SEQ ID NO: 476) |
| FLNA_39 | TACCATCTCGTGTTCGTACC GGTAC CCCATTACC TAT GATGCCACTTTGCCTTGACCACCAG GAATTC |
| | GTGGTGAGTAGCGGAACGAA (SEQ ID NO: 477) |
| FLNA_40 | TACCATCTCGTGTTCGTACC GGTAC CCCATTACC TAT ACGGGCACGCCGTCATAGGTCACCT GAATTC |
| | GTGGTGAGTAGCGGAACGAA (SEQ ID NO: 478) |
| FLNA_41 | TACCATCTCGTGTTCGTACC GGTAC CCCATTACC TAT GGCCCAGGCCACCTGTGCCGGCGCC GAATTC |
| | GTGGTGAGTAGCGGAACGAA (SEQ ID NO: 479) |
| FLNA_42 | TACCATCTCGTGTTCGTACC GGTAC CCCATTACC TAT GCACTTGACTTTGGATGCGTCAAAG GAATTC |
| | GTGGTGAGTAGCGGAACGAA (SEQ ID NO: 480) |
| FLNA_43 | TACCATCTCGTGTTCGTACC GGTAC CCCATTACC TAT AGCTCCGCGCTGCCCGCGCTCGAGC GAATTC |
| | GTGGTGAGTAGCGGAACGAA (SEQ ID NO: 481) |
| FLNA_44 | TACCATCTCGTGTTCGTACC GGTAC CCCATTACC TAT TGGTGTGCGTGCCATCACCGTGGTC GAATTC |
| | GTGGTGAGTAGCGGAACGAA (SEQ ID NO: 482) |
| FLNA_45 | TACCATCTCGTGTTCGTACC GGTAC CCCATTACC TAT GGGAAGTTGGGCACGGGCTGGCCGC GAATTC |
| | GTGGTGAGTAGCGGAACGAA (SEQ ID NO: 483) |
| FLNA_46 | TACCATCTCGTGTTCGTACC GGTAC CCCATTACC TAT CCATAGCACTGGACACCGGAAGTGT GAATTC |
| | GTGGTGAGTAGCGGAACGAA (SEQ ID NO: 484) |
| FLNA_47 | TACCATCTCGTGTTCGTACC GGTAC CCCATTACC TAT ACACTGAACTCAGTGGTGGCCTCAC GAATTC |
| | GTGGTGAGTAGCGGAACGAA (SEQ ID NO: 485) |
| FLNA_48 | TACCATCTCGTGTTCGTACC GGTAC CCCATTACC TAT GCCACACGGGCCTTGACGTGCGGCC GAATTC |
| | GTGGTGAGTAGCGGAACGAA (SEQ ID NO: 486) |
| FLNA_49 | TACCATCTCGTGTTCGTACC GGTAC CCCATTACC TAT ACATGCCATCGCCACGGTCCTGAAC GAATTC |
| | GTGGTGAGTAGCGGAACGAA (SEQ ID NO: 487) |
| FLNA_50 | TACCATCTCGTGTTCGTACC GGTAC CCCATTACC TAT AGGTCACGTCCACGGAGTGCAGTCC GAATTC |
| | GTGGTGAGTAGCGGAACGAA (SEQ ID NO: 488) |
| FLNA_51 | TACCATCTCGTGTTCGTACC GGTAC CCCATTACC TAT CCGAGCAGCTGCCGTCCTTGTTATC GAATTC |
| | GTGGTGAGTAGCGGAACGAA (SEQ ID NO: 489) |
| FLNA_52 | TACCATCTCGTGTTCGTACC GGTAC CCCATTACC TAT CACCATAGGTGACGTTGAGGCTGTA GAATTC |
| | GTGGTGAGTAGCGGAACGAA (SEQ ID NO: 490) |
| FLNA_53 | TACCATCTCGTGTTCGTACC GGTAC CCCATTACC TAT CATCTGTCACATCATGCACAGGGAC GAATTC |
| | GTGGTGAGTAGCGGAACGAA (SEQ ID NO: 491) |
| FLNA_54 | TACCATCTCGTGTTCGTACC GGTAC CCCATTACC TAT TGGCACGAACCATGCCTGGGCTCAG GAATTC |
| | GTGGTGAGTAGCGGAACGAA (SEQ ID NO: 492) |
| FLNA_55 | TACCATCTCGTGTTCGTACC GGTAC CCCATTACC TAT GTCCACTGGCTCCACCAGGCCTTTG GAATTC |
| | GTGGTGAGTAGCGGAACGAA (SEQ ID NO: 493) |
| FLNA_56 | TACCATCTCGTGTTCGTACC GGTAC CCCATTACC TAT TTCTCGGCTGGGCACATAATTGACG GAATTC |
| | GTGGTGAGTAGCGGAACGAA (SEQ ID NO: 494) |
| FLNA_57 | TACCATCTCGTGTTCGTACC GGTAC CCCATTACC TAT TGGCCTTCACCTTGCTGGCATCATG GAATTC |
| | GTGGTGAGTAGCGGAACGAA (SEQ ID NO: 495) |
| FLNA_58 | TACCATCTCGTGTTCGTACC GGTAC CCCATTACC TAT TGGTGAACTCCACGGGCAGGCTGGC GAATTC |
| | GTGGTGAGTAGCGGAACGAA (SEQ ID NO: 496) |
| FLNA_59 | TACCATCTCGTGTTCGTACC GGTAC CCCATTACC TAT TTCTTCGGCTTGCCTTCGGGATCCG GAATTC |
| | GTGGTGAGTAGCGGAACGAA (SEQ ID NO: 497) |
| FLNA_60 | TACCATCTCGTGTTCGTACC GGTAC CCCATTACC TAT TCTGGCACGTAGGCCACTGTATACG GAATTC |
| | GTGGTGAGTAGCGGAACGAA (SEQ ID NO: 498) |
| FLNA_61 | TACCATCTCGTGTTCGTACC GGTAC CCCATTACC TAT ACTTTGCCTTTGCCTGCCGCCTTAG GAATTC |
| | GTGGTGAGTAGCGGAACGAA (SEQ ID NO: 499) |
| FLNA_62 | TACCATCTCGTGTTCGTACC GGTAC CCCATTACC TAT TCCACCACGTCCACATCCACCTCTG GAATTC |
| | GTGGTGAGTAGCGGAACGAA (SEQ ID NO: 500) |
| FLNA_63 | TACCATCTCGTGTTCGTACC GGTAC CCCATTACC TAT GGCACGTGCTCGCCACCAAAGCGCA GAATTC |
| | GTGGTGAGTAGCGGAACGAA (SEQ ID NO: 501) |
| FLNA_64 | TACCATCTCGTGTTCGTACC GGTAC CCCATTACC TAT TTGTCAGTGATGGTGGGCTGCGCCA GAATTC |
| | GTGGTGAGTAGCGGAACGAA (SEQ ID NO: 502) |
| FLNA_65 | TACCATCTCGTGTTCGTACC GGTAC CCCATTACC TAT TCGTGCAGGCCAGCCTCGCTGGGTG GAATTC |
| | GTGGTGAGTAGCGGAACGAA (SEQ ID NO: 503) |
| FLNA_66 | TACCATCTCGTGTTCGTACC GGTAC CCCATTACC TAT CATAGGCAGTGACATGGCCACAGTT GAATTC |
| | GTGGTGAGTAGCGGAACGAA (SEQ ID NO: 504) |
| FLNA_67 | TACCATCTCGTGTTCGTACC GGTAC CCCATTACC TAT CTGCATCCTTGGTGTTGACGGTGAA GAATTC |
| | GTGGTGAGTAGCGGAACGAA (SEQ ID NO: 505) |
| FLNA_68 | TACCATCTCGTGTTCGTACC GGTAC CCCATTACC TAT AGTGCAGCTGATTTCTGCTTTGGAC GAATTC |
| | GTGGTGAGTAGCGGAACGAA (SEQ ID NO: 506) |
| FLNA_69 | TACCATCTCGTGTTCGTACC GGTAC CCCATTACC TAT GGGCTGCCTGGGACGTGCTGTTCAT GAATTC |
| | GTGGTGAGTAGCGGAACGAA (SEQ ID NO: 507) |
| FLNA_70 | TACCATCTCGTGTTCGTACC GGTAC CCCATTACC TAT GGACCACAGTGGCCGTCAGCAGGCT GAATTC |
| | GTGGTGAGTAGCGGAACGAA (SEQ ID NO: 508) |
| FLNA_71 | TACCATCTCGTGTTCGTACC GGTAC CCCATTACC TAT CGTGGCCATTACGCAGCCGCTTCAG GAATTC |
| | GTGGTGAGTAGCGGAACGAA (SEQ ID NO: 509) |
| FLNA_72 | TACCATCTCGTGTTCGTACC GGTAC CCCATTACC TAT GTGTGGCCTTCGTGAAGGCCCTGAC GAATTC |
| | GTGGTGAGTAGCGGAACGAA (SEQ ID NO: 510) |
| FLNA_73 | TACCATCTCGTGTTCGTACC GGTAC CCCATTACC TAT AGCCCACCATAGCCTGCATCGCGGG GAATTC |
| | GTGGTGAGTAGCGGAACGAA (SEQ ID NO: 511) |
| FLNA_74 | TACCATCTCGTGTTCGTACC GGTAC CCCATTACC TAT CGTGCTGGTCGGCAAACTTGATGTT GAATTC |
| | GTGGTGAGTAGCGGAACGAA (SEQ ID NO: 512) |
| FLNA_75 | TACCATCTCGTGTTCGTACC GGTAC CCCATTACC TAT GGGTGATGCTCTCTTTCACCCGGCC GAATTC |
| | GTGGTGAGTAGCGGAACGAA (SEQ ID NO: 513) |
| FLNA_76 | TACCATCTCGTGTTCGTACC GGTACC CTGAGAGCGACCGGTGACCGATGAC TAT ATTTAACCG AATTC |
| | GTGGTGAGTAGCGGAACGAA (SEQ ID NO: 514) |
| FLNA_77 | TACCATCTCGTGTTCGTACC GGTACC GCCCGAGAGTGGGAGCTACTCATTT TAT ATTTAACCG AATTC |
| | GTGGTGAGTAGCGGAACGAA (SEQ ID NO: 515) |
| FLNA_78 | TACCATCTCGTGTTCGTACC GGTACC GCGTCCCGCGTGTCGACGCCGCCGC TAT ATTTAACCG AATTC |
| | GTGGTGAGTAGCGGAACGAA (SEQ ID NO: 516) |
| FLNA_79 | TACCATCTCGTGTTCGTACC GGTACC ATCTTCTTCCACGGCGCGTCCTCCG TAT ATTTAACCG AATTC |
| | GTGGTGAGTAGCGGAACGAA (SEQ ID NO: 517) |
| FLNA_80 | TACCATCTCGTGTTCGTACC GGTACC TTGCTCACGCACTTCAGGTGCTCGT TAT ATTTAACCG AATTC |
| | GTGGTGAGTAGCGGAACGAA (SEQ ID NO: 518) |
| FLNA_81 | TACCATCTCGTGTTCGTACC GGTACC AGCGCGATAAGCCGCAGCCCGTCGC TAT ATTTAACCG AATTC |
| | GTGGTGAGTAGCGGAACGAA (SEQ ID NO: 519) |
| FLNA_82 | TACCATCTCGTGTTCGTACC GGTACC GTGGGCCGCTGGTTGTGCTTGCGGT TAT ATTTAACCG AATTC |
| | GTGGTGAGTAGCGGAACGAA (SEQ ID NO: 520) |
| FLNA_83 | TACCATCTCGTGTTCGTACC GGTACC CGGTCCAGGAACTCGAGCGCCACCG TAT ATTTAACCG AATTC |
| | GTGGTGAGTAGCGGAACGAA (SEQ ID NO: 521) |
| FLNA_84 | TACCATCTCGTGTTCGTACC GGTACC AGGCCCAGGATCAGCTTCAGGTTCC TAT ATTTAACCG AATTC |
| | GTGGTGAGTAGCGGAACGAA (SEQ ID NO: 522) |
| FLNA_85 | TACCATCTCGTGTTCGTACC GGTACC TCCTCGTCCCACATGGGCATGGAGA TAT ATTTAACCG AATTC |
| | GTGGTGAGTAGCGGAACGAA (SEQ ID NO: 523) |
| FLNA_86 | TACCATCTCGTGTTCGTACC GGTACC ATCCAGCCCAGGAGCCTCTGCTTGG TAT ATTTAACCG AATTC |
| | GTGGTGAGTAGCGGAACGAA (SEQ ID NO: 524) |
| FLNA_87 | TACCATCTCGTGTTCGTACC GGTACC CTCTGCCAGTCCCGGCTGAAGTTGG TAT ATTTAACCG AATTC |
| | GTGGTGAGTAGCGGAACGAA (SEQ ID NO: 525) |
| FLNA_88 | TACCATCTCGTGTTCGTACC GGTACC GTCCCAGTCAGGACACAGGCCCGGG TAT ATTTAACCG AATTC |
| | GTGGTGAGTAGCGGAACGAA (SEQ ID NO: 526) |
| FLNA_89 | TACCATCTCGTGTTCGTACC GGTACC TTCAGTTTGGGCCGCAAGGGAGCCC TAT ATTTAACCG AATTC |
| | GTGGTGAGTAGCGGAACGAA (SEQ ID NO: 527) |
| FLNA_90 | TACCATCTCGTGTTCGTACC GGTACC TCTTGTCGTTATTGGCGGTCACTTT TAT ATTTAACCG AATTC |
| | GTGGTGAGTAGCGGAACGAA (SEQ ID NO: 528) |
| FLNA_91 | TACCATCTCGTGTTCGTACC GGTACC AGAGCACAGTAACCTTATGAGTCCC TAT ATTTAACCG AATTC |
| | GTGGTGAGTAGCGGAACGAA (SEQ ID NO: 529) |
| FLNA_92 | TACCATCTCGTGTTCGTACC GGTACC TTGTTGGCGATGTTGCCACTGGGCT TAT ATTTAACCG AATTC |
| | GTGGTGAGTAGCGGAACGAA (SEQ ID NO: 530) |
| FLNA_93 | TACCATCTCGTGTTCGTACC GGTACC GTGCACGGTGTGGACGCCCTCCATG TAT ATTTAACCG AATTC |
| | GTGGTGAGTAGCGGAACGAA (SEQ ID NO: 531) |
| FLNA_94 | TACCATCTCGTGTTCGTACC GGTACC CTTGGCCAACAGTGACAGTGTAGGG TAT ATTTAACCG AATTC |
| | GTGGTGAGTAGCGGAACGAA (SEQ ID NO: 532) |
| FLNA_95 | TACCATCTCGTGTTCGTACC GGTACC CCGCACACCCTTGGGCTGGAGGCCC TAT ATTTAACCG AATTC |
| | GTGGTGAGTAGCGGAACGAA (SEQ ID NO: 533) |
| FLNA_96 | TACCATCTCGTGTTCGTACC GGTACC ACTGCGCCCGATGTTCTGACCACCC TAT ATTTAACCG AATTC |
| | GTGGTGAGTAGCGGAACGAA (SEQ ID NO: 534) |
| FLNA_97 | TACCATCTCGTGTTCGTACC GGTACC GACGCCGCCCTCCAGCCCAGGGCCC TAT ATTTAACCG AATTC |
| | GTGGTGAGTAGCGGAACGAA (SEQ ID NO: 535) |
| FLNA_98 | TACCATCTCGTGTTCGTACC GGTACC AAGCCCAGCGTGCCCACGTCGTCCC TAT ATTTAACCG AATTC |
| | GTGGTGAGTAGCGGAACGAA (SEQ ID NO: 536) |
| FLNA_99 | TACCATCTCGTGTTCGTACC GGTACC CCGTCGCCCTTGTCGTCACATTCGA TAT ATTTAACCG AATTC |
| | GTGGTGAGTAGCGGAACGAA (SEQ ID NO: 537) |
| FLNA_100 | TACCATCTCGTGTTCGTACC GGTACC ACGTGAACGGCATACTCGCCAGCCT TAT ATTTAACCG AATTC |
| | GTGGTGAGTAGCGGAACGAA (SEQ ID NO: 538) |
| FLNA_101 | TACCATCTCGTGTTCGTACC GGTACC ACCCTGTCTGGGTGGAAGTCCTGGG TAT ATTTAACCG AATTC |
| | GTGGTGAGTAGCGGAACGAA (SEQ ID NO: 539) |
| FLNA_102 | TACCATCTCGTGTTCGTACC GGTACC GCTGGCTTGTTGACGGCCACACCTG TAT ATTTAACCG AATTC |
| | GTGGTGAGTAGCGGAACGAA (SEQ ID NO: 540) |
| FLNA_103 | TACCATCTCGTGTTCGTACC GGTACC GTCCTGGACTTGGACCCGAAGTGGG TAT ATTTAACCG AATTC |
| | GTGGTGAGTAGCGGAACGAA (SEQ ID NO: 541) |
| FLNA_104 | TACCATCTCGTGTTCGTACC GGTACC GTAAGTGCCATTGCCGTTGTCCTTG TAT ATTTAACCG AATTC |
| | GTGGTGAGTAGCGGAACGAA (SEQ ID NO: 542) |
| FLNA_105 | TACCATCTCGTGTTCGTACC GGTACC GTAGGTGGGCTCGTGGGCCTTGAGC TAT ATTTAACCG AATTC |
| | GTGGTGAGTAGCGGAACGAA (SEQ ID NO: 543) |
| FLNA_106 | TACCATCTCGTGTTCGTACC GGTACC CGAAGTCGATGTCAGCTTCGGCGGG TAT ATTTAACCG AATTC |
| | GTGGTGAGTAGCGGAACGAA (SEQ ID NO: 544) |
| FLNA_107 | TACCATCTCGTGTTCGTACC GGTACC ACCATAATGGTGTAGCTGCCAGCCC TAT ATTTAACCG AATTC |
| | GTGGTGAGTAGCGGAACGAA (SEQ ID NO: 545) |
| FLNA_108 | TACCATCTCGTGTTCGTACC GGTACC AGGGCTCCACCTTGACTCGGATGGG TAT ATTTAACCG AATTC |
| | GTGGTGAGTAGCGGAACGAA (SEQ ID NO: 546) |
| FLNA_109 | TACCATCTCGTGTTCGTACC GGTACC CACCAGTGCGACTGAGGCCAGGGCC TAT ATTTAACCG AATTC |
| | GTGGTGAGTAGCGGAACGAA (SEQ ID NO: 547) |
| FLNA_110 | TACCATCTCGTGTTCGTACC GGTACC ATGTCCACATCTCGCACTGCATCCC TAT ATTTAACCG AATTC |
| | GTGGTGAGTAGCGGAACGAA (SEQ ID NO: 548) |
| FLNA_111 | TACCATCTCGTGTTCGTACC GGTACC CCCTGCTGGACAGGCGTGTACTTGA TAT ATTTAACCG AATTC |
| | GTGGTGAGTAGCGGAACGAA (SEQ ID NO: 549) |
| FLNA_112 | TACCATCTCGTGTTCGTACC GGTACC AAAGGGCTCTTAGGGATGGGATCCC TAT ATTTAACCG AATTC |
| | GTGGTGAGTAGCGGAACGAA (SEQ ID NO: 550) |
| FLNA_113 | TACCATCTCGTGTTCGTACC GGTACC AGGCCAGACACCTTGATCTTGCTGA TAT ATTTAACCG AATTC |
| | GTGGTGAGTAGCGGAACGAA (SEQ ID NO: 551) |
| FLNA_114 | TACCATCTCGTGTTCGTACC GGTACC CCCTTTGATTTGACTGTGAACTCCT TAT ATTTAACCG AATTC |
| | GTGGTGAGTAGCGGAACGAA (SEQ ID NO: 552) |
| FLNA_115 | TACCATCTCGTGTTCGTACC GGTACC ACCTCATAGGGCCCTTCCTCACGGG TAT ATTTAACCG AATTC |
| | GTGGTGAGTAGCGGAACGAA (SEQ ID NO: 553) |
| FLNA_116 | TACCATCTCGTGTTCGTACC GGTACC TGGTGTCGATGGTGAAGCGGGCGGG TAT ATTTAACCG AATTC |
| | GTGGTGAGTAGCGGAACGAA (SEQ ID NO: 554) |
| FLNA_117 | TACCATCTCGTGTTCGTACC GGTACC GGGAACCACGTGGGCCTTGAATGGG TAT ATTTAACCG AATTC |
| | GTGGTGAGTAGCGGAACGAA (SEQ ID NO: 555) |
| FLNA_118 | TACCATCTCGTGTTCGTACC GGTACC TCCACTTGGAATTGGCCCACCTCCC TAT ATTTAACCG AATTC |
| | GTGGTGAGTAGCGGAACGAA (SEQ ID NO: 556) |
| FLNA_119 | TACCATCTCGTGTTCGTACC GGTACC GGATGTACACCTCGGCCGGAAGCCC TAT ATTTAACCG AATTC |
| | GTGGTGAGTAGCGGAACGAA (SEQ ID NO: 557) |
| FLNA_120 | TACCATCTCGTGTTCGTACC GGTACC TACTTGATGGTGACGGTGTAGGCCC TAT ATTTAACCG AATTC |
| | GTGGTGAGTAGCGGAACGAA (SEQ ID NO: 558) |
| FLNA_121 | TACCATCTCGTGTTCGTACC GGTACC ACCGCAGGTTCCACCTGCAGCTTGC TAT ATTTAACCG AATTC |
| | GTGGTGAGTAGCGGAACGAA (SEQ ID NO: 559) |
| FLNA_122 | TACCATCTCGTGTTCGTACC GGTACC AAGACACCCTGGCCCTCAATACCAG TAT ATTTAACCG AATTC |
| | GTGGTGAGTAGCGGAACGAA (SEQ ID NO: 560) |
| FLNA_123 | TACCATCTCGTGTTCGTACC GGTACC CCGGTCTGTGTCAGAGCCCGGGCGT TAT ATTTAACCG AATTC |
| | GTGGTGAGTAGCGGAACGAA (SEQ ID NO: 561) |
| FLNA_124 | TACCATCTCGTGTTCGTACC GGTACC AGGTCTCCGTCAGGTTGCCTGAGGG TAT ATTTAACCG AATTC |
| | GTGGTGAGTAGCGGAACGAA (SEQ ID NO: 562) |
| FLNA_125 | TACCATCTCGTGTTCGTACC GGTACC CCTCGTAAGGCGTGTACTCCACTTT TAT ATTTAACCG AATTC |
| | GTGGTGAGTAGCGGAACGAA (SEQ ID NO: 563) |
| FLNA_126 | TACCATCTCGTGTTCGTACC GGTACC TGCAGGACATCTTGGCCTCGGAGGG TAT ATTTAACCG AATTC |
| | GTGGTGAGTAGCGGAACGAA (SEQ ID NO: 564) |
| FLNA_127 | TACCATCTCGTGTTCGTACC GGTACC TGCCAGCCTCATAAGGGATGTACTC TAT ATTTAACCG AATTC |
| | GTGGTGAGTAGCGGAACGAA (SEQ ID NO: 565) |
| FLNA_128 | TACCATCTCGTGTTCGTACC GGTACC TGAAAGGACTGCCTGGCACTTGATG TAT ATTTAACCG AATTC |
| | GTGGTGAGTAGCGGAACGAA (SEQ ID NO: 566) |
| FLNA_129 | TACCATCTCGTGTTCGTACC GGTACC CGGGCCCAGAGCACTTGACCTTGGA TAT ATTTAACCG AATTC |
| | GTGGTGAGTAGCGGAACGAA (SEQ ID NO: 567) |
| FLNA_130 | TACCATCTCGTGTTCGTACC GGTACC CCCTTGCACTTTGACCTGCAATGGG TAT ATTTAACCG AATTC |
| | GTGGTGAGTAGCGGAACGAA (SEQ ID NO: 568) |
| FLNA_131 | TACCATCTCGTGTTCGTACC GGTACC CTGGGTGCCATCAGCGTTGTCTACC TAT ATTTAACCG AATTC |
| | GTGGTGAGTAGCGGAACGAA (SEQ ID NO: 569) |
| FLNA_132 | TACCATCTCGTGTTCGTACC GGTACC TAGGCAGCACCTTGACCTTGAAGGG TAT ATTTAACCG AATTC |
| | GTGGTGAGTAGCGGAACGAA (SEQ ID NO: 570) |
| FLNA_133 | TACCATCTCGTGTTCGTACC GGTACC GCACGCCAGTGGTGTTGAGCCCGGG TAT ATTTAACCG AATTC |
| | GTGGTGAGTAGCGGAACGAA (SEQ ID NO: 571) |
| FLNA_134 | TACCATCTCGTGTTCGTACC GGTACC ATCTGGACAGCCAGCAGGCCCTCCC TAT ATTTAACCG AATTC |
| | GTGGTGAGTAGCGGAACGAA (SEQ ID NO: 572) |
| FLNA_135 | TACCATCTCGTGTTCGTACC GGTACC CCGTCATGGTTGTCTTGGATGTGTG TAT ATTTAACCG AATTC |
| | GTGGTGAGTAGCGGAACGAA (SEQ ID NO: 573) |
| FLNA_136 | TACCATCTCGTGTTCGTACC GGTACC TCCACAGTGATCACCGTCTCCTCCC TAT ATTTAACCG AATTC |
| | GTGGTGAGTAGCGGAACGAA (SEQ ID NO: 574) |
| FLNA_137 | TACCATCTCGTGTTCGTACC GGTACC CCATCAGGCGTGCACACGGTGCACG TAT ATTTAACCG AATTC |
| | GTGGTGAGTAGCGGAACGAA (SEQ ID NO: 575) |
| FLNA_138 | TACCATCTCGTGTTCGTACC GGTACC CAGATGACGTATTTGCCCGGCTGGG TAT ATTTAACCG AATTC |
| | GTGGTGAGTAGCGGAACGAA (SEQ ID NO: 576) |
| FLNA_139 | TACCATCTCGTGTTCGTACC GGTACC TTGCCTGAGGGCATCCGAACCTCCC TAT ATTTAACCG AATTC |
| | GTGGTGAGTAGCGGAACGAA (SEQ ID NO: 577) |
| FLNA_140 | TACCATCTCGTGTTCGTACC GGTACC TACCGCACGGTCACGGTGCCGTCTT TAT ATTTAACCG AATTC |
| | GTGGTGAGTAGCGGAACGAA (SEQ ID NO: 578) |
| FLNA_141 | TACCATCTCGTGTTCGTACC GGTACC CGTAATCCACATAGAACTGCAAGGG TAT ATTTAACCG AATTC |
| | GTGGTGAGTAGCGGAACGAA (SEQ ID NO: 579) |
| FLNA_142 | TACCATCTCGTGTTCGTACC GGTACC TGGCAGGCTTGTTCACTACTCCATG TAT ATTTAACCG AATTC |
| | GTGGTGAGTAGCGGAACGAA (SEQ ID NO: 580) |
| FLNA_143 | TACCATCTCGTGTTCGTACC GGTACC GCCCTCAATGGCCAGAGACAGGCCC TAT ATTTAACCG AATTC |
| | GTGGTGAGTAGCGGAACGAA (SEQ ID NO: 581) |
| FLNA_144 | TACCATCTCGTGTTCGTACC GGTACC TACTTGACTAGAATGCTGTAGTCCC TAT ATTTAACCG AATTC |
| | GTGGTGAGTAGCGGAACGAA (SEQ ID NO: 582) |
| FLNA_145 | TACCATCTCGTGTTCGTACC GGTACC GATCCGTCTCTGAGATGTTGATGGG TAT ATTTAACCG AATTC |
| | GTGGTGAGTAGCGGAACGAA (SEQ ID NO: 583) |
| FLNA_146 | TACCATCTCGTGTTCGTACC GGTACC AACAGGGCTCCTCCCGGCCCGAGGG TAT ATTTAACCG AATTC |
| | GTGGTGAGTAGCGGAACGAA (SEQ ID NO: 584) |
| FLNA_147 | TACCATCTCGTGTTCGTACC GGTACC GAGACCCGAACACGACTGGCATCCC TAT ATTTAACCG AATTC |
| | GTGGTGAGTAGCGGAACGAA (SEQ ID NO: 585) |
| FLNA_148 | TACCATCTCGTGTTCGTACC GGTACC TCAATGATAAACTCTGCAGGCTCAA TAT ATTTAACCG AATTC |
| | GTGGTGAGTAGCGGAACGAA (SEQ ID NO: 586) |
| FLNA_149 | TACCATCTCGTGTTCGTACC GGTACC TGATGTAGTTGCCTGGCTCTGTGGG TAT ATTTAACCG AATTC |
| | GTGGTGAGTAGCGGAACGAA (SEQ ID NO: 587) |
| FLNA_150 | TACCATCTCGTGTTCGTACC GGTACC CGCCTGTCACCTTCACAGAGAAGGG TAT ATTTAACCG AATTC |
| | GTGGTGAGTAGCGGAACGAA (SEQ ID NO: 588) |

FLNA split probe bridge sequence

| Name | Bridge sequence |
|---|---|
| FLNA_bridge | ATTGTAAAGCGTGAGAAA GGTAATGGGCGGTTAAAT ATTGTAAAGCGTGAGAAA (SEQ ID NO: 589) |

FLNA split probe readout sequence

| | |
|---|---|
| Name | Readout sequence |

| | |
|---|---|
| Cy5_Readout | /5Cy5/TTTCTCACGCTTTACAAT (SEQ ID NO: 590) |

TABLE 10
Reference FKPM values for AML12, mouse kidney, liver, frontal cortex and ovary for
the 317 genes in the split library.
Reference FPKM for 317-genes split library

| | | | | | Frontal | |
|---|---|---|---|---|---|---|
| Gene name | Transcript ID | AML-12 | Kidney | Liver | Cortex | Ovary |
| Blank1 | | 0 | 0 | 0 | 0 | 0 |
| Blank2 | | 0 | 0 | 0 | 0 | 0 |
| Blank3 | | 0 | 0 | 0 | 0 | 0 |
| Blank4 | | 0 | 0 | 0 | 0 | 0 |
| Blank5 | | 0 | 0 | 0 | 0 | 0 |
| Blank6 | | 0 | 0 | 0 | 0 | 0 |
| Blank7 | | 0 | 0 | 0 | 0 | 0 |
| Blank8 | | 0 | 0 | 0 | 0 | 0 |
| Ppfia2 | ENSMUST00000029404.11 | 0.15192 | 0.04 | 0.02 | 15.425 | 0.02 |
| Kif5a | ENSMUST00000099172.3 | 0.068099 | 1.295 | 1.235 | 342.67 | 3.45 |
| Camk4 | ENSMUST00000042868.4 | 0.16044 | 0.035 | 0.005 | 10.56 | 0.035 |
| Cenpe | ENSMUST00000062893.7 | 18.8485 | 0.045 | 0.01 | 0.2 | 0.185 |
| Akap6 | ENSMUST00000095737.3 | 0.032227 | 0.14 | 0.025 | 15.605 | 0.365 |
| Kif1a | ENSMUST00000171796.3 | 0.089827 | 0.64 | 0.07 | 120.305 | 4.315 |
| Ptprz1 | ENSMUST00000090568.3 | 0.061093 | 0.06 | 0.01 | 35.63 | 1.46 |
| Ccdc177 | ENSMUST00000073251.6 | 0 | 0 | 0.005 | 2.39 | 0.105 |
| Mki67 | ENSMUST00000033310.7 | 37.822 | 0.135 | 0.03 | 0.235 | 1.435 |
| Map1a | ENSMUST00000094639.5 | 0.035225 | 0.4 | 0.035 | 82.56 | 3.655 |
| Hmcn2 | ENSMUST00000113532.4 | 0.01155 | 0.18 | 0.01 | 0.155 | 7.08 |
| Brip1 | ENSMUST00000044423.3 | 12.0383 | 0.26 | 0.115 | 0.025 | 0.13 |
| Aspm | ENSMUST00000053364.10 | 15.0238 | 0.02 | 0.01 | 0.07 | 0.755 |
| Kntc1 | ENSMUST00000031366.7 | 19.5018 | 0.115 | 0.015 | 0.11 | 0.885 |
| Myof | ENSMUST00000041475.10 | 154.2339 | 4.67 | 0.15 | 0.97 | 4.735 |
| Myom2 | ENSMUST00000033842.3 | 0.081423 | 4.555 | 0.04 | 0.045 | 0.125 |
| Ahnak | ENSMUST00000092956.2 | 236.0856 | 6.225 | 0.805 | 2.77 | 8.6 |
| Cacna1e | ENSMUST00000187541.2 | 1.3159 | 0.065 | 0.02 | 18.545 | 0.19 |
| Zfp831 | ENSMUST00000059452.5 | 0.013624 | 0.105 | 0.04 | 2.68 | 0.03 |
| Rims3 | ENSMUST00000071093.4 | 0.16167 | 0.18 | 0.335 | 23.255 | 1.04 |
| Adamts2 | ENSMUST00000040523.8 | 0.14825 | 3.165 | 1.51 | 0.775 | 71.525 |
| Dzank1 | ENSMUST00000081982.7 | 1.0306 | 0.935 | 0.045 | 30.275 | 1.17 |
| Pik3c2a | ENSMUST00000170430.1 | 74.0708 | 2.65 | 0.875 | 3.015 | 0.835 |
| Prune2 | ENSMUST00000087689.4 | 0.17921 | 0.13 | 0.005 | 9.47 | 0.86 |
| Myh11 | ENSMUST00000090287.3 | 0.003366 | 4.365 | 0.26 | 2.175 | 57.02 |
| Nat8l | ENSMUST00000056355.8 | 0.016113 | 3.56 | 0.03 | 58.45 | 4.895 |
| Myo5c | ENSMUST00000036555.6 | 38.7977 | 2.195 | 0.02 | 0.34 | 3.21 |
| Erc2 | ENSMUST00000090302.5 | 0.18012 | 0.805 | 0.74 | 17.365 | 0.085 |
| Brca2 | ENSMUST00000044620.7 | 6.4182 | 0.125 | 0.065 | 0.415 | 0.31 |
| Tspan7 | ENSMUST00000115526.1 | 19.8019 | 48.37 | 15.395 | 741.31 | 24.435 |
| Col6a6 | ENSMUST00000098441.5 | 0.008119 | 0.135 | 0.03 | 0.09 | 1.71 |
| Atp1a2 | ENSMUST00000085913.6 | 1.116 | 14.055 | 0.195 | 185.775 | 17.37 |
| Galnt15 | ENSMUST00000022460.6 | 0.050628 | 1.6 | 0.315 | 0.57 | 16.08 |
| Trabd2b | ENSMUST00000094894.3 | 0.02677 | 18.39 | 0.06 | 0.555 | 2.84 |
| Smchd1 | ENSMUST00000127430.1 | 34.3279 | 1.195 | 0.345 | 3.54 | 1.165 |
| Mysm1 | ENSMUST00000075872.3 | 16.725 | 0.43 | 0.24 | 2.415 | 0.24 |
| Spag17 | ENSMUST00000164539.1 | 0.024643 | 0 | 0.005 | 0.01 | 0.21 |
| Dock10 | ENSMUST00000077946.7 | 0.20537 | 1.57 | 0.38 | 14.565 | 0.565 |
| Srgap3 | ENSMUST00000088373.6 | 0.73845 | 3.225 | 0.725 | 49.765 | 5.47 |
| Atr | ENSMUST00000034980.7 | 18.0366 | 0.92 | 0.4 | 1.74 | 0.57 |
| Armcx4 | ENSMUST00000124226.2 | 0.043819 | 0.505 | 0.11 | 6.98 | 0.87 |
| Tnxb | ENSMUST00000087399.4 | 0.054933 | 4.07 | 1.98 | 0.635 | 36.685 |
| Utp20 | ENSMUST00000004470.7 | 33.4202 | 1.94 | 0.87 | 2.585 | 1.655 |
| Gas2l3 | ENSMUST00000099374.4 | 9.6628 | 0.11 | 0.22 | 0.47 | 1.325 |
| Veph1 | ENSMUST00000029419.7 | 0.93919 | 3.83 | 0.02 | 0.04 | 0.055 |
| Col4a4 | ENSMUST00000087050.6 | 0.072728 | 17.545 | 0.055 | 0.125 | 4.8 |
| Ly75 | ENSMUST00000112533.3 | 7.5712 | 0.325 | 0.375 | 0.44 | 0.43 |
| Hivep2 | ENSMUST00000187083.2 | 1.2425 | 2.81 | 0.98 | 37.99 | 3.995 |
| Myh7b | ENSMUST00000092995.5 | 0.14153 | 0.015 | 0.005 | 0.905 | 0.08 |
| Map3k9 | ENSMUST00000035987.7 | 0.014592 | 1.37 | 0.015 | 7.035 | 0.565 |
| Foxo1 | ENSMUST00000053764.6 | 0.23454 | 8.175 | 4.485 | 3.645 | 77.605 |
| Ntrk2 | ENSMUST00000079828.5 | 0.20646 | 1.97 | 2.285 | 70.06 | 13.32 |
| Scn10a | ENSMUST00000084787.5 | 0.010614 | 0 | 0.005 | 0.05 | 0.24 |
| Flnc | ENSMUST00000090474.6 | 5.9856 | 0.85 | 0.03 | 0.22 | 21.62 |
| Pygo1 | ENSMUST00000038489.5 | 0.051895 | 0.17 | 0.145 | 5.09 | 1.16 |
| Pask | ENSMUST00000027493.3 | 8.6466 | 0.3 | 0.225 | 0.56 | 1.425 |
| Sptbn2 | ENSMUST00000008991.6 | 0.12332 | 6.575 | 8.4 | 78.255 | 1.355 |
| Ksr2 | ENSMUST00000180430.1 | 0.10047 | 0.99 | 0.04 | 3.68 | 0.1 |
| Slk | ENSMUST00000026043.7 | 81.5086 | 5.51 | 2.59 | 11.565 | 3.79 |
| Sptlc2 | ENSMUST00000021424.4 | 58.8505 | 6.77 | 1.565 | 3.6 | 5.75 |
| Nup210l | ENSMUST00000029548.4 | 1.483 | 0.075 | 0.01 | 0.165 | 0.27 |
| Mrc2 | ENSMUST00000100335.5 | 0.23292 | 13.505 | 0.49 | 2.135 | 40.645 |
| Utp14b | ENSMUST00000053760.7 | 11.6554 | 0.325 | 0.555 | 2.755 | 0.265 |
| Notch3 | ENSMUST00000087723.3 | 0.18334 | 8.185 | 0.87 | 3.265 | 34.575 |
| Cand1 | ENSMUST00000020315.8 | 86.0633 | 5.045 | 3.075 | 12.2 | 8.135 |
| Col6a5 | ENSMUST00000190193.2 | 0.004947 | 0.16 | 0.06 | 0.12 | 1.075 |
| Sdk2 | ENSMUST00000041627.9 | 0.074561 | 0.255 | 0.05 | 2.91 | 0.725 |
| Lgr6 | ENSMUST00000044828.9 | 0.60692 | 0.33 | 0.015 | 8.17 | 19.76 |
| Prtg | ENSMUST00000055535.8 | 0.028414 | 0.035 | 0.005 | 0.54 | 0.15 |
| Dnah1 | ENSMUST00000048603.7 | 0.069171 | 0.08 | 0.015 | 0.68 | 1.895 |
| Zbtb41 | ENSMUST00000039867.8 | 23.8026 | 2.995 | 0.885 | 3.395 | 1.465 |
| Fbxo32 | ENSMUST00000022986.6 | 1.2279 | 0.83 | 0.175 | 2.49 | 11.72 |
| Caskin1 | ENSMUST00000024958.7 | 0.091806 | 0.175 | 0.055 | 32.07 | 18.57 |
| Zfp26 | ENSMUST00000159569.3 | 19.7889 | 1.11 | 0.645 | 4.835 | 1.185 |
| Cacna1c | ENSMUST00000186889.2 | 0.071178 | 0.47 | 0.025 | 10.03 | 4.625 |
| Heatr1 | ENSMUST00000059270.8 | 27.1337 | 1.79 | 1.335 | 3.25 | 3.395 |
| Col4a6 | ENSMUST00000101205.2 | 1.1748 | 0.905 | 0.01 | 0.275 | 5.41 |
| Hspg2 | ENSMUST00000030547.10 | 0.97227 | 10.445 | 2.14 | 0.975 | 37.47 |
| Kpnb1 | ENSMUST00000001479.4 | 193.7558 | 13.28 | 9.59 | 25.755 | 22.54 |
| Zfyve16 | ENSMUST00000022217.8 | 12.7013 | 1.315 | 0.375 | 2.515 | 0.885 |
| Myh10 | ENSMUST00000102611.5 | 1.1369 | 3.245 | 0.36 | 24 | 5.585 |
| Slc4a7 | ENSMUST00000057015.6 | 20.3773 | 0.735 | 0.57 | 5.095 | 2.165 |
| Grik3 | ENSMUST00000030676.7 | 0.030225 | 0.06 | 0.01 | 15.785 | 10.89 |
| Rnf150 | ENSMUST00000078525.5 | 0.13519 | 0.845 | 0.115 | 5.98 | 1.535 |
| Sdk1 | ENSMUST00000085774.6 | 0.017245 | 0.435 | 0.085 | 1.43 | 4.22 |
| Ythdc2 | ENSMUST00000037763.7 | 12.1174 | 0.545 | 0.375 | 4.255 | 0.365 |
| Il17rd | ENSMUST00000035336.3 | 0.23613 | 2.01 | 0.1 | 0.84 | 7.02 |
| Synm | ENSMUST00000074233.6 | 0.21727 | 0.41 | 0.035 | 4.135 | 8.365 |
| Dnah11 | ENSMUST00000084806.6 | 0.021572 | 0.315 | 0.06 | 2.335 | 0.68 |
| Flna | ENSMUST00000114299.3 | 173.0184 | 37.345 | 5.2 | 21.145 | 470.345 |
| Fry | ENSMUST00000087204.5 | 0.62454 | 2.6 | 0.145 | 13.31 | 2.815 |
| Cacna1a | ENSMUST00000121390.3 | 4.1076 | 0.545 | 0.21 | 20.9 | 5.36 |
| Chst2 | ENSMUST00000036267.7 | 0.06983 | 1.705 | 0.29 | 15.45 | 6.065 |
| Psd3 | ENSMUST00000038959.11 | 18.5113 | 1.065 | 2.1 | 46.12 | 1.5 |
| Gpr161 | ENSMUST00000111450.2 | 0.43661 | 0.91 | 0.09 | 1.915 | 6.81 |
| Plxnd1 | ENSMUST00000015511.10 | 3.4124 | 30.905 | 10.29 | 15.525 | 144.685 |
| Rsf1 | ENSMUST00000042399.9 | 12.4442 | 0.82 | 0.275 | 4.82 | 0.79 |
| Slc38a1 | ENSMUST00000100262.2 | 0.95264 | 1.315 | 0.265 | 26.725 | 14.9 |
| Cacna2d1 | ENSMUST00000039370.9 | 20.2413 | 0.61 | 0.05 | 32.18 | 2.805 |
| Ppl | ENSMUST00000035672.3 | 17.2285 | 2.04 | 2.3 | 0.085 | 2.15 |
| Trove2 | ENSMUST00000159879.1 | 13.2001 | 1.315 | 0.155 | 5.505 | 0.845 |
| Sox11 | ENSMUST00000079063.6 | 7.5783 | 0.04 | 0.005 | 4.92 | 0.81 |
| Mlxip | ENSMUST00000068237.7 | 1.539 | 9.295 | 2.63 | 6.43 | 42.29 |
| Pkd1l3 | ENSMUST00000109242.3 | 0.23261 | 0.015 | 0.005 | 0.09 | 0.025 |
| Igsf10 | ENSMUST00000039419.7 | 0.008606 | 0.32 | 0.145 | 0.4 | 1.785 |
| Eif5b | ENSMUST00000027252.7 | 17.3736 | 1.9 | 1.325 | 4.695 | 0.705 |
| Nid1 | ENSMUST00000005532.7 | 1.1068 | 28.78 | 4.265 | 4.25 | 65.63 |
| Alms1 | ENSMUST00000072018.5 | 4.1423 | 0.32 | 0.095 | 1.265 | 0.715 |
| Csmd1 | ENSMUST00000082104.6 | 0.092488 | 0.15 | 0.005 | 1.875 | 1.59 |
| Zfp516 | ENSMUST00000171238.3 | 3.3071 | 3.195 | 1.31 | 3.74 | 22.735 |
| Shroom3 | ENSMUST00000113054.4 | 0.7962 | 10.415 | 2.085 | 0.715 | 24.465 |
| Ddr2 | ENSMUST00000194690.1 | 0.31682 | 5.51 | 0.755 | 2.17 | 14.56 |
| Bend4 | ENSMUST00000169190.1 | 0.038403 | 0.135 | 0.08 | 1.435 | 1.93 |
| Sned1 | ENSMUST00000062202.9 | 0.83901 | 4.685 | 3.3 | 2.385 | 24.19 |
| Piezo1 | ENSMUST00000067252.9 | 6.7555 | 24.03 | 2.055 | 1.71 | 50.425 |
| Adamts17 | ENSMUST00000098382.5 | 0.033952 | 0.095 | 0.025 | 0.94 | 0.775 |
| Rttn | ENSMUST00000023828.7 | 4.6342 | 0.675 | 0.175 | 0.925 | 0.97 |
| Trpm2 | ENSMUST00000105401.4 | 0.55543 | 0.11 | 0.215 | 1.705 | 0.035 |
| Arhgap31 | ENSMUST00000023487.4 | 0.91672 | 3.49 | 0.75 | 4.68 | 15.86 |
| Mkl2 | ENSMUST00000149359.1 | 1.6283 | 3.385 | 2.515 | 26.125 | 6.69 |
| Scn4a | ENSMUST00000021056.7 | 0.073875 | 0.4 | 0.02 | 0.02 | 0.14 |
| Col7a1 | ENSMUST00000026740.5 | 5.4853 | 0.285 | 0.025 | 1.33 | 2.5 |
| Tnpo1 | ENSMUST00000109401.3 | 86.9988 | 13.99 | 6.765 | 13.755 | 13.565 |
| Adam19 | ENSMUST00000011400.7 | 0.050168 | 1.81 | 0.465 | 3.72 | 8.965 |
| Plxna3 | ENSMUST00000004326.3 | 1.2799 | 1.27 | 0.315 | 7.82 | 12.95 |
| Gpr179 | ENSMUST00000093942.4 | 0.030874 | 0.045 | 0.005 | 0.14 | 0.295 |
| Pkdrej | ENSMUST00000064370.4 | 0.006604 | 0.03 | 0.01 | 0.2 | 0.24 |
| Mylk | ENSMUST00000023538.8 | 3.0176 | 20.63 | 12.18 | 3.11 | 74.695 |
| Trim33 | ENSMUST00000029444.8 | 20.9992 | 2.555 | 1.065 | 6.2 | 3.585 |
| Pcdhb22 | ENSMUST00000192409.1 | 0.11783 | 0.965 | 0.23 | 2.755 | 5.28 |
| Pten | ENSMUST00000013807.7 | 73.0409 | 6.86 | 8.685 | 24.82 | 2.76 |
| Pappa | ENSMUST00000084501.3 | 0.02177 | 0.825 | 0.01 | 0.19 | 0.795 |
| Plxna1 | ENSMUST00000163139.3 | 17.1436 | 17.79 | 4.15 | 11.83 | 79.48 |
| Fancm | ENSMUST00000058889.4 | 5.6309 | 0.49 | 0.3 | 1.845 | 1.15 |
| Arhgef28 | ENSMUST00000109426.1 | 35.2521 | 10.59 | 0.005 | 4.455 | 12.16 |
| Tmc3 | ENSMUST00000039317.9 | 0.010088 | 0.51 | 0.06 | 0.045 | 0.22 |
| Plxnc1 | ENSMUST00000099337.3 | 0.11454 | 0.335 | 1.7 | 6.29 | 12.095 |
| Reps2 | ENSMUST00000112334.3 | 0.041798 | 2.75 | 2.3 | 11.375 | 0.78 |
| Nin | ENSMUST00000085314.5 | 10.9574 | 0.995 | 0.23 | 6.64 | 1.835 |
| Pdzd8 | ENSMUST00000099274.2 | 34.8986 | 2.835 | 2.64 | 7.24 | 9.765 |
| Pcdhb19 | ENSMUST00000059571.6 | 0.080284 | 0.17 | 0.02 | 0.985 | 0.94 |
| Tmem2 | ENSMUST00000096194.4 | 18.6815 | 3.865 | 1.69 | 3.035 | 3.18 |
| Lrrk2 | ENSMUST00000060642.6 | 3.1741 | 6.29 | 0.115 | 1.6 | 0.62 |
| Flrt2 | ENSMUST00000057324.3 | 0.65841 | 1.185 | 0.06 | 5.69 | 5.1 |
| Nvl | ENSMUST00000027797.8 | 20.151 | 2.045 | 1.285 | 7.545 | 3.97 |
| Myo9a | ENSMUST00000135298.3 | 7.5305 | 1.775 | 0.16 | 7.275 | 0.625 |
| Fat4 | ENSMUST00000061260.7 | 0.010693 | 0.915 | 0.15 | 1.27 | 2.79 |
| Mllt4 | ENSMUST00000139666.3 | 50.9298 | 7.65 | 3.3 | 16.61 | 9.3 |
| Celsr1 | ENSMUST00000016172.7 | 0.34221 | 5.835 | 3.21 | 0.645 | 16.14 |
| Map4 | ENSMUST00000035055.10 | 14.1386 | 15.42 | 6.95 | 90.515 | 26.885 |
| Dock6 | ENSMUST00000034728.7 | 0.43807 | 15.03 | 6.03 | 5.69 | 40.735 |
| Spon1 | ENSMUST00000046687.11 | 0.3401 | 11.85 | 0.195 | 15.68 | 26.4 |
| Spag9 | ENSMUST00000041956.9 | 25.645 | 8.915 | 3.555 | 52.235 | 4.53 |
| Sh3pxd2b | ENSMUST00000038753.5 | 6.3427 | 2.135 | 0.15 | 4.8 | 14.915 |
| 1700020l14Rik | ENSMUST00000153581.1 | 13.632 | 10.41 | 6 | 52.255 | 6.02 |
| Gm29666 | ENSMUST00000189185.1 | 0.87292 | 0.16 | 0.045 | 0.97 | 0.14 |
| Abca4 | ENSMUST00000013995.8 | 2.2317 | 0.895 | 0.01 | 0.245 | 1.095 |
| Golga4 | ENSMUST00000084820.4 | 23.0089 | 5.2 | 1.635 | 10.11 | 2.39 |
| Nhsl2 | ENSMUST00000101339.6 | 0.90199 | 2.235 | 0.195 | 4.555 | 7.66 |
| Tnrc18 | ENSMUST00000151477.3 | 6.6458 | 9.085 | 4.195 | 6.745 | 36.665 |
| Dnah8 | ENSMUST00000170651.1 | 0.017458 | 0.055 | 0.015 | 0.065 | 0.185 |
| Xirp1 | ENSMUST00000111635.2 | 0.004795 | 0.315 | 0.075 | 0.02 | 0.115 |
| Ptprm | ENSMUST00000037974.8 | 0.68155 | 4.66 | 0.39 | 11.16 | 5.88 |
| RP23-383N15.1 | ENSMUST00000192800.1 | 0 | 0.22 | 0.045 | 0.405 | 0.105 |
| Mgam | ENSMUST00000071535.6 | 0.10509 | 8.81 | 11.215 | 0.095 | 0.065 |
| Zfp334 | ENSMUST00000103084.3 | 0.92428 | 0.66 | 0.325 | 4.01 | 4.58 |
| Scn7a | ENSMUST00000042792.6 | 0.015723 | 0.605 | 0.015 | 0.48 | 0.755 |
| Trpm6 | ENSMUST00000040489.7 | 0.32383 | 1.085 | 0.055 | 0.12 | 0.725 |
| 2410089E03Rik | ENSMUST00000110617.1 | 9.8956 | 1.585 | 0.4 | 7.21 | 2.75 |
| Polr1a | ENSMUST00000055296.8 | 35.0729 | 5.115 | 4.13 | 5.105 | 16.61 |
| Dennd3 | ENSMUST00000043414.7 | 2.2845 | 13.92 | 1.71 | 2.145 | 5.565 |
| Abcc2 | ENSMUST00000026208.5 | 13.3131 | 49.305 | 93.46 | 0.1 | 0.18 |
| Cgnl1 | ENSMUST00000072899.4 | 24.2009 | 47.5 | 7.815 | 3.565 | 6.01 |
| Stab2 | ENSMUST00000035288.10 | 0.055239 | 8.34 | 12.26 | 0.06 | 1.08 |
| Akap11 | ENSMUST00000022593.5 | 11.8471 | 4.295 | 1.945 | 21.08 | 2.875 |
| Notch4 | ENSMUST00000015612.9 | 1.1254 | 6.89 | 0.885 | 1.2 | 7.75 |
| Ppm1I | ENSMUST00000029355.8 | 2.7363 | 2.245 | 1.43 | 12.14 | 3.625 |
| Wdr90 | ENSMUST00000079461.10 | 7.4088 | 2.07 | 1.625 | 1.115 | 12.305 |
| Edaradd | ENSMUST00000179308.1 | 0.031618 | 0.265 | 0.02 | 0.19 | 0.07 |
| Kcnq1ot1 | ENSMUST00000185789.1 | 1.1162 | 0.16 | 0.085 | 0.76 | 0.265 |
| RP24-338G10.1 | ENSMUST00000193744.1 | 0 | 0.03 | 0.03 | 0.105 | 0.165 |
| Crb2 | ENSMUST00000050372.7 | 3.488 | 1.565 | 0.025 | 0.655 | 3.175 |
| Arhgef10 | ENSMUST00000084207.7 | 4.1728 | 4.565 | 0.61 | 9 | 16.8 |
| lrs1 | ENSMUST00000069799.2 | 2.5152 | 4.195 | 5.59 | 3.65 | 22.515 |
| Crocc | ENSMUST00000102491.5 | 2.2642 | 5.925 | 0.335 | 10.785 | 13.925 |
| Ehbp1l1 | ENSMUST00000049295.10 | 14.4396 | 6.89 | 2.62 | 5.335 | 29.7 |
| Ogt | ENSMUST00000044475.4 | 32.7175 | 21.29 | 9.1 | 102.26 | 32.17 |
| Thsd7a | ENSMUST00000046121.8 | 3.4074 | 1.505 | 0.06 | 3.64 | 0.825 |
| Dsp | ENSMUST00000124830.1 | 15.592 | 2.745 | 4.145 | 0.14 | 5.005 |
| Daam2 | ENSMUST00000057610.6 | 0.34713 | 10.75 | 0.975 | 15.99 | 12.57 |
| Sestd1 | ENSMUST00000102660.3 | 5.9251 | 20.865 | 0.375 | 11.73 | 7.215 |
| Sart1 | ENSMUST00000044207.4 | 20.3807 | 3.475 | 2.065 | 8.89 | 5.43 |
| Dab2ip | ENSMUST00000145698.3 | 3.0777 | 19.31 | 5.265 | 27.27 | 49.775 |
| Ago4 | ENSMUST00000084289.4 | 0.35746 | 0.59 | 0.5 | 2.635 | 0.745 |
| Fgd5 | ENSMUST00000089334.4 | 0.088143 | 3.385 | 0.73 | 0.96 | 3.86 |
| Pla2r1 | ENSMUST00000112525.4 | 0.4315 | 1.62 | 0.25 | 0.095 | 0.97 |
| Arfgef1 | ENSMUST00000088615.6 | 22.058 | 6.265 | 2.855 | 11.25 | 2.435 |
| Onecut2 | ENSMUST00000175965.4 | 3.8344 | 0 | 4.59 | 0.155 | 0.77 |
| Tbc1d32 | ENSMUST00000099739.3 | 3.6428 | 1.145 | 0.4 | 1.815 | 0.515 |
| Dpy19l4 | ENSMUST00000084892.7 | 10.0448 | 2.995 | 1.12 | 5.315 | 1.42 |
| Myom3 | ENSMUST00000105854.1 | 0.24026 | 0.305 | 0.05 | 0.005 | 0.105 |
| Gpr116 | ENSMUST00000113599.1 | 0.17574 | 12.39 | 2.08 | 4.765 | 6.35 |
| Tjp1 | ENSMUST00000032729.6 | 42.1997 | 10.51 | 3.99 | 15.84 | 14.075 |
| Plcb2 | ENSMUST00000102524.3 | 0.045128 | 0.67 | 0.31 | 0.38 | 1.44 |
| Aldh1l2 | ENSMUST00000020497.9 | 0.34242 | 0.51 | 0.03 | 1.015 | 0.435 |
| Arhgef5 | ENSMUST00000031750.9 | 30.2495 | 17.605 | 5.43 | 0.89 | 9.855 |
| Helz2 | ENSMUST00000108831.3 | 1.5309 | 7.51 | 31.34 | 0.85 | 18.125 |
| Kif21a | ENSMUST00000088614.7 | 7.6264 | 6.515 | 3.035 | 21.525 | 4.02 |
| ltpr1 | ENSMUST00000032192.6 | 6.1055 | 24.87 | 3.49 | 36.325 | 13.06 |
| Rassf4 | ENSMUST00000035842.4 | 0.27494 | 1.765 | 1.17 | 3.4 | 6.09 |
| Abca3 | ENSMUST00000079594.7 | 13.9371 | 125.74 | 48.585 | 17.55 | 20.73 |
| Nipbl | ENSMUST00000052965.6 | 11.5482 | 2.985 | 1.35 | 4.99 | 3 |
| Wdr7 | ENSMUST00000072726.5 | 6.4182 | 3.8 | 2.795 | 16.735 | 3.345 |
| Phlpp2 | ENSMUST00000179721.3 | 1.4244 | 3.86 | 0.66 | 8.22 | 6.53 |
| Zbtb1 | ENSMUST00000042779.3 | 4.2827 | 0.9 | 0.445 | 1.55 | 2.02 |
| Tmed8 | ENSMUST00000037418.5 | 2.3264 | 4.05 | 0.505 | 8.305 | 4.08 |
| Cnot1 | ENSMUST00000098473.6 | 45.7584 | 12.535 | 8.545 | 13.015 | 10.565 |
| Abca9 | ENSMUST00000044850.3 | 0.006076 | 1.295 | 0.245 | 1.165 | 1.315 |
| Slc12a7 | ENSMUST00000017900.7 | 11.806 | 57.5 | 46.195 | 1.865 | 112.275 |
| Svil | ENSMUST00000126977.3 | 8.6685 | 6.01 | 6.495 | 4.39 | 26.32 |
| Prkaa2 | ENSMUST00000030243.7 | 1.736 | 14.325 | 6.605 | 4.285 | 0.925 |
| Myo6 | ENSMUST00000113268.3 | 25.014 | 19.245 | 2.675 | 11.75 | 5.475 |
| Btrc | ENSMUST00000065601.7 | 7.9632 | 5.42 | 2.41 | 17.965 | 5.55 |
| Nfia | ENSMUST00000075448.8 | 1.9953 | 26.88 | 21.805 | 9.09 | 57.48 |
| Cmah | ENSMUST00000167746.3 | 5.365 | 2.69 | 4.905 | 0.03 | 0.045 |
| Abca1 | ENSMUST00000030010.3 | 0.18044 | 5.2 | 12.885 | 6.225 | 24.35 |
| Osbpl8 | ENSMUST00000105275.3 | 15.1743 | 7.99 | 2.105 | 11.37 | 2.585 |
| Cdyl2 | ENSMUST00000109102.2 | 0.93834 | 0.88 | 0.285 | 2.095 | 0.605 |
| Wdfy4 | ENSMUST00000130509.4 | 0.010188 | 1.575 | 0.885 | 0.36 | 2.06 |
| Plcxd2 | ENSMUST00000130481.1 | 7.246 | 0.89 | 13.905 | 12.515 | 0.335 |
| 4932438A13Rik | ENSMUST00000057272.10 | 13.0887 | 5.6 | 3.495 | 16.97 | 3.325 |
| Dennd4a | ENSMUST00000038890.5 | 2.2408 | 1.585 | 2.565 | 7.945 | 2 |
| Rnf213 | ENSMUST00000093902.7 | 5.0865 | 9.635 | 9.51 | 3.635 | 25.88 |
| Thada | ENSMUST00000047524.8 | 5.4838 | 2.135 | 0.905 | 1.455 | 3.105 |
| Myo18a | ENSMUST00000000645.8 | 58.0374 | 24.075 | 11.82 | 19.285 | 15.54 |
| Arid1a | ENSMUST00000145664.4 | 2.9113 | 22.035 | 12.38 | 22.855 | 46.105 |
| Kdm7a | ENSMUST00000002305.8 | 2.6234 | 1.37 | 0.825 | 5.275 | 2.585 |
| Acacb | ENSMUST00000102582.3 | 0.094988 | 9.805 | 9.89 | 0.695 | 12.765 |
| Szt2 | ENSMUST00000075406.7 | 1.9876 | 11.305 | 6.01 | 12.64 | 23.78 |
| Itpr2 | ENSMUST00000053273.10 | 2.8832 | 12.9 | 3.67 | 2.51 | 9.24 |
| Lsm11 | ENSMUST00000129820.3 | 4.8305 | 1.22 | 0.93 | 3.445 | 5.425 |
| Mlec | ENSMUST00000112121.3 | 47.8277 | 118.53 | 28.07 | 20.58 | 55.865 |
| Dock9 | ENSMUST00000100299.5 | 8.1985 | 15.025 | 1.25 | 14.375 | 14.965 |
| Nedd4l | ENSMUST00000080418.4 | 9.5737 | 7.08 | 2.96 | 13.655 | 2.04 |
| Dock8 | ENSMUST00000025831.6 | 0.77808 | 9.92 | 6.845 | 0.57 | 9.52 |
| Nhsl1 | ENSMUST00000037341.9 | 1.7878 | 5.875 | 1.05 | 4.935 | 2.615 |
| Ppp2ca | ENSMUST00000020608.2 | 68.756 | 19.555 | 13.075 | 39.965 | 26.905 |
| Dicer1 | ENSMUST00000041987.6 | 14.0004 | 4.275 | 2.83 | 7.6 | 4.7 |
| Gt3c4 | ENSMUST00000171404.3 | 13.4732 | 3.705 | 2.995 | 5.125 | 5.525 |
| Kmt2c | ENSMUST00000045291.9 | 4.7035 | 6.69 | 3.035 | 16 | 8.135 |
| Brwd1 | ENSMUST00000113829.3 | 20.4707 | 6.315 | 5.375 | 25.78 | 27.745 |
| Nf1 | ENSMUST00000071325.4 | 7.3464 | 5.055 | 2.545 | 14.415 | 7.08 |
| Macf1 | ENSMUST00000097897.6 | 33.1411 | 33.89 | 10.045 | 69.81 | 39.905 |
| Rnf217 | ENSMUST00000081989.7 | 5.7943 | 0.64 | 1.975 | 2.135 | 4.165 |
| Luzp1 | ENSMUST00000105849.4 | 13.8241 | 7.415 | 1.975 | 8.59 | 6.33 |
| Cdc42bpb | ENSMUST00000041965.3 | 13.1609 | 35.79 | 24.09 | 34.105 | 80.285 |
| Utrn | ENSMUST00000076817.4 | 12.3318 | 6.415 | 1.945 | 5.64 | 6.86 |
| Ep400 | ENSMUST00000112435.4 | 10.5929 | 12.095 | 7.385 | 10.795 | 29.565 |
| Sec16a | ENSMUST00000114082.4 | 10.026 | 44.96 | 49.215 | 11.775 | 77.965 |
| Dip2c | ENSMUST00000174552.3 | 4.5092 | 5.425 | 2.26 | 10.925 | 9.925 |
| Itga4 | ENSMUST00000099972.4 | 0.98389 | 0.745 | 0.22 | 1.455 | 0.86 |
| Jrk | ENSMUST00000050234.3 | 3.0198 | 2.195 | 0.59 | 1.61 | 3.47 |
| Elmsan1 | ENSMUST00000110294.1 | 2.1229 | 3.835 | 2.27 | 3.215 | 8.14 |
| Edem1 | ENSMUST00000089162.3 | 26.1889 | 9.995 | 36.81 | 3.915 | 17.59 |
| Son | ENSMUST00000114037.4 | 42.8623 | 41.73 | 30.405 | 90.75 | 116.81 |
| Sox6 | ENSMUST00000072804.6 | 1.7896 | 3.84 | 1.54 | 1.915 | 0.4 |
| Ctif | ENSMUST00000165559.1 | 0.41227 | 3.855 | 6.235 | 10.86 | 8.125 |
| Uhrf1bp1l | ENSMUST00000020112.5 | 14.9405 | 6.94 | 5.02 | 13.115 | 3.51 |
| Sbf1 | ENSMUST00000123791.3 | 24.207 | 43.31 | 30.32 | 37.735 | 92.27 |
| Rfx7 | ENSMUST00000093820.5 | 6.3641 | 3.42 | 1.79 | 7.645 | 3.875 |
| Nbeal2 | ENSMUST00000167320.3 | 22.7269 | 22.705 | 6.865 | 6.845 | 19.445 |
| Dock4 | ENSMUST00000037488.6 | 3.5462 | 2.305 | 3.745 | 9.67 | 7.92 |
| Trim56 | ENSMUST00000054384.5 | 5.4775 | 7.035 | 3.405 | 0.875 | 7.93 |
| Dync1h1 | ENSMUST00000018851.9 | 31.0827 | 20.345 | 7.335 | 34.97 | 30.03 |
| Crebbp | ENSMUST00000023165.7 | 3.0552 | 10.25 | 5.13 | 12.09 | 15.08 |
| Gm20342 | ENSMUST00000185480.1 | 0.47756 | 0.995 | 0.375 | 1.425 | 1.04 |
| Fign | ENSMUST00000131615.4 | 0.16505 | 1.48 | 1.08 | 0.455 | 0.885 |
| Baz2a | ENSMUST00000170054.4 | 2.8055 | 12.265 | 7.03 | 13.83 | 18.38 |
| Parp14 | ENSMUST00000042665.8 | 0.34536 | 2.465 | 2.58 | 0.805 | 1.85 |
| AU022252 | ENSMUST00000141112.1 | 6.2459 | 3.7 | 10.075 | 3.765 | 2.62 |
| Als2cl | ENSMUST00000155014.1 | 19.0563 | 9.665 | 9.545 | 1.675 | 12.565 |
| Prdm15 | ENSMUST00000095849.5 | 4.6767 | 1.93 | 1.36 | 3.86 | 4.475 |
| Med12 | ENSMUST00000117706.3 | 5.8026 | 6.195 | 3.095 | 6.785 | 11.99 |
| Polr2a | ENSMUST00000058470.11 | 3.9096 | 17.89 | 6.765 | 12.235 | 15.215 |
| Amer1 | ENSMUST00000084535.5 | 0.99038 | 1.025 | 0.605 | 1.385 | 2.12 |
| Prrc2c | ENSMUST00000182660.3 | 24.3771 | 14.05 | 6.365 | 21.175 | 21.51 |
| Setd1b | ENSMUST00000163030.4 | 0.43086 | 4.095 | 4.36 | 4.635 | 6.965 |
| Aars | ENSMUST00000034441.7 | 70.6912 | 17.64 | 35.415 | 25.155 | 33.645 |
| Rapgef1 | ENSMUST00000102872.6 | 8.902 | 23.235 | 16.515 | 30.53 | 39.895 |
| Kif13a | ENSMUST00000056978.7 | 4.8551 | 4.675 | 1.84 | 3.495 | 6.69 |
| Irgq | ENSMUST00000049020.7 | 4.0891 | 9.435 | 8.965 | 18.785 | 14.635 |
| Ep300 | ENSMUST00000068387.6 | 6.7401 | 11.735 | 4.805 | 8.765 | 15.095 |
| Rc3h1 | ENSMUST00000161609.3 | 12.3175 | 8.205 | 4.565 | 4.415 | 10.83 |
| Abhd2 | ENSMUST00000037315.8 | 54.3027 | 18.115 | 49.21 | 15.365 | 32.57 |
| Usp36 | ENSMUST00000106296.4 | 4.8823 | 4.755 | 4.495 | 6.93 | 11.825 |
| Aim1 | ENSMUST00000020017.8 | 3.1261 | 2.185 | 1.94 | 0.835 | 1.015 |
| Impg2 | ENSMUST00000069936.7 | 0.12107 | 0.105 | 0.17 | 0.215 | 0.04 |
| Dock5 | ENSMUST00000039135.4 | 2.3545 | 2.985 | 1.965 | 0.84 | 3.74 |
| Dcaf5 | ENSMUST00000054145.6 | 5.2977 | 9.62 | 11.82 | 16.135 | 19.975 |
| Rprd2 | ENSMUST00000090791.3 | 5.7879 | 4.5 | 2.295 | 6.405 | 5.69 |
| Spg11 | ENSMUST00000036450.7 | 5.166 | 3.78 | 2.47 | 3.825 | 6.405 |
| Smg7 | ENSMUST00000043560.10 | 11.9726 | 18.295 | 6.955 | 16.41 | 14.7 |
| Wdr81 | ENSMUST00000173320.3 | 6.61 | 10.725 | 14.17 | 5.59 | 15.5 |
| F8 | ENSMUST00000033539.8 | 1.036 | 1.03 | 0.725 | 0.255 | 0.87 |
| Atxn1l | ENSMUST00000093162.3 | 6.9561 | 9.77 | 4.06 | 6.12 | 9.485 |
| Ubn1 | ENSMUST00000052449.5 | 8.431 | 10.295 | 5.455 | 9.415 | 14.125 |
| Nav2 | ENSMUST00000064395.8 | 2.7094 | 8.87 | 10.075 | 11.265 | 8.795 |
| Rnf169 | ENSMUST00000080817.4 | 7.4291 | 3.135 | 3.975 | 5.865 | 3.705 |
| Atg2b | ENSMUST00000041055.7 | 7.2585 | 4.42 | 3.16 | 6.6 | 5.605 |
| Qser1 | ENSMUST00000117237.1 | 2.0168 | 2.665 | 1.34 | 2.645 | 2.585 |
| Arhgef12 | ENSMUST00000165665.3 | 20.3522 | 24.855 | 13.04 | 24.395 | 15.285 |
| Letm1 | ENSMUST00000005431.5 | 21.2177 | 24.655 | 14.625 | 14.805 | 12.795 |
| Larp1 | ENSMUST00000178636.1 | 14.0446 | 20.295 | 24.67 | 18.37 | 28.09 |
| Arhgap35 | ENSMUST00000075845.6 | 17.0046 | 15.72 | 14.565 | 19.845 | 25.1 |
| Zfyve26 | ENSMUST00000021547.6 | 3.9207 | 4.605 | 4.46 | 3.245 | 6.19 |
| Prpf8 | ENSMUST00000018449.6 | 51.1044 | 52.23 | 41.555 | 36.85 | 65.435 |
| Nr6a1 | ENSMUST00000112877.3 | 2.2584 | 1.3 | 2.1 | 1.94 | 2.25 |

1. A pair of non-naturally occurring nucleic acid probes for detecting a polynucleotide analyte, comprising:
i. a first nucleic acid probe comprising:
a) a first probe binding arm that is complementary to a first probe target region of a bridge probe; and
b) a first polynucleotide analyte binding arm that is complementary to a first analyte target region of a polynucleotide analyte, and
ii. a second nucleic acid probe comprising:
a) a second probe binding arm that is complementary to a second probe target region of the bridge probe; wherein the first probe target region is located downstream of the second probe target region on the bridge probe, and
b) a second polynucleotide analyte binding arm that is complementary to a second analyte target region of the polynucleotide analyte, wherein the second analyte target region is located downstream of the first analyte target region on the polynucleotide analyte,
wherein binding of the first polynucleotide analyte binding arm to the first analyte target region and binding of the second polynucleotide analyte binding arm to the second analyte target region permit binding of the first probe binding arm to the first bridge probe target region and binding of the second probe binding arm to the second bridge probe target region, thereby detecting the polynucleotide analyte.

2. The pair of non-naturally occurring nucleic acid probes of claim 1, wherein the polynucleotide analyte binding arm in the first and/or second nucleic acid probe consists of 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

3. The pair of non-naturally occurring nucleic acid probes of claim 1 or 2, wherein the probe binding arm in the first and/or second nucleic acid probes consists of 9 or 10 nucleotides.

4. The pair of non-naturally occurring nucleic acid probes of any one of claims 1 to 3, wherein the probe binding arm in the first and/or second nucleic acid probes comprises an identification portion for binding to a unique bridge probe.

5. The pair of non-naturally occurring nucleic acid probes of any one of claims 1 to 4, wherein the first and second nucleic acid probes comprise a linker positioned between the probe binding arm and the polynucleotide analyte binding arm.

6. The pair of non-naturally occurring nucleic acid probes of claim 5, wherein the linker consists of 1, 2, 3, 4, 5, 6, 7, 8, 9 or 10 nucleobases.

7. The pair of non-naturally occurring nucleic acid probes of any one of claims 1 to 6, wherein the bridge probe is a readout probe that is coupled or conjugated to a label (such as a fluorescent label).

8. The pair of non-naturally occurring nucleic acid probes of any one of claims 1 to 6, wherein the bridge probe is detected via hybridization to a readout probe that is conjugated to a label (such as a fluorescent label).

9. The pair of non-naturally occurring nucleic acid probes of claim 8, wherein the readout probe hybridizes to a terminal region of the bridge probe.

10. The pair of non-naturally occurring nucleic acid probes of claim 8, wherein the readout probe hybridizes to a central region of the bridge probe.

11. The pair of non-naturally occurring nucleic acid probes of any one of claims 1 to 10, wherein the first analyte target region is immediately adjacent to the second analyte target region.

12. The pair of non-naturally occurring nucleic acid probes of any one of claims 1 to 11, wherein the first analyte target region is spaced from the second analyte target region by no more than 1, 2, 3, 4, 5, 6, 7, 8, 9, 10, 11, 12, 13, 14, 15, 16, 17, 18, 19 or 20 nucleobases.

13. The pair of non-naturally occurring nucleic acid probes of any one of claims 1 to 12, wherein the first probe target region is immediately adjacent to the second probe target region.

14. The pair of non-naturally occurring nucleic acid probes of any one of claims 1 to 13, wherein the first probe target region is spaced from the second probe target region by no more than 20, 19, 18, 17, 16, 15, 14, 13, 12, 11, 10, 9, 8, 7, 6, 5, 4, 3, 2 or 1 nucleobases.

15. A probe system comprising a pair of non-naturally occurring nucleic acid probes of any one of claims 1 to 14.

16. The probe system of claim 15, wherein the probe system further comprises a bridge probe.

17. A method of detecting a polynucleotide analyte in a sample, the method comprising:
(a) contacting the sample with a pair of non-naturally occurring nucleic acid probes according to any one of claims 1 to 14 or a probe system of claim 15 or 16; and
(b) detecting the polynucleotide analyte based on hybridization to a unique bridge probe in the presence of the polynucleotide analyte.

18. A library for detecting two or more polynucleotide analytes in a sample; the library comprising two or more pairs of non-naturally occurring nucleic acid probes according to any one of claims 1 to 14 or a plurality of probe systems according to claim 15 or 16,
wherein each pair of nucleic acid probes is specific to each polynucleotide analyte; and
wherein each pair of nucleic acid probes is configured to hybridize to a unique bridge probe in the presence of the polynucleotide analyte.

19. A method of detecting two or more polynucleotide analytes in a sample, the method comprising:
a) contacting a sample with a library according to claim 18, and
b) detecting each polynucleotide analyte based on hybridization to a unique bridge probe in the presence of the polynucleotide analyte.

20. The method of claim 19, wherein the method comprises contacting the sample with a unique bridge probe for each polynucleotide analyte.

21. The method of claim 20, wherein the unique bridge probe comprises a specific tag or barcode sequence.

22. The method of any one of claims 19 to 21, wherein the two or more polynucleotide analytes are detected concurrently based on hybridization to a unique bridge probe for each polynucleotide analyte.

23. The method of any one of claims 19 to 22, wherein the two or more polynucleotide analytes are detected sequentially based on multiple rounds of hybridization to a unique bridge probe for each polynucleotide analyte.

24. The method of any one of claims 19 to 23, wherein the method comprises detecting the unique bridge probe via hybridization to a readout probe that is conjugated to a label.

25. The method of claim 24, wherein the method comprises contacting the sample with a unique readout probe for each polynucleotide analyte.

26. The method of any one of claims 19 to 25, wherein the method comprises removing any bound or unbound bridge and/or readout probe in between detection of each polynucleotide analyte.

27. The method of any one of claims 19 to 26, wherein the method comprises removing any signal from any bound or unbound readout probe in between detection of each polynucleotide analyte.

28. A method of detecting or visualising the expression of one or more polynucleotide analytes in a sample, the method comprising a) contacting a sample with a library according to claim 18, and b) detecting or visualising the one or more polynucleotide analytes based on hybridisation to a unique bridge probe in the presence of the one or more polynucleotide analytes.

29. A kit comprising a pair of non-naturally occurring nucleic acid probes according to any one of claims 1 to 14 or a plurality of probe systems according to claims 15 or 16 or a library according to claim 18.

30. The kit of claim 29, wherein the kit further comprises one or more bridge probes.


SEQUENCE LISTING (595 sequences)

SEQ ID NO: 1 - DNA, 6 bp, Artificial Sequence
  Kpn1 restriction site
ggtacc

SEQ ID NO: 2 - DNA, 6 bp, Artificial Sequence
  Kpn1 restriction site
ccatgg

SEQ ID NO: 3 - DNA, 6 bp, Artificial Sequence
  EcorR1 restriction site
gaattc

SEQ ID NO: 4 - DNA, 6 bp, Artificial Sequence
  EcorR1 restriction site
cttaag

SEQ ID NO: 5 - DNA, 12 bp, Artificial Sequence
  library design
cactctacctat

SEQ ID NO: 6 - DNA, 9 bp, Artificial Sequence
  library design
atttaaccg

SEQ ID NO: 7 - DNA, 18 bp, Artificial Sequence
  Readout sequence
ttactcacgcacccatca

SEQ ID NO: 8 - DNA, 18 bp, Artificial Sequence
  Readout sequence
tttctcacgctttacaat

SEQ ID NO: 9 - DNA, 20 bp, Artificial Sequence
  Primer sequence
aacgaacggagggtcattgg

SEQ ID NO: 10 - DNA, 40 bp, Artificial Sequence
  Primer sequence
taatacgactcactatagggaggctctactcgcattaggg

SEQ ID NO: 11 - DNA, 22 bp, Artificial Sequence
  Primer sequence
  misc_feature (21)..(22): n is a, c, g, or t
tactcgcattaggggaattcnn

SEQ ID NO: 12 - DNA, 22 bp, Artificial Sequence
  Primer sequence
  misc_feature (1)..(2): n is a, c, g, or t
nngtaccccaatgaccctccgt

SEQ ID NO: 13 - DNA, 88 bp, Artificial Sequence
  Ccdc177 template sequence
aacgaacggagggtcattggggtaccactctacctatgggttactgccggctgcagcagccagaattcccctaatgcgagtagagcct

SEQ ID NO: 14 - DNA, 88 bp, Artificial Sequence
  Ccdc177 template sequence
aacgaacggagggtcattggggtaccactctacctatcggaggcggaggccgaggaggcagcgaattcccctaatgcgagtagagcct

SEQ ID NO: 15 - DNA, 88 bp, Artificial Sequence
  Ccdc177 template sequence
aacgaacggagggtcattggggtaccactctacctatagcagctccacgggcttgaccgcgcgaattcccctaatgcgagtagagcct

SEQ ID NO: 16 - DNA, 88 bp, Artificial Sequence
  Ccdc177 template sequence
aacgaacggagggtcattggggtaccactctacctatgcgcatgatgcgctcgcgctcggcggaattcccctaatgcgagtagagcct

SEQ ID NO: 17 - DNA, 88 bp, Artificial Sequence
  Ccdc177 template sequence
aacgaacggagggtcattggggtaccactctacctatggctctttctacctgtccgagaaccgaattcccctaatgcgagtagagcct

SEQ ID NO: 18 - DNA, 88 bp, Artificial Sequence
  Ccdc177 template sequence
aacgaacggagggtcattggggtaccactctacctatctgttcctggcggaagctcgaggtggaattcccctaatgcgagtagagcct

SEQ ID NO: 19 - DNA, 88 bp, Artificial Sequence
  Ccdc177 template sequence
aacgaacggagggtcattggggtaccactctacctatagtggctcaggtctccgaggctgaagaattcccctaatgcgagtagagcct

SEQ ID NO: 20 - DNA, 88 bp, Artificial Sequence
  Ccdc177 template sequence
aacgaacggagggtcattggggtaccactctacctattggtgccgggccagcatgagggcgggaattcccctaatgcgagtagagcct

SEQ ID NO: 21 - DNA, 88 bp, Artificial Sequence
  Ccdc177 template sequence
aacgaacggagggtcattggggtaccactctacctatcgctgcttctctcgctcctcgcgctgaattcccctaatgcgagtagagcct

SEQ ID NO: 22 - DNA, 88 bp, Artificial Sequence
  Ccdc177 template sequence
aacgaacggagggtcattggggtaccactctacctatctcccgccgcagcagtccctgacgcgaattcccctaatgcgagtagagcct

SEQ ID NO: 23 - DNA, 88 bp, Artificial Sequence
  Ccdc177 template sequence
aacgaacggagggtcattggggtaccactctacctatgcgccgcccgctgggcgcgctcccggaattcccctaatgcgagtagagcct

SEQ ID NO: 24 - DNA, 88 bp, Artificial Sequence
  Ccdc177 template sequence
aacgaacggagggtcattggggtaccactctacctatgctgctgccggactcggcctctaaggaattcccctaatgcgagtagagcct

SEQ ID NO: 25 - DNA, 88 bp, Artificial Sequence
  Ccdc177 template sequence
aacgaacggagggtcattggggtaccactctacctatcgcgctcccgcagctcccgggcgcggaattcccctaatgcgagtagagcct

SEQ ID NO: 26 - DNA, 88 bp, Artificial Sequence
  Ccdc177 template sequence
aacgaacggagggtcattggggtaccactctacctatgccgtcgttctcccgcccgcgccaggaattcccctaatgcgagtagagcct

SEQ ID NO: 27 - DNA, 88 bp, Artificial Sequence
  Ccdc177 template sequence
aacgaacggagggtcattggggtaccactctacctatgtcttcgtctctctctaccttctccgaattcccctaatgcgagtagagcct

SEQ ID NO: 28 - DNA, 88 bp, Artificial Sequence
  Ccdc177 template sequence
aacgaacggagggtcattggggtaccactctacctatccgggccgtggagcgagcgctctccgaattcccctaatgcgagtagagcct

SEQ ID NO: 29 - DNA, 88 bp, Artificial Sequence
  Ccdc177 template sequence
aacgaacggagggtcattggggtaccactctacctatagagaggccacacctggccatatccgaattcccctaatgcgagtagagcct

SEQ ID NO: 30 - DNA, 88 bp, Artificial Sequence
  Ccdc177 template sequence
aacgaacggagggtcattggggtaccactctacctataataccatgtctgccttctcagtgcgaattcccctaatgcgagtagagcct

SEQ ID NO: 31 - DNA, 88 bp, Artificial Sequence
  Ccdc177 template sequence
aacgaacggagggtcattggggtaccactctacctatctacagggaccttcctcctcccagcgaattcccctaatgcgagtagagcct

SEQ ID NO: 32 - DNA, 88 bp, Artificial Sequence
  Ccdc177 template sequence
aacgaacggagggtcattggggtaccactctacctataagatagagaggtaagtctgggagagaattcccctaatgcgagtagagcct

SEQ ID NO: 33 - DNA, 88 bp, Artificial Sequence
  Ccdc177 template sequence
aacgaacggagggtcattggggtaccactctacctatgggaacaggataatttgttcacgtggaattcccctaatgcgagtagagcct

SEQ ID NO: 34 - DNA, 88 bp, Artificial Sequence
  Ccdc177 template sequence
aacgaacggagggtcattggggtaccactctacctatctggaggttggagggcttccattaagaattcccctaatgcgagtagagcct

SEQ ID NO: 35 - DNA, 88 bp, Artificial Sequence
  Ccdc177 template sequence
aacgaacggagggtcattggggtaccactctacctatgaaggtagctctacttcgtaaggcagaattcccctaatgcgagtagagcct

SEQ ID NO: 36 - DNA, 88 bp, Artificial Sequence
  Ccdc177 template sequence
aacgaacggagggtcattggggtaccactctacctatgcagccaaggatcctaaattgtcttgaattcccctaatgcgagtagagcct

SEQ ID NO: 37 - DNA, 88 bp, Artificial Sequence
  Ccdc177 template sequence
aacgaacggagggtcattggggtaccactctacctatcttcaggtataagtgagagggtaccgaattcccctaatgcgagtagagcct

SEQ ID NO: 38 - DNA, 88 bp, Artificial Sequence
  Ccdc177 template sequence
aacgaacggagggtcattggggtaccactctacctatgcctcccaactccaggtcaagaaaggaattcccctaatgcgagtagagcct

SEQ ID NO: 39 - DNA, 88 bp, Artificial Sequence
  Ccdc177 template sequence
aacgaacggagggtcattggggtaccactctacctatccacacacaccttggatgctaaatggaattcccctaatgcgagtagagcct

SEQ ID NO: 40 - DNA, 88 bp, Artificial Sequence
  Ccdc177 template sequence
aacgaacggagggtcattggggtaccactctacctatgactttccaccttccctggtgaagagaattcccctaatgcgagtagagcct

SEQ ID NO: 41 - DNA, 88 bp, Artificial Sequence
  Ccdc177 template sequence
aacgaacggagggtcattggggtaccactctacctatcattatctcacacgtgacacctaaagaattcccctaatgcgagtagagcct

SEQ ID NO: 42 - DNA, 88 bp, Artificial Sequence
  Ccdc177 template sequence
aacgaacggagggtcattggggtaccactctacctatctgtcttagagacaacaccagttatgaattcccctaatgcgagtagagcct

SEQ ID NO: 43 - DNA, 88 bp, Artificial Sequence
  Ccdc177 template sequence
aacgaacggagggtcattggggtaccactctacctataatgctcatgctttgaagtaagtacgaattcccctaatgcgagtagagcct

SEQ ID NO: 44 - DNA, 88 bp, Artificial Sequence
  Ccdc177 template sequence
aacgaacggagggtcattggggtaccactctacctatgtcggtgaggactaagaaaggtgacgaattcccctaatgcgagtagagcct

SEQ ID NO: 45 - DNA, 88 bp, Artificial Sequence
  Ccdc177 template sequence
aacgaacggagggtcattggggtaccactctacctatccgtgtacggcaactgcaccttcaggaattcccctaatgcgagtagagcct

SEQ ID NO: 46 - DNA, 88 bp, Artificial Sequence
  Ccdc177 template sequence
aacgaacggagggtcattggggtaccactctacctatacgtcacatgtgactctggatctgagaattcccctaatgcgagtagagcct

SEQ ID NO: 47 - DNA, 88 bp, Artificial Sequence
  Ccdc177 template sequence
aacgaacggagggtcattggggtaccactctacctatatatggtctggaacttgaaggtaccgaattcccctaatgcgagtagagcct

SEQ ID NO: 48 - DNA, 88 bp, Artificial Sequence
  Ccdc177 template sequence
aacgaacggagggtcattggggtaccactctacctatctatctggtatgggctgactcaatagaattcccctaatgcgagtagagcct

SEQ ID NO: 49 - DNA, 88 bp, Artificial Sequence
  Ccdc177 template sequence
aacgaacggagggtcattggggtaccgtctctccatgggagactacgaacttatatttaaccgaattcccctaatgcgagtagagcct

SEQ ID NO: 50 - DNA, 88 bp, Artificial Sequence
  Ccdc177 template sequence
aacgaacggagggtcattggggtaccgctgctgggctccctgggcatcgggtatatttaaccgaattcccctaatgcgagtagagcct

SEQ ID NO: 51 - DNA, 88 bp, Artificial Sequence
  Ccdc177 template sequence
aacgaacggagggtcattggggtacccgggcgcaggcctccagagagcgggtatatttaaccgaattcccctaatgcgagtagagcct

SEQ ID NO: 52 - DNA, 88 bp, Artificial Sequence
  Ccdc177 template sequence
aacgaacggagggtcattggggtaccgcactgctgcagtttggccaggcgctatatttaaccgaattcccctaatgcgagtagagcct

SEQ ID NO: 53 - DNA, 88 bp, Artificial Sequence
  Ccdc177 template sequence
aacgaacggagggtcattggggtacccgggcggaggccggctcctggcgggtatatttaaccgaattcccctaatgcgagtagagcct

SEQ ID NO: 54 - DNA, 88 bp, Artificial Sequence
  Ccdc177 template sequence
aacgaacggagggtcattggggtacccagcgaagctcccgaaggctctccctatatttaaccgaattcccctaatgcgagtagagcct

SEQ ID NO: 55 - DNA, 88 bp, Artificial Sequence
  Ccdc177 template sequence
aacgaacggagggtcattggggtacctgcgggccgtgagcggaaccagagctatatttaaccgaattcccctaatgcgagtagagcct

SEQ ID NO: 56 - DNA, 88 bp, Artificial Sequence
  Ccdc177 template sequence
aacgaacggagggtcattggggtaccatcttgcggtcgcgctcgggtaccctatatttaaccgaattcccctaatgcgagtagagcct

SEQ ID NO: 57 - DNA, 88 bp, Artificial Sequence
  Ccdc177 template sequence
aacgaacggagggtcattggggtacccgccgctgttcggcgcgcacgcgcttatatttaaccgaattcccctaatgcgagtagagcct

SEQ ID NO: 58 - DNA, 88 bp, Artificial Sequence
  Ccdc177 template sequence
aacgaacggagggtcattggggtacccgccagctcccgccgccgctcctcgtatatttaaccgaattcccctaatgcgagtagagcct

SEQ ID NO: 59 - DNA, 88 bp, Artificial Sequence
  Ccdc177 template sequence
aacgaacggagggtcattggggtaccggaccagctccgcgcgctcccgccctatatttaaccgaattcccctaatgcgagtagagcct

SEQ ID NO: 60 - DNA, 88 bp, Artificial Sequence
  Ccdc177 template sequence
aacgaacggagggtcattggggtaccgcgcctcgtggcgcgcccgctcggctatatttaaccgaattcccctaatgcgagtagagcct

SEQ ID NO: 61 - DNA, 88 bp, Artificial Sequence
  Ccdc177 template sequence
aacgaacggagggtcattggggtaccgttcctgcagctgctcgtagttctctatatttaaccgaattcccctaatgcgagtagagcct

SEQ ID NO: 62 - DNA, 88 bp, Artificial Sequence
  Ccdc177 template sequence
aacgaacggagggtcattggggtacccctccaagtgcgcctgatgctcccgtatatttaaccgaattcccctaatgcgagtagagcct

SEQ ID NO: 63 - DNA, 88 bp, Artificial Sequence
  Ccdc177 template sequence
aacgaacggagggtcattggggtaccgttggcacgctgggccctctccttttatatttaaccgaattcccctaatgcgagtagagcct

SEQ ID NO: 64 - DNA, 88 bp, Artificial Sequence
  Ccdc177 template sequence
aacgaacggagggtcattggggtacccgcgcttcgccgttctcgagagagctatatttaaccgaattcccctaatgcgagtagagcct

SEQ ID NO: 65 - DNA, 88 bp, Artificial Sequence
  Ccdc177 template sequence
aacgaacggagggtcattggggtacccagcacggtctgtccagggcggctgtatatttaaccgaattcccctaatgcgagtagagcct

SEQ ID NO: 66 - DNA, 88 bp, Artificial Sequence
  Ccdc177 template sequence
aacgaacggagggtcattggggtacccattaatggttcattagtctggagatatatttaaccgaattcccctaatgcgagtagagcct

SEQ ID NO: 67 - DNA, 88 bp, Artificial Sequence
  Ccdc177 template sequence
aacgaacggagggtcattggggtaccctttctattccaacccaaagacacctatatttaaccgaattcccctaatgcgagtagagcct

SEQ ID NO: 68 - DNA, 88 bp, Artificial Sequence
  Ccdc177 template sequence
aacgaacggagggtcattggggtaccttccctccgctgctgctacttaaattatatttaaccgaattcccctaatgcgagtagagcct

SEQ ID NO: 69 - DNA, 88 bp, Artificial Sequence
  Ccdc177 template sequence
aacgaacggagggtcattggggtacccaattcacagtctagcgatagcttttatatttaaccgaattcccctaatgcgagtagagcct

SEQ ID NO: 70 - DNA, 88 bp, Artificial Sequence
  Ccdc177 template sequence
aacgaacggagggtcattggggtaccctaagcagcctcaccgatccagccctatatttaaccgaattcccctaatgcgagtagagcct

SEQ ID NO: 71 - DNA, 88 bp, Artificial Sequence
  Ccdc177 template sequence
aacgaacggagggtcattggggtaccggcttctgtctgcaggagctggccctatatttaaccgaattcccctaatgcgagtagagcct

SEQ ID NO: 72 - DNA, 88 bp, Artificial Sequence
  Ccdc177 template sequence
aacgaacggagggtcattggggtaccgaacgctgaactaagtcacagagggtatatttaaccgaattcccctaatgcgagtagagcct

SEQ ID NO: 73 - DNA, 88 bp, Artificial Sequence
  Ccdc177 template sequence
aacgaacggagggtcattggggtaccgaatttaaagagggaagactccttttatatttaaccgaattcccctaatgcgagtagagcct

SEQ ID NO: 74 - DNA, 88 bp, Artificial Sequence
  Ccdc177 template sequence
aacgaacggagggtcattggggtaccacaaaggagaccaacaaataacacttatatttaaccgaattcccctaatgcgagtagagcct

SEQ ID NO: 75 - DNA, 88 bp, Artificial Sequence
  Ccdc177 template sequence
aacgaacggagggtcattggggtacccatttgtctgtgatgcactgccctgtatatttaaccgaattcccctaatgcgagtagagcct

SEQ ID NO: 76 - DNA, 88 bp, Artificial Sequence
  Ccdc177 template sequence
aacgaacggagggtcattggggtaccaagctgacgggtggatctgagtgggtatatttaaccgaattcccctaatgcgagtagagcct

SEQ ID NO: 77 - DNA, 88 bp, Artificial Sequence
  Ccdc177 template sequence
aacgaacggagggtcattggggtacccagctttcttctacacaagactccctatatttaaccgaattcccctaatgcgagtagagcct

SEQ ID NO: 78 - DNA, 88 bp, Artificial Sequence
  Ccdc177 template sequence
aacgaacggagggtcattggggtacccagctcactgacaaggaaattaaggtatatttaaccgaattcccctaatgcgagtagagcct

SEQ ID NO: 79 - DNA, 88 bp, Artificial Sequence
  Ccdc177 template sequence
aacgaacggagggtcattggggtacctcaagggtagaactttccatggaggtatatttaaccgaattcccctaatgcgagtagagcct

SEQ ID NO: 80 - DNA, 88 bp, Artificial Sequence
  Ccdc177 template sequence
aacgaacggagggtcattggggtaccacctgaagtcgctcagtaaggtgggtatatttaaccgaattcccctaatgcgagtagagcct

SEQ ID NO: 81 - DNA, 88 bp, Artificial Sequence
  Ccdc177 template sequence
aacgaacggagggtcattggggtaccccaaggtgacagaaacaggaaatattatatttaaccgaattcccctaatgcgagtagagcct

SEQ ID NO: 82 - DNA, 88 bp, Artificial Sequence
  Ccdc177 template sequence
aacgaacggagggtcattggggtaccgagcttctcaagactgttccttctgtatatttaaccgaattcccctaatgcgagtagagcct

SEQ ID NO: 83 - DNA, 88 bp, Artificial Sequence
  Ccdc177 template sequence
aacgaacggagggtcattggggtaccttcttactgtcactaggtctgaatttatatttaaccgaattcccctaatgcgagtagagcct

SEQ ID NO: 84 - DNA, 88 bp, Artificial Sequence
  Ccdc177 template sequence
aacgaacggagggtcattggggtaccattatccatctacttcagcatcctctatatttaaccgaattcccctaatgcgagtagagcct

SEQ ID NO: 85 - DNA, 88 bp, Artificial Sequence
  Ccdc177 template sequence
aacgaacggagggtcattggggtaccacttaccctattcttctggcaccgggtccaccataggaattcccctaatgcgagtagagcct

SEQ ID NO: 86 - DNA, 88 bp, Artificial Sequence
  Ccdc177 template sequence
aacgaacggagggtcattggggtaccacttaccctataagagatcgaggtgcagcaacggaggaattcccctaatgcgagtagagcct

SEQ ID NO: 87 - DNA, 88 bp, Artificial Sequence
  Ccdc177 template sequence
aacgaacggagggtcattggggtaccacttaccctatcgcctcgtaggcctcgtacaggccggaattcccctaatgcgagtagagcct

SEQ ID NO: 88 - DNA, 88 bp, Artificial Sequence
  Ccdc177 template sequence
aacgaacggagggtcattggggtaccacttaccctatggcgggcgaggccgggaggctgctggaattcccctaatgcgagtagagcct

SEQ ID NO: 89 - DNA, 88 bp, Artificial Sequence
  Ccdc177 template sequence
aacgaacggagggtcattggggtaccacttaccctatcggactcggagctcagggcaccgtcgaattcccctaatgcgagtagagcct

SEQ ID NO: 90 - DNA, 88 bp, Artificial Sequence
  Ccdc177 template sequence
aacgaacggagggtcattggggtaccacttaccctatgggccgaagggcggcccagtgggttgaattcccctaatgcgagtagagcct

SEQ ID NO: 91 - DNA, 88 bp, Artificial Sequence
  Ccdc177 template sequence
aacgaacggagggtcattggggtaccacttaccctatgctcggcgcgcacttggcggacgatgaattcccctaatgcgagtagagcct

SEQ ID NO: 92 - DNA, 88 bp, Artificial Sequence
  Ccdc177 template sequence
aacgaacggagggtcattggggtaccacttaccctattgctcccactggccgtgggccgcgggaattcccctaatgcgagtagagcct

SEQ ID NO: 93 - DNA, 88 bp, Artificial Sequence
  Ccdc177 template sequence
aacgaacggagggtcattggggtaccacttaccctatgcgctcgcactgctgctgccgcctcgaattcccctaatgcgagtagagcct

SEQ ID NO: 94 - DNA, 88 bp, Artificial Sequence
  Ccdc177 template sequence
aacgaacggagggtcattggggtaccacttaccctatctgctcctgctgcagcttgcgcagcgaattcccctaatgcgagtagagcct

SEQ ID NO: 95 - DNA, 88 bp, Artificial Sequence
  Ccdc177 template sequence
aacgaacggagggtcattggggtaccacttaccctatggctcagctcgcgcttctcgcgctggaattcccctaatgcgagtagagcct

SEQ ID NO: 96 - DNA, 88 bp, Artificial Sequence
  Ccdc177 template sequence
aacgaacggagggtcattggggtaccacttaccctatgcgcgcggcccaagctggcctccaggaattcccctaatgcgagtagagcct

SEQ ID NO: 97 - DNA, 88 bp, Artificial Sequence
  Ccdc177 template sequence
aacgaacggagggtcattggggtaccacttaccctatctttgcgctcggccgcttccttcgcgaattcccctaatgcgagtagagcct

SEQ ID NO: 98 - DNA, 88 bp, Artificial Sequence
  Ccdc177 template sequence
aacgaacggagggtcattggggtaccacttaccctatcgcgccgggccttctgctgcacagcgaattcccctaatgcgagtagagcct

SEQ ID NO: 99 - DNA, 88 bp, Artificial Sequence
  Ccdc177 template sequence
aacgaacggagggtcattggggtaccacttaccctatctcactgcgctccagcttgcgcccagaattcccctaatgcgagtagagcct

SEQ ID NO: 100 - DNA, 88 bp, Artificial Sequence
  Ccdc177 template sequence
aacgaacggagggtcattggggtaccacttaccctatatttgcggtccaggctggcatgtaggaattcccctaatgcgagtagagcct

SEQ ID NO: 101 - DNA, 88 bp, Artificial Sequence
  Ccdc177 template sequence
aacgaacggagggtcattggggtaccacttaccctattcatagtgtggcaactggactggaggaattcccctaatgcgagtagagcct

SEQ ID NO: 102 - DNA, 88 bp, Artificial Sequence
  Ccdc177 template sequence
aacgaacggagggtcattggggtaccacttaccctattggatgtgggtagattgtgccatgtgaattcccctaatgcgagtagagcct

SEQ ID NO: 103 - DNA, 88 bp, Artificial Sequence
  Ccdc177 template sequence
aacgaacggagggtcattggggtaccacttaccctattcttgaacacaattgttagaaagatgaattcccctaatgcgagtagagcct

SEQ ID NO: 104 - DNA, 88 bp, Artificial Sequence
  Ccdc177 template sequence
aacgaacggagggtcattggggtaccacttaccctatagacgagctgtgctttcttgggaaagaattcccctaatgcgagtagagcct

SEQ ID NO: 105 - DNA, 88 bp, Artificial Sequence
  Ccdc177 template sequence
aacgaacggagggtcattggggtaccacttaccctatttattatctgtagatgattgtctaagaattcccctaatgcgagtagagcct

SEQ ID NO: 106 - DNA, 88 bp, Artificial Sequence
  Ccdc177 template sequence
aacgaacggagggtcattggggtaccacttaccctatcatcagacactgcctccacttacgtgaattcccctaatgcgagtagagcct

SEQ ID NO: 107 - DNA, 88 bp, Artificial Sequence
  Ccdc177 template sequence
aacgaacggagggtcattggggtaccacttaccctattccagactgcattcaaggctggagcgaattcccctaatgcgagtagagcct

SEQ ID NO: 108 - DNA, 88 bp, Artificial Sequence
  Ccdc177 template sequence
aacgaacggagggtcattggggtaccacttaccctataacaatagcacagctccacacaggcgaattcccctaatgcgagtagagcct

SEQ ID NO: 109 - DNA, 88 bp, Artificial Sequence
  Ccdc177 template sequence
aacgaacggagggtcattggggtaccacttaccctatagtgatgctttggatccaagagaccgaattcccctaatgcgagtagagcct

SEQ ID NO: 110 - DNA, 88 bp, Artificial Sequence
  Ccdc177 template sequence
aacgaacggagggtcattggggtaccacttaccctattaatgccagcaatccatgtcaccaggaattcccctaatgcgagtagagcct

SEQ ID NO: 111 - DNA, 88 bp, Artificial Sequence
  Ccdc177 template sequence
aacgaacggagggtcattggggtaccacttaccctataactgaaccttctgaccagggagcggaattcccctaatgcgagtagagcct

SEQ ID NO: 112 - DNA, 88 bp, Artificial Sequence
  Ccdc177 template sequence
aacgaacggagggtcattggggtaccacttaccctatagacacactaacacgtctaatgcaggaattcccctaatgcgagtagagcct

SEQ ID NO: 113 - DNA, 88 bp, Artificial Sequence
  Ccdc177 template sequence
aacgaacggagggtcattggggtaccacttaccctatgaacccacccactcaagaaacaagcgaattcccctaatgcgagtagagcct

SEQ ID NO: 114 - DNA, 88 bp, Artificial Sequence
  Ccdc177 template sequence
aacgaacggagggtcattggggtaccacttaccctattcagaagcaacccagggaccacaaggaattcccctaatgcgagtagagcct

SEQ ID NO: 115 - DNA, 88 bp, Artificial Sequence
  template sequence
aacgaacggagggtcattggggtaccacttaccctattcgctcgcgtgcttcccagtgcttggaattcccctaatgcgagtagagcct

SEQ ID NO: 116 - DNA, 88 bp, Artificial Sequence
  Ccdc177 template sequence
aacgaacggagggtcattggggtaccacttaccctatccgtcattctcgtttgcacaaactagaattcccctaatgcgagtagagcct

SEQ ID NO: 117 - DNA, 88 bp, Artificial Sequence
  Ccdc177 template sequence
aacgaacggagggtcattggggtaccacttaccctatccaaatcctgatggtacattacactgaattcccctaatgcgagtagagcct

SEQ ID NO: 118 - DNA, 88 bp, Artificial Sequence
  Ccdc177 template sequence
aacgaacggagggtcattggggtaccacttaccctatttacatagtggttctctcagtgtctgaattcccctaatgcgagtagagcct

SEQ ID NO: 119 - DNA, 88 bp, Artificial Sequence
  Ccdc177 template sequence
aacgaacggagggtcattggggtaccacttaccctatggttaggtatagagatgtgagccacgaattcccctaatgcgagtagagcct

SEQ ID NO: 120 - DNA, 88 bp, Artificial Sequence
  Ccdc177 template sequence
aacgaacggagggtcattggggtaccacttaccctatgaaagaaacatgaacatttctttaggaattcccctaatgcgagtagagcct

SEQ ID NO: 121 - DNA, 88 bp, Artificial Sequence
  Ccdc177 template sequence
aacgaacggagggtcattggggtaccgaacctcgttctttgttggagtccctatatttaaccgaattcccctaatgcgagtagagcct

SEQ ID NO: 122 - DNA, 88 bp, Artificial Sequence
  Ccdc177 template sequence
aacgaacggagggtcattggggtacctgctcccgacgtccaccttctgccctatatttaaccgaattcccctaatgcgagtagagcct

SEQ ID NO: 123 - DNA, 88 bp, Artificial Sequence
  Ccdc177 template sequence
aacgaacggagggtcattggggtaccggccacgcgcatggagcggcccgggtatatttaaccgaattcccctaatgcgagtagagcct

SEQ ID NO: 124 - DNA, 88 bp, Artificial Sequence
  Ccdc177 template sequence
aacgaacggagggtcattggggtaccgctgctgcagctgctgctgctgccctatatttaaccgaattcccctaatgcgagtagagcct

SEQ ID NO: 125 - DNA, 88 bp, Artificial Sequence
  Ccdc177 template sequence
aacgaacggagggtcattggggtacctccggcgggagagcgagtccagcgatatatttaaccgaattcccctaatgcgagtagagcct

SEQ ID NO: 126 - DNA, 88 bp, Artificial Sequence
  Ccdc177 template sequence
aacgaacggagggtcattggggtaccgtgcagacgacgccgagcccgcgggtatatttaaccgaattcccctaatgcgagtagagcct

SEQ ID NO: 127 - DNA, 88 bp, Artificial Sequence
  Ccdc177 template sequence
aacgaacggagggtcattggggtaccgctccacatgctgggctgtctgcggtatatttaaccgaattcccctaatgcgagtagagcct

SEQ ID NO: 128 - DNA, 88 bp, Artificial Sequence
  Ccdc177 template sequence
aacgaacggagggtcattggggtacccgctgctccagcaacaggcgctctttatatttaaccgaattcccctaatgcgagtagagcct

SEQ ID NO: 129 - DNA, 88 bp, Artificial Sequence
  Ccdc177 template sequence
aacgaacggagggtcattggggtacccgcggcctcgcgctcctcccggccctatatttaaccgaattcccctaatgcgagtagagcct

SEQ ID NO: 130 - DNA, 88 bp, Artificial Sequence
  Ccdc177 template sequence
aacgaacggagggtcattggggtaccgtcgtcccgcgcggcgcgttccacctatatttaaccgaattcccctaatgcgagtagagcct

SEQ ID NO: 131 - DNA, 88 bp, Artificial Sequence
  Ccdc177 template sequence
aacgaacggagggtcattggggtaccgttggccctcttgccgctctcgggctatatttaaccgaattcccctaatgcgagtagagcct

SEQ ID NO: 132 - DNA, 88 bp, Artificial Sequence
  Ccdc177 template sequence
aacgaacggagggtcattggggtaccagctccgcaggccctcccgctcctctatatttaaccgaattcccctaatgcgagtagagcct

SEQ ID NO: 133 - DNA, 88 bp, Artificial Sequence
  Ccdc177 template sequence
aacgaacggagggtcattggggtaccgccgaccctggagctcctctctccgtatatttaaccgaattcccctaatgcgagtagagcct

SEQ ID NO: 134 - DNA, 88 bp, Artificial Sequence
  Ccdc177 template sequence
aacgaacggagggtcattggggtacccttcggccacctgcgccgcgtgctgtatatttaaccgaattcccctaatgcgagtagagcct

SEQ ID NO: 135 - DNA, 88 bp, Artificial Sequence
  Ccdc177 template sequence
aacgaacggagggtcattggggtaccggcctgcagcagctcgcggcggcggtatatttaaccgaattcccctaatgcgagtagagcct

SEQ ID NO: 136 - DNA, 88 bp, Artificial Sequence
  Ccdc177 template sequence
aacgaacggagggtcattggggtaccgggcctctcgcaccatgcggtcaaatatatttaaccgaattcccctaatgcgagtagagcct

SEQ ID NO: 137 - DNA, 88 bp, Artificial Sequence
  Ccdc177 template sequence
aacgaacggagggtcattggggtaccgcctggagtttggtcaaggtgactatatatttaaccgaattcccctaatgcgagtagagcct

SEQ ID NO: 138 - DNA, 88 bp, Artificial Sequence
  Ccdc177 template sequence
aacgaacggagggtcattggggtacctttccaaatggagtaagtctgtaaatatatttaaccgaattcccctaatgcgagtagagcct

SEQ ID NO: 139 - DNA, 88 bp, Artificial Sequence
  Ccdc177 template sequence
aacgaacggagggtcattggggtaccccaaggcagctgtggcagagagccctatatttaaccgaattcccctaatgcgagtagagcct

SEQ ID NO: 140 - DNA, 88 bp, Artificial Sequence
  Ccdc177 template sequence
aacgaacggagggtcattggggtaccttcaagtcctgtgcaacttgtattttatatttaaccgaattcccctaatgcgagtagagcct

SEQ ID NO: 141 - DNA, 88 bp, Artificial Sequence
  Ccdc177 template sequence
aacgaacggagggtcattggggtaccagaaagccaagggactcaactgaaatatatttaaccgaattcccctaatgcgagtagagcct

SEQ ID NO: 142 - DNA, 88 bp, Artificial Sequence
  Ccdc177 template sequence
aacgaacggagggtcattggggtacctcaccttccatggaagcctcatgggtatatttaaccgaattcccctaatgcgagtagagcct

SEQ ID NO: 143 - DNA, 88 bp, Artificial Sequence
  Ccdc177 template sequence
aacgaacggagggtcattggggtacctaagcaagagtggcctgggcaggcatatatttaaccgaattcccctaatgcgagtagagcct

SEQ ID NO: 144 - DNA, 88 bp, Artificial Sequence
  Ccdc177 template sequence
aacgaacggagggtcattggggtaccttctagcccttcagccaccacaggctatatttaaccgaattcccctaatgcgagtagagcct

SEQ ID NO: 145 - DNA, 88 bp, Artificial Sequence
  Ccdc177 template sequence
aacgaacggagggtcattggggtaccgaggtaccagaagagttgatggccctatatttaaccgaattcccctaatgcgagtagagcct

SEQ ID NO: 146 - DNA, 88 bp, Artificial Sequence
  Ccdc177 template sequence
aacgaacggagggtcattggggtaccacaaactaggctaacagctgtcctctatatttaaccgaattcccctaatgcgagtagagcct

SEQ ID NO: 147 - DNA, 88 bp, Artificial Sequence
  Ccdc177 template sequence
aacgaacggagggtcattggggtaccttggtattgcaagtaccaagtaccctatatttaaccgaattcccctaatgcgagtagagcct

SEQ ID NO: 148 - DNA, 88 bp, Artificial Sequence
  Ccdc177 template sequence
aacgaacggagggtcattggggtacccgtctctaccctgattcctttcttttatatttaaccgaattcccctaatgcgagtagagcct

SEQ ID NO: 149 - DNA, 88 bp, Artificial Sequence
  Ccdc177 template sequence
aacgaacggagggtcattggggtacccagtacatagctcctttcctggtactatatttaaccgaattcccctaatgcgagtagagcct

SEQ ID NO: 150 - DNA, 88 bp, Artificial Sequence
  Ccdc177 template sequence
aacgaacggagggtcattggggtacccaaaggaccataatcacattaattttatatttaaccgaattcccctaatgcgagtagagcct

SEQ ID NO: 151 - DNA, 88 bp, Artificial Sequence
  Ccdc177 template sequence
aacgaacggagggtcattggggtacctcagaacacccgattgcctcctcaatatatttaaccgaattcccctaatgcgagtagagcct

SEQ ID NO: 152 - DNA, 88 bp, Artificial Sequence
  Ccdc177 template sequence
aacgaacggagggtcattggggtaccgctcaggaaccatgatctcagttcttatatttaaccgaattcccctaatgcgagtagagcct

SEQ ID NO: 153 - DNA, 88 bp, Artificial Sequence
  Ccdc177 template sequence
aacgaacggagggtcattggggtaccacagtacggcgcaggagccatggagtatatttaaccgaattcccctaatgcgagtagagcct

SEQ ID NO: 154 - DNA, 88 bp, Artificial Sequence
  Ccdc177 template sequence
aacgaacggagggtcattggggtacctttgtcaacattctggcctgggccttatatttaaccgaattcccctaatgcgagtagagcct

SEQ ID NO: 155 - DNA, 88 bp, Artificial Sequence
  Ccdc177 template sequence
aacgaacggagggtcattggggtacctgtccaacttcagtagagtatcctatatatttaaccgaattcccctaatgcgagtagagcct

SEQ ID NO: 156 - DNA, 88 bp, Artificial Sequence
  Ccdc177 template sequence
aacgaacggagggtcattggggtacctatacaggtgattctccttgcattttatatttaaccgaattcccctaatgcgagtagagcct

SEQ ID NO: 157 - DNA, 55 bp, Artificial Sequence
  bridge sequence
attgtaaagcgtgagaaagggatagtggcggttaaatattgtaaagcgtgagaaa

SEQ ID NO: 158 - DNA, 55 bp, Artificial Sequence
  bridge sequence
attgtaaagcgtgagaaaggtagagtggcggttaaatattgtaaagcgtgagaaa

SEQ ID NO: 159 - DNA, 55 bp, Artificial Sequence
  bridge sequence
attgtaaagcgtgagaaaggattgagggcggttaaatattgtaaagcgtgagaaa

SEQ ID NO: 160 - DNA, 55 bp, Artificial Sequence
  bridge sequence
attgtaaagcgtgagaaagggtgtggggcggttaaatattgtaaagcgtgagaaa

SEQ ID NO: 161 - DNA, 55 bp, Artificial Sequence
  bridge sequence
attgtaaagcgtgagaaagggtaagtggcggttaaatattgtaaagcgtgagaaa

SEQ ID NO: 162 - DNA, 55 bp, Artificial Sequence
  bridge sequence
attgtaaagcgtgagaaaggtttggtggcggttaaatattgtaaagcgtgagaaa

SEQ ID NO: 163 - DNA, 55 bp, Artificial Sequence
  bridge sequence
attgtaaagcgtgagaaaggaaggttggcggttaaatattgtaaagcgtgagaaa

SEQ ID NO: 164 - DNA, 55 bp, Artificial Sequence
  bridge sequence
attgtaaagcgtgagaaaggtaatggggcggttaaatattgtaaagcgtgagaaa

SEQ ID NO: 165 - DNA, 55 bp, Artificial Sequence
  bridge sequence
attgtaaagcgtgagaaaggaagaagggcggttaaatattgtaaagcgtgagaaa

SEQ ID NO: 166 - DNA, 55 bp, Artificial Sequence
  bridge sequence
attgtaaagcgtgagaaaggagagttggcggttaaatattgtaaagcgtgagaaa

SEQ ID NO: 167 - DNA, 55 bp, Artificial Sequence
  bridge sequence
attgtaaagcgtgagaaaggtggtttggcggttaaatattgtaaagcgtgagaaa

SEQ ID NO: 168 - DNA, 55 bp, Artificial Sequence
  bridge sequence
attgtaaagcgtgagaaagggagattggcggttaaatattgtaaagcgtgagaaa

SEQ ID NO: 169 - DNA, 55 bp, Artificial Sequence
  bridge sequence
attgtaaagcgtgagaaaggattaggggcggttaaatattgtaaagcgtgagaaa

SEQ ID NO: 170 - DNA, 55 bp, Artificial Sequence
  bridge sequence
tgatgggtgcgtgagtaagggtatgaggcggttaaattgatgggtgcgtgagtaa

SEQ ID NO: 171 - DNA, 55 bp, Artificial Sequence
  bridge sequence
tgatgggtgcgtgagtaagggaagttggcggttaaattgatgggtgcgtgagtaa

SEQ ID NO: 172 - DNA, 55 bp, Artificial Sequence
  bridge sequence
tgatgggtgcgtgagtaaggagatgtggcggttaaattgatgggtgcgtgagtaa

SEQ ID NO: 173 - DNA, 55 bp, Artificial Sequence
  bridge sequence
tgatgggtgcgtgagtaaggaggaaaggcggttaaattgatgggtgcgtgagtaa

SEQ ID NO: 174 - DNA, 55 bp, Artificial Sequence
  bridge sequence
tgatgggtgcgtgagtaagggagaggggcggttaaattgatgggtgcgtgagtaa

SEQ ID NO: 175 - DNA, 55 bp, Artificial Sequence
  bridge sequence
tgatgggtgcgtgagtaaggtaaggtggcggttaaattgatgggtgcgtgagtaa

SEQ ID NO: 176 - DNA, 55 bp, Artificial Sequence
  bridge sequence
tgatgggtgcgtgagtaaggtgtttgggcggttaaattgatgggtgcgtgagtaa

SEQ ID NO: 177 - DNA, 55 bp, Artificial Sequence
  bridge sequence
tgatgggtgcgtgagtaaggttgttgggcggttaaattgatgggtgcgtgagtaa

SEQ ID NO: 178 - DNA, 55 bp, Artificial Sequence
  bridge sequence
tgatgggtgcgtgagtaaggagtgtaggcggttaaattgatgggtgcgtgagtaa

SEQ ID NO: 179 - DNA, 55 bp, Artificial Sequence
  bridge sequence
tgatgggtgcgtgagtaaggtaggtaggcggttaaattgatgggtgcgtgagtaa

SEQ ID NO: 180 - DNA, 55 bp, Artificial Sequence
  bridge sequence
tgatgggtgcgtgagtaaggttgtgtggcggttaaattgatgggtgcgtgagtaa

SEQ ID NO: 181 - DNA, 55 bp, Artificial Sequence
  bridge sequence
tgatgggtgcgtgagtaaggttggaaggcggttaaattgatgggtgcgtgagtaa

SEQ ID NO: 182 - DNA, 55 bp, Artificial Sequence
  bridge sequence
tgatgggtgcgtgagtaaggttgagaggcggttaaattgatgggtgcgtgagtaa

SEQ ID NO: 183 - DNA, 20 bp, Artificial Sequence
  Primer sequence
tggttcaatcgtatgcccgt

SEQ ID NO: 184 - DNA, 40 bp, Artificial Sequence
  Primer sequence
taatacgactcactataggggtcacttagccaacgccgat

SEQ ID NO: 185 - DNA, 130 bp, Artificial Sequence
  Igf1 Template sequence
tggttcaatcgtatgcccgtcccatgatcgtccgatctggtcggatttgtgtctgattcaaataagtcaaaccacaccttttagtcgtcgcaacgccaggcaactcatgcatcggcgttggctaagtgac

SEQ ID NO: 186 - DNA, 130 bp, Artificial Sequence
  Igf1 Template sequence
tggttcaatcgtatgcccgtcccatgatcgtccgatctggtcggatttgtacctgtgattgtaatagatactatttcaaattagtcgtcgcaacgccaggcaactcatgcatcggcgttggctaagtgac

SEQ ID NO: 187 - DNA, 130 bp, Artificial Sequence
  Igf1 Template sequence
tggttcaatcgtatgcccgtcccatgatcgtccgatctggtcggatttgtggagctttcctgtaaacttaatatacactgttagtcgtcgcaacgccaggcaactcatgcatcggcgttggctaagtgac

SEQ ID NO: 188 - DNA, 130 bp, Artificial Sequence
  Igf1 Template sequence
tggttcaatcgtatgcccgtcccatgatcgtccgatctggtcggatttgtaaggttcagacagtcttatctctttcctttttagtcgtcgcaacgccaggcaactcatgcatcggcgttggctaagtgac

SEQ ID NO: 189 - DNA, 130 bp, Artificial Sequence
  Igf1 Template sequence
tggttcaatcgtatgcccgtcccatgatcgtccgatctggtcggatttgtggactaggagatatagttatatatatatttttagtcgtcgcaacgccaggcaactcatgcatcggcgttggctaagtgac

SEQ ID NO: 190 - DNA, 130 bp, Artificial Sequence
  Igf1 Template sequence
tggttcaatcgtatgcccgtcccatgatcgtccgatctggtcggatttgtagctggatccttccttctaagcagacactattagtcgtcgcaacgccaggcaactcatgcatcggcgttggctaagtgac

SEQ ID NO: 191 - DNA, 130 bp, Artificial Sequence
  Igf1 Template sequence
tggttcaatcgtatgcccgtcccatgatcgtccgatctggtcggatttgtgagcgtatgcacgcatgtgcaaatgcacatttagtcgtcgcaacgccaggcaactcatgcatcggcgttggctaagtgac

SEQ ID NO: 192 - DNA, 130 bp, Artificial Sequence
  Igf1 Template sequence
tggttcaatcgtatgcccgtcccatgatcgtccgatctggtcggatttgtcaactggatctctacaacatccatgcatttttagtcgtcgcaacgccaggcaactcatgcatcggcgttggctaagtgac

SEQ ID NO: 193 - DNA, 130 bp, Artificial Sequence
  Igf1 Template sequence
tggttcaatcgtatgcccgtcccatgatcgtccgatctggtcggatttgtaatgaaatgattgagccatctaactttaaattagtcgtcgcaacgccaggcaactcatgcatcggcgttggctaagtgac

SEQ ID NO: 194 - DNA, 130 bp, Artificial Sequence
  Igf1 Template sequence
tggttcaatcgtatgcccgtcccatgatcgtccgatctggtcggatttgtacattcgccttctctctctctccctcttctttagtcgtcgcaacgccaggcaactcatgcatcggcgttggctaagtgac

SEQ ID NO: 195 - DNA, 130 bp, Artificial Sequence
  Igf1 Template sequence
tggttcaatcgtatgcccgtcccatgatcgtccgatctggtcggatttgtaaacccaaacccaacaacaacaacaacaaagtaagcgcaacggtggaccggagacgacggatcggcgttggctaagtgac

SEQ ID NO: 196 - DNA, 130 bp, Artificial Sequence
  Igf1 Template sequence
tggttcaatcgtatgcccgtcccatgatcgtccgatctggtcggatttgttgtgatctctttatcatgtgccttacgaatgtaagcgcaacggtggaccggagacgacggatcggcgttggctaagtgac

SEQ ID NO: 197 - DNA, 130 bp, Artificial Sequence
  Igf1 Template sequence
tggttcaatcgtatgcccgtcccatgatcgtccgatctggtcggatttgtagacagtctttagtctggccagcctctaaagtaagcgcaacggtggaccggagacgacggatcggcgttggctaagtgac

SEQ ID NO: 198 - DNA, 130 bp, Artificial Sequence
  Igf1 Template sequence
tggttcaatcgtatgcccgtcccatgatcgtccgatctggtcggatttgtaatacaagtgttaggaaagggtgtgtctaagtaagcgcaacggtggaccggagacgacggatcggcgttggctaagtgac

SEQ ID NO: 199 - DNA, 130 bp, Artificial Sequence
  Igf1 Template sequence
tggttcaatcgtatgcccgtcccatgatcgtccgatctggtcggatttgttattgttatttggtaggtgtttcgatgtttgtaagcgcaacggtggaccggagacgacggatcggcgttggctaagtgac

SEQ ID NO: 200 - DNA, 130 bp, Artificial Sequence
  Igf1 Template sequence
tggttcaatcgtatgcccgtcccatgatcgtccgatctggtcggatttgtatgaatgaccaaatattactttcaggtttcgtaagcgcaacggtggaccggagacgacggatcggcgttggctaagtgac

SEQ ID NO: 201 - DNA, 130 bp, Artificial Sequence
  Igf1 Template sequence
tggttcaatcgtatgcccgtcccatgatcgtccgatctggtcggatttgttgcgcatcctcccaagtgcacaaaccataagtaagcgcaacggtggaccggagacgacggatcggcgttggctaagtgac

SEQ ID NO: 202 - DNA, 130 bp, Artificial Sequence
  Igf1 Template sequence
tggttcaatcgtatgcccgtcccatgatcgtccgatctggtcggatttgttgtgtttggcaaactgagtagagtctcattgtaagcgcaacggtggaccggagacgacggatcggcgttggctaagtgac

SEQ ID NO: 203 - DNA, 130 bp, Artificial Sequence
  Igf1 Template sequence
tggttcaatcgtatgcccgtcccatgatcgtccgatctggtcggatttgtacaatcttgctatatacatagagaacatttgtaagcgcaacggtggaccggagacgacggatcggcgttggctaagtgac

SEQ ID NO: 204 - DNA, 130 bp, Artificial Sequence
  Igf1 Template sequence
tggttcaatcgtatgcccgtcccatgatcgtccgatctggtcggatttgtttccttgcatcctagcaattcaagggttctgtaagcgcaacggtggaccggagacgacggatcggcgttggctaagtgac

SEQ ID NO: 205 - DNA, 130 bp, Artificial Sequence
  Igf1 Template sequence
tggttcaatcgtatgcccgtcccatgatcgtccgatctggtcggatttgtcatcacccttggcctttaaagtgtgataaacggccgaattacatccgtcgtaatcgaggcatcggcgttggctaagtgac

SEQ ID NO: 206 - DNA, 130 bp, Artificial Sequence
  Igf1 Template sequence
tggttcaatcgtatgcccgtcccatgatcgtccgatctggtcggatttgttaaatgtaagataatgattcagttagttaccggccgaattacatccgtcgtaatcgaggcatcggcgttggctaagtgac

SEQ ID NO: 207 - DNA, 130 bp, Artificial Sequence
  Igf1 Template sequence
tggttcaatcgtatgcccgtcccatgatcgtccgatctggtcggatttgtcttccctttctttgtcagtagataagcccacggccgaattacatccgtcgtaatcgaggcatcggcgttggctaagtgac

SEQ ID NO: 208 - DNA, 130 bp, Artificial Sequence
  Igf1 Template sequence
tggttcaatcgtatgcccgtcccatgatcgtccgatctggtcggatttgttaagggaaccattcataaaccacctctacacggccgaattacatccgtcgtaatcgaggcatcggcgttggctaagtgac

SEQ ID NO: 209 - DNA, 130 bp, Artificial Sequence
  Igf1 Template sequence
tggttcaatcgtatgcccgtcccatgatcgtccgatctggtcggatttgtagttggagagaattaggtattggataaaggcggccgaattacatccgtcgtaatcgaggcatcggcgttggctaagtgac

SEQ ID NO: 210 - DNA, 130 bp, Artificial Sequence
  Igf1 Template sequence
tggttcaatcgtatgcccgtcccatgatcgtccgatctggtcggatttgtcaaacaagggcaagactacatctgctacagcggccgaattacatccgtcgtaatcgaggcatcggcgttggctaagtgac

SEQ ID NO: 211 - DNA, 130 bp, Artificial Sequence
  Igf1 Template sequence
tggttcaatcgtatgcccgtcccatgatcgtccgatctggtcggatttgtatttcaattgggcagtgacatttagaatttcggccgaattacatccgtcgtaatcgaggcatcggcgttggctaagtgac

SEQ ID NO: 212 - DNA, 130 bp, Artificial Sequence
  Igf1 Template sequence
tggttcaatcgtatgcccgtcccatgatcgtccgatctggtcggatttgtattattatgattaattagtacaattaatttcggccgaattacatccgtcgtaatcgaggcatcggcgttggctaagtgac

SEQ ID NO: 213 - DNA, 130 bp, Artificial Sequence
  Igf1 Template sequence
tggttcaatcgtatgcccgtcccatgatcgtccgatctggtcggatttgtaaatcaacaggaaaccaaacaatcaacaaacggccgaattacatccgtcgtaatcgaggcatcggcgttggctaagtgac

SEQ ID NO: 214 - DNA, 130 bp, Artificial Sequence
  Igf1 Template sequence
tggttcaatcgtatgcccgtcccatgatcgtccgatctggtcggatttgtacaatttacctccatagcaaagtctacaaacggccgaattacatccgtcgtaatcgaggcatcggcgttggctaagtgac

SEQ ID NO: 215 - DNA, 130 bp, Artificial Sequence
  Igf1 Template sequence
tggttcaatcgtatgcccgtttagtcgtcgcaacgccaggcaactcatgcgactgagtccttagcttgtcactaattagtgtaagcgcaacggtggaccggagacgacggatcggcgttggctaagtgac

SEQ ID NO: 216 - DNA, 130 bp, Artificial Sequence
  Igf1 Template sequence
tggttcaatcgtatgcccgtttagtcgtcgcaacgccaggcaactcatgcaaacgaaatgagactaggtgatagatcaacgtaagcgcaacggtggaccggagacgacggatcggcgttggctaagtgac

SEQ ID NO: 217 - DNA, 130 bp, Artificial Sequence
  Igf1 Template sequence
tggttcaatcgtatgcccgtttagtcgtcgcaacgccaggcaactcatgcattccttcctacctatctttatagttctttgtaagcgcaacggtggaccggagacgacggatcggcgttggctaagtgac

SEQ ID NO: 218 - DNA, 130 bp, Artificial Sequence
  Igf1 Template sequence
tggttcaatcgtatgcccgtttagtcgtcgcaacgccaggcaactcatgcacttcagattgggtgaagtattgccaatttgtaagcgcaacggtggaccggagacgacggatcggcgttggctaagtgac

SEQ ID NO: 219 - DNA, 130 bp, Artificial Sequence
  Igf1 Template sequence
tggttcaatcgtatgcccgtttagtcgtcgcaacgccaggcaactcatgcaatgtaattactaaagaaagatataccattgtaagcgcaacggtggaccggagacgacggatcggcgttggctaagtgac

SEQ ID NO: 220 - DNA, 130 bp, Artificial Sequence
  Igf1 Template sequence
tggttcaatcgtatgcccgtttagtcgtcgcaacgccaggcaactcatgccccagttgaatgcatccaacacatttccaggtaagcgcaacggtggaccggagacgacggatcggcgttggctaagtgac

SEQ ID NO: 221 - DNA, 130 bp, Artificial Sequence
  Igf1 Template sequence
tggttcaatcgtatgcccgtttagtcgtcgcaacgccaggcaactcatgccagctagacaagatgattagactctgtaaagtaagcgcaacggtggaccggagacgacggatcggcgttggctaagtgac

SEQ ID NO: 222 - DNA, 130 bp, Artificial Sequence
  Igf1 Template sequence
tggttcaatcgtatgcccgtttagtcgtcgcaacgccaggcaactcatgcatactataggattaagcactagcattgaaagtaagcgcaacggtggaccggagacgacggatcggcgttggctaagtgac

SEQ ID NO: 223 - DNA, 130 bp, Artificial Sequence
  Igf1 Template sequence
tggttcaatcgtatgcccgtttagtcgtcgcaacgccaggcaactcatgctcaagtactttcttaaagaaacaatagcacgtaagcgcaacggtggaccggagacgacggatcggcgttggctaagtgac

SEQ ID NO: 224 - DNA, 130 bp, Artificial Sequence
  Igf1 Template sequence
tggttcaatcgtatgcccgtttagtcgtcgcaacgccaggcaactcatgctcttaggctccaggctttcgtttgttgtttgtaagcgcaacggtggaccggagacgacggatcggcgttggctaagtgac

SEQ ID NO: 225 - DNA, 130 bp, Artificial Sequence
  Igf1 Template sequence
tggttcaatcgtatgcccgtttagtcgtcgcaacgccaggcaactcatgcagcaaaggatcctgcggtgatgtggcatttcggccgaattacatccgtcgtaatcgaggcatcggcgttggctaagtgac

SEQ ID NO: 226 - DNA, 130 bp, Artificial Sequence
  Igf1 Template sequence
tggttcaatcgtatgcccgtttagtcgtcgcaacgccaggcaactcatgcagtgtttagcagtaggtacaatgtaaatatcggccgaattacatccgtcgtaatcgaggcatcggcgttggctaagtgac

SEQ ID NO: 227 - DNA, 130 bp, Artificial Sequence
  Igf1 Template sequence
tggttcaatcgtatgcccgtttagtcgtcgcaacgccaggcaactcatgctaaataattgagttggaaggctgctgatttcggccgaattacatccgtcgtaatcgaggcatcggcgttggctaagtgac

SEQ ID NO: 228 - DNA, 130 bp, Artificial Sequence
  Igf1 Template sequence
tggttcaatcgtatgcccgtttagtcgtcgcaacgccaggcaactcatgcctggcggaaggagagctaaaggtgtcctttcggccgaattacatccgtcgtaatcgaggcatcggcgttggctaagtgac

SEQ ID NO: 229 - DNA, 130 bp, Artificial Sequence
  Igf1 Template sequence
tggttcaatcgtatgcccgtttagtcgtcgcaacgccaggcaactcatgctttacacaagttcactttgggcaagagaaacggccgaattacatccgtcgtaatcgaggcatcggcgttggctaagtgac

SEQ ID NO: 230 - DNA, 130 bp, Artificial Sequence
  Igf1 Template sequence
tggttcaatcgtatgcccgtttagtcgtcgcaacgccaggcaactcatgcgagaacctcaggcttgagaaggaagaatttcggccgaattacatccgtcgtaatcgaggcatcggcgttggctaagtgac

SEQ ID NO: 231 - DNA, 130 bp, Artificial Sequence
  Igf1 Template sequence
tggttcaatcgtatgcccgtttagtcgtcgcaacgccaggcaactcatgccatgtacacagacatgccacatttcacattcggccgaattacatccgtcgtaatcgaggcatcggcgttggctaagtgac

SEQ ID NO: 232 - DNA, 130 bp, Artificial Sequence
  Igf1 Template sequence
tggttcaatcgtatgcccgtttagtcgtcgcaacgccaggcaactcatgcacttccttctgagtcttgggcatgtcagtgcggccgaattacatccgtcgtaatcgaggcatcggcgttggctaagtgac

SEQ ID NO: 233 - DNA, 130 bp, Artificial Sequence
  Igf1 Template sequence
tggttcaatcgtatgcccgtttagtcgtcgcaacgccaggcaactcatgccaaatgtctcctgccgtgtgacatgctgggcggccgaattacatccgtcgtaatcgaggcatcggcgttggctaagtgac

SEQ ID NO: 234 - DNA, 130 bp, Artificial Sequence
  Igf1 Template sequence
tggttcaatcgtatgcccgtttagtcgtcgcaacgccaggcaactcatgcacttgtgagtgacacatagttgaaaggtggcggccgaattacatccgtcgtaatcgaggcatcggcgttggctaagtgac

SEQ ID NO: 235 - DNA, 130 bp, Artificial Sequence
  Igf1 Template sequence
tggttcaatcgtatgcccgtgtaagcgcaacggtggaccggagacgacggtaaatgtccctttctatcaatcttgagtcacggccgaattacatccgtcgtaatcgaggcatcggcgttggctaagtgac

SEQ ID NO: 236 - DNA, 130 bp, Artificial Sequence
  Igf1 Template sequence
tggttcaatcgtatgcccgtgtaagcgcaacggtggaccggagacgacggtaattaattaccctttaatgagagtggatacggccgaattacatccgtcgtaatcgaggcatcggcgttggctaagtgac

SEQ ID NO: 237 - DNA, 130 bp, Artificial Sequence
  Igf1 Template sequence
tggttcaatcgtatgcccgtgtaagcgcaacggtggaccggagacgacggagatgttagagcaatgtcacatttcaatttcggccgaattacatccgtcgtaatcgaggcatcggcgttggctaagtgac

SEQ ID NO: 238 - DNA, 130 bp, Artificial Sequence
  Igf1 Template sequence
tggttcaatcgtatgcccgtgtaagcgcaacggtggaccggagacgacggacagatctggagaaggccacctatgtgtttcggccgaattacatccgtcgtaatcgaggcatcggcgttggctaagtgac

SEQ ID NO: 239 - DNA, 130 bp, Artificial Sequence
  Igf1 Template sequence
tggttcaatcgtatgcccgtgtaagcgcaacggtggaccggagacgacggggagctacattgttagtgggttatttacagcggccgaattacatccgtcgtaatcgaggcatcggcgttggctaagtgac

SEQ ID NO: 240 - DNA, 130 bp, Artificial Sequence
  Igf1 Template sequence
tggttcaatcgtatgcccgtgtaagcgcaacggtggaccggagacgacggcacagcttcaccaataagaacttggatattcggccgaattacatccgtcgtaatcgaggcatcggcgttggctaagtgac

SEQ ID NO: 241 - DNA, 130 bp, Artificial Sequence
  Igf1 Template sequence
tggttcaatcgtatgcccgtgtaagcgcaacggtggaccggagacgacggctttatcttcaagaagtcacagaggcagatcggccgaattacatccgtcgtaatcgaggcatcggcgttggctaagtgac

SEQ ID NO: 242 - DNA, 130 bp, Artificial Sequence
  Igf1 Template sequence
tggttcaatcgtatgcccgtgtaagcgcaacggtggaccggagacgacggtagattataatggagagatgtattacatttcggccgaattacatccgtcgtaatcgaggcatcggcgttggctaagtgac

SEQ ID NO: 243 - DNA, 130 bp, Artificial Sequence
  Igf1 Template sequence
tggttcaatcgtatgcccgtgtaagcgcaacggtggaccggagacgacggaaggattgaaccagatatggcttgttatttcggccgaattacatccgtcgtaatcgaggcatcggcgttggctaagtgac

SEQ ID NO: 244 - DNA, 130 bp, Artificial Sequence
  Igf1 Template sequence
tggttcaatcgtatgcccgtgtaagcgcaacggtggaccggagacgacggagccatagcctgtgggcttgttgaagtaaacggccgaattacatccgtcgtaatcgaggcatcggcgttggctaagtgac

SEQ ID NO: 245 - DNA, 30 bp, Artificial Sequence
  readout probe
cgcaacgcttgggacggttccaatcggatc

SEQ ID NO: 246 - DNA, 30 bp, Artificial Sequence
  readout probe
cgaatgctctggcctcgaacgaacgatagc

SEQ ID NO: 247 - DNA, 30 bp, Artificial Sequence
  readout probe
acaaatccgaccagatcggacgatcatggg

SEQ ID NO: 248 - DNA, 30 bp, Artificial Sequence
  readout probe
caagtatgcagcgcgattgaccgtctcgtt

SEQ ID NO: 249 - DNA, 30 bp, Artificial Sequence
  readout probe
gcgggaagcacgtggattagggcatcgacc

SEQ ID NO: 250 - DNA, 30 bp, Artificial Sequence
  readout probe
aagtcgtacgccgatgcgcagcaattcact

SEQ ID NO: 251 - DNA, 30 bp, Artificial Sequence
  readout probe
cgaaacatcggccacggtcccgttgaactt

SEQ ID NO: 252 - DNA, 30 bp, Artificial Sequence
  readout probe
acgaatccaccgtccagcgcgtcaaacaga

SEQ ID NO: 253 - DNA, 30 bp, Artificial Sequence
  readout probe
cgcgaaatccccgtaacgagcgtcccttgc

SEQ ID NO: 254 - DNA, 30 bp, Artificial Sequence
  readout probe
gcatgagttgcctggcgttgcgacgactaa

SEQ ID NO: 255 - DNA, 30 bp, Artificial Sequence
  readout probe
ccgtcgtctccggtccaccgttgcgcttac

SEQ ID NO: 256 - DNA, 30 bp, Artificial Sequence
  readout probe
ggccaatggcccaggtccgtcacgcaattt

SEQ ID NO: 257 - DNA, 30 bp, Artificial Sequence
  readout probe
ttgatcgaatcggagcgtagcggaatctgc

SEQ ID NO: 258 - DNA, 30 bp, Artificial Sequence
  readout probe
cgcgcggatccgcttgtcgggaacggatac

SEQ ID NO: 259 - DNA, 30 bp, Artificial Sequence
  readout probe
gcctcgattacgacggatgtaattcggccg

SEQ ID NO: 260 - DNA, 30 bp, Artificial Sequence
  readout probe
gcccgtattcccgcttgcgagtagggcaat

SEQ ID NO: 261 - DNA, 38 bp, Artificial Sequence
  XLOC_1
cattcccagggacattcatttatccaccgaacccttac

SEQ ID NO: 262 - DNA, 38 bp, Artificial Sequence
  XLOC_2
gatgacttctctatcccacttatccaccgaacccttac

SEQ ID NO: 263 - DNA, 38 bp, Artificial Sequence
  XLOC_3
gaagtgcctcgtcattctggtatccaccgaacccttac

SEQ ID NO: 264 - DNA, 38 bp, Artificial Sequence
  XLOC_4
gctccagtaactagctgacatatccaccgaacccttac

SEQ ID NO: 265 - DNA, 38 bp, Artificial Sequence
  XLOC_5
aagcccaggggtactccttatatccaccgaacccttac

SEQ ID NO: 266 - DNA, 38 bp, Artificial Sequence
  XLOC_6
ggagccttgtaaatactgattatccaccgaacccttac

SEQ ID NO: 267 - DNA, 38 bp, Artificial Sequence
  XLOC_7
gctggaacccttaggaaatatatccaccgaacccttac

SEQ ID NO: 268 - DNA, 38 bp, Artificial Sequence
  XLOC_8
gagaatggcaagaactccattatccaccgaacccttac

SEQ ID NO: 269 - DNA, 38 bp, Artificial Sequence
  XLOC_9
atgaacaggcagaggctagatatccaccgaacccttac

SEQ ID NO: 270 - DNA, 38 bp, Artificial Sequence
  XLOC_10
tactgatctgaagcagcaggtatccaccgaacccttac

SEQ ID NO: 271 - DNA, 38 bp, Artificial Sequence
  XLOC_11
cagactaaaaggaggccaggtatccaccgaacccttac

SEQ ID NO: 272 - DNA, 38 bp, Artificial Sequence
  XLOC_12
agtgaattgagaatatgaggtatccaccgaacccttac

SEQ ID NO: 273 - DNA, 38 bp, Artificial Sequence
  XLOC_13
aggatagtaactcagtagaatatccaccgaacccttac

SEQ ID NO: 274 - DNA, 38 bp, Artificial Sequence
  XLOC_14
tgataggaggcattgtcatttatccaccgaacccttac

SEQ ID NO: 275 - DNA, 38 bp, Artificial Sequence
  XLOC_15
ttgtatcaaaccaagtctcttatccaccgaacccttac

SEQ ID NO: 276 - DNA, 38 bp, Artificial Sequence
  XLOC_16
acgagcagttgtatgtgaattatccaccgaacccttac

SEQ ID NO: 277 - DNA, 38 bp, Artificial Sequence
  XLOC_17
ttccccagttatagaaatcatatccaccgaacccttac

SEQ ID NO: 278 - DNA, 38 bp, Artificial Sequence
  XLOC_18
tatctggggtaatggagacatatccaccgaacccttac

SEQ ID NO: 279 - DNA, 38 bp, Artificial Sequence
  XLOC_19
cgaatttcctgcactggtaatatccaccgaacccttac

SEQ ID NO: 280 - DNA, 38 bp, Artificial Sequence
  XLOC_20
tcaggttgtatgatatagtgtatccaccgaacccttac

SEQ ID NO: 281 - DNA, 38 bp, Artificial Sequence
  XLOC_21
actgtctacttcaccattgatatccaccgaacccttac

SEQ ID NO: 282 - DNA, 38 bp, Artificial Sequence
  XLOC_22
taaaacatccattgccctggtatccaccgaacccttac

SEQ ID NO: 283 - DNA, 38 bp, Artificial Sequence
  XLOC_23
acagaccggttaaccatccatatccaccgaacccttac

SEQ ID NO: 284 - DNA, 32 bp, Artificial Sequence
  XLOC_SplitLeft_1
aacccttactataaattaacatgctcggccag

SEQ ID NO: 285 - DNA, 32 bp, Artificial Sequence
  XLOC_SplitLeft_2
aacccttactataattctccttgaagatcatt

SEQ ID NO: 286 - DNA, 32 bp, Artificial Sequence
  XLOC_SplitLeft_3
aacccttactattcagacacaggtgcagatag

SEQ ID NO: 287 - DNA, 32 bp, Artificial Sequence
  XLOC_SplitLeft_4
aacccttactattccagtaactagctgacatg

SEQ ID NO: 288 - DNA, 32 bp, Artificial Sequence
  XLOC_SplitLeft_5
aacccttactatctgatgaagtgattaaggag

SEQ ID NO: 289 - DNA, 32 bp, Artificial Sequence
  XLOC_SplitLeft_6
aacccttactataattacactgcctggcacat

SEQ ID NO: 290 - DNA, 32 bp, Artificial Sequence
  XLOC_SplitLeft_7
aacccttactatagtgcctcagacactgcctg

SEQ ID NO: 291 - DNA, 32 bp, Artificial Sequence
  XLOC_SplitLeft_8
aacccttactattatccatgggctgctggaac

SEQ ID NO: 292 - DNA, 32 bp, Artificial Sequence
  XLOC_SplitLeft_9
aacccttactattctgagaatggcaagaactc

SEQ ID NO: 293 - DNA, 32 bp, Artificial Sequence
  XLOC_SplitLeft_10
aacccttactatgcagaggctagagatgctca

SEQ ID NO: 294 - DNA, 32 bp, Artificial Sequence
  XLOC_SplitLeft_11
aacccttactatagcaggtccacgcccaggtg

SEQ ID NO: 295 - DNA, 32 bp, Artificial Sequence
  XLOC_SplitLeft_12
aacccttactatccatccaactgagttcattc

SEQ ID NO: 296 - DNA, 32 bp, Artificial Sequence
  XLOC_SplitLeft_13
aacccttactatgtagaaagagtgaattgaga

SEQ ID NO: 297 - DNA, 32 bp, Artificial Sequence
  XLOC_SplitLeft_14
aacccttactattagatgataggaggcattgt

SEQ ID NO: 298 - DNA, 32 bp, Artificial Sequence
  XLOC_SplitLeft_15
aacccttactatcgagcagttgtatgtgaatg

SEQ ID NO: 299 - DNA, 32 bp, Artificial Sequence
  XLOC_SplitLeft_16
aacccttactatacatcctccattgcaccgca

SEQ ID NO: 300 - DNA, 32 bp, Artificial Sequence
  XLOC_SplitLeft_17
aacccttactattcgaacgaatttcctgcact

SEQ ID NO: 301 - DNA, 32 bp, Artificial Sequence
  XLOC_SplitLeft_18
aacccttactattttcaggttgtatgatatag

SEQ ID NO: 302 - DNA, 32 bp, Artificial Sequence
  XLOC_SplitLeft_19
aacccttactattccacctcatggccacagac

SEQ ID NO: 303 - DNA, 32 bp, Artificial Sequence
  XLOC_SplitLeft_20
aacccttactattaacttgattgtgatgatgg

SEQ ID NO: 304 - DNA, 32 bp, Artificial Sequence
  XLOC_SplitLeft_21
aacccttactattttaggagggaggaattacc

SEQ ID NO: 305 - DNA, 32 bp, Artificial Sequence
  XLOC_SplitLeft_22
aacccttactatacaaacaaatctatagtgat

SEQ ID NO: 306 - DNA, 32 bp, Artificial Sequence
  XLOC_SplitLeft_23
aacccttactatccgtggtggtttcggggcag

SEQ ID NO: 307 - DNA, 32 bp, Artificial Sequence
  XLOC_SplitRight_1
gcgcggtggctcacacctgttattatccaccg

SEQ ID NO: 308 - DNA, 32 bp, Artificial Sequence
  XLOC_SplitRight_2
cccagggacattcatttgcttattatccaccg

SEQ ID NO: 309 - DNA, 32 bp, Artificial Sequence
  XLOC_SplitRight_3
atgacttctctatcccactttattatccaccg

SEQ ID NO: 310 - DNA, 32 bp, Artificial Sequence
  XLOC_SplitRight_4
gaagtgcctcgtcattctggtattatccaccg

SEQ ID NO: 311 - DNA, 32 bp, Artificial Sequence
  XLOC_SplitRight_5
ccttgtaaatactgattaaatattatccaccg

SEQ ID NO: 312 - DNA, 32 bp, Artificial Sequence
  XLOC_SplitRight_6
agtaggtgctcaatgaattgtattatccaccg

SEQ ID NO: 313 - DNA, 32 bp, Artificial Sequence
  XLOC_SplitRight_7
gcacacagcaggtgctcaattattatccaccg

SEQ ID NO: 314 - DNA, 32 bp, Artificial Sequence
  XLOC_SplitRight_8
ccttaggaaataatagctagtattatccaccg

SEQ ID NO: 315 - DNA, 32 bp, Artificial Sequence
  XLOC_SplitRight_9
cattaactctcttctccaaatattatccaccg

SEQ ID NO: 316 - DNA, 32 bp, Artificial Sequence
  XLOC_SplitRight_10
ttcctaaagggatcaaataatattatccaccg

SEQ ID NO: 317 - DNA, 32 bp, Artificial Sequence
  XLOC_SplitRight_11
cagattgtcccagatgaacatattatccaccg

SEQ ID NO: 318 - DNA, 32 bp, Artificial Sequence
  XLOC_SplitRight_12
tgatacctactgatctgaagtattatccaccg

SEQ ID NO: 319 - DNA, 32 bp, Artificial Sequence
  XLOC_SplitRight_13
atatgaggaatgagagtgggtattatccaccg

SEQ ID NO: 320 - DNA, 32 bp, Artificial Sequence
  XLOC_SplitRight_14
cattactaggatagtaactctattatccaccg

SEQ ID NO: 321 - DNA, 32 bp, Artificial Sequence
  XLOC_SplitRight_15
cattgtatcaaaccaagtcttattatccaccg

SEQ ID NO: 322 - DNA, 32 bp, Artificial Sequence
  XLOC_SplitRight_16
tatttgctttctgtattccctattatccaccg

SEQ ID NO: 323 - DNA, 32 bp, Artificial Sequence
  XLOC_SplitRight_17
ggtaactaggtgaagaattttattatccaccg

SEQ ID NO: 324 - DNA, 32 bp, Artificial Sequence
  XLOC_SplitRight_18
tgcctgacaactctccattttattatccaccg

SEQ ID NO: 325 - DNA, 32 bp, Artificial Sequence
  XLOC_SplitRight_19
cggttaaccatccatataaatattatccaccg

SEQ ID NO: 326 - DNA, 32 bp, Artificial Sequence
  XLOC_SplitRight_20
cttcacaggtgtacacagcttattatccaccg

SEQ ID NO: 327 - DNA, 32 bp, Artificial Sequence
  XLOC_SplitRight_21
aaagggcatgagaacacttttattatccaccg

SEQ ID NO: 328 - DNA, 32 bp, Artificial Sequence
  XLOC_SplitRight_22
agaaagttggtcagcggttttattatccaccg

SEQ ID NO: 329 - DNA, 32 bp, Artificial Sequence
  XLOC_SplitRight_23
aagcccaggggtactccttatattatccaccg

SEQ ID NO: 330 - DNA, 18 bp, Artificial Sequence
  XLOC_Cy5_Readout
gtaagggttcggtggata

SEQ ID NO: 331 - DNA, 37 bp, Artificial Sequence
  12nt_Split_probe_2
acagggctgggagtagttccagtattatccaccgaac

SEQ ID NO: 332 - DNA, 36 bp, Artificial Sequence
  11nt_Split_probe_2
acagggctgggagtagttccagtattatccaccgaa

SEQ ID NO: 333 - DNA, 35 bp, Artificial Sequence
  10nt_Split_probe_2
acagggctgggagtagttccagtattatccaccga

SEQ ID NO: 334 - DNA, 34 bp, Artificial Sequence
  9nt_Split_probe_2
acagggctgggagtagttccagtattatccaccg

SEQ ID NO: 335 - DNA, 33 bp, Artificial Sequence
  8nt_Split_probe_2
acagggctgggagtagttccagtatatccaccg

SEQ ID NO: 336 - DNA, 32 bp, Artificial Sequence
  7nt_Split_probe_2
acagggctgggagtagttccagtattccaccg

SEQ ID NO: 337 - DNA, 34 bp, Artificial Sequence
  9nt_Split_probe_1
aacccttactatagaggttgtgctggtggtggga

SEQ ID NO: 338 - DNA, 34 bp, Artificial Sequence; identical to SEQ ID NO: 334
  9nt_Split_probe_2

SEQ ID NO: 339 - DNA, 33 bp, Artificial Sequence
  8nt_Split_probe_1
aacccttatatagaggttgtgctggtggtggga

SEQ ID NO: 340 - DNA, 33 bp, Artificial Sequence; identical to SEQ ID NO: 335
  8nt_Split_probe_2

SEQ ID NO: 341 - DNA, 32 bp, Artificial Sequence
  7nt_Split_probe_1
aaccctttatagaggttgtgctggtggtggga

SEQ ID NO: 342 - DNA, 32 bp, Artificial Sequence; identical to SEQ ID NO: 336
  7nt_Split_probe_2

SEQ ID NO: 343 - DNA, 18 bp, Artificial Sequence
  Readout sequence
gtaagggttcggtggata

SEQ ID NO: 344 - DNA, 34 bp, Artificial Sequence; identical to SEQ ID NO: 337
  Circular_probe_1

SEQ ID NO: 345 - DNA, 34 bp, Artificial Sequence; identical to SEQ ID NO: 334
  Circular_probe_2

SEQ ID NO: 346 - DNA, 34 bp, Artificial Sequence
  Cruciform_probe_1
agaggttgtgctggtggtgggatataacccttac

SEQ ID NO: 347 - DNA, 34 bp, Artificial Sequence
  Cruciform_probe_2
tatccaccgtatacagggctgggagtagttccag

SEQ ID NO: 348 - DNA, 34 bp, Artificial Sequence; identical to SEQ ID NO: 346
  Double_′C′_probe_1

SEQ ID NO: 349 - DNA, 34 bp, Artificial Sequence; identical to SEQ ID NO: 334
  Double_′C′_probe_2

SEQ ID NO: 350 - DNA, 34 bp, Artificial Sequence
  Double_′Z′_probe_1
agaggttgtgctggtggtgggatattatccaccg

SEQ ID NO: 351 - DNA, 34 bp, Artificial Sequence
  Double_′Z′_probe_2
acagggctgggagtagttccagtataacccttac

SEQ ID NO: 352 - DNA, 62 bp, Artificial Sequence
  Conventional_probe
tatccaccgaacccttacagaggttgtgctggtggtgggaacagggctgggagtagttccag

SEQ ID NO: 353 - DNA, 34 bp, Artificial Sequence; identical to SEQ ID NO: 337
  Negative control

SEQ ID NO: 354 - DNA, 18 bp, Artificial Sequence
  Cy5_bridge
gtaagggttcggtggata

SEQ ID NO: 355 - DNA, 41 bp, Artificial Sequence
  MUC5AC_Cy3_1
tgtccctcagcagcctctgatatatttaaccgcactatccc

SEQ ID NO: 356 - DNA, 41 bp, Artificial Sequence
  MUC5AC_Cy3_2
actcattgtgtggacggcggtatatttaaccgcactatccc

SEQ ID NO: 357 - DNA, 41 bp, Artificial Sequence
  MUC5AC_Cy3_3
tcctgggcatggcctgtatgtatatttaaccgcactatccc

SEQ ID NO: 358 - DNA, 41 bp, Artificial Sequence
  MUC5AC_Cy3_4
gtagctggattcggaggagctatatttaaccgcactatccc

SEQ ID NO: 359 - DNA, 41 bp, Artificial Sequence
  MUC5AC_Cy3_5
ataggagagagggcagggtgtatatttaaccgcactatccc

SEQ ID NO: 360 - DNA, 41 bp, Artificial Sequence
  MUC5AC_Cy3_6
agatgggaagacagtcgccctatatttaaccgcactatccc

SEQ ID NO: 361 - DNA, 41 bp, Artificial Sequence
  MUC5AC_Cy3_7
ttggaggctcgtaccacaggtatatttaaccgcactatccc

SEQ ID NO: 362 - DNA, 41 bp, Artificial Sequence
  MUC5AC_Cy3_8
ggtcttgtagtggaagctgctatatttaaccgcactatccc

SEQ ID NO: 363 - DNA, 41 bp, Artificial Sequence
  MUC5AC_Cy3_9
gaacacgtagttgcagaggctatatttaaccgcactatccc

SEQ ID NO: 364 - DNA, 41 bp, Artificial Sequence
  MUC5AC_Cy3_10
aaatcctcgtaggcggcacctatatttaaccgcactatccc

SEQ ID NO: 365 - DNA, 41 bp, Artificial Sequence
  MUC5AC_Cy3_11
atgaggaccctgctcagcgttatatttaaccgcactatccc

SEQ ID NO: 366 - DNA, 41 bp, Artificial Sequence
  MUC5AC_Cy3_12
gatgaccacgccatccaccttatatttaaccgcactatccc

SEQ ID NO: 367 - DNA, 41 bp, Artificial Sequence
  MUC5AC_Cy3_13
ttgaccaggacggagccctttatatttaaccgcactatccc

SEQ ID NO: 368 - DNA, 41 bp, Artificial Sequence
  MUC5AC_Cy3_14
agactggctgaagggcagcatatatttaaccgcactatccc

SEQ ID NO: 369 - DNA, 41 bp, Artificial Sequence
  MUC5AC_Cy3_15
tgctctgctgaatgaggacctatatttaaccgcactatccc

SEQ ID NO: 370 - DNA, 41 bp, Artificial Sequence
  MUC5AC_Cy3_16
gcctccaccttggtgtagcttatatttaaccgcactatccc

SEQ ID NO: 371 - DNA, 41 bp, Artificial Sequence
  MUC5AC_Cy3_17
acatgaggacaaggcccagctatatttaaccgcactatccc

SEQ ID NO: 372 - DNA, 41 bp, Artificial Sequence
  MUC5AC_Cy3_18
agcaggctgtcatcgtggtttatatttaaccgcactatccc

SEQ ID NO: 373 - DNA, 41 bp, Artificial Sequence
  MUC5AC_Cy3_19
gtatttggtgtccagctccatatatttaaccgcactatccc

SEQ ID NO: 374 - DNA, 41 bp, Artificial Sequence
  MUC5AC_Cy3_20
acgggcatcccgttgaagtctatatttaaccgcactatccc

SEQ ID NO: 375 - DNA, 41 bp, Artificial Sequence
  MUC5AC_Cy3_21
gcttggtgttgtgggagaggtatatttaaccgcactatccc

SEQ ID NO: 376 - DNA, 41 bp, Artificial Sequence
  MUC5AC_Cy3_22
ttcccgaattccatgggtgttatatttaaccgcactatccc

SEQ ID NO: 377 - DNA, 41 bp, Artificial Sequence
  MUC5AC_Cy3_23
acagggtcctgacactggtctatatttaaccgcactatccc

SEQ ID NO: 378 - DNA, 41 bp, Artificial Sequence
  MUC5AC_Cy3_24
agccagtggagcagttcctctatatttaaccgcactatccc

SEQ ID NO: 379 - DNA, 41 bp, Artificial Sequence
  MUC5AC_Cy3_25
aggagctcctcacagatgcctatatttaaccgcactatccc

SEQ ID NO: 380 - DNA, 41 bp, Artificial Sequence
  MUC5AC_Cy3_26
acgcagccagagaacagctgtatatttaaccgcactatccc

SEQ ID NO: 381 - DNA, 41 bp, Artificial Sequence
  MUC5AC_Cy3_27
tgcaagcctccaggtagctgtatatttaaccgcactatccc

SEQ ID NO: 382 - DNA, 41 bp, Artificial Sequence
  MUC5AC_Cy3_28
tcacagaagcagaggtcttgtatatttaaccgcactatccc

SEQ ID NO: 383 - DNA, 41 bp, Artificial Sequence
  MUC5AC_Cy3_29
tactcggcaagggtgtggcatatatttaaccgcactatccc

SEQ ID NO: 384 - DNA, 41 bp, Artificial Sequence
  MUC5AC_Cy3_30
tggtactgcatgttgttgggtatatttaaccgcactatccc

SEQ ID NO: 385 - DNA, 41 bp, Artificial Sequence
  MUC5AC_Cy3_31
ttggagcaggtgtctgcgcatatatttaaccgcactatccc

SEQ ID NO: 386 - DNA, 41 bp, Artificial Sequence
  MUC5AC_Cy3_32
acacagtggtcctcacaggctatatttaaccgcactatccc

SEQ ID NO: 387 - DNA, 41 bp, Artificial Sequence
  MUC5AC_Cy3_33
atgtcgtcaagcaccgtccctatatttaaccgcactatccc

SEQ ID NO: 388 - DNA, 41 bp, Artificial Sequence
  MUC5AC_Cy3_34
ttgacacagggacacagccgtatatttaaccgcactatccc

SEQ ID NO: 389 - DNA, 41 bp, Artificial Sequence
  MUC5AC_Cy3_35
ccgttgtagacgcaggcacatatatttaaccgcactatccc

SEQ ID NO: 390 - DNA, 41 bp, Artificial Sequence
  MUC5AC_Cy3_36
agtctgtggagtaggtggcctatatttaaccgcactatccc

SEQ ID NO: 391 - DNA, 41 bp, Artificial Sequence
  MUC5AC_Cy3_37
atggaacctcctggcagctctatatttaaccgcactatccc

SEQ ID NO: 392 - DNA, 41 bp, Artificial Sequence
  MUC5AC_Cy3_38
aagcacagagcaggtacccgtatatttaaccgcactatccc

SEQ ID NO: 393 - DNA, 41 bp, Artificial Sequence
  MUC5AC_Cy3_39
acgttgagaagtgggcaccttatatttaaccgcactatccc

SEQ ID NO: 394 - DNA, 41 bp, Artificial Sequence
  MUC5AC_Cy3_40
accgtgtattgcttcccgtctatatttaaccgcactatccc

SEQ ID NO: 395 - DNA, 41 bp, Artificial Sequence
  MUC5AC_Cy3_41
cacatagctgcagtcgccgttatatttaaccgcactatccc

SEQ ID NO: 396 - DNA, 41 bp, Artificial Sequence
  MUC5AC_Cy3_42
tgctgtcacagggcttggtctatatttaaccgcactatccc

SEQ ID NO: 397 - DNA, 41 bp, Artificial Sequence
  MUC5AC_Cy3_43
tcagccagtacagtgaaggctatatttaaccgcactatccc

SEQ ID NO: 398 - DNA, 41 bp, Artificial Sequence
  MUC5AC_Cy3_44
ttcaggcaggtctcgctgtctatatttaaccgcactatccc

SEQ ID NO: 399 - DNA, 41 bp, Artificial Sequence
  MUC5AC_Cy3_45
atccaggctcagtgtcacgctatatttaaccgcactatccc

SEQ ID NO: 400 - DNA, 41 bp, Artificial Sequence
  MUC5AC_Cy3_46
tgatcaccaccaccgtctgctatatttaaccgcactatccc

SEQ ID NO: 401 - DNA, 41 bp, Artificial Sequence
  MUC5AC_Cy3_47
ctggttcaggaacacttccctatatttaaccgcactatccc

SEQ ID NO: 402 - DNA, 41 bp, Artificial Sequence
  MUC5AC_Cy3_48
agatgggcagctgggtgtagtatatttaaccgcactatccc

SEQ ID NO: 403 - DNA, 41 bp, Artificial Sequence
  MUC5AC_Cy3_49
aagatggtgacgttggctgctatatttaaccgcactatccc

SEQ ID NO: 404 - DNA, 41 bp, Artificial Sequence
  MUC5AC_Cy3_50
gatgaagaaggttgagggtctatatttaaccgcactatccc

SEQ ID NO: 405 - DNA, 41 bp, Artificial Sequence
  MUC5AC_Cy3_51
agctgcaggttcagctgcagtatatttaaccgcactatccc

SEQ ID NO: 406 - DNA, 41 bp, Artificial Sequence
  MUC5AC_Cy3_52
gaacagctgcatggtgggcatatatttaaccgcactatccc

SEQ ID NO: 407 - DNA, 41 bp, Artificial Sequence
  MUC5AC_Cy3_53
ttcccacagagaccgcaggttatatttaaccgcactatccc

SEQ ID NO: 408 - DNA, 41 bp, Artificial Sequence
  MUC5AC_Cy3_54
atcggcctggatgctgttgatatatttaaccgcactatccc

SEQ ID NO: 409 - DNA, 41 bp, Artificial Sequence
  MUC5AC_Cy3_55
ttgaagaaggccgcagcggttatatttaaccgcactatccc

SEQ ID NO: 410 - DNA, 41 bp, Artificial Sequence
  MUC5AC_Cy3_56
aagctgttcctgatgttgggtatatttaaccgcactatccc

SEQ ID NO: 411 - DNA, 41 bp, Artificial Sequence
  MUC5AC_Cy3_57
tctccacgctcagagagcagtatatttaaccgcactatccc

SEQ ID NO: 412 - DNA, 41 bp, Artificial Sequence
  MUC5AC_Cy3_58
cagtgctgagcatacttctctatatttaaccgcactatccc

SEQ ID NO: 413 - DNA, 41 bp, Artificial Sequence
  MUC5AC_Cy3_59
agtaggttcccggcttcacgtatatttaaccgcactatccc

SEQ ID NO: 414 - DNA, 41 bp, Artificial Sequence
  MUC5AC_Cy3_60
gtgtcaaacatgcagttcgatatatttaaccgcactatccc

SEQ ID NO: 415 - DNA, 41 bp, Artificial Sequence
  MUC5AC_Cy3_61
tcctcgctccgctcacagtttatatttaaccgcactatccc

SEQ ID NO: 416 - DNA, 41 bp, Artificial Sequence
  MUC5AC_Cy3_62
ataggcttcgtgcagacgcctatatttaaccgcactatccc

SEQ ID NO: 417 - DNA, 41 bp, Artificial Sequence
  MUC5AC_Cy3_63
tggtacgtcattgacttgggtatatttaaccgcactatccc

SEQ ID NO: 418 - DNA, 41 bp, Artificial Sequence
  MUC5AC_Cy3_64
ctggcaggtgctgacatggttatatttaaccgcactatccc

SEQ ID NO: 419 - DNA, 41 bp, Artificial Sequence
  MUC5AC_Cy3_65
aacactgcaggtgatgtccctatatttaaccgcactatccc

SEQ ID NO: 420 - DNA, 41 bp, Artificial Sequence
  MUC5AC_Cy3_66
acagatgcagccatccacggtatatttaaccgcactatccc

SEQ ID NO: 421 - DNA, 41 bp, Artificial Sequence
  MUC5AC_Cy3_67
tcgtccaggaaggtgccctttatatttaaccgcactatccc

SEQ ID NO: 422 - DNA, 41 bp, Artificial Sequence
  MUC5AC_Cy3_68
tgctggcctgcacacacttgtatatttaaccgcactatccc

SEQ ID NO: 423 - DNA, 41 bp, Artificial Sequence
  MUC5AC_Cy3_69
cctctgtggtagcagggacatatatttaaccgcactatccc

SEQ ID NO: 424 - DNA, 41 bp, Artificial Sequence
  MUC5AC_Cy3_70
tgtgcaggtgcagatagccctatatttaaccgcactatccc

SEQ ID NO: 425 - DNA, 41 bp, Artificial Sequence
  MUC5AC_Cy3_71
cgatgcagctcagcttcccatatatttaaccgcactatccc

SEQ ID NO: 426 - DNA, 41 bp, Artificial Sequence
  MUC5AC_Cy3_72
aacaccatgggcgcagcacatatatttaaccgcactatccc

SEQ ID NO: 427 - DNA, 41 bp, Artificial Sequence
  MUC5AC_Cy3_73
cgtggcatttcggcagtcaatatatttaaccgcactatccc

SEQ ID NO: 428 - DNA, 41 bp, Artificial Sequence
  MUC5AC_Cy3_74
agctcttctgacagccagcctatatttaaccgcactatccc

SEQ ID NO: 429 - DNA, 41 bp, Artificial Sequence
  MUC5AC_Cy3_75
caggtcatgtccagtgtgtgtatatttaaccgcactatccc

SEQ ID NO: 430 - DNA, 18 bp, Artificial Sequence
  Readout sequence
gggatagtgcggttaaat

SEQ ID NO: 431 - DNA, 34 bp, Artificial Sequence; identical to SEQ ID NO: 337
  MUC5AC_Left_1

SEQ ID NO: 432 - DNA, 34 bp, Artificial Sequence; identical to SEQ ID NO: 334
  MUC5AC_Right_1

SEQ ID NO: 433 - DNA, 40 bp, Artificial Sequence
  MUC5AC_Conventional
acagggctgggagtagttccagtatccaccgaacccttac

SEQ ID NO: 434 - DNA, 54 bp, Artificial Sequence
  Bridge_1
tgatgggtgcgtgagtaagtaagggttcggtggatatgatgggtgcgtgagtaa

SEQ ID NO: 435 - DNA, 18 bp, Artificial Sequence
  Bridge Readout
ttactcacgcacccatca

SEQ ID NO: 436 - DNA, 18 bp, Artificial Sequence
  Conventional Readout
gtaagggttcggtggata

SEQ ID NO: 437 - DNA, 20 bp, Artificial Sequence
  Primer
taccatctcgtgttcgtacc

SEQ ID NO: 438 - DNA, 38 bp, Artificial Sequence
  Primer
taatacgactcactatagttcgttccgctactcaccac

SEQ ID NO: 439 - DNA, 88 bp, Artificial Sequence
  FLNA_1
taccatctcgtgttcgtaccggtaccccattacctatcagggagcagaggttgcgctgctgtgaattcgtggtgagtagcggaacgaa

SEQ ID NO: 440 - DNA, 88 bp, Artificial Sequence
  FLNA_2
taccatctcgtgttcgtaccggtaccccattacctatggagccgcgcctgctgcgctctggcgaattcgtggtgagtagcggaacgaa

SEQ ID NO: 441 - DNA, 88 bp, Artificial Sequence
  FLNA_3
taccatctcgtgttcgtaccggtaccccattacctataggtccttctcggtggccggcatctgaattcgtggtgagtagcggaacgaa

SEQ ID NO: 442 - DNA, 88 bp, Artificial Sequence
  FLNA_4
taccatctcgtgttcgtaccggtaccccattacctatcaccagcgcgtgaaagtgttctgctgaattcgtggtgagtagcggaacgaa

SEQ ID NO: 443 - DNA, 88 bp, Artificial Sequence
  FLNA_5
taccatctcgtgttcgtaccggtaccccattacctataggtccgtctgcaggttggcgatgcgaattcgtggtgagtagcggaacgaa

SEQ ID NO: 444 - DNA, 88 bp, Artificial Sequence
  FLNA_6
taccatctcgtgttcgtaccggtaccccattacctatatcttcttctggctgagcacctccagaattcgtggtgagtagcggaacgaa

SEQ ID NO: 445 - DNA, 88 bp, Artificial Sequence
  FLNA_7
taccatctcgtgttcgtaccggtaccccattacctatacgttctcaagctgcatttggcggagaattcgtggtgagtagcggaacgaa

SEQ ID NO: 446 - DNA, 88 bp, Artificial Sequence
  FLNA_8
taccatctcgtgttcgtaccggtaccccattacctattcgatggacaccagtttgatgctctgaattcgtggtgagtagcggaacgaa

SEQ ID NO: 447 - DNA, 88 bp, Artificial Sequence
  FLNA_9
taccatctcgtgttcgtaccggtaccccattacctatgagtagtgcaggatcagggtccagagaattcgtggtgagtagcggaacgaa

SEQ ID NO: 448 - DNA, 88 bp, Artificial Sequence
  FLNA_10
taccatctcgtgttcgtaccggtaccccattacctatgtctgcttcttggcctcctcatcctgaattcgtggtgagtagcggaacgaa

SEQ ID NO: 449 - DNA, 88 bp, Artificial Sequence
  FLNA_11
taccatctcgtgttcgtaccggtaccccattacctatatgggcagctgcggcagcttgttctgaattcgtggtgagtagcggaacgaa

SEQ ID NO: 450 - DNA, 88 bp, Artificial Sequence
  FLNA_12
taccatctcgtgttcgtaccggtaccccattacctattccaccagggcgcccagggcccggcgaattcgtggtgagtagcggaacgaa

SEQ ID NO: 451 - DNA, 88 bp, Artificial Sequence
  FLNA_13
taccatctcgtgttcgtaccggtaccccattacctatggtaacgggcttgctggcgtcccaagaattcgtggtgagtagcggaacgaa

SEQ ID NO: 452 - DNA, 88 bp, Artificial Sequence
  FLNA_14
taccatctcgtgttcgtaccggtaccccattacctatggcccgtaggcacgggctttcttcggaattcgtggtgagtagcggaacgaa

SEQ ID NO: 453 - DNA, 88 bp, Artificial Sequence
  FLNA_15
taccatctcgtgttcgtaccggtaccccattacctatggacgtaccagacggagaaggtgcggaattcgtggtgagtagcggaacgaa

SEQ ID NO: 454 - DNA, 88 bp, Artificial Sequence
  FLNA_16
taccatctcgtgttcgtaccggtaccccattacctatggctcttggcgatgtgctggccagcgaattcgtggtgagtagcggaacgaa

SEQ ID NO: 455 - DNA, 88 bp, Artificial Sequence
  FLNA_17
taccatctcgtgttcgtaccggtaccccattacctatgccgtaaagatctcaaagtaggtgggaattcgtggtgagtagcggaacgaa

SEQ ID NO: 456 - DNA, 88 bp, Artificial Sequence
  FLNA_18
taccatctcgtgttcgtaccggtaccccattacctatagggatgggcacgccggcaaacgtggaattcgtggtgagtagcggaacgaa

SEQ ID NO: 457 - DNA, 88 bp, Artificial Sequence
  FLNA_19
taccatctcgtgttcgtaccggtaccccattacctatccgcccggcaggcactcgggttacagaattcgtggtgagtagcggaacgaa

SEQ ID NO: 458 - DNA, 88 bp, Artificial Sequence
  FLNA_20
taccatctcgtgttcgtaccggtaccccattacctatcaccttgaagtcagctgtctccttcgaattcgtggtgagtagcggaacgaa

SEQ ID NO: 459 - DNA, 88 bp, Artificial Sequence
  FLNA_21
taccatctcgtgttcgtaccggtaccccattacctatctcggtgcccaccttcacttcgaaggaattcgtggtgagtagcggaacgaa

SEQ ID NO: 460 - DNA, 88 bp, Artificial Sequence
  FLNA_22
taccatctcgtgttcgtaccggtaccccattacctatcaccacaaagtctgctgacttgccagaattcgtggtgagtagcggaacgaa

SEQ ID NO: 461 - DNA, 88 bp, Artificial Sequence
  FLNA_23
taccatctcgtgttcgtaccggtaccccattacctatttagcctgcgatggcccttccaccggaattcgtggtgagtagcggaacgaa

SEQ ID NO: 462 - DNA, 88 bp, Artificial Sequence
  FLNA_24
taccatctcgtgttcgtaccggtaccccattacctattgcggccagtagcgcacatcacagggaattcgtggtgagtagcggaacgaa

SEQ ID NO: 463 - DNA, 88 bp, Artificial Sequence
  FLNA_25
taccatctcgtgttcgtaccggtaccccattacctataggcggatgtcttcgctgttgcacagaattcgtggtgagtagcggaacgaa

SEQ ID NO: 464 - DNA, 88 bp, Artificial Sequence
  FLNA_26
taccatctcgtgttcgtaccggtaccccattacctatttctccaatccaggcccacgtgcctgaattcgtggtgagtagcggaacgaa

SEQ ID NO: 465 - DNA, 88 bp, Artificial Sequence
  FLNA_27
taccatctcgtgttcgtaccggtaccccattacctatccgtgcttggcatccactgtgaactgaattcgtggtgagtagcggaacgaa

SEQ ID NO: 466 - DNA, 88 bp, Artificial Sequence
  FLNA_28
taccatctcgtgttcgtaccggtaccccattacctatcaacgcctccacagggcagccttcagaattcgtggtgagtagcggaacgaa

SEQ ID NO: 467 - DNA, 88 bp, Artificial Sequence
  FLNA_29
taccatctcgtgttcgtaccggtaccccattacctatcggcttcctgggcacgtaggagcaggaattcgtggtgagtagcggaacgaa

SEQ ID NO: 468 - DNA, 88 bp, Artificial Sequence
  FLNA_30
taccatctcgtgttcgtaccggtaccccattacctatgccagcctcggcgcagtccacagtggaattcgtggtgagtagcggaacgaa

SEQ ID NO: 469 - DNA, 88 bp, Artificial Sequence
  FLNA_31
taccatctcgtgttcgtaccggtaccccattacctataggtgtcattgtcattgcggatgatgaattcgtggtgagtagcggaacgaa

SEQ ID NO: 470 - DNA, 88 bp, Artificial Sequence
  FLNA_32
taccatctcgtgttcgtaccggtaccccattacctatgtgggcgtggcctggtcagcaaagagaattcgtggtgagtagcggaacgaa

SEQ ID NO: 471 - DNA, 88 bp, Artificial Sequence
  FLNA_33
taccatctcgtgttcgtaccggtaccccattacctatcggccttcaccttactggcgtcatggaattcgtggtgagtagcggaacgaa

SEQ ID NO: 472 - DNA, 88 bp, Artificial Sequence
  FLNA_34
taccatctcgtgttcgtaccggtaccccattacctattgaagtgggtgggcttgccaagctcgaattcgtggtgagtagcggaacgaa

SEQ ID NO: 473 - DNA, 88 bp, Artificial Sequence
  FLNA_35
taccatctcgtgttcgtaccggtaccccattacctatgtgtaggtgttgtcatggtggtcgagaattcgtggtgagtagcggaacgaa

SEQ ID NO: 474 - DNA, 88 bp, Artificial Sequence
  FLNA_36
taccatctcgtgttcgtaccggtaccccattacctatccataagtgacattgacgcctactggaattcgtggtgagtagcggaacgaa

SEQ ID NO: 475 - DNA, 88 bp, Artificial Sequence
  FLNA_37
taccatctcgtgttcgtaccggtaccccattacctattccaggcttggagatactgccactggaattcgtggtgagtagcggaacgaa

SEQ ID NO: 476 - DNA, 88 bp, Artificial Sequence
  FLNA_38
taccatctcgtgttcgtaccggtaccccattacctattctttgccaacgtccaccttctctcgaattcgtggtgagtagcggaacgaa

SEQ ID NO: 477 - DNA, 88 bp, Artificial Sequence
  FLNA_39
taccatctcgtgttcgtaccggtaccccattacctatgatgccactttgccttgaccaccaggaattcgtggtgagtagcggaacgaa

SEQ ID NO: 478 - DNA, 88 bp, Artificial Sequence
  FLNA_40
taccatctcgtgttcgtaccggtaccccattacctatacgggcacgccgtcataggtcacctgaattcgtggtgagtagcggaacgaa

SEQ ID NO: 479 - DNA, 88 bp, Artificial Sequence
  FLNA_41
taccatctcgtgttcgtaccggtaccccattacctatggcccaggccacctgtgccggcgccgaattcgtggtgagtagcggaacgaa

SEQ ID NO: 480 - DNA, 88 bp, Artificial Sequence
  FLNA_42
taccatctcgtgttcgtaccggtaccccattacctatgcacttgactttggatgcgtcaaaggaattcgtggtgagtagcggaacgaa

SEQ ID NO: 481 - DNA, 88 bp, Artificial Sequence
  FLNA_43
taccatctcgtgttcgtaccggtaccccattacctatagctccgcgctgcccgcgctcgagcgaattcgtggtgagtagcggaacgaa

SEQ ID NO: 482 - DNA, 88 bp, Artificial Sequence
  FLNA_44
taccatctcgtgttcgtaccggtaccccattacctattggtgtgcgtgccatcaccgtggtcgaattcgtggtgagtagcggaacgaa

SEQ ID NO: 483 - DNA, 88 bp, Artificial Sequence
  FLNA_45
taccatctcgtgttcgtaccggtaccccattacctatgggaagttgggcacgggctggccgcgaattcgtggtgagtagcggaacgaa

SEQ ID NO: 484 - DNA, 88 bp, Artificial Sequence
  FLNA_46
taccatctcgtgttcgtaccggtaccccattacctatccatagcactggacaccggaagtgtgaattcgtggtgagtagcggaacgaa

SEQ ID NO: 485 - DNA, 88 bp, Artificial Sequence
  FLNA_47
taccatctcgtgttcgtaccggtaccccattacctatacactgaactcagtggtggcctcacgaattcgtggtgagtagcggaacgaa

SEQ ID NO: 486 - DNA, 88 bp, Artificial Sequence
  FLNA_48
taccatctcgtgttcgtaccggtaccccattacctatgccacacgggccttgacgtgcggccgaattcgtggtgagtagcggaacgaa

SEQ ID NO: 487 - DNA, 88 bp, Artificial Sequence
  FLNA_49
taccatctcgtgttcgtaccggtaccccattacctatacatgccatcgccacggtcctgaacgaattcgtggtgagtagcggaacgaa

SEQ ID NO: 488 - DNA, 88 bp, Artificial Sequence
  FLNA_50
taccatctcgtgttcgtaccggtaccccattacctataggtcacgtccacggagtgcagtccgaattcgtggtgagtagcggaacgaa

SEQ ID NO: 489 - DNA, 88 bp, Artificial Sequence
  FLNA_51
taccatctcgtgttcgtaccggtaccccattacctatccgagcagctgccgtccttgttatcgaattcgtggtgagtagcggaacgaa

SEQ ID NO: 490 - DNA, 88 bp, Artificial Sequence
  FLNA_52
taccatctcgtgttcgtaccggtaccccattacctatcaccataggtgacgttgaggctgtagaattcgtggtgagtagcggaacgaa

SEQ ID NO: 491 - DNA, 88 bp, Artificial Sequence
  FLNA_53
taccatctcgtgttcgtaccggtaccccattacctatcatctgtcacatcatgcacagggacgaattcgtggtgagtagcggaacgaa

SEQ ID NO: 492 - DNA, 88 bp, Artificial Sequence
  FLNA_54
taccatctcgtgttcgtaccggtaccccattacctattggcacgaaccatgcctgggctcaggaattcgtggtgagtagcggaacgaa

SEQ ID NO: 493 - DNA, 88 bp, Artificial Sequence
  FLNA_55
taccatctcgtgttcgtaccggtaccccattacctatgtccactggctccaccaggcctttggaattcgtggtgagtagcggaacgaa

SEQ ID NO: 494 - DNA, 88 bp, Artificial Sequence
  FLNA_56
taccatctcgtgttcgtaccggtaccccattacctatttctcggctgggcacataattgacggaattcgtggtgagtagcggaacgaa

SEQ ID NO: 495 - DNA, 88 bp, Artificial Sequence
  FLNA_57
taccatctcgtgttcgtaccggtaccccattacctattggccttcaccttgctggcatcatggaattcgtggtgagtagcggaacgaa

SEQ ID NO: 496 - DNA, 88 bp, Artificial Sequence
  FLNA_58
taccatctcgtgttcgtaccggtaccccattacctattggtgaactccacgggcaggctggcgaattcgtggtgagtagcggaacgaa

SEQ ID NO: 497 - DNA, 88 bp, Artificial Sequence
  FLNA_59
taccatctcgtgttcgtaccggtaccccattacctatttcttcggcttgccttcgggatccggaattcgtggtgagtagcggaacgaa

SEQ ID NO: 498 - DNA, 88 bp, Artificial Sequence
  FLNA_60
taccatctcgtgttcgtaccggtaccccattacctattctggcacgtaggccactgtatacggaattcgtggtgagtagcggaacgaa

SEQ ID NO: 499 - DNA, 88 bp, Artificial Sequence
  FLNA_61
taccatctcgtgttcgtaccggtaccccattacctatactttgcctttgcctgccgccttaggaattcgtggtgagtagcggaacgaa

SEQ ID NO: 500 - DNA, 88 bp, Artificial Sequence
  FLNA_62
taccatctcgtgttcgtaccggtaccccattacctattccaccacgtccacatccacctctggaattcgtggtgagtagcggaacgaa

SEQ ID NO: 501 - DNA, 88 bp, Artificial Sequence
  FLNA_63
taccatctcgtgttcgtaccggtaccccattacctatggcacgtgctcgccaccaaagcgcagaattcgtggtgagtagcggaacgaa

SEQ ID NO: 502 - DNA, 88 bp, Artificial Sequence
  FLNA_64
taccatctcgtgttcgtaccggtaccccattacctatttgtcagtgatggtgggctgcgccagaattcgtggtgagtagcggaacgaa

SEQ ID NO: 503 - DNA, 88 bp, Artificial Sequence
  FLNA_65
taccatctcgtgttcgtaccggtaccccattacctattcgtgcaggccagcctcgctgggtggaattcgtggtgagtagcggaacgaa

SEQ ID NO: 504 - DNA, 88 bp, Artificial Sequence
  FLNA_66
taccatctcgtgttcgtaccggtaccccattacctatcataggcagtgacatggccacagttgaattcgtggtgagtagcggaacgaa

SEQ ID NO: 505 - DNA, 88 bp, Artificial Sequence
  FLNA_67
taccatctcgtgttcgtaccggtaccccattacctatctgcatccttggtgttgacggtgaagaattcgtggtgagtagcggaacgaa

SEQ ID NO: 506 - DNA, 88 bp, Artificial Sequence
  FLNA_68
taccatctcgtgttcgtaccggtaccccattacctatagtgcagctgatttctgctttggacgaattcgtggtgagtagcggaacgaa

SEQ ID NO: 507 - DNA, 88 bp, Artificial Sequence
  FLNA_69
taccatctcgtgttcgtaccggtaccccattacctatgggctgcctgggacgtgctgttcatgaattcgtggtgagtagcggaacgaa

SEQ ID NO: 508 - DNA, 88 bp, Artificial Sequence
  FLNA_70
taccatctcgtgttcgtaccggtaccccattacctatggaccacagtggccgtcagcaggctgaattcgtggtgagtagcggaacgaa

SEQ ID NO: 509 - DNA, 88 bp, Artificial Sequence
  FLNA_71
taccatctcgtgttcgtaccggtaccccattacctatcgtggccattacgcagccgcttcaggaattcgtggtgagtagcggaacgaa

SEQ ID NO: 510 - DNA, 88 bp, Artificial Sequence
  FLNA_72
taccatctcgtgttcgtaccggtaccccattacctatgtgtggccttcgtgaaggccctgacgaattcgtggtgagtagcggaacgaa

SEQ ID NO: 511 - DNA, 88 bp, Artificial Sequence
  FLNA_73
taccatctcgtgttcgtaccggtaccccattacctatagcccaccatagcctgcatcgcggggaattcgtggtgagtagcggaacgaa

SEQ ID NO: 512 - DNA, 88 bp, Artificial Sequence
  FLNA_74
taccatctcgtgttcgtaccggtaccccattacctatcgtgctggtcggcaaacttgatgttgaattcgtggtgagtagcggaacgaa

SEQ ID NO: 513 - DNA, 88 bp, Artificial Sequence
  FLNA_75
taccatctcgtgttcgtaccggtaccccattacctatgggtgatgctctctttcacccggccgaattcgtggtgagtagcggaacgaa

SEQ ID NO: 514 - DNA, 88 bp, Artificial Sequence
  FLNA_76
taccatctcgtgttcgtaccggtaccctgagagcgaccggtgaccgatgactatatttaaccgaattcgtggtgagtagcggaacgaa

SEQ ID NO: 515 - DNA, 88 bp, Artificial Sequence
  FLNA_77
taccatctcgtgttcgtaccggtaccgcccgagagtgggagctactcattttatatttaaccgaattcgtggtgagtagcggaacgaa

SEQ ID NO: 516 - DNA, 88 bp, Artificial Sequence
  FLNA_78
taccatctcgtgttcgtaccggtaccgcgtcccgcgtgtcgacgccgccgctatatttaaccgaattcgtggtgagtagcggaacgaa

SEQ ID NO: 517 - DNA, 88 bp, Artificial Sequence
  FLNA_79
taccatctcgtgttcgtaccggtaccatcttcttccacggcgcgtcctccgtatatttaaccgaattcgtggtgagtagcggaacgaa

SEQ ID NO: 518 - DNA, 88 bp, Artificial Sequence
  FLNA_80
taccatctcgtgttcgtaccggtaccttgctcacgcacttcaggtgctcgttatatttaaccgaattcgtggtgagtagcggaacgaa

SEQ ID NO: 519 - DNA, 88 bp, Artificial Sequence
  FLNA_81
taccatctcgtgttcgtaccggtaccagcgcgataagccgcagcccgtcgctatatttaaccgaattcgtggtgagtagcggaacgaa

SEQ ID NO: 520 - DNA, 88 bp, Artificial Sequence
  FLNA_82
taccatctcgtgttcgtaccggtaccgtgggccgctggttgtgcttgcggttatatttaaccgaattcgtggtgagtagcggaacgaa

SEQ ID NO: 521 - DNA, 88 bp, Artificial Sequence
  FLNA_83
taccatctcgtgttcgtaccggtacccggtccaggaactcgagcgccaccgtatatttaaccgaattcgtggtgagtagcggaacgaa

SEQ ID NO: 522 - DNA, 88 bp, Artificial Sequence
  FLNA_84
taccatctcgtgttcgtaccggtaccaggcccaggatcagcttcaggttcctatatttaaccgaattcgtggtgagtagcggaacgaa

SEQ ID NO: 523 - DNA, 88 bp, Artificial Sequence
  FLNA_85
taccatctcgtgttcgtaccggtacctcctcgtcccacatgggcatggagatatatttaaccgaattcgtggtgagtagcggaacgaa

SEQ ID NO: 524 - DNA, 88 bp, Artificial Sequence
  FLNA_86
taccatctcgtgttcgtaccggtaccatccagcccaggagcctctgcttggtatatttaaccgaattcgtggtgagtagcggaacgaa

SEQ ID NO: 525 - DNA, 88 bp, Artificial Sequence
  FLNA_87
taccatctcgtgttcgtaccggtaccctctgccagtcccggctgaagttggtatatttaaccgaattcgtggtgagtagcggaacgaa

SEQ ID NO: 526 - DNA, 88 bp, Artificial Sequence
  FLNA_88
taccatctcgtgttcgtaccggtaccgtcccagtcaggacacaggcccgggtatatttaaccgaattcgtggtgagtagcggaacgaa

SEQ ID NO: 527 - DNA, 88 bp, Artificial Sequence
  FLNA_89
taccatctcgtgttcgtaccggtaccttcagtttgggccgcaagggagccctatatttaaccgaattcgtggtgagtagcggaacgaa

SEQ ID NO: 528 - DNA, 88 bp, Artificial Sequence
  FLNA_90
taccatctcgtgttcgtaccggtacctcttgtcgttattggcggtcacttttatatttaaccgaattcgtggtgagtagcggaacgaa

SEQ ID NO: 529 - DNA, 88 bp, Artificial Sequence
  FLNA_91
taccatctcgtgttcgtaccggtaccagagcacagtaaccttatgagtccctatatttaaccgaattcgtggtgagtagcggaacgaa

SEQ ID NO: 530 - DNA, 88 bp, Artificial Sequence
  FLNA_92
taccatctcgtgttcgtaccggtaccttgttggcgatgttgccactgggcttatatttaaccgaattcgtggtgagtagcggaacgaa

SEQ ID NO: 531 - DNA, 88 bp, Artificial Sequence
  FLNA_93
taccatctcgtgttcgtaccggtaccgtgcacggtgtggacgccctccatgtatatttaaccgaattcgtggtgagtagcggaacgaa

SEQ ID NO: 532 - DNA, 88 bp, Artificial Sequence
  FLNA_94
taccatctcgtgttcgtaccggtacccttggccaacagtgacagtgtagggtatatttaaccgaattcgtggtgagtagcggaacgaa

SEQ ID NO: 533 - DNA, 88 bp, Artificial Sequence
  FLNA_95
taccatctcgtgttcgtaccggtaccccgcacacccttgggctggaggccctatatttaaccgaattcgtggtgagtagcggaacgaa

SEQ ID NO: 534 - DNA, 88 bp, Artificial Sequence
  FLNA_96
taccatctcgtgttcgtaccggtaccactgcgcccgatgttctgaccaccctatatttaaccgaattcgtggtgagtagcggaacgaa

SEQ ID NO: 535 - DNA, 88 bp, Artificial Sequence
  FLNA_97
taccatctcgtgttcgtaccggtaccgacgccgccctccagcccagggccctatatttaaccgaattcgtggtgagtagcggaacgaa

SEQ ID NO: 536 - DNA, 88 bp, Artificial Sequence
  FLNA_98
taccatctcgtgttcgtaccggtaccaagcccagcgtgcccacgtcgtccctatatttaaccgaattcgtggtgagtagcggaacgaa

SEQ ID NO: 537 - DNA, 88 bp, Artificial Sequence
  FLNA_99
taccatctcgtgttcgtaccggtaccccgtcgcccttgtcgtcacattcgatatatttaaccgaattcgtggtgagtagcggaacgaa

SEQ ID NO: 538 - DNA, 88 bp, Artificial Sequence
  FLNA_100
taccatctcgtgttcgtaccggtaccacgtgaacggcatactcgccagccttatatttaaccgaattcgtggtgagtagcggaacgaa

SEQ ID NO: 539 - DNA, 88 bp, Artificial Sequence
  FLNA_101
taccatctcgtgttcgtaccggtaccaccctgtctgggtggaagtcctgggtatatttaaccgaattcgtggtgagtagcggaacgaa

SEQ ID NO: 540 - DNA, 88 bp, Artificial Sequence
  FLNA_102
taccatctcgtgttcgtaccggtaccgctggcttgttgacggccacacctgtatatttaaccgaattcgtggtgagtagcggaacgaa

SEQ ID NO: 541 - DNA, 88 bp, Artificial Sequence
  FLNA_103
taccatctcgtgttcgtaccggtaccgtcctggacttggacccgaagtgggtatatttaaccgaattcgtggtgagtagcggaacgaa

SEQ ID NO: 542 - DNA, 88 bp, Artificial Sequence
  FLNA_104
taccatctcgtgttcgtaccggtaccgtaagtgccattgccgttgtccttgtatatttaaccgaattcgtggtgagtagcggaacgaa

SEQ ID NO: 543 - DNA, 88 bp, Artificial Sequence
  FLNA_105
taccatctcgtgttcgtaccggtaccgtaggtgggctcgtgggccttgagctatatttaaccgaattcgtggtgagtagcggaacgaa

SEQ ID NO: 544 - DNA, 88 bp, Artificial Sequence
  FLNA_106
taccatctcgtgttcgtaccggtacccgaagtcgatgtcagcttcggcgggtatatttaaccgaattcgtggtgagtagcggaacgaa

SEQ ID NO: 545 - DNA, 88 bp, Artificial Sequence
  FLNA_107
taccatctcgtgttcgtaccggtaccaccataatggtgtagctgccagccctatatttaaccgaattcgtggtgagtagcggaacgaa

SEQ ID NO: 546 - DNA, 88 bp, Artificial Sequence
  FLNA_108
taccatctcgtgttcgtaccggtaccagggctccaccttgactcggatgggtatatttaaccgaattcgtggtgagtagcggaacgaa

SEQ ID NO: 547 - DNA, 88 bp, Artificial Sequence
  FLNA_109
taccatctcgtgttcgtaccggtacccaccagtgcgactgaggccagggcctatatttaaccgaattcgtggtgagtagcggaacgaa

SEQ ID NO: 548 - DNA, 88 bp, Artificial Sequence
  FLNA_110
taccatctcgtgttcgtaccggtaccatgtccacatctcgcactgcatccctatatttaaccgaattcgtggtgagtagcggaacgaa

SEQ ID NO: 549 - DNA, 88 bp, Artificial Sequence
  FLNA_111
taccatctcgtgttcgtaccggtaccccctgctggacaggcgtgtacttgatatatttaaccgaattcgtggtgagtagcggaacgaa

SEQ ID NO: 550 - DNA, 88 bp, Artificial Sequence
  FLNA_112
taccatctcgtgttcgtaccggtaccaaagggctcttagggatgggatccctatatttaaccgaattcgtggtgagtagcggaacgaa

SEQ ID NO: 551 - DNA, 88 bp, Artificial Sequence
  FLNA_113
taccatctcgtgttcgtaccggtaccaggccagacaccttgatcttgctgatatatttaaccgaattcgtggtgagtagcggaacgaa

SEQ ID NO: 552 - DNA, 88 bp, Artificial Sequence
  FLNA_114
taccatctcgtgttcgtaccggtaccccctttgatttgactgtgaactccttatatttaaccgaattcgtggtgagtagcggaacgaa

SEQ ID NO: 553 - DNA, 88 bp, Artificial Sequence
  FLNA_115
taccatctcgtgttcgtaccggtaccacctcatagggcccttcctcacgggtatatttaaccgaattcgtggtgagtagcggaacgaa

SEQ ID NO: 554 - DNA, 88 bp, Artificial Sequence
  FLNA_116
taccatctcgtgttcgtaccggtacctggtgtcgatggtgaagcgggcgggtatatttaaccgaattcgtggtgagtagcggaacgaa

SEQ ID NO: 555 - DNA, 88 bp, Artificial Sequence
  FLNA_117
taccatctcgtgttcgtaccggtaccgggaaccacgtgggccttgaatgggtatatttaaccgaattcgtggtgagtagcggaacgaa

SEQ ID NO: 556 - DNA, 88 bp, Artificial Sequence
  FLNA_118
taccatctcgtgttcgtaccggtacctccacttggaattggcccacctccctatatttaaccgaattcgtggtgagtagcggaacgaa

SEQ ID NO: 557 - DNA, 88 bp, Artificial Sequence
  FLNA_119
taccatctcgtgttcgtaccggtaccggatgtacacctcggccggaagccctatatttaaccgaattcgtggtgagtagcggaacgaa

SEQ ID NO: 558 - DNA, 88 bp, Artificial Sequence
  FLNA_120
taccatctcgtgttcgtaccggtacctacttgatggtgacggtgtaggccctatatttaaccgaattcgtggtgagtagcggaacgaa

SEQ ID NO: 559 - DNA, 88 bp, Artificial Sequence
  FLNA_121
taccatctcgtgttcgtaccggtaccaccgcaggttccacctgcagcttgctatatttaaccgaattcgtggtgagtagcggaacgaa

SEQ ID NO: 560 - DNA, 88 bp, Artificial Sequence
  FLNA_122
taccatctcgtgttcgtaccggtaccaagacaccctggccctcaataccagtatatttaaccgaattcgtggtgagtagcggaacgaa

SEQ ID NO: 561 - DNA, 88 bp, Artificial Sequence
  FLNA_123
taccatctcgtgttcgtaccggtaccccggtctgtgtcagagcccgggcgttatatttaaccgaattcgtggtgagtagcggaacgaa

SEQ ID NO: 562 - DNA, 88 bp, Artificial Sequence
  FLNA_124
taccatctcgtgttcgtaccggtaccaggtctccgtcaggttgcctgagggtatatttaaccgaattcgtggtgagtagcggaacgaa

SEQ ID NO: 563 - DNA, 88 bp, Artificial Sequence
  FLNA_125
taccatctcgtgttcgtaccggtacccctcgtaaggcgtgtactccacttttatatttaaccgaattcgtggtgagtagcggaacgaa

SEQ ID NO: 564 - DNA, 88 bp, Artificial Sequence
  FLNA_126
taccatctcgtgttcgtaccggtacctgcaggacatcttggcctcggagggtatatttaaccgaattcgtggtgagtagcggaacgaa

SEQ ID NO: 565 - DNA, 88 bp, Artificial Sequence
  FLNA_127
taccatctcgtgttcgtaccggtacctgccagcctcataagggatgtactctatatttaaccgaattcgtggtgagtagcggaacgaa

SEQ ID NO: 566 - DNA, 88 bp, Artificial Sequence
  FLNA_128
taccatctcgtgttcgtaccggtacctgaaaggactgcctggcacttgatgtatatttaaccgaattcgtggtgagtagcggaacgaa

SEQ ID NO: 567 - DNA, 88 bp, Artificial Sequence
  FLNA_129
taccatctcgtgttcgtaccggtacccgggcccagagcacttgaccttggatatatttaaccgaattcgtggtgagtagcggaacgaa

SEQ ID NO: 568 - DNA, 88 bp, Artificial Sequence
  FLNA_130
taccatctcgtgttcgtaccggtacccccttgcactttgacctgcaatgggtatatttaaccgaattcgtggtgagtagcggaacgaa

SEQ ID NO: 569 - DNA, 88 bp, Artificial Sequence
  FLNA_131
taccatctcgtgttcgtaccggtaccctgggtgccatcagcgttgtctacctatatttaaccgaattcgtggtgagtagcggaacgaa

SEQ ID NO: 570 - DNA, 88 bp, Artificial Sequence
  FLNA_132
taccatctcgtgttcgtaccggtacctaggcagcaccttgaccttgaagggtatatttaaccgaattcgtggtgagtagcggaacgaa

SEQ ID NO: 571 - DNA, 88 bp, Artificial Sequence
  FLNA_133
taccatctcgtgttcgtaccggtaccgcacgccagtggtgttgagcccgggtatatttaaccgaattcgtggtgagtagcggaacgaa

SEQ ID NO: 572 - DNA, 88 bp, Artificial Sequence
  FLNA_134
taccatctcgtgttcgtaccggtaccatctggacagccagcaggccctccctatatttaaccgaattcgtggtgagtagcggaacgaa

SEQ ID NO: 573 - DNA, 88 bp, Artificial Sequence
  FLNA_135
taccatctcgtgttcgtaccggtaccccgtcatggttgtcttggatgtgtgtatatttaaccgaattcgtggtgagtagcggaacgaa

SEQ ID NO: 574 - DNA, 88 bp, Artificial Sequence
  FLNA_136
taccatctcgtgttcgtaccggtacctccacagtgatcaccgtctcctccctatatttaaccgaattcgtggtgagtagcggaacgaa

SEQ ID NO: 575 - DNA, 88 bp, Artificial Sequence
  FLNA_137
taccatctcgtgttcgtaccggtaccccatcaggcgtgcacacggtgcacgtatatttaaccgaattcgtggtgagtagcggaacgaa

SEQ ID NO: 576 - DNA, 88 bp, Artificial Sequence
  FLNA_138
taccatctcgtgttcgtaccggtacccagatgacgtatttgcccggctgggtatatttaaccgaattcgtggtgagtagcggaacgaa

SEQ ID NO: 577 - DNA, 88 bp, Artificial Sequence
  FLNA_139
taccatctcgtgttcgtaccggtaccttgcctgagggcatccgaacctccctatatttaaccgaattcgtggtgagtagcggaacgaa

SEQ ID NO: 578 - DNA, 88 bp, Artificial Sequence
  FLNA_140
taccatctcgtgttcgtaccggtacctaccgcacggtcacggtgccgtctttatatttaaccgaattcgtggtgagtagcggaacgaa

SEQ ID NO: 579 - DNA, 88 bp, Artificial Sequence
  FLNA_141
taccatctcgtgttcgtaccggtacccgtaatccacatagaactgcaagggtatatttaaccgaattcgtggtgagtagcggaacgaa

SEQ ID NO: 580 - DNA, 88 bp, Artificial Sequence
  FLNA_142
taccatctcgtgttcgtaccggtacctggcaggcttgttcactactccatgtatatttaaccgaattcgtggtgagtagcggaacgaa

SEQ ID NO: 581 - DNA, 88 bp, Artificial Sequence
  FLNA_143
taccatctcgtgttcgtaccggtaccgccctcaatggccagagacaggccctatatttaaccgaattcgtggtgagtagcggaacgaa

SEQ ID NO: 582 - DNA, 88 bp, Artificial Sequence
  FLNA_144
taccatctcgtgttcgtaccggtacctacttgactagaatgctgtagtccctatatttaaccgaattcgtggtgagtagcggaacgaa

SEQ ID NO: 583 - DNA, 88 bp, Artificial Sequence
  FLNA_145
taccatctcgtgttcgtaccggtaccgatccgtctctgagatgttgatgggtatatttaaccgaattcgtggtgagtagcggaacgaa

SEQ ID NO: 584 - DNA, 88 bp, Artificial Sequence
  FLNA_146
taccatctcgtgttcgtaccggtaccaacagggctcctcccggcccgagggtatatttaaccgaattcgtggtgagtagcggaacgaa

SEQ ID NO: 585 - DNA, 88 bp, Artificial Sequence
  FLNA_147
taccatctcgtgttcgtaccggtaccgagacccgaacacgactggcatccctatatttaaccgaattcgtggtgagtagcggaacgaa

SEQ ID NO: 586 - DNA, 88 bp, Artificial Sequence
  FLNA_148
taccatctcgtgttcgtaccggtacctcaatgataaactctgcaggctcaatatatttaaccgaattcgtggtgagtagcggaacgaa

SEQ ID NO: 587 - DNA, 88 bp, Artificial Sequence
  FLNA_149
taccatctcgtgttcgtaccggtacctgatgtagttgcctggctctgtgggtatatttaaccgaattcgtggtgagtagcggaacgaa

SEQ ID NO: 588 - DNA, 88 bp, Artificial Sequence
  FLNA_150
taccatctcgtgttcgtaccggtacccgcctgtcaccttcacagagaagggtatatttaaccgaattcgtggtgagtagcggaacgaa

SEQ ID NO: 589 - DNA, 54 bp, Artificial Sequence
  FLNA bridge
attgtaaagcgtgagaaaggtaatgggcggttaaatattgtaaagcgtgagaaa

SEQ ID NO: 590 - DNA, 18 bp, Artificial Sequence
  Cy5 Readout
tttctcacgctttacaat

SEQ ID NO: 591 - DNA, 44 bp, Artificial Sequence
  MUC5AC transcript polymorphic repeat region
ctggaactactcccagccctgttcccaccaccagcacaacctct

SEQ ID NO: 592 - DNA, 9 bp, Artificial Sequence
  First probe binding arm
atttaaccg

SEQ ID NO: 593 - DNA, 9 bp, Artificial Sequence
  Second probe binding arm
cccattacc

SEQ ID NO: 594 - DNA, 18 bp, Artificial Sequence
  Bridge probe
ggtaatgggcggttaaat

SEQ ID NO: 595 - DNA, 18 bp, Artificial Sequence
  Readout sequence
attgtaaagcgtgagaaa